(19) United States
(12) Patent Application Publication
(10) Pub. No.: US 2024/0310389 A1
(43) Pub. Date: Sep. 19, 2024

(54) METHODS AND COMPOSITIONS FOR SCREENING AND TREATING ALZHEIMER'S DISEASE

(71) Applicant: Population Bio, Inc., New York, NY (US)

(72) Inventors: Eli HATCHWELL, Winchester (GB); Peggy S. EIS, Fitchburg, WI (US); Edward B. SMITH, III, New York, NY (US); Shapour JALILZADEH, Headington, Oxford (GB)

(21) Appl. No.: 18/411,834

(22) Filed: Jan. 12, 2024

Related U.S. Application Data
(60) Provisional application No. 63/438,710, filed on Jan. 12, 2023.

(51) Int. Cl. G01N 33/68

(52) U.S. Cl. CPC G01N 33/6896; G01N 2800/2821

(57) ABSTRACT

This document provides methods and materials related to screening for and treating Alzheimer's disease (AD), including late-onset Alzheimer's disease (LOAD).

### CROSS-REFERENCE TO RELATED APPLICATIONS

This application claims priority to U.S. Provisional Patent Application No. 63/438,710, filed on Jan. 12, 2023, the entire content of which is entirely incorporated herein by reference.

### REFERENCE TO A SEQUENCE LISTING

The instant application contains a Sequence Listing which has been submitted in XML format via Patent Center and is hereby incorporated by reference in its entirety. Said XML copy, created on Jan. 2, 2024, is named 33655_715_201_SL.xml and is 11,482,807 bytes in size. The aforementioned file was created on Jan. 2, 2024, and is hereby incorporated by reference in its entirety.

### BACKGROUND

Alzheimer's Disease (AD) is a chronic, incurable, neurodegenerative disease that affects an estimated 5.4 million people in the United States. AD is a highly heritable complex disorder, for which the majority of genetic causes have yet to be identified. While a number of genes have been identified in early onset families (e.g., APP, PSEN1, PSEN2), early onset Alzheimer's Disease (EOAD), diagnosed at <65 years of age, affects only 5-10% of cases. Late Onset Alzheimer's Disease (LOAD) shares the same clinical and pathological features as AD but is diagnosed in those >65 years of age. The most important genetic risk factor for the development of LOAD and sporadic EOAD that has been identified to date is presence of the E4 variant of the APOE gene.

The primary function of the urea cycle is to aid the body in metabolizing ammonia (a by-product of amino acid breakdown) into the final product urea. Ammonia is a highly toxic compound, with known effects on the central nervous system. There is no mechanism for its direct excretion from the body. Urea is a relatively benign molecule which is excreted from the kidneys. Rare disorders, such as autosomal recessive (AR) and chromosome X-linked recessive (XLR) disease models, have been reported that affect one of the urea cycle genes (including CPS1) and result in variable phenotypes in neonates, infants and adults. Many of the features are a direct result of hyperammonemia, due to the failure of the urea cycle to effectively remove ammonia (Walker, 2014). In the most severe forms, neonates fail to thrive, vomit and suffer seizures. In some cases, the urea cycle disorder is lethal.

While there is no clear-cut phenotype or disease associated with dominant mutations in urea cycle genes, hyperammonemia has been posited as an etiological factor in Alzheimer's disease (Branconnier et al., 1986; Seiler, 2002; Adlimoghaddam et al., 2016; Jin et al., 2018). However, this finding has not been tied to heterozygous mutations in urea cycle disorder genes, or other genes involved in ammonia metabolism.

The ability to accurately predict who is at risk of developing LOAD will be of enormous benefit in the context of drug treatment with compounds that are highly effective in their disease context but carry a risk of a devastating disorder. There is a need to identify genetic variants, such as copy number variants (CNVs) and single nucleotide variants (SNVs), associated with developing LOAD and the genes affected by them. For example, a proportion of individuals with AD may have hyperammonemia as a causal factor and some of these cases may be due to mutations in urea cycle disorders genes. Any therapeutic approaches developed for the treatment of classical (AR) urea cycle disorders would have relevance for the treatment of AD in these cases. For example, the drugs glycerol phenylbutyrate (RAVICTI®) or taste-masked sodium phenylbutyrate (OLPRUVA™) for the treatment of children and adults with a urea cycle disorder might prove to be valuable in AD, even if only in a specific, genetically-defined subset.

### SUMMARY

Provided herein are methods and materials related to screening for and treating Alzheimer's disease (AD), including late-onset Alzehimer's disease (LOAD) in human subjects.

Provided herein is a method of treating or preventing Alzheimer's Disease (AD) in a subject in need thereof, comprising: administering a therapeutically effective amount of a urea cycle agent to the subject, wherein the subject is identified as having AD or a risk of developing AD.

Provided herein is a method of treating Alzheimer's Disease (AD) in a subject in need thereof, comprising: administering a therapeutically effective amount of a urea cycle agent to the subject, wherein the subject has an AD or a risk of developing AD, wherein the subject's risk of developing AD is due to the presence of one or more genetic variations that occur at a frequency of 10% or less in a population of human subjects with AD.

In some embodiments, the urea cycle agent is a therapeutic agent listed in Table 13 or Table 14, or any combination thereof.

In some embodiments, the AD is Late-Onset Alzheimer's Disease (LOAD).

In some embodiments, the subject is a mild cognitively impaird (MCI) subject.

In some embodiments, the subject is identified as having AD or a risk of developing AD.

In some embodiments, the subject is identified as having AD or a risk of developing AD by a genetic test.

In some embodiments, the subject has one or more genetic variations associated with the AD or a risk of developing the AD.

In some embodiments, the genetic test comprises detecting one or more genetic variations associated with the AD or a risk of developing the AD in a polynucleic acid sample from the subject.

In some embodiments, the one or more genetic variations associated with the AD or a risk of developing the AD is a genetic variation that causes a mutation in a protein encoded by a urea cycle disorder (UCD) gene, a hyperammonemia (HA) gene, a gene in Table 8 or a gene in Table 9.

In some embodiments, the mutation is a deleterious mutation.

In some embodiments, the mutation is a loss-of-function (LOF) mutation.

In some embodiments, the protein encoded by the UCD or HA gene is not expressed or expressed at a lower level compared to the protein encoded by the UCD or HA gene without the mutation.

In some embodiments, the one or more genetic variations associated with the AD or a risk of developing the AD is a genetic variation in a regulatory element of a UCD or HA gene.

In some embodiments, a protein encoded by the UCD or HA gene is not expressed or expressed at a lower level compared to the protein encoded by the UCD or HA gene without the genetic variation in the regulatory element of the UCD or HA gene.

In some embodiments, the one or more genetic variations associated with the AD or a risk of developing the AD is a genetic variation that causes a mutation in a protein encoded by a UCD gene.

In some embodiments, the one or more genetic variations associated with the AD or a risk of developing the AD is a genetic variation that causes a mutation in a protein encoded by a HA gene.

In some embodiments, the one or more genetic variations associated with the AD or a risk of developing the AD comprise a point mutation, polymorphism, single nucleotide polymorphism (SNP), single nucleotide variation (SNV), translocation, insertion, deletion, amplification, inversion, interstitial deletion, copy number variation (CNV), structural variation (SV), loss of heterozygosity, or any combination thereof.

In some embodiments, the one or more genetic variations disrupt or modulate a gene, expression of a gene and/or a regulatory element of a gene selected from the group consisting of ARG1, ASL, ASS1, CPS1, NAGS, OTC, SLC25A13, and SLC25A15.

In some embodiments, the one or more genetic variations disrupt or modulate a gene, expression of a gene and/or a regulatory element of a gene selected from the group consisting of ACADM, ACADVL, ALDH18A1, AMT, ATP5F1D, ATPAF2, CA5A, CPT1A, CPT2, CYC1, DLAT, ETFA, ETFB, ETFDH, FBXL4, FH, GLUD1, GLUL, HADHA, HADHB, HLCS, HMGCL, IVD, LMBRD1, MCCC1, MCCC2, MCEE, MLYCD, MMAA, MMAB, MMACHC, MMADHC, MMUT, OAT, PC, PCCA, PCCB, PDHA1, SLC22A5, SLC25A20, SLC25A42, SLC7A7, TAFAZZIN, TANGO2, TMEM70, TUFM, UQCRC2 and YARS2.

In some embodiments, the one or more genetic variations disrupt or modulate a gene, expression of a gene and/or a regulatory element of a gene selected from the group consisting of GLUL, CPS1, LMBRD1, ASL, SLC25A13, TMEM70, ASS1, MMAB, PCCA, SLC7A7, ACADVL, ATPAF2, ATP5FID, OTC and TAFAZZIN.

In some embodiments, the one or more genetic variations disrupt or modulate a gene, expression of a gene and/or a regulatory element of a gene selected from the group consisting of GLUL, CPS1, LMBRD1, ASL, SLC25A13, TMEM70, ASS1, MMAB, PCCA, SLC7A7, ACADVL, ATPAF2, ATP5FID, OTC, TAFAZZIN, UBE2W, ASGR2, MIR33B, RAI1, SREBF1, ABCA7, CNN2 and TSPAN7.

In some embodiments, the one or more genetic variations is in an exon or intron of a gene and disrupts or modulates expression of a gene and/or a regulatory element of a gene that encodes a transcript with at least 80% sequence identity to any one of SEQ ID NOs: 26-96.

In some embodiments, the one or more genetic variations is in an intergenic genomic region and disrupts or modulates expression of a gene and/or a regulatory element of a gene that encodes a transcript with at least 80% sequence identity to any one of SEQ ID NOs: 55 and 97-111.

In some embodiments, the one or more genetic variations is in an intergenic genomic region.

In some embodiments, the one or more genetic variations disrupt or modulate expression and/or a regulatory element of a gene selected from the group consisting of GLUL, SLC25A13, SLC7A7, OTC and TAFAZZIN.

In some embodiments, the one or more genetic variations is in an exon of a gene.

In some embodiments, the one or more genetic variations is in an exon of a gene selected from the group consisting of LMBRD1, UBE2W, ASS1, MIR33B, RAI1, SREBF1, ABCA7, CNN2 and TSPAN7.

In some embodiments, the one or more genetic variations disrupt or modulate expression and/or a regulatory element of a gene selected from the group consisting of LMBRD1, TMEM70, ASS1, ATPAF2, ATP5FID and OTC.

In some embodiments, the one or more genetic variations is in an intron of a gene.

In some embodiments, the one or more genetic variations is in an intron of a gene selected from the group consisting of CPS1, LMBRD1, ASL, MMAB, PCCA, ASGR2, OTC and TSPAN7.

In some embodiments, the one or more genetic variations disrupt or modulate expression and/or a regulatory element of a gene selected from the group consisting of CPS1, LMBRD1, ASL, MMAB, PCCA, ACADVL and OTC.

In some embodiments, wherein the one or more genetic variations comprise a CNV loss.

In some embodiments, the one or more genetic variations comprise a CNV loss of a sequence with at least 80% sequence identity to any one of SEQ ID NOs: 1-10, 12-14 and 16-20.

In some embodiments, the genetic variation is a CNV gain.

In some embodiments, the genetic variation is a CNV gain of a sequence with at least 80% sequence identity to any one of SEQ ID NOs: 11, 15 and 21-25.

In some embodiments, the urea cycle disorder (UCD) is selected from the group consisting of Argininemia, Argininosuccinic aciduria, Citrullinemia, Carbamoylphosphate synthetase I deficiency, N-acetylglutamate synthase deficiency, Ornithine transcarbamylase deficiency, Citrullinemia, type II, neonatal-onset; Citrullinemia, adult-onset type II and Hyperomithinemia-hyperammonemia-homocitrullinemia syndrome.

In some embodiments, the one or more genetic variations disrupt or modulate a gene, expression of a gene and/or a regulatory element of a gene selected from the group consisting of ARG 1, ASL, ASS1, CPS1, NAGS, OTC, SLC25A13 and SLC25A15.

In some embodiments, the hyperammonemia (HA) is selected from the group consisting of Acyl-CoA dehydrogenase, medium chain, deficiency of; VLCAD deficiency; Cutis laxa, autosomal recessive, type IIIA; Glycine encephalopathy; Mitochondrial complex V (ATP synthase) deficiency; Mitochondrial complex V (ATP synthase) deficiency, nuclear type 1; Carbonic anydrase VA deficiency; CPT deficiency, hepatic, type IA; CPT II deficiency, infantile; Mitochondrial complex III deficiency, nuclear type 6; Pyruvate dehydrogenase E2 deficiency; Glutaric acidemia IIA; Glutaric acidemia IIB; Glutaric acidemia IIC; Mitochondrial DNA depletion syndrome 13 (encephalomyopathic type); Fumarase deficiency; Hyperinsulinism-hyperammonemia syndrome; Glutamine deficiency, congenital; Mitochondrial trifunctional protein deficiency; Trifunctional protein deficiency; Holocarboxylase synthetase deficiency; HMG-CoA lyase deficiency; Isovaleric acidemia; Methylmalonic aciduria and homocystinuria, cblF type; 3-Methylcrotonyl-CoA carboxylase 1 deficiency; 3-Methylcrotonyl-CoA carboxylase 2 deficiency; Methylmalonyl-CoA epimerase deficiency; Malonyl-CoA decarboxylase deficiency; Methylmalonic aciduria, vitamin B 12-responsive; Methylmalonic aciduria, vitamin B 12-responsive, cblB type; Methylmalonic aciduria and homocystinuria, cblC type; Homocystinuria, cblD type, variant 1; Methylmalonic aciduria and homocystinuria, cblD type; Methylmalonic aciduria, cblD type, variant 2; Methylmalonic aciduria, mut(0) type; Gyrate atrophy of choroid and retina with or without omithinemia; Pyruvate carboxylase deficiency; Propionicacidemia; Propionicacidemia; Pyruvate dehydrogenase E1-alpha deficiency; Camitine deficiency, systemic primary; Carnitine-acylcarnitine translocase deficiency; Metabolic crises, recurrent, with variable encephalomyopathic features and neurologic regression; Lysinuric protein intolerance; Barth syndrome; Metabolic encephalomyopathic crises, recurrent, with rhabdomyolysis, cardiac arrhythmias, and neurodegeneration; Mitochondrial complex V (ATP synthase) deficiency, nuclear type 2; Combined oxidative phosphorylation deficiency 4; Mitochondrial complex III deficiency, nuclear type 5; Myopathy, lactic acidosis, and sideroblastic anemia 2.

In some embodiments, the one or more genetic variations disrupt or modulate a gene, expression of a gene and/or a regulatory element of a gene selected from the group consisting of ACADM, ACADVL, ALDH18A1, AMT, ATP5FID, ATPAF2, CASA, CPT1A, CPT2, CYC1, DLAT, ETFA, ETFB, ETFDH, FBXL4, FH, GLUD1, GLUL, HADHA, HADHB, HLCS, HMGCL, IVD, LMBRD1, MCCC1, MCCC2, MCEE, MLYCD, MMAA, MMAB, MMACHC, MMADHC, MMUT, OAT, PC, PCCA, PCCB, PDHA1, SLC22A5, SLC25A20, SLC25A42, SLC7A7, TAFAZZIN, TANGO2, TMEM70, TUFM, UQCRC2 and YARS2.

In some embodiments, the one or more genetic variations comprise a genetic variation according to any one of Tables 1-11 and 13-15.

In some embodiments, the one or more genetic variations comprise an SNV.

In some embodiments, the SNV is in an exon of a gene.

In some embodiments, the SNV is in an exon of a gene selected from the group consisting of MMACHC, HADHA, MMADHC, SLC25A20, AMT, PCCB, MCCC1, MMAA, MMUT, FBXL4, ASL, SLC25A13, PC, DLAT, SLC7A7, TUFM, MLYCD, ACADVL, NAGS and HLCS.

In some embodiments, the one or more genetic variations comprise a sequence according to any one of SEQ ID NOs: 117-119, 125-154 and 159-183.

In some embodiments, the one or more genetic variations comprise an indel.

In some embodiments, the indel is in an intron of a gene.

In some embodiments, the indel is in an intron of a CPS1 gene.

In some embodiments, the one or more genetic variations comprise a sequence according to any one of SEQ ID NOs: 120-124 and 155-157.

In some embodiments, the SNV is in an intron of a CPS1 gene.

In some embodiments, the one or more genetic variations comprise an SNV within a CPS1 GeneHancer enhancer GH02J210503.

In some embodiments, the SNV within a CPS1 GeneHancer enhancer GH02J210503 comprises a sequence according to SEQ ID NO: 125.

In some embodiments, the one or more genetic variations comprise a CNV loss of a sequence with at least 80% sequence identity to SEQ ID NO: 3.

In some embodiments, the urea cycle agent targets a gene selected from the group consisting of ARG1, ASS1, CPS1, NAGS, OTC, SLC25A13 and SLC25A15, or an RNA transcript or protein encoded by the gene.

In some embodiments, the urea cycle agent is a small molecule, fusogen, gene therapy, microbiome metabolic therapy, enzyme replacement therapy or mRNA therapy.

In some embodiments, the urea cycle agent is selected from the group consisting of carglumic acid, glycerol phenylbutyrate, sodium phenylacetate and sodium benzoate, sodium phenylbutyrate, taste-masked sodium phenylbutyrate and sodium benzoate.

In some embodiments, the urea cycle agent is selected from the group consisting of ACER-001, AEB 1102 (pegzilarginase), ARCT-810, BB-OTC, DTX301, KB-195, P-OTC-101, PRX-OTC, SEL-313, SG328 and P-OTC-101.

In some embodiments, the method comprises administration co-administration of a urea cycle agent and an antibiotic of an additional agent.

In some embodiments, the additional agent is an antibiotic or taurine.

In some embodiments, the subject's risk of developing AD is due to the presence of one or more genetic variations that occur at a frequency of 10% or less in a population of human subjects with AD and without a UCD.

In some embodiments, the subject's risk of developing AD is due to the presence of one or more genetic variations that occur at a frequency of 10% or less in a population of human subjects with AD and with a UCD.

In some embodiments, the subject's risk of developing AD is due to the presence of one or more genetic variations that occur at a frequency of 1% or less in a population of human subjects with AD.

In some embodiments, the subject's risk of developing AD is due to the presence of one or more genetic variations that occur at a frequency of 1% or less in a population of human subjects with AD and without a UCD.

In some embodiments, the subject's risk of developing AD is due to the presence of one or more genetic variations that occur at a frequency of 1% or less in a population of human subjects with AD and with a UCD.

In some embodiments, the one or more genetic variations have an odds ratio (OR) of 3 or more, and wherein the OR is:

[D_{D}/D_{N}]/[N_{D}/N_{N}],
wherein:
D_{D} is the number of subjects in a diseased cohort of subjects with the one or more genetic variations;
D_{N} the number of subjects in the diseased cohort without the one or more genetic variations;
N_{D} is the number of subjects in a non-diseased cohort of subjects with the one or more genetic variations; and
N_{N} is the number of subjects in the non-diseased cohort without the one or more genetic variations.

In some embodiments, the diseased cohort or non-diseased cohort comprises at least 100 human subjects.

In some embodiments, the at least 100 human subjects comprises at least 100 human subjects with AD.

In some embodiments, the at least 100 human subjects comprises at least 10 human subjects without a UCD.

In some embodiments, the subject's risk of developing AD is due to the presence of one or more genetic variations that has an odds ratio (OR) of at least 3, 4, 5, 6, 7, 8, 9, 10, 15, 20, 25, 30, 35, 40, 45 or 50.

In some embodiments, the one or more genetic variations comprise at least 2, 3, 4, 5, 6, 7, 8, 9, 10, 11, 12, 13, 14, 15, 20, 25, 30, 35, 40, 45, 50, 75, 100 genetic variations.

In an aspect, the present disclosure provides a method of treating or preventing AD comprising:
(a) testing a subject with AD for the presence of one or more genetic variations that disrupt or modulate a corresponding gene according to any one of Tables 1-11 and 13-15,
(b) determining that the subject has one or more genetic variations that disrupt or modulate a corresponding gene according to any one of Tables 1-11 and 13-15, and
(c) administering a urea cycle agent to the subject that was determined to have the one or more genetic variations that disrupt or modulate a corresponding gene according to any one of Tables 1-11 and 13-15.

In some embodiments, the genetic test or the testing comprises microarray analysis, PCR, sequencing, nucleic acid hybridization, or any combination thereof.

In some embodiments, the genetic test or the testing comprises microarray analysis selected from the group consisting of a Comparative Genomic Hybridization (CGH) array analysis and an SNP array analysis.

In some embodiments, the genetic test or the testing comprises sequencing, wherein the sequencing is selected from the group consisting of Massively Parallel Signature Sequencing (MPSS), polony sequencing, 454 pyrosequencing, Illumina sequencing, Illumina (Solexa) sequencing using 10× Genomics library preparation, SOLID sequencing, ion semiconductor sequencing, DNA nanoball sequencing, heliscope single molecule sequencing, single molecule real time (SMRT) sequencing, RNAP sequencing, Nanopore DNA sequencing, sequencing by hybridization, and microfluidic Sanger sequencing.

In some embodiments, the genetic test or the testing comprises analyzing a whole genome of the subject.

In some embodiments, the genetic test or the testing comprises analyzing a whole exome of the subject.

In some embodiments, the genetic test or the testing comprises analyzing nucleic acid information that has already been obtained for a whole genome or a whole exome of the subject.

In some embodiments, the nucleic acid information is obtained from an in sit/co analysis.

In some embodiments, the subject is a human subject.

In some embodiments, the polynucleic acid sample comprises a polynucleic acid from blood, saliva, urine, serum, tears, skin, tissue, or hair of the subject.

In an aspect, the present disclosure provides a method of identifying a subject as having a risk of developing AD, comprising:
(a) analyzing a polynucleic acid sample from the subject for one or more genetic variations that disrupt or modulate a corresponding gene according to any one of Tables 1-14, wherein a genetic variation of the one or more genetic variations that disrupt or modulate a corresponding gene according to any one of Tables 1-11 and 13-15 is present in the polynucleic acid sample;
(b) identifying the subject as having a risk of developing AD.

In an aspect, the present disclosure provides a method of identifying a subject as having a reduced risk of developing AD, comprising:
(a) analyzing a polynucleic acid sample from the subject for one or more genetic variations that disrupt or modulate a corresponding gene according to Table 12, wherein a genetic variation of the one or more genetic variations that disrupt or modulate a corresponding gene according to Table 12 is present in the polynucleic acid sample;
(b) identifying the subject as having a reduced risk of developing AD compared to the risk of developing AD in a subject without the one or more genetic variations that disrupt or modulate a corresponding gene according to Table 12.

In some embodiments, the one or more genetic variations comprise a sequence according to any one of SEQ ID NOs: 184 and 185.

In an aspect, the present disclosure provides a kit, comprising reagents for assaying a polynucleic acid sample from a subject in need thereof for the presence of one or more genetic variations that disrupt or modulate a corresponding gene according to any one of Tables 1-11 and 13-15.

In some embodiments, the reagents comprise at least one contiguous oligonucleotide that hybridizes to a fragment of the polynucleic acid sample.

In some embodiments, the reagents comprise at least one pair of oligonucleotides that hybridize to opposite strands of a fragment of the polynucleic acid sample.

In some embodiments, the kit further comprises one or more urea cycle agents.

In some embodiments, the kit further comprises a set of instructions for administration of the one or more urea cycle agents.

In an aspect, the present disclosure provides a method of screening for an Alzheimer's disease (AD) biomarker comprising:
(a) obtaining biological samples from subjects with AD;
(b) screening the biological samples to obtain nucleic acid information;
(c) detecting one or more genetic variations that disrupt or modulate a corresponding gene according to any one of Tables 1-11 and 13-15 in a polynucleic acid sample from a subject suspected of having AD; and
(d) using that detection as a biomarker for predicting a response of the subject to a therapy to be beneficial, wherein the therapy is one or more urea cycle agents.

In some embodiments, the detecting one or more genetic variations further comprises using polymerase chain reaction (PCR), sequencing, nucleic acid hybridization, microarray analysis, northern blot, or any combination thereof.

In some embodiments, the microarray analysis is selected from the group consisting of a comparative genomic hybridization (CGH) analysis and a SNP array analysis.

In some embodiments, the sequencing comprises a sequencing method selected from the group consisting of massively parallel signature sequencing, polony sequencing, high throughput pyrose-quencing, bead array sequencing, ion semiconductor sequencing, DNA nanoball sequencing, single molecule sequencing, single molecule real time sequencing, RNAP sequencing, nanopore DNA se quencing, sequencing by hybridization, and microfluidic Sanger sequencing.

In some embodiments, the one or more urea cycle agents is a therapeutic agent listed in Table 13 or Table 14, or any combination thereof.

In some embodiments, the using further comprises comparing the nucleic acid information to a panel of nucleic acid biomarkers.

In some embodiments, the panel of nucleic acid biomarkers comprises at least about 100 nucleic acid biomarkers.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In the event of a conflict between a term herein and a term incorporated by reference, the term herein controls.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1 represents an example of an aCGH-detected 3-probe intronic deletion that impacts the UCD gene CPS1. The deletion was observed in 3/100 LOAD cases and 1/1,000 Normal (NVE) subjects. All 4 deletions appear to be identical, with a genomic span of 6,430 bp. Genome coordinates of the deletion are: hg18, chr2:211070190-211076620; hg19, chr2:211361945-211368375. Log 2 ratios for the y-axes (4 data tracks) report deletions as negatively shifted probes. Genome coordinates for the x-axis are based on the NCBI36/hg18 freeze.

FIG. 2 represents an example of the extent of the CPS1 intronic deletion compared to different public data sources for CNVs/SVs in the UCSC genome browser. The deletion reported by Population Bio (CPS1_del_PBio) is defined by the location of 3 aCGH probes (see FIG. 1). The deletion reported in other data sources (IKG Ph3 labeled as CPS1 del_IKG_Ph3, DGV_Gold labeled as gssvL69036, and gnomAD labeled as DEL_2_26674) is based on analysis of WGS data, which allows (in this instance) for a more accurate delineation of the endpoints. Five aCGH probes are shown (central 3 mapping to the deletion plus 1 flanking on each side). The endpoints of the WGS mapped deletion do not extend to the flanking probes, which confirms the aCGH data. Genome coordinates are based on the NCBI36/hg18 freeze.

FIG. 3 represents an example of UCSC genome browser regulatory site annotations that are encompassed by, or overlap with, the CPS1 deletion detected in 3 LOAD cases. Displayed are the 3 Agilent probes (identified by dashed lines and are the same 3 aCGH probes shown in FIG. 1), CNV/SV public data (DGV GS, IKG Ph3, and gnomAD) that is also shown in FIG. 2, and regulatory site annotation from two public data sources: ENCODE transcription factor binding sites; GeneHancer mapping of enhancer and promoter sites. This demonstrates that the deletion overlaps an enhancer regulatory element (GH02J210503) with a functional role for expression of the CPS1 gene. Genome coordinates are based on the GRCh37/hg 19 freeze.

FIG. 4 represents an example of UCSC genome browser regulatory site annotations for the full extent of the CPS1 gene (zoomed out view of FIG. 3, the intronic deletion is demarcated by the pair of vertical dashed lines). Besides GeneHancer enhancer GH02J210503, the location of 5 other promoter/enhancer elements are shown. Also displayed are the interactions between GeneHancer regulatory elements and genes (Double Elite), which shows that enhancer GH02J210503 (located in an intron of CPS1) interacts with CPS1 promoter GH02J210475.

FIG. 5 represents an example of a PCR assay designed to detect the CPS1 intronic deletion (see FIG. 1 and FIG. 2). Shown is a 1% agarose gel with a PCR product band (350 bp in size) obtained for a positive (Pos.) control genomic DNA sample (i.e., from a subject known to harbor the CPS1 deletion) using PCR primers CPS1_delF and CPS1_delR (see Table 6). No PCR product is observed for two different negative (Neg.) control genomic DNA samples, thereby demonstrating the specificity of the DNA assay to detect the CPS1 deletion.

FIG. 6 represents an example of a PCR assay used to validate the aCGH-detected CPS1 deletion found in 3 LOAD cases (see FIG. 1). Each of the 3 positive LOAD samples (Expt IDs 2693, 2696, and 2719) was run in duplicate (lanes 1-6 after the DNA ladder) on a 2% agarose gel. Two negative controls (samples known not to harbor the deletion on the basis of aCGH, lanes 7 and 8) are also shown, plus a non-template control (NTC) wherein the assay was run with no input DNA. This demonstrates that all 3 LOAD samples yield a positive product at the expected size of 350 bp (see FIG. 5).

FIG. 7 shows examples of aCGH-detected CNVs nearby UCD gene OTC (gene locations are shown at the top) in 5 LOAD cases (all female). The CNVs (a deletion in track 1, a duplication in track 2, and an identical duplication in tracks 3-5) are also nearby or directly impact the TSPAN7 gene. Log 2 ratios for the y-axes (5 tracks) report deletions as negatively shifted probes and duplications as positively shifted probes. Genome coordinates for the x-axis are based on the NCBI36/hg18 freeze. See Table 1 for the NCBI36/hg 18 genome coordinates of the CNVs.

FIG. 8 represents examples of aCGH-detected CNVs (deletion and duplication) compared to public data sources for CNVs/SVs (DGV GS and gnomAD SV) in the UCSC genome browser. Two aCGH-detected CNVs (see FIG. 7) were confirmed in the gnomAD SV database: DEL_X_185726 corresponds to the deletion found in 1 LOAD case (Expt ID 2732); DUP_X_52986 corresponds to the duplication found in 3 LOAD cases (Expt IDs 2724, 2726, and 2744). The duplication found in LOAD case Expt ID 2687 is not found in public CNV/SV data sources. Genome coordinates are based on the GRCh37/hg 19 freeze. See Table 1 for the NCBI36/hg18 genome coordinates of the CNVs.

FIG. 9 represents an example of an aCGH-detected 1-probe (Agilent probe A_16_P21444361) intronic deletion that impacts the UCD gene OTC. The deletion was observed in 1/100 LOAD cases and 0/1,000 Normal (NVE) subjects (the dotted vertical line demarcates the 1,000 Normal subjects from the 100 LOAD cases). Genome coordinates for the aCGH probe: hg18, chrX:38109666-38109725; hg19, chrX:38224722-38224781. The y-axis Log 2 ratio reports deletions as negatively shifted probes. The plot demonstrates that (i) the probe is well-behaved (very little noise across 1,100 total aCGH experiments) and (ii) that there exists an individual with a very low log 2 ratio (consistent with a homozygous deletion). This individual is male and thus this result demonstrates that this individual is deleted at this locus (the log 2 ratio is lower than expected for a deletion affecting only one allele, because males only have one Chr X).

FIG. 10 represents an example of a plot of aCGH data for an individual with the deletion shown in FIG. 9. The probes (dots) in this plot are all from the same LOAD case (Expt ID 2764) and span the OTC gene (located at chrX:38,096,680-38,165,647), demonstrating the deleted probe in the context of the full extent of the gene. Genome coordinates for the x-axis are based on the NCBI36/hg 18 freeze.

FIG. 11 represents an example of an aCGH-detected 1-probe (Agilent probe A_16 P15364898) intergenic deletion ˜226Kb downstream of HA gene GLUL. The deletion was observed in 2/100 LOAD cases and 1/1,000 Normal (NVE) subjects (the dotted vertical line demarcates the 1,000 Normal subjects from the 100 LOAD cases). Genome coordinates for the aCGH probe: hg18, chr1:180387776-180387835; hg19, chr1:182121153-182121212. The y-axis Log 2 ratio reports deletions as negatively shifted probes.

FIG. 12 represents an example of UCSC genome browser regulatory site annotations that are immediately adjacent to the intergenic deletion (˜226Kb downstream of the GLUL gene) detected in 2 LOAD cases (see FIG. 11). Displayed are the deletion-reporting aCGH Agilent probe (A_16 P15364898, see FIG. 11), CNV/SV public data (DGV GS and gnomAD), and regulatory site annotation from two public data sources: GeneHancer mapping of enhancer and promoter sites; ENCODE transcription factor binding sites. The 1-probe deletion maps to gnomAD DEL 19677, which is immediately adjacent to an enhancer regulatory element (GH01J182142) with a functional role for expression of the GLUL gene. There are numerous ENCODE transcription factor binding sites (bottom track) that map to this enhancer element. Genome coordinates are based on the GRCh37/hg 19 freeze.

FIG. 13 represents an example of UCSC genome browser regulatory site annotations for the full extent of the GLUL gene and downstream region encompassing the intergenic deletion (zoomed out view of FIG. 12, the deletion detected by Agilent probe A16P15364898 and mapping to gnomAD DEL_1_9677). Besides GeneHancer enhancer GH01J182142, the location of 10 other promoter/enhancer elements downstream of the GLUL gene are shown. This demonstrates that enhancer and promoter elements do not need to be contained within the gene whose expression they are regulating and that they can be located far away from the gene.

### DETAILED DESCRIPTION OF THE DISCLOSURE

The details of one or more inventive embodiments are set forth in the accompanying drawings, the claims, and in the description herein. Other features, objects, and advantages of inventive embodiments disclosed and contemplated herein will be apparent from the description and drawings, and from the claims.

As used herein, unless otherwise indicated, the article “a” means one or more unless explicitly otherwise provided for.

As used herein, unless otherwise indicated, terms such as “contain,” “containing,” “include,” “including,” and the like mean “comprising.”

As used herein, unless otherwise indicated, the term “or” can be conjunctive or disjunctive. As used herein, unless otherwise indicated, any embodiment can be combined with any other embodiment.

As used herein, unless otherwise indicated, some inventive embodiments herein contemplate numerical ranges. When ranges are present, the ranges include the range endpoints. Additionally, every subrange and value within the range is present as if explicitly written out.

As used herein, unless otherwise indicated, the term “about” in relation to a reference numerical value and its grammatical equivalents include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11.

As used herein, unless otherwise indicated, the term “biological product” refers to a virus, therapeutic serum, toxin, antitoxin, vaccine, blood, blood component or derivative, allergenic product, protein (any alpha amino acid polymer with a specific defined sequence that is greater than 40 amino acids in size), or analogous product, or arsphenamine or derivative of arsphenamine (or any trivalent organic arsenic compound), applicable to the prevention, treatment, or cure of a disease or condition of human beings.

As used herein, unless otherwise indicated, the term “biosimilar product” refers to 1) a biological product having an amino acid sequence that is identical to a reference product; 2) a biological product having a different amino acid sequence (e.g., N- or C-terminal truncations) from a reference product; or 3) a biological product having a different posttranslational modification (e.g., glycosylation or phosphorylation) from a reference product, wherein the biosimilar product and the reference product utilize the same mechanism or mechanisms of action for the prevention, treatment, or cure of a disease or condition.

As used herein, “mechanism of action” refers to an interaction or activity through which a drug product (e.g., a biological product) produces a pharmacological effect.

As used herein, unless otherwise indicated, the term “interchangeable product” refers to a biosimilar product, wherein a response rate of a human subject administered the interchangeable product is from 80% to 120% of the response rate of the human subject administered the reference product.

As used herein, unless otherwise indicated, the term “reference product” refers to 1) a biological product having an amino acid sequence that is identical to a biosimilar product; 2) a biological product having a different amino acid sequence (e.g., N- or C-terminal truncations) from a biosimilar product; or 3) a biological product having a different posttranslational modification (e.g., glycosylation or phosphorylation) from a biosimilar product, wherein the reference product and the biosimilar product utilize the same mechanism or mechanisms of action for the prevention, treatment, or cure of a disease or condition.

### Late-Onset Alzheimer's Disease (LOAD)

Alzheimer's disease (AD) is the most common form of dementia in the elderly, accounting for a majority of cases at autopsy and in clinical series. The number of people affected by AD is increasing and are characterized by deterioration of memory and other cognitive domains, and leading to death 3-9 years after diagnosis. The number of patients with AD, the most common cause of disability in the elderly, rises dramatically. Late Onset Alzheimer's Disease (LOAD) shares the same clinical and pathological features as AD but is diagnosed in those >65 years of age. Therefore, it is important for clinicians to recognize early signs and symptoms of dementia and to note potentially modifiable risk factors and early disease markers.

AD and LOAD may be characterized by extensive atrophy of the brain caused by a series neuropathologic changes, including neuronal loss, formation of amyloid plaques, appearance of neurofibrillary tangles, and synaptic loss. Amyloid plaques and neurofibrillary tangles result from an aberration in deposition of the Aβ peptide and the hyperphosphorylated tau protein, respectively, and these depositions lead to neuronal loss and neurotoxicity in the brain affected by AD. These changes in the brain may not be found throughout the brain and preferentially affect specific brain areas in a manner that is essentially consistent from patient to patient. Data obtained by electron microscopy and immunocytochemical and biochemical analysis on synaptic marker proteins in AD biopsies and autopsies indicate that synaptic loss in the hippocampus and neocortex may be an early event and the major structural correlate of cognitive dysfunction. From all cortical areas analyzed, the hippocampus may be the most severely affected by the loss of synaptic proteins, while the occipital cortex is affected least. In addition, synaptic loss may be currently the best neurobiological correlate of cognitive deficits in AD. In some cases, living neurons may lose their synapses in AD. Furthermore, synaptic function may be impaired in living neurons, as demonstrated by decrements in transcripts related to synaptic vesicle trafficking.

In some cases, genetic factors leading to AD or LOAD may be part of the urea cycle.

Any therapeutic agent in Table 13 and Table 14 may be used for treatment of AD or at least for ameliorating one or more symptoms of AD. Several drugs have been reported in the context of UCD or HA and may be used for treatment of LOAD or at least for ameliorating one or more symptoms of LOAD. Several drugs/therapies (i.e., urea cycle agents) have been reported in the context of Urea cycle disorders (UCDs) or hyperammonemia (HA) and may be used for treatment of AD or at least for ameliorating one or more symptoms of AD. Several drugs have been reported in the context of UCD or HA and may be used for treatment of LOAD or at least for ameliorating one or more symptoms of LOAD. Medications can include, but are not limited to, carglumic acid, glycerol phenylbutyrate, sodium phenylacetate, sodium benzoate and sodium phenylbutyrate. In some embodiments, a therapy is classified as an experimental therapy, such as a small molecule therapy (for example, ACER-001 (taste masked sodium phenylbutyrate)), an enzyme replacement therapy (for example, AEB 1102 also known as pegzilarginase, BB-OTC, or PRX-OTC), a mRNA therapy (for example, ARCT-810), a gene therapy (for instance, DTX301 (AAV8), P-OTC-101, or SEL-313), a microbiome metabolic therapy (for instance, KB-195), or a fusogen therapy (for instance, SG328). In some cases, drugs developed for use in treatment of classical (AR) urea cycle may be used for treatment of AD. Examples of this include but are not limited to glycerol phenylbutyrate (RAVICTI®) and taste-masked sodium phenylbutyrate (OLPRUVA™).

In some cases, earlier treatment of LOAD patients may include treatment of symptoms such as hyperammonemia. In some cases, an earlier treatment of AD or LOAD may include dietary plans (e.g., the Mediterranean diet, see PMID 29734664, Jin et al. Nutrients. 2018 May 4; 10(5):564) and/or supplements (e.g., medical food brands Milupa UCD 2 and UCD Anamix Junior by Nutricia North America). These may be typically available as over-the-counter (OTC) treatments and include, but are not limited to: low protein diet, low carbohydrate and high protein and fat diet, medium-chain triglyceride (MCT), sodium pyruvate, essential amino acids, L-arginine, L-citrulline (e.g., Cytolline by Solace Nutrition), D-ribose, uridine, and S-adenosyl-1-methionine. Critical care treatments may sometimes be included in the treatment regimen and include, but are not limited to, hemodialysis and liver transplants (e.g., orthotic or cells). Other therapeutic approaches under development include, but are not limited to, nitric oxide (NO) supplementation, mesenchymal stem cells, codon-optimized human OTC mRNA complexed with lipid-based nanoparticles, ammonia consuming bioengineered bacteria (e.g., SYNB 1020 by Synlogic), lactulose, autophagy enhancers, and farnesoid X receptor (FXR) agonists. Another experimental therapy is taurine supplementation, which has support from rodent models in the context of taurine deficiency (mouse knockout of SLC6A6, gene alias TAUT) as a cause of HA (e.g., see PMID 30862735, Qvartskhava N. et al. Proc Natl Acad Sci USA. 2019 Mar. 26; 116(13):6313-6318) and HA due to chronic liver injury (i.e., a non-genetic cause of HA) in rats (see PMID 28959615, Heidari R et al. Toxicol Rep. 2016 Apr. 13; 3:870 879). In humans, taurine supplementation (e.g., in the form of homotaurine, which is also known as ALZ-801, Alzhemed, tramiprosate, and Vivimind) is being investigated for treatment of MCI and AD (e.g., see PMID 32733362, Manzano S et al. Front Neurol. 2020 Jul. 7; 11:614 for a review on reported findings) and clinical trials suggest specific subsets of patients may see a greater benefit, such as AD patients homozygous for the APOE4/4 variant (e.g., see PMID 29199323, Abushakra S et al. J Prev Alzheimers Dis. 2016; 3(4):219-228). In MCI patients, homotaurine was found to favorably impact cytokine profiles, which was correlated with an improvement of episodic memory (see PMID 35515001, Toppi E et al. Front Immunol. 2022 Apr. 19; 13:813951). Evidence suggests that AD and/or MCI patients testing positive for one or more deleterious variants in a UCD/HA gene may see a greater benefit from homotaurine supplementation than AD and/or MCI patients without a deleterious variant in a UCD/HA gene. Since lipopolysaccharides (LPSs) from bacteria (e.g., that reside in the gastrointestinal tract of humans) are neurotoxic and have been linked to AD (e.g., see PMID 36293528, Zhao Y et al. Int J Mol Sci. 2022 Oct. 21; 23(20):12671), another potential treatment for AD or to prevent AD in those at risk for developing AD is to administer antibiotics (e.g., see PMID 34946536, Hurkacz M et al. Molecules. 2021 Dec. 9; 26(24):7456). Examples of antibiotics (reviewed by Hurkacz M et al. 2021) that may be useful for treating or preventing AD include, but are not limited to, tetracyclines (e.g., doxycycline and minocycline), cephalosporins (e.g., ceftriaxone), and antibiotics in the ansamycin family (e.g., rifampicin, a rifamycin derivative that is a subclass of the ansamycin family). A hypothesis for the mechanism of action of antibiotic therapy in treatment of AD or preventing AD is reduction of blood levels of ammonia due to secretion by the gut bacteria. Since HA can result from genetic (e.g., presence of one or more deleterious variants in one or more UCD or HA genes) and/or non-genetic factors (e.g., acute/chronic liver injury or gut bacteria), treatment modality for AD patients or subjects at risk of developing AD could include determining if the patient has a UCD/HA genetic subtype. If an AD patient (or MCI patient who is at risk for developing AD) is found to have HA due to genetic and non-genetic factors, it may be be beneficial to treat both sources of HA (e.g., with a urea cycle agent and an antibiotic).

The “diagnostic yield” as used herein refers to the percentage of cases with the presence of one or more genetic variations (e.g., CNV, SNV, indel, etc.) in a LOAD cohort using an assay. For example, if 40 cases were found with the presence of one or more genetic variations (e.g., CNV, SNV, indel, etc.) in a cohort of 100 LOAD patients, the diagnostic yield of the assay is 40%. In some cases, the patients in the LOAD cohort are clinically diagnosed with LOAD. In some cases, a patient is clinically diagnosed with LOAD when the patient presents with memory issues such as dementia, learning deficiencies, disorientation, cognitive damage, disorganized thinking, confusion. In some cases, the LOAD cohort has at least 5 LOAD cases, for example, at least 5, 6, 7, 8, 9, 10, 20, 30, 40, 50, 60, 70, 80, 90, 100, 200, 300, 400, 500, 600, 700, 800, 900, or 1000 LOAD cases. In some cases, the LOAD cohort is a cohort listed herein. For example, the LOAD cohort is the LOAD patient cohort listed in Table 7. In some cases, the assay is a genetic assay. In some cases, the genetic assay tests the genetic predisposition for LOAD.

The genetic assay can comprise any method disclosed herein. In some cases, the genetic assay has a diagnostic yield of at least about 0.1%, 0.2%, 0.3%, 04%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. In some cases, the genetic assay has a diagnostic yield of about 1%-5%, 1%-10%, 1%-20%, 5%-10%, 5%-20%, 10%-20%, 10%-30%, 20%-30%, 20%-40%, 30%-40%, 30%-50%, 40%-50%, 40%-60%, 50% 60%, 50%-70%, 60%-70%, 60%-80%, 70%-80%, 70%-90%, 80%-90%, 80%-95%, 90%-95%, 90%-99%, 90%-100%, 95%-99%, or 99%-100%.

Genetic Variations Associated with LOAD

Described herein, are methods that can be used to detect genetic variations. Detecting specific genetic variations, for example polymorphic markers and/or haplotypes, copy number, absence or presence of an allele, or genotype associated with a condition (e.g., disease or disorder) as described herein, can be accomplished by methods known in the art for analyzing nucleic acids and/or detecting sequences at polymorphic or genetically variable sites, for example, amplification techniques, hybridization techniques, sequencing, microarrays/arrays, or any combination thereof. Thus, by use of these methods disclosed herein or other methods available to the person skilled in the art, one or more alleles at polymorphic markers, including microsatellites, single nucleotide polymorphisms (SNPs), single nucleotide variations (SNVs), insertions/deletions (indels), copy number variations (CNVs), or other types of genetic variations, can be identified in a sample obtained from a subject.

Genomic sequences within populations exhibit variability between individuals at many locations in the genome. For example, the human genome exhibits sequence variations that occur on average every 500 base pairs. Such genetic variations in polynucleic acid sequences are commonly referred to as polymorphisms or polymorphic sites. As used herein, a polymorphism, e.g., genetic variation, includes a variation in the sequence of the genome amongst a population, such as allelic variations and other variations that arise or are observed. Thus, a polymorphism refers to the occurrence of two or more genetically determined alternative sequences or alleles in a population. These differences can occur in coding (e.g., exonic) and non-coding (e.g., intronic or intergenic) portions of the genome, and can be manifested or detected as differences in polynucleic acid sequences, gene expression, including, for example transcription, processing, translation, transport, protein processing, trafficking, DNA synthesis; expressed proteins, other gene products or products of biochemical pathways or in post-translational modifications and any other differences manifested amongst members of a population. Polymorphisms that arise as the result of a single base change, such as single nucleotide polymorphisms (SNPs) or single nucleotide variations (SNVs), can include an insertion, deletion or change in one nucleotide. A polymorphic marker or site is the locus at which divergence occurs. Such sites can be as small as one base pair (an SNP or SNV). Polymorphic markers include, but are not limited to, restriction fragment length polymorphisms (RFLPs), variable number of tandem repeats (VNTRs), hypervariable regions, minisatellites, dinucleotide repeats, trinucleotide repeats, tetranucleotide repeats and other repeating patterns, simple sequence repeats and insertional elements, such as Alu. Polymorphic forms also are manifested as different mendelian alleles for a gene. Polymorphisms can be observed by differences in proteins, protein modifications, RNA expression modification, DNA and RNA methylation, regulatory factors that alter gene expression and DNA replication, and any other manifestation of alterations in genomic polynucleic acid or organelle polynucleic acids. Those skilled in the art can appreciate that polymorphisms are sometimes considered to be a subclass of variations, defined on the basis of a particular frequency cutoff in a population. For example, in some embodiments, polymorphisms are considered to genetic variants/variations that occur at >1%, or >5%, frequency in the population.

In some embodiments, these genetic variations can be found to be associated with one or more disorders and/or diseases using the methods disclosed herein. In some embodiments, these genetic variations can be found to be associated with absence of one or more disorders and/or diseases (i.e. the one or more variants are protective against development of the disorder and/or diseases) using the methods disclosed herein.

In some embodiments, these genetic variations comprise point mutations, polymorphisms, single nucleotide polymorphisms (SNPs), single nucleotide variations (SNVs), translocations, insertions, deletions, amplifications, inversions, interstitial deletions, copy number variations (CNVs), structural variation (SV), loss of heterozygosity, or any combination thereof. As genetic variation includes any deletion, insertion or base substitution of the genomic DNA of one or more individuals in a first portion of a total population which thereby results in a difference at the site of the deletion, insertion or base substitution relative to one or more individuals in a second portion of the total population. Thus, the term “genetic variation” encompasses “wild type” or the most frequently occurring variation, and also includes “mutant,” or the less frequently occurring variation. In some embodiments, a wild type allele may be referred to as an ancestral allele.

As used herein, a target molecule that is “associated with” or “correlates with” a particular genetic variation is a molecule that can be functionally distinguished in its structure, activity, concentration, compartmentalization, degradation, secretion, and the like, as a result of such genetic variation. In some embodiments polymorphisms (e.g., polymorphic markers, genetic variations, or genetic variants) can comprise any nucleotide position at which two or more sequences are possible in a subject population. In some embodiments, each version of a nucleotide sequence, with respect to the polymorphism/variation, can represent a specific allele of the polymorphism/variation. In some embodiments, genomic DNA from a subject can contain two alleles for any given polymorphic marker, representative of each copy of the marker on each chromosome. In some embodiments, an allele can be a nucleotide sequence of a given location on a chromosome. Polymorphisms/variations can comprise any number of specific alleles. In some embodiments of the disclosure, a polymorphism/variation can be characterized by the presence of two or more alleles in a population. In some embodiments, the polymorphism/variation can be characterized by the presence of three or more alleles. In some embodiments, the polymorphism/variation can be characterized by four or more alleles, five or more alleles, six or more alleles, seven or more alleles, nine or more alleles, or ten or more alleles. In some embodiments an allele can be associated with one or more diseases or disorders, for example, a LOAD risk allele can be an allele that is associated with increased or decreased risk of developing LOAD. In some embodiments, genetic variations and alleles can be used to associate an inherited phenotype with a responsible genotype. In some embodiments, a LOAD risk allele can be a variant allele that is statistically associated with a screening of LOAD. In some embodiments, genetic variations can be of any measurable frequency in the population, for example, a frequency higher than 10%, a frequency from 5-10%, a frequency from 1-5%, a frequency from 0.1-1%, or a frequency below 0.1%. As used herein, variant alleles can be alleles that differ from a reference allele. As used herein, a variant can be a segment of DNA that differs from the reference DNA, such as a genetic variation. In some embodiments, genetic variations can be used to track the inheritance of a gene that has not yet been identified, but whose approximate location is known.

As used herein, a “haplotype” can be information regarding the presence or absence of one or more genetic markers in a given chromosomal region in a subject. In some embodiments, a haplotype can be a segment of DNA characterized by one or more alleles arranged along the segment, for example, a haplotype can comprise one member of the pair of alleles for each genetic variation or locus. In some embodiments, the haplotype can comprise two or more alleles, three or more alleles, four or more alleles, five or more alleles, or any combination thereof, wherein, each allele can comprise one or more genetic variations along the segment.

In some embodiments, a genetic variation can be a functional aberration that can alter gene function, gene expression, polypeptide expression, polypeptide function, or any combination thereof. In some embodiments, a genetic variation can be a loss-of-function mutation, gain-of-function mutation, dominant negative mutation, or reversion. In some embodiments, a genetic variation can be part of a gene's coding region or regulatory region. Regulatory regions can control gene expression and thus polypeptide expression. In some embodiments, a regulatory region can be a segment of DNA wherein regulatory polypeptides, for example, transcription or splicing factors, can bind. In some embodiments a regulatory region can be positioned near the gene being regulated, for example, positions upstream or downstream of the gene being regulated. In some embodiments, a regulatory region (e.g., enhancer element) can be several thousands of base pairs upstream or downstream of a gene.

In some embodiments, variants can include changes that affect a polypeptide, such as a change in expression level, sequence, function, localization, binding partners, or any combination thereof. In some embodiments, a genetic variation can be a frameshift mutation, nonsense mutation, missense mutation, neutral mutation, or silent mutation. For example, sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid, for example, if the change in the nucleic acid sequence causes a frame shift, the frame shift can result in a change in the encoded amino acids, and/or can result in the generation of a premature stop codon, causing generation of a truncated polypeptide. In some embodiments, a genetic variation associated with LOAD can be a synonymous change in one or more nucleotides, for example, a change that does not result in a change in the amino acid sequence. Such a polymorphism can, for example, alter splice sites, affect the stability or transport of mRNA, or otherwise affect the transcription or translation of an encoded polypeptide. In some embodiments, a synonymous mutation can result in the polypeptide product having an altered structure due to rare codon usage that impacts polypeptide folding during translation, which in some cases may alter its function and/or drug binding properties if it is a drug target. In some embodiments, the changes that can alter DNA increase the possibility that structural changes, such as amplifications or deletions, occur at the somatic level. A polypeptide encoded by the reference nucleotide sequence can be a reference polypeptide with a particular reference amino acid sequence, and polypeptides encoded by variant nucleotide sequences can be variant polypeptides with variant amino acid sequences.

The most common sequence variants comprise base variations at a single base position in the genome, and such sequence variants, or polymorphisms, are commonly called single nucleotide polymorphisms (SNPs) or single nucleotide variants (SNVs). In some embodiments, a SNP represents a genetic variant present at greater than or equal to 1% occurrence in a population and in some embodiments a SNP or an SNV can represent a genetic variant present at any frequency level in a population. A SNP can be a nucleotide sequence variation occurring when a single nucleotide at a location in the genome differs between members of a species or between paired chromosomes in a subject. SNPs can include variants of a single nucleotide, for example, at a given nucleotide position, some subjects can have a ‘G’, while others can have a ‘C’. SNPs can occur in a single mutational event, and therefore there can be two possible alleles at each SNP site; the original allele and the mutated allele. SNPs that are found to have two different bases in a single nucleotide position are referred to as biallelic SNPs, those with three are referred to as triallelic, and those with all four bases represented in the population are quadallelic. In some embodiments, SNPs can be considered neutral. In some embodiments SNPs can affect susceptibility to a condition (e.g., LOAD). SNP polymorphisms can have two alleles, for example, a subject can be homozygous for one allele of the polymorphism wherein both chromosomal copies of the individual have the same nucleotide at the SNP location, or a subject can be heterozygous wherein the two sister chromosomes of the subject contain different nucleotides. The SNP nomenclature as reported herein is the official Reference SNP (rs) ID identification tag as assigned to each unique SNP by the National Center for Biotechnological Information (NCBI).

Another genetic variation of the disclosure can be copy number variations (CNVs). As used herein, “CNVs” include alterations of the DNA of a genome that results in an abnormal number of copies of one or more sections of DNA. In some embodiments, a CNV comprises a CNV-subregion. As used herein, a “CNV-subregion” includes a continuous nucleotide sequence within a CNV. In some embodiments, the nucleotide sequence of a CNV-subregion can be shorter than the nucleotide sequence of the CNV, and in another embodiment the CNV-subregion can be equivalent to the CNV (e.g., such as for some CNVs). CNVs can be inherited or caused by de novo mutation and can be responsible for a substantial amount of human phenotypic variability, behavioral traits, and disease susceptibility. In some embodiments, CNVs of the current disclosure can be associated with susceptibility to one or more conditions, for example, LOAD. In some embodiments, CNVs can include a single gene or include a contiguous set of genes. In some embodiments, CNVs can be caused by structural rearrangements of the genome, for example, unbalanced translocations or inversions, insertions, deletions, amplifications, and interstitial deletions. In some embodiments, these structural rearrangements occur on one or more chromosomes. Low copy repeats (LCRs), which are region-specific repeat sequences (also known as segmental duplications), can be susceptible to these structural rearrangements, resulting in CNVs. Factors such as size, orientation, percentage similarity and the distance between the copies can influence the susceptibility of LCRs to genomic rearrangement. In addition, rearrangements may be mediated by the presence of high copy number repeats, such as long interspersed elements (LINES) and short interspersed elements (SINEs), often via non-homologous recombination. For example, chromosomal rearrangements can arise from non-allelic homologous recombination during meiosis or via a replication-based mechanism such as fork stalling and template switching (FoSTeS) (Zhang F. et al., Nat. Genet. (2009)) or microhomology-mediated break-induced repair (MMBIR) (Hastings P. J. et al., PLoS Genetics (2009)). In some embodiments, CNVs are referred to as structural variants, which are a broader class of variant that also includes copy number neutral alterations such as balanced inversions and balanced translocations.

CNVs can account for genetic variation affecting a substantial proportion of the human genome, for example, known CNVs can cover over 15% of the human genome sequence (Estivill and Armengol, PLoS Genetics (2007)). CNVs can affect gene expression, phenotypic variation and adaptation by disrupting or impairing gene dosage, and can cause disease, for example, microdeletion and microduplication disorders, and can confer susceptibility to diseases and disorders. Updated information about the location, type, and size of known CNVs can be found in one or more databases, for example, the Database of Genomic Variants (See, MacDonald J R et al., Nucleic Acids Res., 42, D986-92 (2014), which currently contains data for over 500,000 CNVs (as of May, 2016).

Other types of sequence variants can be found in the human genome and can be associated with a disease or disorder, including but not limited to, microsatellites. Microsatellite markers are stable, polymorphic, easily analyzed, and can occur regularly throughout the genome, making them especially suitable for genetic analysis. A polymorphic microsatellite can comprise multiple small repeats of bases, for example, CA repeats, at a particular site wherein the number of repeat lengths varies in a population. In some embodiments, microsatellites, for example, variable number of tandem repeats (VNTRs), can be short segments of DNA that have one or more repeated sequences, for example, about 2 to 5 nucleotides long, that can occur in non-coding DNA. In some embodiments, changes in microsatellites can occur during genetic recombination of sexual reproduction, increasing or decreasing the number of repeats found at an allele, or changing allele length.

The genetic variations disclosed herein can be associated with a risk of developing LOAD or MCI in a subject. In some cases, the subject can have a decreased risk due to the absence of one or more genetic variations that disrupt or modulate a corresponding gene according to Tables 1 to 11 or 15-18. For example, the subject can have a decreased risk due to the absence of one or more genetic variations that disrupt or modulate a corresponding gene according to Tables 1-5. In some cases, the subject can have an increased risk due to the presence of one or more genetic variations that disrupt or modulate a corresponding gene according to Tables 1 to 11 or 15-18. For example, the subject can have an increased risk due to the presence of one or more genetic variations that disrupt or modulate a corresponding gene according to Tables 1-5. In some cases, the subject can have a decreased risk due to the presence of one or both variants in Table 12. In some cases, the subject can have an increased risk due to the absence of one or both variants in Table 12.

### Subjects

A “subject”, as used herein, can be an individual of any age or sex from whom a sample containing polynucleotides is obtained for analysis by one or more methods described herein so as to obtain polynucleic acid information; for example, a male or female adult, child, newborn, or fetus. In some embodiments, a subject can be any target of therapeutic administration. In some embodiments, a subject can be a test subject or a reference subject.

As used herein, a “cohort” can represent an ethnic group, a patient group, a particular age group, a group not associated with a particular condition (e.g., disease or disorder), a group associated with a particular condition (e.g., disease or disorder), a group of asymptomatic subjects, a group of symptomatic subjects, or a group or subgroup of subjects associated with a particular response to a treatment regimen or enrolled in a clinical trial. In some embodiments, a patient can be a subject afflicted with a condition (e.g., disease or disorder). In some embodiments, a patient can be a subject not afflicted with a condition (e.g., disease or disorder) and is considered apparently healthy, or a normal or control subject. In some embodiments, a subject can be a test subject, a patient or a candidate for a therapeutic, wherein genomic DNA from the subject, patient, or candidate is obtained for analysis by one or more methods of the present disclosure herein, so as to obtain genetic variation information of the subject, patient or candidate.

In some embodiments, the polynucleic acid sample can be obtained prenatally from a fetus or embryo or from the mother, for example, from fetal or embryonic cells in the maternal circulation. In some embodiments, the polynucleic acid sample can be obtained with the assistance of a health care provider, for example, to draw blood. In some embodiments, the polynucleic acid sample can be obtained without the assistance of a health care provider, for example, where the polynucleic acid sample is obtained non-invasively, such as a saliva sample, or a sample comprising buccal cells that is obtained using a buccal swab or brush, or a mouthwash sample.

The present disclosure also provides methods for assessing genetic variations in subjects who are members of a target population. Such a target population is in some embodiments a population or group of subjects at risk of developing the condition (e.g., disease or disorder), based on, for example, other genetic factors, biomarkers, biophysical parameters, diagnostic testing such as magnetic resonance imaging (MRI), family history of the condition, previous screening or medical history, or any combination thereof.

The genetic variations of the present disclosure found to be associated with a condition (e.g., disease or disorder) can show similar association in other human populations. Particular embodiments comprising subject human populations are thus also contemplated and within the scope of the disclosure. Such embodiments relate to human subjects that are from one or more human populations including, but not limited to, Caucasian, Ashkenazi Jewish, Sephardi Jewish, European, American, Eurasian, Asian, Central/South Asian, East Asian, Middle Eastern, African, Hispanic, Caribbean, and Oceanic populations. European populations include, but are not limited to, Swedish, Norwegian, Finnish, Russian, Danish, Icelandic, Irish, Celt, English, Scottish, Dutch, Belgian, French, German, Spanish, Portuguese, Italian, Polish, Bulgarian, Slavic, Serbian, Bosnian, Czech, Greek and Turkish populations. The ethnic contribution in subjects can also be determined by genetic analysis, for example, genetic analysis of ancestry can be carried out using unlinked microsatellite markers or single nucleotide polymorphisms (SNPs) such as those set out in Smith et al., (Smith M. W. et al., Am. J. Hum. Genet., 74:1001 (2004)).

Certain genetic variations can have different population frequencies in different populations, or are polymorphic in one population but not in another. The methods available and as thought herein can be applied to practice the present disclosure in any given human population. This can include assessment of genetic variations of the present disclosure, so as to identify those markers that give strongest association within the specific population. Thus, the at-risk variants of the present disclosure can reside on different haplotype background and in different frequencies in various human populations.

### Samples

Samples that are suitable for use in the methods described herein can be polynucleic acid samples from a subject. A “polynucleic acid sample” as used herein can include RNA or DNA, or a combination thereof. In another embodiment, a “polypeptide sample” (e.g., peptides or proteins, or fragments therefrom) can be used to ascertain information that an amino acid change has occurred, which is the result of a genetic variant. Polynucleic acids and polypeptides can be extracted from one or more samples including but not limited to, blood, saliva, urine, mucosal scrapings of the lining of the mouth, expectorant, serum, tears, skin, tissue, or hair. A polynucleic acid sample can be assayed for polynucleic acid information. “Polynucleic acid information,” as used herein, includes a polynucleic acid sequence itself, the presence/absence of genetic variation in the polynucleic acid sequence, a physical property which varies depending on the polynucleic acid sequence (e.g., Tm), and the amount of the polynucleic acid (e.g., number of mRNA copies). A “polynucleic acid” means any one of DNA, RNA, DNA including artificial nucleotides, or RNA including artificial nucleotides. As used herein, a “purified polynucleic acid” includes cDNAs, fragments of genomic polynucleic acids, polynucleic acids produced using the polymerase chain reaction (PCR), polynucleic acids formed by restriction enzyme treatment of genomic polynucleic acids, recombinant polynucleic acids, and chemically synthesized polynucleic acid molecules. A “recombinant” polynucleic acid molecule includes a polynucleic acid molecule made by an artificial combination of two otherwise separated segments of sequence, e.g., by chemical synthesis or by the manipulation of isolated segments of polynucleic acids by genetic engineering techniques. As used herein, a “polypeptide” includes proteins, fragments of proteins, and peptides, whether isolated from natural sources, produced by recombinant techniques, or chemically synthesized. A polypeptide may have one or more modifications, such as a post-translational modification (e.g., glycosylation, phosphorylation, etc.) or any other modification (e.g., pegylation, etc.). The polypeptide may contain one or more non-naturally-occurring amino acids (e.g., such as an amino acid with a side chain modification).

In some embodiments, the polynucleic acid sample can comprise cells or tissue, for example, cell lines. Exemplary cell types from which nucleic acids can be obtained using the methods described herein include, but are not limited to, the following: a blood cell such as a B lymphocyte, T lymphocyte, leukocyte, erythrocyte, macrophage, or neutrophil; a muscle cell such as a skeletal cell, smooth muscle cell or cardiac muscle cell; a germ cell, such as a sperm or egg; an epithelial cell; a connective tissue cell, such as an adipocyte, chondrocyte; fibroblast or osteoblast; a neuron; an astrocyte; a stromal cell; an organ specific cell, such as a kidney cell, pancreatic cell, liver cell, or a keratinocyte; a stem cell; or any cell that develops therefrom. A cell from which nucleic acids can be obtained can be a blood cell or a particular type of blood cell including, for example, a hematopoietic stem cell or a cell that arises from a hematopoietic stem cell such as a red blood cell, B lymphocyte, T lymphocyte, natural killer cell, neutrophil, basophil, eosinophil, monocyte, macrophage, or platelet. Generally, any type of stem cell can be used including, without limitation, an embryonic stem cell, adult stem cell, or pluripotent stem cell.

In some embodiments, a polynucleic acid sample can be processed for RNA or DNA isolation, for example, RNA or DNA in a cell or tissue sample can be separated from other components of the polynucleic acid sample. Cells can be harvested from a polynucleic acid sample using standard techniques, for example, by centrifuging a cell sample and resuspending the pelleted cells, for example, in a buffered solution, for example, phosphate-buffered saline (PBS). In some embodiments, after centrifuging the cell suspension to obtain a cell pellet, the cells can be lysed to extract DNA. In some embodiments, the nucleic acid sample can be concentrated and/or purified to isolate DNA. All nucleic acid samples obtained from a subject, including those subjected to any sort of further processing, are considered to be obtained from the subject. In some embodiments, standard techniques and kits known in the art can be used to extract RNA or DNA from a nucleic acid sample, including, for example, phenol extraction, a QIAAMP® Tissue Kit (Qiagen, Chatsworth, Calif.), a WIZARD® Genomic DNA purification kit (Promega), or a Qiagen Autopure method using Puregene chemistry, which can enable purification of highly stable DNA well-suited for archiving.

In some embodiments, determining the identity of an allele or determining copy number can, but need not, include obtaining a polynucleic acid sample comprising RNA and/or DNA from a subject, and/or assessing the identity, copy number, presence or absence of one or more genetic variations and their chromosomal locations within the genomic DNA (i.e. subject's genome) derived from the polynucleic acid sample.

The individual or organization that performs the determination need not actually carry out the physical analysis of a nucleic acid sample from a subject. In some embodiments, the methods can include using information obtained by analysis of the polynucleic acid sample by a third party. In some embodiments, the methods can include steps that occur at more than one site. For example, a polynucleic acid sample can be obtained from a subject at a first site, such as at a health care provider or at the subject's home in the case of a self-testing kit. The polynucleic acid sample can be analyzed at the same or a second site, for example, at a laboratory or other testing facility.

### Nucleic Acids

The nucleic acids and polypeptides described herein can be used in methods and kits of the present disclosure. In some embodiments, aptamers that specifically bind the nucleic acids and polypeptides described herein can be used in methods and kits of the present disclosure. As used herein, a nucleic acid can comprise a deoxyribonucleotide (DNA) or ribonucleotide (RNA), whether singular or in polymers, naturally occurring or non-naturally occurring, double-stranded or single-stranded, coding, for example a translated gene, or non-coding, for example a regulatory region, or any fragments, derivatives, mimetics or complements thereof. In some embodiments, nucleic acids can comprise oligonucleotides, nucleotides, polynucleotides, nucleic acid sequences, genomic sequences, complementary DNA (cDNA), antisense nucleic acids, DNA regions, probes, primers, genes, regulatory regions, introns, exons, open-reading frames, binding sites, target nucleic acids and allele-specific nucleic acids.

A “probe,” as used herein, includes a nucleic acid fragment for examining a nucleic acid in a specimen using the hybridization reaction based on the complementarity of nucleic acid.

A “hybrid” as used herein, includes a double strand formed between any one of the abovementioned nucleic acid, within the same type, or across different types, including DNA-DNA, DNA-RNA, RNA-RNA or the like.

“Isolated” nucleic acids, as used herein, are separated from nucleic acids that normally flank the gene or nucleotide sequence (as in genomic sequences) and/or has been completely or partially purified from other transcribed sequences (e.g., as in an RNA library). For example, isolated nucleic acids of the disclosure can be substantially isolated with respect to the complex cellular milieu in which it naturally occurs, or culture medium when produced by recombinant techniques, or chemical precursors or other chemicals when chemically synthesized. In some instances, the isolated material can form part of a composition, for example, a crude extract containing other substances, buffer system or reagent mix. In some embodiments, the material can be purified to essential homogeneity using methods known in the art, for example, by polyacrylamide gel electrophoresis (PAGE) or column chromatography (e.g., HPLC). With regard to genomic DNA (gDNA), the term “isolated” also can refer to nucleic acids that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2kb, 1 kb, 0.5 kb or 0.1 kb of the nucleotides that flank the nucleic acid molecule in the gDNA of the cell from which the nucleic acid molecule is derived.

Nucleic acids can be fused to other coding or regulatory sequences can be considered isolated. For example, recombinant DNA contained in a vector is included in the definition of “isolated” as used herein. In some embodiments, isolated nucleic acids can include recombinant DNA molecules in heterologous host cells or heterologous organisms, as well as partially or substantially purified DNA molecules in solution. Isolated nucleic acids also encompass in vivo and in vitro RNA transcripts of the DNA molecules of the present disclosure. An isolated nucleic acid molecule or nucleotide sequence can be synthesized chemically or by recombinant means. Such isolated nucleotide sequences can be useful, for example, in the manufacture of the encoded polypeptide, as probes for isolating homologous sequences (e.g., from other mammalian species), for gene mapping (e.g., by in situ hybridization with chromosomes), or for detecting expression of the gene, in tissue (e.g., human tissue), such as by Northern blot analysis or other hybridization techniques disclosed herein. The disclosure also pertains to nucleic acid sequences that hybridize under high stringency hybridization conditions, such as for selective hybridization, to a nucleotide sequence described herein Such nucleic acid sequences can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al., John Wiley & Sons, (1998), and Kraus, M. and Aaronson, S., Methods Enzymol., 200:546-556 (1991), the entire teachings of which are incorporated by reference herein.

Calculations of “identity” or “percent identity” between two or more nucleotide or amino acid sequences can be determined by aligning the sequences for optimal comparison purposes (e.g., gaps can be introduced in the sequence of a first sequence). The nucleotides at corresponding positions are then compared, and the percent identity between the two sequences is a function of the number of identical positions shared by the sequences (i.e. % identity=#of identical positions/total #of positions×100). For example, a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

In some embodiments, the length of a sequence aligned for comparison purposes is at least 30%, at least 40%, at least 50%, at least 60%, at least 70%, at least 80%, at least 90%, or at least 95% of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90-5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In some embodiments, the percent identity between two amino acid sequences can be accomplished using, for example, the GAP program in the GCG software package (Accelrys, Cambridge, UK).

“Probes” or “primers” can be oligonucleotides that hybridize in a base-specific manner to a complementary strand of a nucleic acid molecule. Probes can include primers, which can be a single-stranded oligonucleotide probe that can act as a point of initiation of template-directed DNA synthesis using methods including but not limited to, polymerase chain reaction (PCR) and ligase chain reaction (LCR) for amplification of a target sequence. Oligonucleotides, as described herein, can include segments or fragments of nucleic acid sequences, or their complements. In some embodiments, DNA segments can be between 5 and 10,000 contiguous bases, and can range from 5, 10, 12, 15, 20, or 25 nucleotides to 10, 15, 20, 25, 30, 40, 50, 100, 200, 500, 1000 or 10,000 nucleotides. In addition to DNA and RNA, probes and primers can include polypeptide nucleic acids (PNA), as described in Nielsen, P. et al., Science 254: 1497 1500 (1991). A probe or primer can comprise a region of nucleotide sequence that hybridizes to at least about 15, typically about 20-25, and in certain embodiments about 40, 50, 60 or 75, consecutive nucleotides of a nucleic acid molecule.

The present disclosure also provides isolated nucleic acids, for example, probes or primers, that contain a fragment or portion that can selectively hybridize to a nucleic acid that comprises, or consists of, a nucleotide sequence, wherein the nucleotide sequence can comprise at least one polymorphism or polymorphic allele contained in the genetic variations described herein or the wild-type nucleotide that is located at the same position, or the complements thereof. In some embodiments, the probe or primer can be at least 70% identical, at least 80% identical, at least 85% identical, at least 90% identical, or at least 95% identical, to the contiguous nucleotide sequence or to the complement of the contiguous nucleotide sequence.

In some embodiments, a nucleic acid probe can be an oligonucleotide capable of hybridizing with a complementary region of a gene associated with a condition (e.g., LOAD) containing a genetic variation described herein. The nucleic acid fragments of the disclosure can be used as probes or primers in assays such as those described herein.

The nucleic acids of the disclosure, such as those described above, can be identified and isolated using standard molecular biology techniques well known to the skilled person. In some embodiments, DNA can be amplified and/or can be labeled (e.g., radiolabeled, fluorescently labeled) and used as a probe for screening, for example, a cDNA library derived from an organism. cDNA can be derived from mRNA and can be contained in a suitable vector. For example, corresponding clones can be isolated, DNA obtained fallowing in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

In some embodiments, nucleic acid can comprise one or more polymorphisms, variations, or mutations, for example, single nucleotide polymorphisms (SNPs), single nucleotide variations (SNVs), copy number variations (CNVs), for example, insertions, deletions, inversions, and translocations. In some embodiments, nucleic acids can comprise analogs, for example, phosphorothioates, phosphoramidates, methyl phosphonate, chiralmethyl phosphonates, 2-0-methyl ribonucleotides, or modified nucleic acids, for example, modified backbone residues or linkages, or nucleic acids combined with carbohydrates, lipids, polypeptide or other materials, or peptide nucleic acids (PNAs), for example, chromatin, ribosomes, and transcriptosomes. In some embodiments nucleic acids can comprise nucleic acids in various structures, for example, A DNA, B DNA, Z-form DNA, siRNA, tRNA, and ribozymes. In some embodiments, the nucleic acid may be naturally or non-naturally polymorphic, for example, having one or more sequence differences, for example, additions, deletions and/or substitutions, as compared to a reference sequence. In some embodiments, a reference sequence can be based on publicly available information, for example, the U.C. Santa Cruz Human Genome Browser Gateway (genome.ucsc.edu/cgi-bin/hgGateway) or the NCBI website (ncbi.nlm.nih.gov). In some embodiments, a reference sequence can be determined by a practitioner of the present disclosure using methods well known in the art, for example, by sequencing a reference nucleic acid.

In some embodiments, a probe can hybridize to an allele, SNP, SNV, or CNV as described herein. In some embodiments, the probe can bind to another marker sequence associated with LOAD as described herein.

One of skill in the art would know how to design a probe so that sequence specific hybridization can occur only if a particular allele is present in a genomic sequence from a test nucleic acid sample. The disclosure can also be reduced to practice using any convenient genotyping method, including commercially available technologies and methods for genotyping particular genetic variations

Control probes can also be used, for example, a probe that binds a less variable sequence, for example, a repetitive DNA associated with a centromere of a chromosome, can be used as a control. In some embodiments, probes can be obtained from commercial sources. In some embodiments, probes can be synthesized, for example, chemically or in vitro, or made from chromosomal or genomic DNA through standard techniques. In some embodiments sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification using PCR.

One or more nucleic acids for example, a probe or primer, can also be labeled, for example, by direct labeling, to comprise a detectable label. A detectable label can comprise any label capable of detection by a physical, chemical, or a biological process for example, a radioactive label, such as ³²P or ³H, a fluorescent label, such as FITC, a chromophore label, an affinity-ligand label, an enzyme label, such as alkaline phosphatase, horseradish peroxidase, or I2 galactosidase, an enzyme cofactor label, a hapten conjugate label, such as digoxigenin or dinitrophenyl, a Raman signal generating label, a magnetic label, a spin label, an epitope label, such as the FLAG or HA epitope, a luminescent label, a heavy atom label, a nanoparticle label, an electrochemical label, a light scattering label, a spherical shell label, semiconductor nanocrystal label, such as quantum dots (described in U.S. Pat. No. 6,207,392), and probes labeled with any other signal generating label known to those of skill in the art, wherein a label can allow the probe to be visualized with or without a secondary detection molecule. A nucleotide can be directly incorporated into a probe with standard techniques, for example, nick translation, random priming, and PCR labeling. A “signal,” as used herein, include a signal suitably detectable and measurable by appropriate means, including fluorescence, radioactivity, chemiluminescence, and the like.

Non-limiting examples of label moieties useful for detection include, without limitation, suitable enzymes such as horseradish peroxidase, alkaline phosphatase, beta-galactosidase, or acetylcholinesterase; members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, Texas Red, dichlorotriazinylamine fluorescein, dansyl chloride, phycoerythrin, fluorescent lanthanide complexes such as those including Europium and Terbium, cyanine dye family members, such as Cy3 and Cy5, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the 6th Edition of the Molecular Probes Handbook by Richard P. Hoagland; a luminescent material such as luminol; light scattering or plasmon resonant materials such as gold or silver particles or quantum dots; or radioactive material include ¹⁴C, ¹²³I, ¹²⁴I, ¹²⁵I, Tc99m, ³²P, ³³P, ³⁵S or ³H.

Other labels can also be used in the methods of the present disclosure, for example, backbone labels. Backbone labels comprise nucleic acid stains that bind nucleic acids in a sequence independent manner. Non-limiting examples include intercalating dyes such as phenanthridines and acridines (e.g., ethidium bromide, propidium iodide, hexidium iodide, dihydroethidium, ethidium homodimer-1 and -2, ethidium monoazide, and ACMA); some minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS75 1, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-S, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, 44, -45 (blue), SYTO-13, -16, -24, -21, -23, -12, -11, -20, -22, -15, -14, -25 (green), SYTO-81, -80, -82, 83, -84, -85 (orange), SYTO-64, -17, -59, -61, -62, -60, -63 (red).

In some embodiments, fluorophores of different colors can be chosen, for example, 7-amino-4-methylcoumarin-3-acetic acid (AMCA), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)—isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, TRITC, rhodamine, tetramethylrhodamine, R-phycoerythrin, Cy-3, Cy-5, Cy-7, Texas Red, Phar-Red, allophycocyanin (APC), and CASCADE™ blue acetylazide, such that each probe in or not in a set can be distinctly visualized. In some embodiments, fluorescently labeled probes can be viewed with a fluorescence microscope and an appropriate filter for each fluorophore, or by using dual or triple band-pass filter sets to observe multiple fluorophores. In some embodiments, techniques such as flow cytometry can be used to examine the hybridization pattern of the probes.

In other embodiments, the probes can be indirectly labeled, for example, with biotin or digoxygenin, or labeled with radioactive isotopes such as ³²P and/or ³H. As a non-limiting example, a probe indirectly labeled with biotin can be detected by avidin conjugated to a detectable marker. For example, avidin can be conjugated to an enzymatic marker such as alkaline phosphatase or horseradish peroxidase. In some embodiments, enzymatic markers can be detected using colorimetric reactions using a substrate and/or a catalyst for the enzyme. In some embodiments, catalysts for alkaline phosphatase can be used, for example, 5-bromo-4-chloro-3-indolylphosphate and nitro blue tetrazolium. In some embodiments, a catalyst can be used for horseradish peroxidase, for example, diaminobenzoate.

### Methods of Screening

As used herein, screening a subject comprises diagnosing or determining, theranosing, or determining the susceptibility to developing (prognosing) a condition, for example, LOAD. In particular embodiments, the disclosure is a method of determining a presence of, or a susceptibility to, LOAD, by detecting at least one genetic variation in a sample from a subject as described herein. In some embodiments, detection of particular alleles, markers, variations, or haplotypes is indicative of a presence or susceptibility to a condition (e.g., LOAD).

While means for screening LOAD using cognitive functions exist, LOAD risk is not adequately assessed by the neuropsychologic tests alone. Thus there exists a need for an improved screening test for assessing the risk of developing LOAD. Described herein are methods of screening an individual for a risk of developing LOAD, including but not limited to, determining the identity and location of genetic variations, such as variations in nucleotide sequence and copy number, and the presence or absence of alleles or genotypes in one or more samples from one or more subjects using any of the methods described herein. In some embodiments, determining an association to having or developing LOAD can be performed by detecting particular variations that appear more frequently in test subjects compared to reference subjects and analyzing the molecular and physiological pathways these variations can affect.

Within any given population, there can be an absolute susceptibility of developing a disease or trait, defined as the chance of a person developing the specific disease or trait over a specified time-period. Susceptibility (e.g., being at-risk) is typically measured by looking at very large numbers of people, rather than at a particular individual. As described herein, certain copy number variations (genetic variations) and/or single nucleotide variations are found to be useful for susceptibility assessment of LOAD. Susceptibility assessment can involve detecting particular genetic variations in the genome of individuals undergoing assessment. Particular genetic variations are found more frequently in individuals with LOAD, than in individuals without LOAD. Therefore, these genetic variations have predictive value for detecting LOAD, or a susceptibility to LOAD, in an individual. Without intending to be limited by theory, it is believed that the genetic variations described herein to be associated with susceptibility of LOAD represent functional variants predisposing to the disease. In some embodiments, a genetic variation can confer a susceptibility of the condition, for example carriers of the genetic variation are at a different risk of the condition than non-carriers. In some embodiments, the presence of a genetic variation is indicative of increased susceptibility to LOAD.

In some embodiments, screening can be performed using any of the methods disclosed, alone or in combination. In some embodiments, screening can be performed using Polymerase Chain Reaction (PCR). In some embodiments screening can be performed using Array Comparative Genomic Hybridization (aCGH) to detect CNVs. In another preferred embodiment screening can be performed using whole exome sequencing (WES) to detect SNVs, indels, and in some cases CNVs using appropriate analysis algorithms. In another preferred embodiment screening is performed using high-throughput (also known as next generation) whole genome sequencing (WGS) methods and appropriate algorithms to detect all or nearly all genetic variations present in a genomic DNA sample. In some embodiments, the genetic variation information as it relates to the current disclosure can be used in conjunction with any of the above mentioned symptomatic screening tests to screen a subject for LOAD, for example, using a combination of aCGH and/or sequencing with a neuropsychologic test such as cognitive function test, memory analysis, patient history.

In some embodiments, information from any of the above screening methods (e.g., specific symptoms, scoring matrix, or genetic variation data) can be used to define a subject as a test subject or reference subject. In some embodiments, information from any of the above screening methods can be used to associate a subject with a test or reference population, for example, a subject in a population.

In one embodiment, an association with LOAD can be determined by the statistical likelihood of the presence of a genetic variation in a subject with LOAD, for example, an unrelated individual or a first or second-degree relation of the subject. In some embodiments, an association with LOAD can be decided by determining the statistical likelihood of the absence of a genetic variation in an unaffected reference subject, for example, an unrelated individual or a first or second-degree relation of the subject. The methods described herein can include obtaining and analyzing a nucleic acid sample from one or more suitable reference subjects.

In the present context, the term screening comprises diagnosis, prognosis, and theranosis. Screening can refer to any available screening method, including those mentioned herein. As used herein, susceptibility can be proneness of a subject towards the development of LOAD, or towards being less able to resist LOAD than one or more control subjects. In some embodiments, susceptibility can encompass increased susceptibility. For example, particular nucleic acid variations of the disclosure as described herein can be characteristic of increased susceptibility to LOAD. In some embodiments, particular nucleic acid variations can confer decreased susceptibility, for example particular nucleic variations of the disclosure as described herein can be characteristic of decreased susceptibility to development of LOAD.

As described herein, a genetic variation predictive of susceptibility to or presence of LOAD can be one where the particular genetic variation is more frequently present in a group of subjects with the condition (affected), compared to the frequency of its presence in a reference group (control), such that the presence of the genetic variation is indicative of susceptibility to or presence of LOAD. In some embodiments, the reference group can be a population nucleic acid sample, for example, a random nucleic acid sample from the general population or a mixture of two or more nucleic acid samples from a population. In some embodiments, disease-free controls can be characterized by the absence of one or more specific disease-associated symptoms, for example, individuals who have not experienced symptoms associated with LOAD. In some embodiments, the disease-free control group is characterized by the absence of one or more disease-specific risk factors, for example, at least one genetic and/or environmental risk factor. In some embodiments, a reference sequence can be referred to for a particular site of genetic variation. In some embodiments, a reference allele can be a wild-type allele and can be chosen as either the first sequenced allele or as the allele from a control individual. In some embodiments, one or more reference subjects can be characteristically matched with one or more affected subjects, for example, with matched aged, gender or ethnicity.

A person skilled in the art can appreciate that for genetic variations with two or more alleles present in the population being studied, and wherein one allele can be found in increased frequency in a group of individuals with LOAD in the population, compared with controls, the other allele of the marker can be found in decreased frequency in the group of individuals with the trait or disease, compared with controls. In such a case, one allele of the marker, for example, the allele found in increased frequency in individuals with LOAD, can be the at-risk allele, while the other allele(s) can be a neutral or protective allele.

A genetic variant associated with LOAD can be used to predict the susceptibility of the disease for a given genotype. For any genetic variation, there can be one or more possible genotypes, for example, homozygote for the at-risk variant (e.g., in autosomal recessive disorders), heterozygote, and non-carrier of the at-risk variant. Autosomal recessive disorders can also result from two distinct genetic variants impacting the same gene such that the individual is a compound heterozygote (e.g., the maternal allele contains a different mutation than the paternal allele). Compound heterozygosity may result from two different SNVs, two different CNVs, an SNV and a CNV, or any combination of two different genetic variants but each present on a different allele for the gene. For X-linked genes, males who possess one copy of a variant-containing gene may be affected, while carrier females, who also possess a wild-type gene, may remain unaffected. In some embodiments, susceptibility associated with variants at multiple loci can be used to estimate overall susceptibility. For multiple genetic variants, there can be k (k=3\hat{ }n*2\hat{ }P) possible genotypes; wherein n can be the number of autosomal loci and p can be the number of gonosomal (sex chromosomal) loci. Overall susceptibility assessment calculations can assume that the relative susceptibilities of different genetic variants multiply, for example, the overall susceptibility associated with a particular genotype combination can be the product of the susceptibility values for the genotype at each locus. If the susceptibility presented is the relative susceptibility for a person, or a specific genotype for a person, compared to a reference population, then the combined susceptibility can be the product of the locus specific susceptibility values and can correspond to an overall susceptibility estimate compared with a population. If the susceptibility for a person is based on a comparison to non-carriers of the at-risk allele, then the combined susceptibility can correspond to an estimate that compares the person with a given combination of genotypes at all loci to a group of individuals who do not carry at-risk variants at any of those loci. The group of non-carriers of any at-risk variant can have the lowest estimated susceptibility and can have a combined susceptibility, compared with itself, for example, non-carriers, of 1.0, but can have an overall susceptibility, compared with the population, of less than 1.0.

Overall risk for multiple risk variants can be performed using standard methodology. Genetic variations described herein can form the basis of risk analysis that combines other genetic variations known to increase risk of LOAD, or other genetic risk variants for LOAD. In certain embodiments of the disclosure, a plurality of variants (genetic variations, variant alleles, and/or haplotypes) can be used for overall risk assessment. These variants are in some embodiments selected from the genetic variations as disclosed herein. Other embodiments include the use of the variants of the present disclosure in combination with other variants known to be useful for screening a susceptibility to LOAD. In such embodiments, the genotype status of a plurality of genetic variations, markers and/or haplotypes is determined in an individual, and the status of the individual compared with the population frequency of the associated variants, or the frequency of the variants in clinically healthy subjects, such as age-matched and sex-matched subjects.

Methods such as the use of available algorithms and software can be used to identify, or call, significant genetic variations, including but not limited to, algorithms of DNA Analytics or DNA copy, iPattern and/or QuantiSNP. In some embodiments, a threshold log ratio value can be used to determine losses and gains. For example, using DNA Analytics, a log₂ ratio cutoff of ≥0.5 and ≤0.5 to classify CNV gains and losses respectively can be used. For example, using DNA Analytics, a log₂ ratio cutoff of ≥0.25 and ≤0.25 to classify CNV gains and losses respectively can be used. As a further example, using DNAcopy, a log₂ ratio cutoff of ≥0.35 and ≤0.35 to classify CNV gains and losses respectively can be used. For example, an Aberration Detection Module 2 (ADM2) algorithm, such as that of DNA Analytics 4.0.85 can be used to identify, or call, significant genetic variations. In some embodiments, two or more algorithms can be used to identify, or call, significant genetic variations. For example, 2, 3, 4, 5, 6, 7, 8, 9, or 10 or more algorithms can be used to identify, or call, significant genetic variations. In another embodiment, the log 2 ratio of one or more individual probes on a microarray can be used to identify significant genetic variations, such as the presence of homozygously deleted regions in a subject's genome. In some embodiments, significant genetic variations can be CNVs.

CNVs detected by two or more algorithms can be defined as stringent and can be utilized for further analyses. In some embodiments, the information and calls from two or more of the methods described herein can be compared to each other to identify significant genetic variations more or less stringently. For example, CNV calls generated by two or more of DNA Analytics, Aberration Detection Module 2 (ADM2) algorithms, and DNAcopy algorithms can be defined as stringent CNVs. In some embodiments significant or stringent genetic variations can be tagged as identified or called if it can be found to have a minimal reciprocal overlap to a genetic variation detected by one or more platforms and/or methods described herein. For example, a minimum of 50% reciprocal overlap can be used to tag the CNVs as identified or called. For example, significant or stringent genetic variations can be tagged as identified or called if it can be found to have a reciprocal overlap of more than about 50%, 55% 60%, 65%, 70%, 75%, 80%, 85%, 90%, or 95%, 99%, or equal to 100%, to a genetic variation detected by one or more platforms and/or methods described herein. For example, significant or stringent genetic variations can be tagged as identified or called if it can be found to have a reciprocal overlap of more than about 50% reciprocal overlap to a genetic variation detected by one or more platforms and/or methods described herein. In another embodiment, genetic variations can be detected from the log 2 ratio values calculated for individual probes present on an aCGH microarray via a statistical comparison of the probe's log 2 ratio value in a cohort of subjects with LOAD to the probe's log 2 ratio value in a cohort of subjects without LOAD.

In some embodiments, a threshold log ratio value can be used to determine losses and gains. A log ratio value can be any log ratio value; for example, a log ratio value can be a log 2 ratio or a log 10 ratio. In some embodiments, a CNV segment whose median log 2 ratio is less than or equal to a log 2 ratio threshold value can be classified as a loss. For example, any segment whose median log 2 ratio is less than or equal to −0.1, −0.11, −0.12, −0.13, −0.14, −0.15, −0.16, −0.17, −0.18, −0.19, −0.2, −0.21, −0.22, −0.23, −0.24, −0.25, 0.26, −0.27, −0.28, −0.29, −0.3, −0.31, −0.32, −0.33, −0.34, −0.35, −0.36, −0.37, −0.38, −0.39, −0.4, −0.41, −0.42, −0.43, −0.44, −0.45, −0.46, −0.47, −0.48, −0.49, −0.5, −0.55, −0.6, −0.65, −0.7, −0.75, −0.8, −0.85, −0.9, −0.95, −1, −1.1, −1.2, −1.3, −1.4, −1.5, −1.6, −1.7, −1.8, −1.9, −2, −2.1, −2.2, −2.3, −2.4, −2.5, −2.6, −2.7, −2.8, −2.9, −3, −3.1, 3.2, −3.3, −3.4, −3.5, −3.6, −3.7, −3.8, −3.9, −4, −4.1, −4.2, −4.3, −4.4, −4.5, −4.6, −4.7, −4.8, −4.9, −5, −5.5, −6, −6.5, −7, −7.5, −8, −8.5, −9, −9.5, −10, −11, −12, −13, −14, −15, −16, −17, −18, −19, −20 or less, can be classified as a loss.

In some embodiments, one algorithm can be used to call or identify significant genetic variations, wherein any segment whose median log 2 ratio was less than or equal to −0.1, −0.11, −0.12, −0.13, −0.14, 0.15, −0.16, −0.17, −0.18, −0.19, −0.2, −0.21, −0.22, −0.23, −0.24, −0.25, −0.26, −0.27, −0.28, −0.29, −0.3, −0.31, −0.32, −0.33, −0.34, −0.35, −0.36, −0.37, −0.38, −0.39, −0.4, −0.41, −0.42, −0.43, −0.44, −0.45, −0.46, −0.47, −0.48, −0.49, −0.5, −0.55, −0.6, −0.65, −0.7, −0.75, −0.8, −0.85, −0.9, −0.95, −1, −1.1, −1.2, −1.3, −1.4, −1.5, −1.6, −1.7, 1.8, −1.9, −2, −2.1, −2.2, −2.3, −2.4, −2.5, −2.6, −2.7, −2.8, −2.9, −3, −3.1, −3.2, −3.3, −3.4, −3.5, −3.6, −3.7, −3.8, 3.9, −4, −4.1, −4.2, −4.3, −4.4, −4.5, −4.6, −4.7, −4.8, −4.9, −5, −5.5, −6, −6.5, −7, −7.5, −8, −8.5, −9, −9.5, −10, −11, −12, −13, −14, −15, −16, −17, −18, −19, −20 or less, can be classified as a loss. For example, any CNV segment whose median log 2 ratio is less than −0.35 as determined by DNAcopy can be classified as a loss. For example, losses can be determined according to a threshold log 2 ratio, which can be set at −0.35. In another embodiment, losses can be determined according to a threshold log 2 ratio, which can be set at −0.5.

In some embodiments, two algorithms can be used to call or identify significant genetic variations, wherein any segment whose median log 2 ratio is less than or equal to −0.1, −0.11, −0.12, −0.13, −0.14, −0.15, −0.16, −0.17, −0.18, −0.19, −0.2, −0.21, −0.22, −0.23, −0.24, −0.25, −0.26, −0.27, −0.28, −0.29, −0.3, −0.31, −0.32, −0.33, −0.34, −0.35, −0.36, −0.37, −0.38, −0.39, −0.4, −0.41, −0.42, −0.43, −0.44, −0.45, −0.46, −0.47, −0.48, −0.49, −0.5, −0.55, −0.6, −0.65, −0.7, −0.75, −0.8, −0.85, −0.9, −0.95, −1, −1.1, −1.2, −1.3, −1.4, −1.5, −1.6, −1.7, −1.8, 1.9, −2, −2.1, −2.2, −2.3, −2.4, −2.5, −2.6, −2.7, −2.8, −2.9, −3, −3.1, −3.2, −3.3, −3.4, −3.5, −3.6, −3.7, −3.8, −3.9, 4, −4.1, −4.2, −4.3, −4.4, −4.5, −4.6, −4.7, −4.8, −4.9, −5, −5.5, −6, −6.5, −7, −7.5, −8, −8.5, −9, −9.5, −10, −11, −12, 13, −14, −15, −16, −17, −18, −19, −20 or less, as determined by one algorithm, and wherein any segment whose median log 2 ratio is less than or equal to −0.1, −0.11, −0.12, −0.13, −0.14, −0.15, −0.16, −0.17, −0.18, −0.19, 0.2, −0.21, −0.22, −0.23, −0.24, −0.25, −0.26, −0.27, −0.28, −0.29, −0.3, −0.31, −0.32, −0.33, −0.34, −0.35, −0.36, −0.37, −0.38, −0.39, −0.4, −0.41, −0.42, −0.43, −0.44, −0.45, −0.46, −0.47, −0.48, −0.49, −0.5, −0.55, −0.6, −0.65, 0.7, −0.75, −0.8, −0.85, −0.9, −0.95, −1, −1.1, −1.2, −1.3, −1.4, −1.5, −1.6, −1.7, −1.8, −1.9, −2, −2.1, −2.2, −2.3, −2.4, −2.5, −2.6, −2.7, −2.8, −2.9, −3, −3.1, −3.2, −3.3, −3.4, −3.5, −3.6, −3.7, −3.8, −3.9, −4, −4.1, −4.2, −4.3, −4.4, 4.5, −4.6, −4.7, −4.8, −4.9, −5, −5.5, −6, −6.5, −7, −7.5, −8, −8.5, −9, −9.5, −10, −11, −12, −13, −14, −15, −16, −17, 18, −19, −20, or less, as determined by the other algorithm can be classified as a loss. For example, CNV calling can comprise using the Aberration Detection Module 2 (ADM2) algorithm and the DNAcopy algorithm, wherein losses can be determined according to a two threshold log 2 ratios, wherein the Aberration Detection Module 2 (ADM2) algorithm log 2 ratio can be −0.25 and the DNAcopy algorithm log 2 ratio can be −0.41.

In some embodiments, the use of two algorithms to call or identify significant genetic variations can be a stringent method. In some embodiments, the use of two algorithms to call or identify significant genetic variations can be a more stringent method compared to the use of one algorithm to call or identify significant genetic variations.

In some embodiments, any CNV segment whose median log 2 ratio is greater than a log 2 ratio threshold value can be classified as a gain. For example, any segment whose median log 2 ratio is greater than 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or more can be classified as a gain.

In some embodiments, one algorithm can be used to call or identify significant genetic variations, wherein any segment whose median log 2 ratio is greater than or equal to 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or more can be classified as a gain. For example, any CNV segment whose median log 2 ratio is greater than 0.35 as determined by DNAcopy can be classified as a gain. For example, gains can be determined according to a threshold log 2 ratio, which can be set at 0.35. In another embodiment, gains can be determined according to a threshold log 2 ratio, which can be set at 0.5.

In some embodiments, two algorithms can be used to call or identify significant genetic variations, wherein any segment whose median log 2 ratio is greater than or equal to 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 or more, as determined by one algorithm, and wherein any segment whose median log 2 ratio is greater than or equal to 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or more, as determined by the other algorithm the can be classified as a gain. For example, CNV calling can comprise using the Aberration Detection Module 2 (ADM2) algorithm and the DNAcopy algorithm, wherein gains can be determined according to a two threshold log 2 ratios, wherein the Aberration Detection Module 2 (ADM2) algorithm log 2 ratio can be 0.25 and the DNAcopy algorithm log 2 ratio can be 0.32.

Any CNV segment whose absolute (median log-ratio/mad) value is less than 2 can be excluded (not identified as a significant genetic variation). For example, any CNV segment whose absolute (median log-ratio/mad) value is less than 2, 1.9, 1.8, 1.7, 1.6, 1.5, 1.4, 1.3, 1.2, 1.1, 1, 0.9, 0.8, 0.7, 0.6, or 0.5 or less can be excluded.

In some embodiments, multivariate analyses or joint risk analyses, including the use of multiplicative model for overall risk assessment, can subsequently be used to determine the overall risk conferred based on the genotype status at the multiple loci. Use of a multiplicative model, for example, assuming that the risk of individual risk variants multiply to establish the overall effect, allows fora straight forward calculation of the overall risk for multiple markers. The multiplicative model is a parsimonious model that usually fits the data of complex traits reasonably well. Deviations from multiplicity have been rarely described in the context of common variants for common diseases, and if reported are usually only suggestive since very large sample sizes can be required to be able to demonstrate statistical interactions between loci. Assessment of risk based on such analysis can subsequently be used in the methods, uses and kits of the disclosure, as described herein.

In some embodiments, the significance of increased or decreased susceptibility can be measured by a percentage. In some embodiments, a significant increased susceptibility can be measured as a relative susceptibility of at least 1.2, including but not limited to: at least 1.3, at least 1.4, at least 1.5, at least 1.6, at least 1.7, at least 1.8, at least 1.9, at least 2.0, at least 2.5, at least 3.0, at least 4.0, at least 5.0, at least 6.0, at least 7.0, at least 8.0, at least 9.0, at least 10.0, and at least 15.0. In some embodiments, a relative susceptibility of at least 2.0, at least 3.0, at least 4.0, at least, 5.0, at least 6.0, or at least 10.0 is significant. Other values for significant susceptibility are also contemplated, for example, at least 2.5, 3.5, 4.5, 5.5, or any suitable other numerical values, wherein the values are also within scope of the present disclosure. In some embodiments, a significant increase in susceptibility is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, 1000%, and 1500%. In one particular embodiment, a significant increase in susceptibility is at least 100%. In other embodiments, a significant increase in susceptibility is at least 200%, at least 300%, at least 400%, at least 500%, at least 700%, at least 800%, at least 900% and at least 1000%. Other cutoffs or ranges as deemed suitable by the person skilled in the art to characterize the disclosure are also contemplated, and those are also within scope of the present disclosure. In certain embodiments, a significant increase in susceptibility is characterized by a p-value, such as a p-value of less than 0.5, less than 0.4, less than 0.3, less than 0.2, less than 0.1, less than 0.05, less than 0.01, less than 0.001, less than 0.0001, less than 0.00001, less than 0.000001, less than 0.0000001, less than 0.00000001, or less than 0.000000001.

In some embodiments, an individual who is at a decreased susceptibility for or the lack of presence of a condition (e.g., LOAD) can be an individual in whom at least one genetic variation, conferring decreased susceptibility for or the lack of presence of the condition is identified. In some embodiments, the genetic variations conferring decreased susceptibility are also protective. In one aspect, the genetic variations can confer a significant decreased susceptibility of or lack of presence of LOAD.

In some embodiments, significant decreased susceptibility can be measured as a relative susceptibility of less than 0.9, including but not limited to less than 0.9, less than 0.8, less than 0.7, less than 0.6, less than 0.5, less than 0.4, less than 0.3, less than 0.2 and less than 0.1. In some embodiments, the decrease in susceptibility is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. Other cutoffs or ranges as deemed suitable by the person, skilled in the art to characterize the disclosure are however also contemplated, and those are also within scope of the present disclosure. In certain embodiments, a significant decrease in susceptibility is characterized by a p-value, such as a p-value of less than 0.05, less than 0.01, less than 0.001, less than 0.0001, less than 0.00001, less than 0.000001, less than 0.0000001, less than 0.00000001, or less than 0.000000001. Other tests for significance can be used, for example, a Fisher-exact test. Other statistical tests of significance known to the skilled person are also contemplated and are also within scope of the disclosure.

In some preferred embodiments, the significance of increased or decreased susceptibility can be determined according to the ratio of measurements from a test subject to a reference subject. In some embodiments, losses or gains of one or more CNVs can be determined according to a threshold log₂ ratio determined by these measurements. In some embodiments, a log₂ ratio value greater than 0.35, or 0.5, is indicative of a gain of one or more CNVs. In some embodiments, a logₑ ratio value less than −0.35, or −0.5, is indicative of a loss of one or more CNVs. In some embodiments, the ratio of measurements from a test subject to a reference subject may be inverted such that the log 2 ratios of copy number gains are negative and the log 2 ratios of copy number losses are positive.

In some embodiments, the combined or overall susceptibility associated with a plurality of variants associated with LOAD can also be assessed; for example, the genetic variations described herein to be associated with susceptibility to LOAD can be combined with other common genetic risk factors. Combined risk for such genetic variants can be estimated in an analogous fashion to the methods described herein.

Calculating risk conferred by a particular genotype for the individual can be based on comparing the genotype of the individual to previously determined risk expressed, for example, as a relative risk (RR) or an odds ratio (OR), for the genotype, for example, for a heterozygous carrier of an at-risk variant for LOAD. An odds ratio can be a statistical measure used as a metric of causality. For example, in genetic disease research it can be used to convey the significance of a variant in a disease cohort relative to an unaffected/normal cohort. The calculated risk for the individual can be the relative risk for a subject, or for a specific genotype of a subject, compared to the average population. The average population risk can be expressed as a weighted average of the risks of different genotypes, using results from a reference population, and the appropriate calculations to calculate the risk of a genotype group relative to the population can then be performed. Alternatively, the risk for an individual can be based on a comparison of particular genotypes, for example, heterozygous and/or homozygous carriers of an at-risk allele of a marker compared with non-carriers of the at-risk allele (or pair of alleles in the instance of compound heterozygous variants, wherein one variant impacts the maternally inherited allele and the other impacts the paternally inherited allele). Using the population average can, in certain embodiments, be more convenient, since it provides a measure that can be easy to interpret for the user, for example, a measure that gives the risk for the individual, based on his/her genotype, compared with the average in the population.

In some embodiments, the OR value can be calculated as follows: OR=(A/(N1−A))/(U/(N2−U)), where A=number of affected cases with variant, N1=total number of affected cases, U=number of unaffected cases with variant and N2=total number of unaffected cases. In circumstances where U=0, it is conventional to set U=1, so as to avoid infinities. In some preferred embodiments, the OR can be calculated essentially as above, except that where U or A=0, 0.5 is added to all of A, N1, U, N2. In another embodiment, a Fisher's Exact Test (FET) can be calculated using standard methods. In another embodiment, the p-values can be corrected for false discovery rate (FDR) using the Benjamini-Hochberg method (Benjamini Y. and Hochberg Y., J. Royal Statistical Society 57:289 (1995); Osborne J. A. and Barker C. A. (2007)).

In certain embodiments of the disclosure, a genetic variation is correlated to LOAD by referencing genetic variation data to a look-up table that comprises correlations between the genetic variation and LOAD. The genetic variation in certain embodiments comprises at least one indication of the genetic variation. In some embodiments, the table comprises a correlation for one genetic variation. In other embodiments, the table comprises a correlation for a plurality of genetic variations in both scenarios, by referencing to a look-up table that gives an indication of a correlation between a genetic variation and LOAD, a risk for LOAD, or a susceptibility to LOAD, can be identified in the individual from whom the nucleic acid sample is derived.

The present disclosure also pertains to methods of clinical screening, for example, diagnosis, prognosis, or theranosis of a subject performed by a medical professional using the methods disclosed herein. In other embodiments, the disclosure pertains to methods of screening performed by a layman. The layman can be a customer of a genotyping, microarray, exome sequencing, or whole genome sequencing service provider. The layman can also be a genotype, microarray, exome sequencing, or whole genome sequencing service provider, who performs genetic analysis on a DNA sample from an individual, in order to provide service related to genetic risk factors for particular traits or diseases, based on the genotype status of the subject obtained from use of the methods described herein. The resulting genotype or genetic information can be made available to the individual and can be compared to information about LOAD or risk of developing LOAD associated with one or various genetic variations, including but not limited to, information from public or private genetic variation databases or literature and scientific publications. The screening applications of LOAD-associated genetic variations, as described herein, can, for example, be performed by an individual, a health professional, or a third party, for example a service provider who interprets genotype information from the subject. In some embodiments the genetic analysis is performed in a CLIA-certified laboratory (i.e. the federal regulatory standards of the U.S. that are specified in the Clinical Laboratory Improvement Amendments, administered by the Centers for Medicare and Medicaid Services) or equivalent laboratories in Europe and elsewhere in the world.

The information derived from analyzing sequence data can be communicated to any particular body, including the individual from which the nucleic acid sample or sequence data is derived, a guardian or representative of the individual, clinician, research professional, medical professional, service provider, and medical insurer or insurance company. Medical professionals can be, for example, doctors, nurses, medical laboratory technologists, and pharmacists. Research professionals can be, for example, principle investigators, research technicians, postdoctoral trainees, and graduate students.

In some embodiments, a professional can be assisted by determining whether specific genetic variants are present in a nucleic acid sample from a subject, and communicating information about genetic variants to a professional. After information about specific genetic variants is reported, a medical professional can take one or more actions that can affect subject care. For example, a medical professional can record information in the subject's medical record (e.g., electronic health record or electronic medical record, including, but not limited to, country-scale health services such as the National Health Service in the United Kingdom) regarding the subject's risk of developing LOAD. In some embodiments, a medical professional can record information regarding risk assessment, or otherwise transform the subject's medical record, to reflect the subject's current medical condition. In some embodiments, a medical professional can review and evaluate a subject's entire medical record and assess multiple treatment strategies for clinical intervention of a subject's condition. In another embodiment, information can be recorded in the context of the system developed by the World Health Organization (WHO), the International Statistical Classification of Diseases and Related Health Problems (ICD), which is currently using the 10th revision (ICD-10 codes). For example, the ICD-10 code for LOAD is A81.2, whereas the ICD-10 code for multiple sclerosis is G35.

A medical professional can initiate or modify treatment after receiving information regarding a subject's screening for LOAD, for example. In some embodiments, a medical professional can recommend a change in therapy or exclude a therapy. In some embodiments, a medical professional can enroll a subject in a clinical trial for, by way of example, detecting correlations between a haplotype as described herein and any measurable or quantifiable parameter relating to the outcome of the treatment as described above.

In some embodiments, a medical professional can communicate information regarding a subject's screening of developing LOAD to a subject or a subject's family. In some embodiments, a medical professional can provide a subject and/or a subject's family with information regarding LOAD and risk assessment information, including treatment options, and referrals to specialists. In some embodiments, a medical professional can provide a copy of a subject's medical records to a specialist. In some embodiments, a research professional can apply information regarding a subject's risk of developing LOAD to advance scientific research. In some embodiments, a research professional can obtain a subject's haplotype as described herein to evaluate a subject's enrollment, or continued participation, in a research study or clinical trial. In some embodiments, a research professional can communicate information regarding a subject's screening of LOAD to a medical professional. In some embodiments, a research professional can refer a subject to a medical professional.

Any appropriate method can be used to communicate information to another person. For example, information can be given directly or indirectly to a professional and a laboratory technician can input a subject's genetic variation as described herein into a computer-based record. In some embodiments, information is communicated by making a physical alteration to medical or research records. For example, a medical professional can make a permanent notation or flag a medical record for communicating the risk assessment to other medical professionals reviewing the record. In addition, any type of communication can be used to communicate the risk assessment information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

Results of these tests, and optionally interpretive information, can be returned to the subject, the health care provider or to a third party. The results can be communicated to the tested subject, for example, with a prognosis and optionally interpretive materials that can help the subject understand the test results and prognosis; used by a health care provider, for example, to determine whether to administer a specific drug, or whether a subject should be assigned to a specific category, for example, a category associated with a specific disease endophenotype, or with drug response or non-response; used by a third party such as a healthcare payer, for example, an insurance company or HMO, or other agency, to determine whether or not to reimburse a health care provider for services to the subject, or whether to approve the provision of services to the subject. For example, the healthcare payer can decide to reimburse a health care provider for treatments for LOAD if the subject has LOAD or has an increased risk of developing LOAD.

Also provided herein are databases that include a list of genetic variations as described herein, and wherein the list can be largely or entirely limited to genetic variations identified as useful for screening LOAD as described herein. The list can be stored, for example, on a flat file or computer-readable medium. The databases can further include information regarding one or more subjects, for example, whether a subject is affected or unaffected, clinical information such as endophenotype, age of onset of symptoms, any treatments administered and outcomes, for example, data relevant to pharmacogenomics, diagnostics, prognostics or theranostics, and other details, for example, data about the disorder in the subject, or environmental or other genetic factors. The databases can be used to detect correlations between a particular haplotype and the information regarding the subject.

The methods described herein can also include the generation of reports for use, for example, by a subject, care giver, or researcher, that include information regarding a subject's genetic variations, and optionally further information such as treatments administered, treatment history, medical history, predicted response, and actual response. The reports can be recorded in a tangible medium, e.g., a computer-readable disk, a solid state memory device, or an optical storage device.

Methods of Screening Using Variations in RNA and/or Polypeptides

In some embodiments of the disclosure, screening of LOAD can be made by examining or comparing changes in expression, localization, binding partners, and composition of a polypeptide encoded by a nucleic acid variant associated with LOAD, for example, in those instances where the genetic variations of the present disclosure results in a change in the composition or expression of the polypeptide and/or RNA, for example, mRNAs, microRNAs (miRNAs), and other noncoding RNAs (ncRNAs). Thus, screening of LOAD can be made by examining expression and/or composition of one of these polypeptides and/or RNA, or another polypeptide and/or RNA encoded by a nucleic acid associated with LOAD, in those instances where the genetic variation of the present disclosure results in a change in the expression, localization, binding partners, and/or composition of the polypeptide and/or RNA. In some embodiments, screening can comprise diagnosing a subject. In some embodiments, screening can comprise determining a prognosis of a subject, for example determining the susceptibility of developing LOAD. In some embodiments, screening can comprise theranosing a subject.

The genetic variations described herein that show association to LOAD can play a role through their effect on one or more of these genes, either by directly impacting one or more genes or influencing the expression of one or more nearby genes. For example, while not intending to be limited by theory, it is generally expected that a deletion of a chromosomal segment comprising a particular gene, or a fragment of a gene, can either result in an altered composition or expression, or both, of the encoded polypeptide and/or mRNA. Likewise, duplications, or high number copy number variations, are in general expected to result in increased expression of encoded polypeptide and/or RNA if the gene they are expressed from is fully encompassed within the duplicated (or triplicated, or even higher copy number gains) genomic segment, or conversely can result in decreased expression or a disrupted RNA or polypeptide if one or both breakpoints of the copy number gain disrupt a given gene. Other possible mechanisms affecting genes within a genetic variation region include, for example, effects on transcription, effects on RNA splicing, alterations in relative amounts of alternative splice forms of mRNA, effects on RNA stability, effects on transport from the nucleus to cytoplasm, and effects on the efficiency and accuracy of translation. Thus, DNA variations can be detected directly, using the subjects unamplified or amplified genomic DNA, or indirectly, using RNA or DNA obtained from the subject's tissue(s) that are present in an aberrant form or expression level as a result of the genetic variations of the disclosure showing association to LOAD. In another embodiment, DNA variations can be detected indirectly using a polypeptide or protein obtained from the subject's tissue(s) that is present in an aberrant form or expression level as a result of genetic variations of the disclosure showing association to the LOAD. In another embodiment, an aberrant form or expression level of a polypeptide or protein that results from one or more genetic variations of the disclosure showing association to LOAD can be detected indirectly via another polypeptide or protein present in the same biological/cellular pathway that is modulated or interacts with said polypeptide or protein that results from one or more genetic variations of the disclosure. In some embodiments, the genetic variations of the disclosure showing association to LOAD can affect the expression of a gene within the genetic variation region. In some embodiments, a genetic variation affecting an exonic region of a gene can affect, disrupt, or modulate the expression of the gene. In some embodiments, a genetic variation affecting an intronic or intergenic region of a gene can affect, disrupt, or modulate the expression of the gene.

Certain genetic variation regions can have flanking duplicated segments, and genes within such segments can have altered expression and/or composition as a result of such genomic alterations. Regulatory elements affecting gene expression can be located far away, even as far as tens or hundreds of kilobases away, from the gene that is regulated by said regulatory elements. Thus, in some embodiments, regulatory elements for genes that are located outside the gene (e.g., upstream or downstream of the gene) can be located within the genetic variation, and thus be affected by the genetic variation. It is thus contemplated that the detection of the genetic variations described herein, can be used for assessing expression for one or more of associated genes not directly impacted by the genetic variations. In some embodiments, a genetic variation affecting an intergenic region of a gene can affect, disrupt, or modulate the expression of a gene located elsewhere in the genome, such as described above. For example, a genetic variation affecting an intergenic region of a gene can affect, disrupt, or modulate the expression of a transcription factor, located elsewhere in the genome, which regulates the gene. Regulatory elements can also be located within a gene, such as within intronic regions, and similarly impact the expression level of the gene and ultimately the protein expression level without changing the structure of the protein. The effects of genetic variants on regulatory elements can manifest in a tissue-specific manner; for example, one or more transcription factors that bind to the regulatory element that is impacted by one or more genetic variations may be expressed at higher concentration in neurons as compared to skin cells (i.e., the impact of the one or more genetic variations may be primarily evident in neuronal cells).

In some embodiments, genetic variations of the disclosure showing association to LOAD can affect protein expression at the translational level. It can be appreciated by those skilled in the art that this can occur by increased or decreased expression of one or more microRNAs (miRNAs) that regulates expression of a protein known to be important, or implicated, in the cause, onset, or progression of LOAD. Increased or decreased expression of the one or more miRNAs can result from gain or loss of the whole miRNA gene, disruption or impairment of a portion of the gene (e.g., by an indel or CNV), or even a single base change (SNP or SNV) that produces an altered, non-functional or aberrant functioning miRNA sequence. It can also be appreciated by those skilled in the art that the expression of protein, for example, one known to cause LOAD by increased or decreased expression, can result due to a genetic variation that results in alteration of an existing miRNA binding site within the polypeptide's mRNA transcript, or even creates a new miRNA binding site that leads to aberrant polypeptide expression.

A variety of methods can be used for detecting polypeptide composition and/or expression levels, including but not limited to enzyme linked immunosorbent assays (ELISA), Western blots, spectroscopy, mass spectrometry, peptide arrays, colorimetry, electrophoresis, isoelectric focusing, immunoprecipitations, immunoassays, and immunofluorescence and other methods well-known in the art. A test nucleic acid sample from a subject can be assessed for the presence of an alteration in the expression and/or an alteration in composition of the polypeptide encoded by a nucleic acid associated with LOAD. An “alteration” in the polypeptide expression or composition, as used herein, refers to an alteration in expression or composition in a test nucleic acid sample, as compared to the expression or composition of the polypeptide in a control nucleic acid sample. Such alteration can, for example, be an alteration in the quantitative polypeptide expression or can be an alteration in the qualitative polypeptide expression, for example, expression of a mutant polypeptide or of a different splicing variant, or a combination thereof. In some embodiments, screening of LOAD can be made by detecting a particular splicing variant encoded by a nucleic acid associated with LOAD, or a particular pattern of splicing variants.

Antibodies can be polyclonal or monoclonal and can be labeled or unlabeled. An intact antibody or a fragment thereof can be used. The term “labeled”, with regard to the probe or antibody, is intended to encompass direct labeling of the probe or antibody by coupling a detectable substance to the probe or antibody, as well as indirect labeling of the probe or antibody by reactivity with another reagent that is directly labeled as previously described herein. Other non-limiting examples of indirect labeling include detection of a primary antibody using a labeled secondary antibody, for example, a fluorescently-labeled secondary antibody and end-labeling of a DNA probe with biotin such that it can be detected with fluorescently-labeled streptavidin.

### Methods of Detecting Genetic Variations

In some embodiments, standard techniques for genotyping for the presence genetic variations, for example, amplification, can be used. Amplification of nucleic acids can be accomplished using methods known in the art. Generally, sequence information from the region of interest can be used to design oligonucleotide primers that can be identical or similar in sequence to opposite strands of a template to be amplified. In some embodiments, amplification methods can include but are not limited to, fluorescence-based techniques utilizing PCR, for example, ligase chain reaction (LCR), Nested PCR, transcription amplification, self-sustained sequence replication, nucleic acid based sequence amplification (NASBA), and multiplex ligation-dependent probe amplification (MLPA). Guidelines for selecting primers for PCR amplification are well known in the art. In some embodiments, a computer program can be used to design primers, for example, Oligo (National Biosciences, Inc, Plymouth Minn), MacVector (Kodak/IBI), and GCG suite of sequence analysis programs.

In some embodiments, commercial methodologies available for genotyping, for example, SNP genotyping, can be used, but are not limited to, TaqMan genotyping assays (Applied Biosystems), SNPlex platforms (Applied Biosystems), gel electrophoresis, capillary electrophoresis, size exclusion chromatography, mass spectrometry, for example, MassARRAY system (Sequenom), minisequencing methods, real-time Polymerase Chain Reaction (PCR), Bio-Plex system (BioRad), CEQ and SNPstream systems (Beckman), array hybridization technology, for example, Affymetrix GeneChip (Perlegen), BeadArray Technologies, for example, Illumina GoldenGate and Infinium assays, array tag technology, Multiplex Ligation-dependent Probe Amplification (MLPA), and endonuclease-based fluorescence hybridization technology (Invader assay, either using unamplified or amplified genomic DNA, or unamplified total RNA, or unamplified or amplified cDNA; Third Wave/Hologic). PCR can be a procedure in which target nucleic acid is amplified in a manner similar to that described in U.S. Pat. No. 4,683,195 and subsequent modifications of the procedure described therein. PCR can include a three phase temperature cycle of denaturation of DNA into single strands, annealing of primers to the denatured strands, and extension of the primers by a thermostable DNA polymerase enzyme. This cycle can be repeated so that there are enough copies to be detected and analyzed. In some embodiments, real-time quantitative PCR can be used to determine genetic variations, wherein quantitative PCR can permit both detection and quantification of a DNA sequence in a nucleic acid sample, for example, as an absolute number of copies or as a relative amount when normalized to DNA input or other normalizing genes. In some embodiments, methods of quantification can include the use of fluorescent dyes that can intercalate with double-stranded DNA, and modified DNA oligonucleotide probes that can fluoresce when hybridized with a complementary DNA.

In some embodiments of the disclosure, a nucleic acid sample obtained from the subject can be collected and PCR can be used to amplify a fragment of nucleic acid that comprises one or more genetic variations that can be indicative of a susceptibility to LOAD. In some embodiments, detection of genetic variations can be accomplished by expression analysis, for example, by using quantitative PCR. In some embodiments, this technique can assess the presence or absence of a genetic alteration in the expression or composition of one or more polypeptides or splicing variants encoded by a nucleic acid associated with LOAD.

In some embodiments, the nucleic acid sample from a subject containing a SNP can be amplified by PCR prior to detection with a probe. In such an embodiment, the amplified DNA serves as the template for a detection probe and, in some embodiments, an enhancer probe. Certain embodiments of the detection probe, the enhancer probe, and/or the primers used for amplification of the template by PCR can comprise the use of modified bases, for example, modified A, T, C, G, and U, wherein the use of modified bases can be useful for adjusting the melting temperature of the nucleotide probe and/or primer to the template DNA, In some embodiments, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In some embodiments, identification of genetic variations can be accomplished using hybridization methods. The presence of a specific marker allele or a particular genomic segment comprising a genetic variation, or representative of a genetic variation, can be indicated by sequence-specific hybridization of a nucleic acid probe specific for the particular allele or the genetic variation in a nucleic acid sample that has or has not been amplified but methods described herein. The presence of more than one specific marker allele or several genetic variations can be indicated by using two or more sequence-specific nucleic acid probes, wherein each is specific for a particular allele and/or genetic variation.

Hybridization can be performed by methods well known to the person skilled in the art, for example, hybridization techniques such as fluorescent in situ hybridization (FISH), Southern analysis, Northern analysis, or in situ hybridization. In some embodiments, hybridization refers to specific hybridization, wherein hybridization can be performed with no mismatches. Specific hybridization, if present, can be using standard methods. In some embodiments, if specific hybridization occurs between a nucleic acid probe and the nucleic acid in the nucleic acid sample, the nucleic acid sample can contain a sequence that can be complementary to a nucleotide present in the nucleic acid probe. In some embodiments, if a nucleic acid probe can contain a particular allele of a polymorphic marker, or particular alleles for a plurality of markers, specific hybridization is indicative of the nucleic acid being completely complementary to the nucleic acid probe, including the particular alleles at polymorphic markers within the probe. In some embodiments a probe can contain more than one marker alleles of a particular haplotype, for example, a probe can contain alleles complementary to 2, 3, 4, 5 or all of the markers that make up a particular haplotype. In some embodiments detection of one or more particular markers of the haplotype in the nucleic acid sample is indicative that the source of the nucleic acid sample has the particular haplotype.

In some embodiments, PCR conditions and primers can be developed that amplify a product only when the variant allele is present or only when the wild type allele is present, for example, allele-specific PCR. In some embodiments of allele-specific PCR, a method utilizing a detection oligonucleotide probe comprising a fluorescent moiety or group at its 3′ terminus and a quencher at its 5′ terminus, and an enhancer oligonucleotide, can be employed (see e.g., Kutyavin et al., Nucleic Acid Res. 34:e 128 (2006)).

An allele-specific primer/probe can be an oligonucleotide that is specific for particular a polymorphism can be prepared using standard methods. In some embodiments, allele-specific oligonucleotide probes can specifically hybridize to a nucleic acid region that contains a genetic variation. In some embodiments, hybridization conditions can be selected such that a nucleic acid probe can specifically bind to the sequence of interest, for example, the variant nucleic acid sequence.

In some embodiments, allele-specific restriction digest analysis can be used to detect the existence of a polymorphic variant of a polymorphism, if alternate polymorphic variants of the polymorphism can result in the creation or elimination of a restriction site. Allele-specific restriction digests can be performed, for example, with the particular restriction enzyme that can differentiate the alleles. In some embodiments, PCR can be used to amplify a region comprising the polymorphic site, and restriction fragment length polymorphism analysis can be conducted. In some embodiments, for sequence variants that do not alter a common restriction site, mutagenic primers can be designed that can introduce one or more restriction sites when the variant allele is present or when the wild type allele is present.

In some embodiments, fluorescence polarization template-directed dye-terminator incorporation (FP-TDI) can be used to determine which of multiple polymorphic variants of a polymorphism can be present in a subject. Unlike the use of allele-specific probes or primers, this method can employ primers that can terminate adjacent to a polymorphic site, so that extension of the primer by a single nucleotide can result in incorporation of a nucleotide complementary to the polymorphic variant at the polymorphic site.

In some embodiments, DNA containing an amplified portion can be dot-blotted, using standard methods and the blot contacted with the oligonucleotide probe. The presence of specific hybridization of the probe to the DNA can then be detected. The methods can include determining the genotype of a subject with respect to both copies of the polymorphic site present in the genome, wherein if multiple polymorphic variants exist at a site, this can be appropriately indicated by specifying which variants are present in a subject. Any of the detection means described herein can be used to determine the genotype of a subject with respect to one or both copies of the polymorphism present in the subject's genome.

In some embodiments, a peptide nucleic acid (PNA) probe can be used in addition to, or instead of, a nucleic acid probe in the methods described herein. A PNA can be a DNA mimic having a peptide-like, inorganic backbone, for example, N-(2-aminoethyl) glycine units with an organic base (A, G, C, T or U) attached to the glycine nitrogen via a methylene carbonyl linker.

Nucleic acid sequence analysis can also be used to detect genetic variations, for example, genetic variations can be detected by sequencing exons, introns, 5′ untranslated sequences, or 3′ untranslated sequences. One or more methods of nucleic acid analysis that are available to those skilled in the art can be used to detect genetic variations, including but not limited to, direct manual sequencing, automated fluorescent sequencing, single-stranded conformation polymorphism assays (SSCP); clamped denaturing gel electrophoresis (CDGE); denaturing gradient gel electrophoresis (DGGE), two-dimensional gel electrophoresis (2DGE or TDGE); conformational sensitive gel electrophoresis (CSGE); denaturing high performance liquid chromatography (DHPLC), infrared matrix-assisted laser desorption/ionization (IR-MALDI) mass spectrometry, mobility shift analysis, quantitative real-time PCR, restriction enzyme analysis, heteroduplex analysis; chemical mismatch cleavage (CMC), RNase protection assays, use of polypeptides that recognize nucleotide mismatches, allele-specific PCR, real-time pyrophosphate DNA sequencing, PCR amplification in combination with denaturing high performance liquid chromatography (dHPLC), and combinations of such methods.

Sequencing can be accomplished through classic Sanger sequencing methods, which are known in the art. In some embodiments sequencing can be performed using high-throughput sequencing methods some of which allow detection of a sequenced nucleotide immediately after or upon its incorporation into a growing strand, for example, detection of sequence in substantially real time or real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read (or 500-1,000 bases per read for 454).

High-throughput sequencing methods can include but are not limited to, Massively Parallel Signature Sequencing (MPSS, Lynx Therapeutics), Polony sequencing, 454 pyrosequencing, Illumina (Solexa) sequencing, Illumina (Solexa) sequencing using 10× Genomics library preparation, SOLID sequencing, on semiconductor sequencing, DNA nanoball sequencing, Helioscope™ single molecule sequencing, Single Molecule SMRT™ sequencing, Single Molecule real time (RNAP) sequencing, Nanopore DNA sequencing, and/or sequencing by hybridization, for example, a non-enzymatic method that uses a DNA microarray, or microfluidic Sanger sequencing.

In some embodiments, high-throughput sequencing can involve the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. This fast sequencing method also allows for detection of a SNP/nucleotide in a sequence in substantially real time or real time. Finally, SMSS is powerful because, like the MIP technology, it does not use a pre-amplification step prior to hybridization. SMSS does not use any amplification. SMSS is described in US Publication Application Nos. 20060024711; 20060024678; 20060012793; 20060012784; and 20050100932. In some embodiments, high-throughput sequencing involves the use of technology available by 454 Life Sciences, Inc. (a Roche company, Branford, Conn.) such as the PicoTiterPlate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

In some embodiments, PCR-amplified single-strand nucleic acid can be hybridized to a primer and incubated with a polymerase, ATP sulfurylase, luciferase, apyrase, and the substrates luciferin and adenosine 5′ phosphosulfate. Next, deoxynucleotide triphosphates corresponding to the bases A, C, G, and T (U) can be added sequentially. A base incorporation can be accompanied by release of pyrophosphate, which can be converted to ATP by sulfurylase, which can drive synthesis of oxyluciferin and the release of visible light. Since pyrophosphate release can be equimolar with the number of incorporated bases, the light given off can be proportional to the number of nucleotides adding in any one step. The process can repeat until the entire sequence can be determined. In some embodiments, pyrosequencing can be utilized to analyze amplicons to determine whether breakpoints are present. In some embodiments, pyrosequencing can map surrounding sequences as an internal quality control.

Pyrosequencing analysis methods are known in the art. Sequence analysis can include a four-color sequencing by ligation scheme (degenerate ligation), which involves hybridizing an anchor primer to one of four positions. Then an enzymatic ligation reaction of the anchor primer to a population of degenerate nonamers that are labeled with fluorescent dyes can be performed. At any given cycle, the population of nonamers that is used can be structured such that the identity of one of its positions can be correlated with the identity of the fluorophore attached to that nonamer. To the extent that the ligase discriminates for complementarily at that queried position, the fluorescent signal can allow the inference of the identity of the base. After performing the ligation and four-color imaging, the anchor primer: nonamer complexes can be stripped and a new cycle begins. Methods to image sequence information after performing ligation are known in the art.

In some embodiments, analysis by restriction enzyme digestion can be used to detect a particular genetic variation if the genetic variation results in creation or elimination of one or more restriction sites relative to a reference sequence. In some embodiments, restriction fragment length polymorphism (RFLP) analysis can be conducted, wherein the digestion pattern of the relevant DNA fragment indicates the presence or absence of the particular genetic variation in the nucleic acid sample.

In some embodiments, arrays of oligonucleotide probes that can be complementary to target nucleic acid sequence segments from a subject can be used to identify genetic variations. In some embodiments, an array of oligonucleotide probes comprises an oligonucleotide array, for example, a microarray. In some embodiments, the present disclosure features arrays that include a substrate having a plurality of addressable areas, and methods of using them. At least one area of the plurality includes a nucleic acid probe that binds specifically to a sequence comprising a genetic variation, and can be used to detect the absence or presence of the genetic variation, for example, one or more SNPs, microsatellites, or CNVs, as described herein, to determine or identify an allele or genotype. For example, the array can include one or more nucleic acid probes that can be used to detect a genetic variation associated with a gene and/or gene product. In some embodiments, the array can further comprise at least one area that includes a nucleic acid probe that can be used to specifically detect another marker associated with LOAD as described herein.

Microarray hybridization can be performed by hybridizing a nucleic acid of interest, for example, a nucleic acid encompassing a genetic variation, with the array and detecting hybridization using nucleic acid probes. In some embodiments, the nucleic acid of interest is amplified prior to hybridization. Hybridization and detecting can be carried out according to standard methods described in Published PCT Applications: WO 92/10092 and WO 95/11995, and U.S. Pat. No. 5,424,186. For example, an array can be scanned to determine the position on the array to which the nucleic acid hybridizes. The hybridization data obtained from the scan can be, for example, in the form of fluorescence intensities as a function of location on the array.

Arrays can be formed on substrates fabricated with materials such as paper; glass; plastic, for example, polypropylene, nylon, or polystyrene; polyacrylamide; nitrocellulose; silicon; optical fiber; or any other suitable solid or semisolid support; and can be configured in a planar, for example, glass plates or silicon chips); or three dimensional, for example, pins, fibers, beads, particles, microtiter wells, and capillaries, configuration.

Methods for generating arrays are known in the art and can include for example; photolithographic methods (U.S. Pat. Nos. 5,143,854, 5,510,270 and 5,527,681); mechanical methods, for example, directed-flow methods (U.S. Pat. No. 5,384,261); pin-based methods (U.S. Pat. No. 5; 288; 514); bead-based techniques (PCT US/93/04145); solid phase oligonucleotide synthesis methods; or by other methods known to a person skilled in the art (see, e.g., Bier, F. F., et al., Adv Biochem Eng Biotechnol 109:433-53 (2008); Hoheisel, J. D., Nat Rev Genet 7: 200-10 (2006); Fan, J. B., et al., Methods Enzymol 410:57-73 (2006); Raqoussis, J. & Elvidge, G., Expert Rev Mol Design 6: 145-52 (2006); Mockler, T. C., et al., Genomics 85: 1-15 (2005), and references cited therein, the entire teachings of each of which are incorporated by reference herein). Many additional descriptions of the preparation and use of oligonucleotide arrays for detection of polymorphisms can be found, for example, in U.S. Pat. Nos. 6,858,394, 6,429,027, 5,445,934, 5,700,637, 5,744,305, 5,945,334, 6,054,270, 6,300,063, 6,733,977, 7,364,858, EP 619 321, and EP 373 203, the entire teachings of which are incorporated by reference herein. Methods for array production, hybridization, and analysis are also described in Snijders et al., Nat. Genetics 29:263-264 (2001); Klein et al., Proc. Natl. Acad. Sci. USA 96:4494-4499 (1999); Albertson et al., Breast Cancer Research and Treatment 78:289-298 (2003); and Snijders et al., “BAC microarray based comparative genomic hybridization,” in: Zhao et al., (eds), Bacterial Artificial Chromosomes: Methods and Protocols, Methods in Molecular Biology, Humana Press (2002).

In some embodiments, oligonucleotide probes forming an array can be attached to a substrate by any number of techniques, including, but not limited to, in situ synthesis, for example, high-density oligonucleotide arrays, using photolithographic techniques; spotting/printing a medium to low density on glass, nylon, or nitrocellulose; by masking; and by dot-blotting on a nylon or nitrocellulose hybridization membrane. In some embodiments, oligonucleotides can be immobilized via a linker, including but not limited to, by covalent, ionic, or physical linkage. Linkers for immobilizing nucleic acids and polypeptides, including reversible or cleavable linkers, are known in the art (U.S. Pat. No. 5,451,683 and WO98/20019). In some embodiments, oligonucleotides can be non-covalently immobilized on a substrate by hybridization to anchors, by means of magnetic beads, or in a fluid phase, for example, in wells or capillaries.

An array can comprise oligonucleotide hybridization probes capable of specifically hybridizing to different genetic variations. In some embodiments, oligonucleotide arrays can comprise a plurality of different oligonucleotide probes coupled to a surface of a substrate in different known locations. In some embodiments, oligonucleotide probes can exhibit differential or selective binding to polymorphic sites, and can be readily designed by one of ordinary skill in the art, for example, an oligonucleotide that is perfectly complementary to a sequence that encompasses a polymorphic site, for example, a sequence that includes the polymorphic site, within it, or at one end, can hybridize preferentially to a nucleic acid comprising that sequence, as opposed to a nucleic acid comprising an alternate polymorphic variant.

In some embodiments, arrays can include multiple detection blocks, for example, multiple groups of probes designed for detection of particular polymorphisms. In some embodiments, these arrays can be used to analyze multiple different polymorphisms. In some embodiments, detection blocks can be grouped within a single array or in multiple, separate arrays, wherein varying conditions, for example, conditions optimized for particular polymorphisms, can be used during hybridization. General descriptions of using oligonucleotide arrays for detection of polymorphisms can be found, for example, in U.S. Pat. Nos. 5,858,659 and 5,837,832. In addition to oligonucleotide arrays, cDNA arrays can be used similarly in certain embodiments.

The methods described herein can include but are not limited to providing an array as described herein; contacting the array with a nucleic acid sample, and detecting binding of a nucleic acid from the nucleic acid sample to the array. In some embodiments, the method can comprise amplifying nucleic acid from the nucleic acid sample, for example, a region associated with LOAD or a region that includes another region associated with LOAD. In some embodiments, the methods described herein can include using an array that can identify differential expression patterns or copy numbers of one or more genes in nucleic acid samples from control and affected individuals. For example, arrays of probes to a marker described herein can be used to identify genetic variations between DNA from an affected subject, and control DNA obtained from an individual that does not have LOAD. Since the nucleotides on the array can contain sequence tags, their positions on the array can be accurately known relative to the genomic sequence.

In some embodiments, it can be desirable to employ methods that can detect the presence of multiple genetic variations, for example, polymorphic variants at a plurality of polymorphic sites, in parallel or substantially simultaneously. In some embodiments, these methods can comprise oligonucleotide arrays and other methods, including methods in which reactions, for example, amplification and hybridization, can be performed in individual vessels, for example, within individual wells of a multi-well plate or other vessel.

Determining the identity of a genetic variation can also include or consist of reviewing a subject's medical history, where the medical history includes information regarding the identity, copy number, presence or absence of one or more alleles or SNPs in the subject, e.g., results of a genetic test.

In some embodiments extended runs of homozygosity (ROH) may be useful to map recessive disease genes in outbred populations. Furthermore, even in complex disorders, a high number of affected individuals may have the same haplotype in the region surrounding a disease mutation. Therefore, a rare pathogenic variant and surrounding haplotype can be enriched in frequency in a group of affected individuals compared with the haplotype frequency in a cohort of unaffected controls. Homozygous haplotypes (HH) that are shared by multiple affected individuals can be important for the discovery of recessive disease genes in a condition such as LOAD. In some embodiments, the traditional homozygosity mapping method can be extended by analyzing the haplotype within shared ROH regions to identify homozygous segments of identical haplotype that are present uniquely or at a higher frequency in LOAD probands compared to parental controls. Such regions are termed risk homozygous haplotypes (rHH), which may contain low-frequency recessive variants that contribute to LOAD risk in a subset of LOAD patients.

Genetic variations can also be identified using any of a number of methods well known in the art. For example, genetic variations available in public databases, which can be searched using methods and custom algorithms or algorithms known in the art, can be used. In some embodiments, a reference sequence can be from, for example, the human draft genome sequence, publicly available in various databases, or a sequence deposited in a database such as GenBank.

A comparison of one or more genomes relative to one or more other genomes with array CGH, or a variety of other genetic variation detection methods, can reveal the set of genetic variations between two genomes, between one genome in comparison to multiple genomes, or between one set of genomes in comparison to another set of genomes. In some embodiments, an array CGH experiment can be performed by hybridizing a single test genome against a pooled nucleic acid sample of two or more genomes, which can result in minimizing the detection of higher frequency variants in the experiment. In some embodiments, a test genome can be hybridized alone (i.e., one-color detection) to a microarray, for example, using array CGH or SNP genotyping methods, and the comparison step to one or more reference genomes can be performed in silico to reveal the set of genetic variations in the test genome relative to the one or more reference genomes. In one embodiment, a single test genome is compared to a single reference genome in a 2-color experiment wherein both genomes are cohybridized to the microarray. In some embodiments, the whole genome or whole exome from one or more subjects is analyzed. In some embodiments, nucleic acid information has already been obtained for the whole genome or whole exome from one or more individuals and the nucleic acid information is obtained from in silico analysis.

Any of the polynucleotides described, including polynucleotides comprising a genetic variation, can be made synthetically using methods known in the art.

### Methods of Detecting CNVs

Detection of genetic variations, specifically CNVs, can be accomplished by one or more suitable techniques described herein. Generally, techniques that can selectively determine whether a particular chromosomal segment is present or absent in an individual can be used for genotyping CNVs. Identification of novel copy number variations can be done by methods for assessing genomic copy number changes.

In some embodiments, methods include but are not limited to, methods that can quantitatively estimate the number of copies of a particular genomic segment, but can also include methods that indicate whether a particular segment is present in a nucleic acid sample or not. In some embodiments, the technique to be used can quantify the amount of segment present, for example, determining whether a DNA segment is deleted, duplicated, or triplicated in subject, for example, Fluorescent In Situ Hybridization (FISH) techniques, and other methods described herein. In some embodiments, methods include detection of copy number variation from array intensity and sequencing read depth using a stepwise Bayesian model (Zhang, et al., BMC Bioinformatics, 11:539 (2010)). In some embodiments, methods include detecting copy number variations using shotgun sequencing, CNV-seq (Xie C., et al., BMC Bioinformatics, 10:80 (2009)). In some embodiments, methods include analyzing next-generation sequencing (NGS) data for CNV detection using any one of several algorithms developed for each of the four broad methods for CNV detection using NGS, namely the depth of coverage (DOC), read-pair (RP), split-read (SR) and assembly-based (AS) methods. (Teo et al., Bioinformatics (2012)). In some embodiments, methods include combining coverage with map information for the identification of deletions and duplications in targeted sequence data (Nord et al., BMC Genomics, 12:184 (2011)).

In some embodiments, other genotyping technologies can be used for detection of CNVs, including but not limited to, karyotype analysis, Molecular Inversion Probe array technology, for example, Affymetrix SNP Array 6.0, and BeadArray Technologies, for example, Illumina GoldenGate and Infinium assays, as can other platforms such as NimbleGen HD2.1 or HD4.2, High-Definition Comparative Genomic Hybridization (CGH) arrays (Agilent Technologies), tiling array technology (Affymetrix), multiplex ligation-dependent probe amplification (MLPA), Invader assay, fluorescence in situ hybridization, and, in one embodiment, Array Comparative Genomic Hybridization (aCGH) methods. As described herein, karyotype analysis can be a method to determine the content and structure of chromosomes in a nucleic acid sample. In some embodiments, karyotyping can be used, in lieu of aCGH, to detect translocations or inversions, which can be copy number neutral, and, therefore, not detectable by aCGH. Information about amplitude of particular probes, which can be representative of particular alleles, can provide quantitative dosage information for the particular allele, and by consequence, dosage information about the CNV in question, since the marker can be selected as a marker representative of the CNV and can be located within the CNV. In some embodiments, if the CNV is a deletion, the absence of particular marker allele is representative of the deletion. In some embodiments, if the CNV is a duplication or a higher order copy number variation, the signal intensity representative of the allele correlating with the CNV can represent the copy number. A summary of methodologies commonly used is provided in Perkel (Perkel J. Nature Methods 5:447-453 (2008)).

PCR assays can be utilized to detect CNVs and can provide an alternative to array analysis. In particular, PCR assays can enable detection of precise boundaries of gene/chromosome variants, at the molecular level, and which boundaries are identical in different individuals. PCR assays can be based on the amplification of a junction fragment present only in individuals that carry a deletion. This assay can convert the detection of a loss by array CGH to one of a gain by PCR.

Examples of PCR techniques that can be used in the present disclosure include, but are not limited to quantitative PCR, real-time quantitative PCR (qPCR), quantitative fluorescent PCR (QF-PCR), multiplex fluorescent PCR (MF-PCR), real time PCR (RT-PCR), single cell PCR, PCR-RFLP/RT-PCR-RFLP, hot start PCR and Nested PCR. Other suitable amplification methods include the ligase chain reaction (LCR), ligation mediated PCR (LM-PCR), degenerate oligonucleotide probe PCR (DOP-PCR), transcription amplification, self-sustained sequence replication, selective amplification of target polynucleotide sequences, consensus sequence primed polymerase chain reaction (CP-PCR), arbitrarily primed polymerase chain reaction (AP-PCR) and nucleic acid sequence based amplification (NASBA).

Alternative methods for the simultaneous interrogation of multiple regions include quantitative multiplex PCR of short fluorescent fragments (QMPSF), multiplex amplifiable probe hybridization (MAPH) and multiplex ligation-dependent probe amplification (MLPA), in which copy-number differences for up to 40 regions can be scored in one experiment. Another approach can be to specifically target regions that harbor known segmental duplications, which are often sites of copy-number variation. By targeting the variable nucleotides between two copies of a segmental duplication (called paralogous sequence variants) using a SNP-genotyping method that provides independent fluorescence intensities for the two alleles, it is possible to detect an increase in intensity of one allele compared with the other.

In some embodiments, the amplified piece of DNA can be bound to beads using the sequencing element of the nucleic acid tag under conditions that favor a single amplified piece of DNA molecule to bind a different bead and amplification occurs on each bead. In some embodiments, such amplification can occur by PCR. Each bead can be placed in a separate well, which can be a picoliter-sized well. In some embodiments, each bead is captured within a droplet of a PCR-reaction-mixture-in-oil-emulsion and PCR amplification occurs within each droplet. The amplification on the bead results in each bead carrying at least one million, at least 5 million, or at least 10 million copies of the single amplified piece of DNA molecule.

In embodiments where PCR occurs in oil-emulsion mixtures, the emulsion droplets are broken, the DNA is denatured and the beads carrying single-stranded nucleic acids clones are deposited into a well, such as a picoliter-sized well, for further analysis according to the methods described herein. These amplification methods allow for the analysis of genomic DNA regions. Methods for using bead amplification followed by fiber optics detection are described in Margulies et al., Nature, 15; 437(7057):376-80 (2005), and as well as in US Publication Application Nos. 20020012930; 20030068629; 20030100102; 20030148344; 20040248161; 20050079510, 20050124022; and 20060078909.

Another variation on the array-based approach can be to use the hybridization signal intensities that are obtained from the oligonucleotides employed on Affymetrix SNP arrays or in Illumina Bead Arrays. Here hybridization intensities are compared with average values that are derived from controls, such that deviations from these averages indicate a change in copy number. As well as providing information about copy number, SNP arrays have the added advantage of providing genotype information. For example, they can reveal loss of heterozygosity, which could provide supporting evidence for the presence of a deletion, or might indicate segmental uniparental disomy (which can recapitulate the effects of structural variation in some genomic regions—Prader-Willi and Angelman syndromes, for example).

Many of the basic procedures followed in microarray-based genome profiling are similar, if not identical, to those followed in expression profiling and SNP analysis, including the use of specialized microarray equipment and data-analysis tools. Since microarray-based expression profiling has been well established in the last decade, much can be learned from the technical advances made in this area. Examples of the use of microarrays in nucleic acid analysis that can be used are described in U.S. Pat. Nos. 6,300,063, 5,837,832, 6,969,589, 6,040,138, 6,858,412, U.S. application Ser. No. 08/529,115, U.S. application Ser. No. 10/272,384, U.S. application Ser. No. 10/045,575, U.S. application Ser. No. 10/264,571 and U.S. application Ser. No. 10/264,574. It should be noted that there are also distinct differences such as target and probe complexity, stability of DNA over RNA, the presence of repetitive DNA and the need to identify single copy number alterations in genome profiling.

In some embodiments, the genetic variations detected comprise CNVs and can be detected using array CGH. In some embodiments, array CGH can be implemented using a wide variety of techniques. The initial approaches used arrays produced from large-insert genomic clones such as bacterial artificial chromosomes (BACs). Producing sufficient BAC DNA of adequate purity to make arrays is arduous, so several techniques to amplify small amounts of starting material have been employed. These techniques include ligation-mediated PCR (Snijders et al., Nat. Genet. 29:263-64), degenerate primer PCR using one or several sets of primers, and rolling circle amplification. BAC arrays that provide complete genome tiling paths are also available. Arrays made from less complex nucleic acids such as cDNAs, selected PCR products, and oligonucleotides can also be used. Although most CGH procedures employ hybridization with total genomic DNA, it is possible to use reduced complexity representations of the genome produced by PCR techniques. Computational analysis of the genome sequence can be used to design array elements complementary to the sequences contained in the representation. Various SNP genotyping platforms, some of which use reduced complexity genomic representations, can be useful for their ability to determine both DNA copy number and allelic content across the genome. In some embodiments, small amounts of genomic DNA can be amplified with a variety of whole genome or whole exome amplification methods prior to CGH analysis of the nucleic acid sample. A “whole exome,” as used herein, includes exons throughout the whole genome that are expressed in genes. Since exon selection has tissue and cell type specificity, these positions may be different in the various cell types resulting from a splice variant or alternative splicing. A “whole genome,” as used herein, includes the entire genetic code of a genome.

The different basic approaches to array CGH provide different levels of performance, so some are more suitable for particular applications than others. The factors that determine performance include the magnitudes of the copy number changes, their genomic extents, the state and composition of the specimen, how much material is available for analysis, and how the results of the analysis can be used. Many applications use reliable detection of copy number changes of much less than 50%, a more stringent requirement than for other microarray technologies. Note that technical details are extremely important and different implementations of methods using the same array CGH approach can yield different levels of performance. Various CGH methods are known in the art and are equally applicable to one or more methods of the present disclosure. For example, CGH methods are disclosed in U.S. Pat. Nos. 7,030,231; 7,011,949; 7,014,997; 6,977,148; 6,951,761; and 6,916,621, the disclosure from each of which is incorporated by reference herein in its entirety.

The data provided by array CGH are quantitative measures of DNA sequence dosage. Array CGH provides high-resolution estimates of copy number aberrations, and can be performed efficiently on many nucleic acid samples. The advent of array CGH technology makes it possible to monitor DNA copy number changes on a genomic scale and many projects have been launched for studying the genome in specific diseases.

In some embodiments, whole genome array-based comparative genome hybridization (array CGH) analysis, or array CGH on a subset of genomic regions, can be used to efficiently interrogate human genomes for genomic imbalances at multiple loci within a single assay. The development of comparative genomic hybridization (CGH) (Kallioniemi et al., Science 258: 818-21 (1992)) provided the first efficient approach to scanning entire genomes for variations in DNA copy number. The importance of normal copy number variation involving large segments of DNA has been unappreciated. Array CGH is a breakthrough technique in human genetics, which is attracting interest from clinicians working in fields as diverse as cancer and IVF (In Vitro Fertilization). The use of CGH microarrays in the clinic holds great promise for identifying regions of genomic imbalance associated with disease. Advances from identifying chromosomal critical regions associated with specific phenotypes to identifying the specific dosage sensitive genes can lead to therapeutic opportunities of benefit to patients. Array CGH is a specific, sensitive and rapid technique that can enable the screening of the whole genome in a single test. It can facilitate and accelerate the screening process in human genetics and is expected to have a profound impact on the screening and counseling of patients with genetic disorders. It is now possible to identify the exact location on the chromosome where an aberration has occurred and it is possible to map these changes directly onto the genomic sequence.

An array CGH approach provides a robust method for carrying out a genome-wide scan to find novel copy number variants (CNVs). The array CGH methods can use labeled fragments from a genome of interest, which can be competitively hybridized with a second differentially labeled genome to arrays that are spotted with cloned DNA fragments, revealing copy-number differences between the two genomes. Genomic clones (for example, BACs), cDNAs, PCR products and oligonucleotides, can all be used as array targets. The use of array CGH with BACs was one of the earliest employed methods and is popular, owing to the extensive coverage of the genome it provides, the availability of reliable mapping data and ready access to clones. The last of these factors is important both for the array experiments themselves, and for confirmatory FISH experiments.

In a typical CGH measurement, total genomic DNA is isolated from control and reference subjects, differentially labeled, and hybridized to a representation of the genome that allows the binding of sequences at different genomic locations to be distinguished. More than two genomes can be compared simultaneously with suitable labels. Hybridization of highly repetitive sequences is typically suppressed by the inclusion of unlabeled Cot-1 DNA in the reaction. In some embodiments of array CGH, it is beneficial to mechanically shear the genomic DNA in a nucleic acid sample, for example, with sonication, prior to its labeling and hybridization step. In another embodiment, array CGH may be performed without use of Cot-1 DNA or a sonication step in the preparation of the genomic DNA in a nucleic acid sample. The relative hybridization intensity of the test and reference signals at a given location can be proportional to the relative copy number of those sequences in the test and reference genomes. If the reference genome is normal then increases and decreases in signal intensity ratios directly indicate DNA copy number variation within the genome of the test cells. Data are typically normalized so that the modal ratio for the genome is set to some standard value, typically 1.0 on a linear scale or 0.0 on a logarithmic scale. Additional measurements such as FISH or flow cytometry can be used to determine the actual copy number associated with a ratio level.

In some embodiments, an array CGH procedure can include the following steps. First, large-insert clones, for example, BACs can be obtained from a supplier of clone libraries. Then, small amounts of clone DNA can be amplified, for example, by degenerate oligonucleotide-primed (DOP) PCR or ligation-mediated PCR in order to obtain sufficient quantities needed for spotting. Next, PCR products can be spotted onto glass slides using, for example, microarray robots equipped with high-precision printing pins. Depending on the number of clones to be spotted and the space available on the microarray slide, clones can either be spotted once per array or in replicate. Repeated spotting of the same clone on an array can increase precision of the measurements if the spot intensities are averaged, and allows for a detailed statistical analysis of the quality of the experiments. Subject and control DNAs can be labeled, for example, with either Cy3 or Cy5-dUTP using random priming and can be subsequently hybridized onto the microarray in a solution containing an excess of Cott-DNA to block repetitive sequences. Hybridizations can either be performed manually under a coverslip, in a gasket with gentle rocking or, automatically using commercially available hybridization stations. These automated hybridization stations can allow for an active hybridization process, thereby improving the reproducibility as well as reducing the actual hybridization time, which increases throughput. The hybridized DNAs can be detected through the two different fluorochromes using standard microarray scanning equipment with either a scanning confocal laser or a charge coupled device (CCD) camera-based reader, followed by spot identification using commercially or freely available software packages.

The use of CGH with arrays that comprise long oligonucleotides (60-100 bp) can improve the detection resolution (in some embodiments, as small as ˜3-5 kb sized CNVs on arrays designed for interrogation of human whole genomes) over that achieved using BACs (limited to 50-100 kb or larger sized CNVs due to the large size of BAC clones). In some embodiments, the resolution of oligonucleotide CGH arrays is achieved via in situ synthesis of 1-2 million unique features/probes per microarray, which can include microarrays available from Roche NimbleGen and Agilent Technologies. In addition to array CGH methods for copy number detection, other embodiments for partial or whole genome analysis of CNVs within a genome include, but are not limited to, use of SNP genotyping microarrays and sequencing methods.

Another method for copy number detection that uses oligonucleotides can be representational oligonucleotide microarray analysis (ROMA). It is similar to that applied in the use of BAC and CGH arrays, but to increase the signal-to-noise ratio, the ‘complexity’ of the input DNA is reduced by a method called representation or whole-genome sampling. Here the DNA that is to be hybridized to the array can be treated by restriction digestion and then ligated to adapters, which results in the PCR-based amplification of fragments in a specific size-range. As a result, the amplified DNA can make up a fraction of the entire genomic sequence—that is, it is a representation of the input DNA that has significantly reduced complexity, which can lead to a reduction in background noise. Other suitable methods available to the skilled person can also be used, and are within scope of the present disclosure.

A comparison of one or more genomes relative to one or more other genomes with array CGH, or a variety of other CNV detection methods, can reveal the set of CNVs between two genomes, between one genome in comparison to multiple genomes, or between one set of genomes in comparison to another set of genomes. In some embodiments, an array CGH experiment can be performed by hybridizing a single test genome against a pooled nucleic acid sample of two or more genomes, which can result in minimizing the detection of higher frequency variants in the experiment. In some embodiments, a test genome can be hybridized alone (i.e. one-color detection) to a microarray, for example, using array CGH or SNP genotyping methods, and the comparison step to one or more reference genomes can be performed in silico to reveal the set of CNVs in the test genome relative to the one or more reference genomes. In one preferred embodiment, a single test genome is compared to a single reference genome in a 2-color experiment wherein both genomes are cohybridized to the microarray.

Array CGH can be used to identify genes that are causative or associated with a particular phenotype, condition, or disease by comparing the set of CNVs found in the affected cohort to the set of CNVs found in an unaffected cohort. An unaffected cohort may consist of any individual unaffected by the phenotype, condition, or disease of interest, but in one preferred embodiment is comprised of individuals or subjects that are apparently healthy (normal). Methods employed for such analyses are described in U.S. Pat. Nos. 7,702,468 and 7,957,913. In some embodiments, candidate genes that are causative or associated (e.g., a biomarker) with a phenotype, condition, or disease will be identified by CNVs that occur in the affected cohort but not in the unaffected cohort. In some embodiments, candidate genes that are causative or associated (e.g., a biomarker) with a phenotype, condition, or disease will be identified by CNVs that occur at a statistically significant higher frequency in the affected cohort as compared their frequency in the unaffected cohort. Thus, CNVs preferentially detected in the affected cohort as compared to the unaffected cohort can serve as beacons of genes that are causative or associated with a particular phenotype, condition, or disease. Methods employed for such analyses are described in U.S. Pat. No. 8,862,410. In some embodiments, CNV detection and comparison methods can result in direct identification of the gene that is causative or associated with phenotype, condition, or disease if the CNVs are found to overlap with or encompass the gene(s). In some embodiments, CNV detection and comparison methods can result in identification of regulatory regions of the genome (e.g., promoters, enhancers, transcription factor binding sites) that regulate the expression of one or more genes that are causative or associated with the phenotype, condition, or disease of interest. In some embodiments, CNV detection and comparison methods can result in identification of a region in the genome in linkage disequilibrium with a genetic variant that is causative or associated with the phenotype, condition, or disease of interest. In another embodiment, CNV detection and comparison methods can result in identification of a region in the genome in linkage disequilibrium with a genetic variant that is protective against the condition or disease of interest.

Due to the large amount of genetic variation between any two genomes, or two sets (cohorts) of genomes, being compared, one preferred embodiment is to reduce the genetic variation search space by interrogating only CNVs, as opposed to the full set of genetic variants that can be identified in an individual's genome or exome. The set of CNVs that occur only, or at a statistically higher frequency, in the affected cohort as compared to the unaffected cohort can then be further investigated in targeted sequencing experiments to reveal the full set of genetic variants (of any size or type) that are causative or associated (e.g., a biomarker) with a phenotype, condition, or disease. It can be appreciated to those skilled in the art that the targeted sequencing experiments are performed in both the affected and unaffected cohorts in order to identify the genetic variants (e.g., SNVs and indels) that occur only, or at a statistically significant higher frequency, in the affected individual or cohort as compared to the unaffected cohort. Methods employed for such analyses are described in U.S. Pat. No. 8,862,410.

A method of screening a subject for a disease or disorder can comprise assaying a nucleic acid sample from the subject to detect sequence information for more than one genetic locus and comparing the sequence information to a panel of nucleic acid biomarkers and screening the subject for the presence or absence of the disease or disorder if one or more of low frequency biomarkers in the panel are present in the sequence information.

The panel can comprise at least one nucleic acid biomarker (e.g., genetic variation) for each of the more than one genetic loci. For example, the panel can comprise 2, 3, 4, 5, 6, 7, 8, 9, 10, 15, 20, 25, 30, 35, 40, 45, 50, 75, 100, 150, 200 or more nucleic acid biomarkers for each of the more than one genetic locus. In some embodiments, the panel can comprise from about 2-1000 nucleic acid biomarkers. For example, the panel can comprise from about 2-900, 2-800, 2-700, 2-600, 2-500, 2-400, 2-300, 2-200, 2-100, 25-900, 25-800, 25-700, 25-600, 25-500, 25-400, 25-300, 25-200, 25-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800-900, or 900-1000 nucleic acid biomarkers.

In some embodiments, a biomarker (e.g., genetic variation) can occur at a frequency of 1% or more in a population of subjects without the disease or disorder. For example, a biomarker can occur at a frequency of 10%, 9%, 8%, 7%, 6%, 5%, 4%, 3%, 2%, 1%, or more in a population of subjects without the disease or disorder. In some embodiments, a biomarker can occur at a frequency from about 1%-20% in a population of subjects without the disease or disorder. For example, a biomarker can occur at a frequency of from about 1%-5% or 1%-10%, in a population of subjects without the disease or disorder.

The panel can comprise at least 2 low frequency biomarkers (e.g., low frequency genetic variations). For example, the panel can comprise at least 2, 3, 4, 5, 6, 7, 8, 9, 10, 11, 12, 3, 14, 15, 15, 17, 18, 19, 20, 25, 30, 35, 40, 45, 50, 55, 60, 65, 70, 75, 80, 85, 90, 95, 100, 125, 150, 175, 200, 250, 500, or 1000 or more low frequency biomarkers. In some embodiments, the panel can comprise from about 2-1000 low frequency biomarkers. For example, the panel can comprise from about 2-900, 2-800, 2-700, 2-600, 2 500, 2-400, 2-300, 2-200, 2-100, 25-900, 25-800, 25-700, 25-600, 25-500, 25-400, 25-300, 25-200, 25-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800 900, or 900-1000 low frequency biomarkers.

In some embodiments, a low frequency biomarker can occur at a frequency of 1% or less in a population of subjects without the disease or disorder. For example, a low frequency biomarker can occur at a frequency of 0.5%, 0.1%, 0.05%, 0.01%, 0.005%, 0.001%, 0.0005%, or 0.0001% or less in a population of subjects without the disease or disorder. In some embodiments, a low frequency biomarker can occur at a frequency from about 0.0001%-0.1% in a population of subjects without the disease or disorder. For example, a low frequency biomarker can occur at a frequency of from about 0.0001%-0.0005%, 0.0001% 0.001%, 0.0001%-0.005%, 0.0001%-0.01%, 0.0001%-0.05%, 0.0001%-0.1%, 0.0001%-0.5%, 0.0005%-0.001%, 0.0005%-0.005%, 0.0005%-0.01%, 0.0005%-0.05%, 0.0005%-0.1%, 0.0005%-0.5%, 0.0005%-1%, 0.001%-0.005%, 0.001%-0.01%, 0.001%-0.05%, 0.001%-0.1%, 0.001%-0.5%, 0.001%-1%, 0.005%-0.01%, 0.005%-0.05%, 0.005%-0.1%, 0.005%-0.5%, 0.005%-1%, 0.01%-0.05%, 0.01%-0.1%, 0.01%-0.5%, 0.01%-1%, 0.05%-0.1%, 0.05%-0.5%, 0.05%-1%, 0.1%-0.5%, 0.1%-1%, or 0.5%-1% in a population of subjects without the disease or disorder. In another embodiment, genetic biomarker frequencies can range higher (e.g., 0.5% to 5%) and have utility for diagnostic testing or drug development targeting the genes that harbor such variants. Genetic variants of appreciable frequency and phenotypic effect in the general population are sometimes described as goldilocks variants (e.g., see Cohen J Clin Lipidol. 2013 May-June; 7(3 Suppl):S1-5 and Price et al. Am J Hum Genet. 2010 Jun. 11; 86(6):832-8).

In some embodiments, the presence or absence of the disease or disorder in the subject can be determined with at least 50% confidence. For example, the presence or absence of the disease or disorder in the subject can be determined with at least 50%, 55%, 60%, 65%, 70%, 75%, 80%, 85%, 90%, 95%, 98%, 99%, or 100% confidence. In some embodiments, the presence or absence of the disease or disorder in the subject can be determined with a 50%-100% confidence. For example, the presence or absence of the disease or disorder in the subject can be determined with a 60%-100%, 70%-100%, 80%-100%, 90% 100%, 50%-90%, 50%-80%, 50%-70%, 50%-60%, 60%-90%, 60%-80%, 60%-70%, 70%-90%, 70%-80%, or 80%-90%. In one embodiment, LOAD candidate CNVs and genes or regulatory loci associated with these CNVs can be determined or identified by comparing genetic data from a cohort of normal individuals to that of an individual or a cohort of individuals known to have, or be susceptible to LOAD.

In one embodiment, LOAD candidate CNV-subregions and genes associated with these regions can be determined or identified by comparing genetic data from a cohort of normal individuals, such as a pre-existing database of CNVs found in normal individuals termed the Normal Variation Engine (NVE), to that of a cohort of individual known to have, or be susceptible to LOAD.

In some embodiments, a nucleic acid sample from one individual or nucleic acid samples from a pool of 2 or more individuals without LOAD can serve as the reference nucleic acid sample(s) and the nucleic acid sample from an individual known to have LOAD or being tested to determine if they have LOAD can serve as the test nucleic acid sample. In one preferred embodiment, the reference and test nucleic acid samples are sex-matched and co-hybridized on the CGH array. For example, reference nucleic acid samples can be labeled with a fluorophore such as Cy5, using methods described herein, and test subject nucleic acid samples can be labeled with a different fluorophore, such as Cy3. After labeling, nucleic acid samples can be combined and can be co-hybridized to a microarray and analyzed using any of the methods described herein, such as aCGH. Arrays can then be scanned and the data can be analyzed with software. Genetic alterations, such as CNVs, can be called using any of the methods described herein. A list of the genetic alterations, such as CNVs, can be generated for one or more test subjects and/or for one or more reference subjects. Such lists of CNVs can be used to generate a master list of non-redundant CNVs and/or CNV-subregions for each type of cohort. In one embodiment, a cohort of test nucleic acid samples, such as individuals known to have or suspected to have LOAD, can be cohybridized with an identical sex-matched reference individual or sex-matched pool of reference individuals to generate a list of redundant or non-redundant CNVs. Such lists can be based on the presence or absence of one or more CNVs and/or CNV subregions present in individuals within the cohort. In this manner, a master list can contain a number of distinct CNVs and/or CNV-subregions, some of which are uniquely present in a single individual and some of which are present in multiple individuals.

In some embodiments, CNVs and/or CNV-subregions of interest can be obtained by annotation of each CNV and/or CNV-subregion with relevant information, such as overlap with known genes and/or exons or intergenic regulatory regions such as transcription factor binding sites. In some embodiments, CNVs and/or CNV-subregions of interest can be obtained by calculating the OR for a CNV and/or CNV-subregion according to the following formula: OR=(LOAD/((#individuals in LOAD cohort)−LOAD))/(NVE/((#individuals in NVE cohort)−NVE)), where: LOAD=number of LOAD individuals with a CNV-subregion of interest and NVE=number of NVE subjects with the CNV-subregion of interest. If NVE=0, it can be set to 1 to avoid dealing with infinities in cases where no CNVs are seen in the NVE. In some embodiments, a set of publicly available CNVs (e.g., the Database of Genomic Variants) can be used as the Normal cohort for comparison to the affected cohort CNVs. In another embodiment, the set of Normal cohort CNVs may comprise a private database generated by the same CNV detection method, such as array CGH, or by a plurality of CNV detection methods that include, but are not limited to, array CGH, SNP genotyping arrays, custom CGH arrays, custom genotyping arrays, exome sequencing, whole genome sequencing, targeted sequencing, FISH, q-PCR, or MLPA.

The number of individuals in any given cohort can be at least about 10, 50, 100, 200, 300, 400, 500, 600, 700, 800, 900, 1000, 2500, 5000, 7500, 10,000, 100,000, or more. In some embodiments, the number of individuals in any given cohort can be from 25-900, 25-800, 25-700, 25-600, 25-500, 25-400, 25-300, 25-200, 25-100, 100-1000, 100-900, 100-800, 100-700, 100-600, 100-500, 100-400, 100-300, 100 200, 200-1000, 200-900, 200-800, 200-700, 200-600, 200-500, 200-400, 200-300, 300-1000, 300-900, 300 800, 300-700, 300-600, 300-500, 300-400, 400-1000, 400-900, 400-800, 400-700, 400-600, 400-500, 500 1000, 500-900, 500-800, 500-700, 500-600, 600-1000, 600-900, 600-800, 600-700, 700-1000, 700-900, 700-800, 800-1000, 800-900, or 900-1000.

In some embodiments, a method of determining relevance or statistical significance of a genetic variant in a human subject to a disease or a condition associated with a genotype comprising screening a genome of a human subject with the disease or condition, such as by array Comparative Genomic Hybridization, sequencing, or SNP genotyping, to provide information on one or more genetic variants, such as those in Tables 1 and 2. The method can further comprise comparing, such as via a computer, information of said one or more genetic variants from the genome of said subject to a compilation of data comprising frequencies of genetic variants in at least 100 normal human subjects, such as those without the disease or condition. The method can further comprise determining a statistical significance or relevance of said one or more genetic variants from said comparison to the condition or disease or determining whether a genetic variant is present in said human subject but not present in said compilation of data from said comparison, or an algorithm can be used to call or identify significant genetic variations, such as a genetic variation whose median log 2 ratio is above or below a computed value. A computer can comprise computer executable logic that provides instructions for executing said comparison.

Different categories for CNVs of interest can be defined. In some embodiments, CNVs/CNV-subregions can be of interest if the CNVs/CNV-subregions occur within intergenic regions and are associated with an OR of at least 0.7. For example, CNVs/CNV-subregions can be of interest if the CNVs/CNV-subregions occur within intergenic regions and are associated with an OR of at least 0.7, 0.8, 0.9, 1, 2, 3, 4, 5, 6, 7, 8, 9, 10, 12, 14, 16, 18, 20, 25, 30, 35, 40, 45, 50, 60, 70, 80, 90, 100, 110, 120, 130, 140, 150, 160, 170, 175, or more. In some embodiments, CNVs/CNV-subregions can be of interest if the CNVs/CNV-subregions occur within intergenic regions and are associated with an OR from about 0.7-200, 0.7-200, 0.7-90, 0.7-80, 0.7-70, 0.7-60, 0.7-50, 0.7-40, 0.7-30, 0.7-20, 0.7-10, 0.7-5, 10-200, 10-180, 10 160, 10-140, 10-120, 10-100, 10-80, 10-60, 10-40, 10-20, 20-200, 20-180, 20-160, 20-140, 20-120, 20-100, 20-80, 20-60, 20-40, 30-200, 30-180, 30-160, 30-140, 30-120, 30-100, 30-80, 30-60, 30-40, 40-200, 40 180, 40-160, 40-140, 40-120, 40-100, 40-90, 40-80, 40-70, 40-60, 40-50, 50-200, 50-180, 50-160, 50-140, 50-120, 50-100, 50-90, 50-80, 50-70, 50-60, 60-200, 60-180, 60-160, 60-140, 60-120, 60-100, 60-90, 60-80, 60-70, 70-200, 70-180, 70-160, 70-140, 70-120, 70-100, 70-90, 70-80, 80-200, 80-180, 80-160, 80-140, 80-120, 80-100, 80-90, 90-200, 90-180, 90-160, 90-140, 90-120, or 90-100.

In some embodiments, CNVs/CNV-subregions can be of interest if the CNV/CNV-subregion overlaps a known gene, and is associated with an OR of at least 1.8. For example, CNVs/CNV-subregions can be of interest if the CNVs/CNV-subregions occur within intergenic regions and are associated with an OR of at least 1.8, 2, 3, 4, 5, 6, 7, 8, 9, 10, 12, 14, 16, 18, 20, 25, 30, 35, 40, 45, 50, 60, 70, 80, 90, 100, 110, 120, 130, 140, 150, 160, 170, 175, or more. In some embodiments, CNVs/CNV-subregions can be of interest if the CNVs/CNV-subregions occur within exonic regions and are associated with an OR from about 1.8-200, 1.8-200, 1.8-90, 1.8-80, 1.8-70, 1.8-60, 1.8-50, 1.8-40, 1.8-30, 1.8-20, 1.8-10, 1.8-5, 10-200, 10-180, 10-160, 10-140, 10-120, 10-100, 10-80, 10-60, 10-40, 10-20, 20-200, 20-180, 20-160, 20-140, 20 120, 20-100, 20-80, 20-60, 20-40, 30-200, 30-180, 30-160, 30-140, 30-120, 30-100, 30-80, 30-60, 30-40, 40-200, 40-180, 40-160, 40-140, 40-120, 40-100, 40-90, 40-80, 40-70, 40-60, 40-50, 50-200, 50-180, 50 160, 50-140, 50-120, 50-100, 50-90, 50-80, 50-70, 50-60, 60-200, 60-180, 60-160, 60-140, 60-120, 60-100, 60-90, 60-80, 60-70, 70-200, 70-180, 70-160, 70-140, 70-120, 70-100, 70-90, 70-80, 80-200, 80-180, 80 160, 80-140, 80-120, 80-100, 80-90, 90-200, 90-180, 90-160, 90-140, 90-120, or 90-100.

In some embodiments, CNVs/CNV-subregions can be of interest if the CNVs/CNV-subregions are overlapping and/or non-overlapping, impact an exon, and they affect 1 or more LOAD cases but only 0 Normal subjects. In some embodiments, CNVs/CNV-subregions can be of interest if the CNVs/CNV-subregions are overlapping and/or non-overlapping, impact an exon, and they affect 2 or more LOAD cases but only 0 or 1 Normal subjects. In some embodiments, CNVs/CNV-subregions can be of interest if the CNVs/CNV-subregions are overlapping and/or non-overlapping, impact an exon, and they affect 1-5 LOAD cases but only 0 or 1 Normal subjects. For example, CNVs/CNV-subregions can be of interest if the CNVs/CNV-subregions are overlapping and/or non-overlapping, impact an exon, and they affect 1 LOAD case but only 0 or 1 Normal subjects. This can enable identification of rarer CNVs in cases with LOAD. In some embodiments, CNVs/CNV-subregions can be of interest if the CNVs/CNV-subregions are overlapping and/or non-overlapping, impact an exon, and they affect 1 LOAD case but only 0 or 1 Normal subjects, and are associated with an OR greater than 0.7, such as 1.8. In some embodiments, CNVs/CNV-subregions can be of interest if the CNVs/CNV-subregions are overlapping and/or non-overlapping, impact an exon, and they affect 2 LOAD cases but only 0 or 1 Normal subjects. In some embodiments, CNVs/CNV-subregions can be of interest if the CNVs/CNV-subregions are overlapping and/or non-overlapping, impact an exon, and they affect 3 LOAD cases but only 0 or 1 Normal subjects. In some embodiments, CNVs/CNV-subregions can be of interest if the CNVs/CNV-subregions are overlapping and/or non-overlapping, impact an exon, and they affect 4 LOAD cases but only 0 or 1 Normal subjects.

In some embodiments, CNVs/CNV-subregions can be of interest if the OR associated with the sum of LOAD cases and the sum of NVE subjects affecting the same gene (including distinct CNVs/CNV-subregions) is at least 0.67. For example, a CNV/CNV-subregion can be of interest if the OR associated with the sum of LOAD cases and the sum of NVE subjects affecting the same gene (including distinct CNVs/CNV-subregions) is at least 0.7, 0.8, 0.9, 1, 2, 3, 4, 5, 6, 7, 8, 9, 10, 12, 14, 16, 18, 20, 25, 30, 35, 40, 45, 50, 60, 70, 80, 90, 100, 110, 120, 130, 140, 150, 160, 170, 175, or more. In some embodiments, a CNVs/CNV-subregions can be of interest if the OR associated with the sum of LOAD cases and the sum of NVE subjects affecting the same gene (including distinct CNVs/CNV-subregions) is from about 0.7 200, 0.7-200, 0.7-90, 0.7-80, 0.7-70, 0.7-60, 0.7-50, 0.7-40, 0.7-30, 0.7-20, 0.7-10, 0.7-5, 10-200, 10-180, 10-160, 10-140, 10-120, 10-100, 10-80, 10-60, 10-40, 10-20, 20-200, 20-180, 20-160, 20-140, 20-120, 20 100, 20-80, 20-60, 20-40, 30-200, 30-180, 30-160, 30-140, 30-120, 30-100, 30-80, 30-60, 30-40, 40-200, 40-180, 40-160, 40-140, 40-120, 40-100, 40-90, 40-80, 40-70, 40-60, 40-50, 50-200, 50-180, 50-160, 50 140, 50-120, 50-100, 50-90, 50-80, 50-70, 50-60, 60-200, 60-180, 60-160, 60-140, 60-120, 60-100, 60-90, 60-80, 60-70, 70-200, 70-180, 70-160, 70-140, 70-120, 70-100, 70-90, 70-80, 80-200, 80-180, 80-160, 80 140, 80-120, 80-100, 80-90, 90-200, 90-180, 90-160, 90-140, 90-120, or 90-100.

In some embodiments, CNVs/CNV-subregions can be of interest if the OR associated with the sum of LOAD cases and the sum of NVE subjects affecting the same gene (including distinct CNVs/CNV-subregions) is at least 1.8. For example, a CNV/CNV-subregion can be of interest if the OR associated with the sum of LOAD cases and the sum of NVE subjects affecting the same gene (including distinct CNVs/CNV-subregions) is at least 1.8, 2, 3, 4, 5, 6, 7, 8, 9, 10, 12, 14, 16, 18, 20, 25, 30, 35, 40, 45, 50, 60, 70, 80, 90, 100, 110, 120, 130, 140, 150, 160, 170, 175, or more. In some embodiments, a CNVs/CNV-subregions can be of interest if the OR associated with the sum of LOAD cases and the sum of NVE subjects affecting the same gene (including distinct CNVs/CNV-subregions) is from about 1.8-200, 1.8-200, 1.8 90, 1.8-80, 1.8-70, 1.8-60, 1.8-50, 1.8-40, 1.8-30, 1.8-20, 1.8-10, 1.8-5, 10-200, 10-180, 10-160, 10-140, 10-120, 10-100, 10-80, 10-60, 10-40, 10-20, 20-200, 20-180, 20-160, 20-140, 20-120, 20-100, 20-80, 20 60, 20-40, 30-200, 30-180, 30-160, 30-140, 30-120, 30-100, 30-80, 30-60, 30-40, 40-200, 40-180, 40-160, 40-140, 40-120, 40-100, 40-90, 40-80, 40-70, 40-60, 40-50, 50-200, 50-180, 50-160, 50-140, 50-120, 50 100, 50-90, 50-80, 50-70, 50-60, 60-200, 60-180, 60-160, 60-140, 60-120, 60-100, 60-90, 60-80, 60-70, 70-200, 70-180, 70-160, 70-140, 70-120, 70-100, 70-90, 70-80, 80-200, 80-180, 80-160, 80-140, 80-120, 80-100, 80-90, 90-200, 90-180, 90-160, 90-140, 90-120, or 90-100.

In some embodiments, CNVs/CNV-subregions can be of interest if the CNVs/CNV-subregions do not overlap (distinct CNV/CNV-subregion), but impact the same gene (or regulatory locus) and are associated with an OR of at least 6 (Genic (distinct CNV-subregions); OR >6). For example, CNVs/CNV-subregions can be of interest if the CNVs/CNV-subregions do not overlap, but impact the same gene (or regulatory locus), and are associated with an OR of at 7, 8, 9, 10, 12, 14, 16, 18, 20, 25, 30, 35, 40, 45, 50, or more. In some embodiments, CNVs/CNV-subregions can be of interest if the CNVs/CNV-subregions do not overlap, but impact the same gene (or regulatory locus), and are associated with an OR from about 6-100, 6-50, 6-40, 6-30, 6-20, 6-10, 6-9, 6-8, 6-7, 8-100, 8-50, 8-40, 8-30, 8-20, 8-10, 10-100, 10-50, 10 40, 10-30, 10-20, 20-100, 20-50, 20-40, 20-30, 30-100, 30-50, 30-40, 40-100, 40-50, 50-100, or 5-7. The CNV-subregion/gene can be an exonic or intronic part of the gene, or both.

In some embodiments, CNVs/CNV-subregions can be of interest if the CNVs/CNV-subregions do not overlap a known gene (e.g., are non-genic or intergenic) and they are associated with an OR of at least 7 (Exon+ve, LOAD >4, NVE<2). For example, CNVs/CNV-subregions can be of interest if the CNVs/CNV-subregion does not overlap a known gene (e.g., is non-genic or intergenic) and/or non-overlapping, impact an exon, affect 2 or more LOAD cases but only 0 or 1 Normal subjects and are associated with an OR of at least 8, 9, 10, 11, 12, 14, 16, 18, 20, 25, 30, 35, 40, 45, 50, or more. In some embodiments, CNVs/CNV-subregions can be of interest if the CNVs/CNV-subregions are overlapping and/or non-overlapping, impact an exon, affect 2 or more LOAD cases but only 0 or 1 Normal subjects and are associated with an OR from about 7-100, 7-50, 7-40, 7-30, 7-20, 20-100, 20-50, 20-40, 20-30, 30-100, 30-50, 30-40, 40-100, 40-50, 50-100, or 7-11.

In some embodiments, CNVs/CNV-subregions can be of interest if the CNVs/CNV-subregions are overlapping and/or non-overlapping, impact an exon, and they affect 1-5 LOAD cases but only 0 or 1 Normal subjects. This can enable identification of rarer CNVs in cases with LOAD. In some embodiments, CNVs/CNV-subregions can be of interest if the CNVs/CNV-subregions are overlapping and/or non-overlapping, impact an exon, and they affect 1 LOAD case but only 0 or 1 Normal subjects, and are associated with an OR greater than 1, such as 1.47, or from 1-2.5. In some embodiments, CNVs/CNV-subregions can be of interest if the CNVs/CNV-subregions are overlapping and/or non-overlapping, impact an exon, and they affect 2 LOAD cases but only 0 or 1 Normal subjects and are associated with an OR greater than 2.5, such as 2.95, or from 2.5-4. In some embodiments, CNVs/CNV-subregions can be of interest if the CNVs/CNV-subregions are overlapping and/or non-overlapping, impact an exon, and they affect 3 LOAD cases but only 0 or 1 Normal subjects and are associated with an OR greater than 4, such as 4.44, or from 4-5.5. In some embodiments, CNVs/CNV-subregions can be of interest if the CNVs/CNV-subregions are overlapping and/or non-overlapping, impact an exon, and they affect 4 LOAD cases but only 0 or 1 Normal subjects and are associated with an OR greater than 5.5, such as 5.92, or from 5.5-6.8.

In some embodiments, CNVs/CNV-subregions can be of interest if the OR associated with the sum of LOAD cases and the sum of NVE subjects affecting the same gene (including distinct CNVs/CNV-subregions) is at least 6. For example, a CNV/CNV-subregion can be of interest if the OR associated with the sum of LOAD cases and the sum of NVE subjects affecting the same gene (including distinct CNVs/CNV-subregions) is at least 7, 8, 9, 10, 12, 14, 16, 18, 20, 25, 30, 35, 40, 45, 50, or more. In some embodiments, a CNVs/CNV-subregions can be of interest if the OR associated with the sum of LOAD cases and the sum of NVE subjects affecting the same gene (including distinct CNVs/CNV-subregions) is from about 6-100, 6-50, 6-40, 6-30, 6-20, 6-10, 6-9, 6-8, 6-7, 8-100, 8-50. 8-40, 8-30, 8-20, 8-10, 10-100, 10-50, 10-40, 10-30, 10-20, 20-100, 20-50, 20-40, 20-30, 30-100, 30-50, 30-40, 40-100, 40-50, 50-100, or 5-7.

In some embodiments, CNVs/CNV-subregions can be of interest if the CNVs/CNV-subregions impact an intron and they affect 5 or more LOAD cases but only 0 or 1 Normal subjects and they are associated with an OR of at least 7 (Intron+ve, LOAD>4, Nonnals<2). For example, CNVs/CNV-subregions can be of interest if the CNVs/CNV-subregions impact an intron and they affect 5 or more LOAD cases but only 0 or 1 Normal subjects and they are associated with an OR of at least 8, 9, 10, 11, 12, 14, 16, 18, 20, 25, 30, 35, 40, 45, 50, or more. In some embodiments, CNVs/CNV-subregions can be of interest if the CNVs/CNV-subregions impact an intron and they affect 5 or more LOAD cases but only 0 or 1 Normal subjects and they are associated with an OR from about 7-100, 7-50, 7-40, 7-30, 7-20, 20-100, 20-50, 20-40, 20-30, 30-100, 30-50, 30-40, 40-100, 40-50, 50-100, or 7-11. CNVs/CNV-subregions impacting introns can be pathogenic (e.g., such variants can result in alternatively spliced mRNAs or loss of a microRNA binding site, which may deleteriously impact the resulting protein's structure or expression level).

In some embodiments, CNVs/CNV-subregions can be of interest if the CNVs/CNV-subregions occur within intergenic regions and are associated with an OR of greater than 30 (High OR intergenic (OR>30)). For example, CNVs/CNV-subregions can be of interest if the CNVs/CNV-subregions occur within intergenic regions and are associated with an OR of greater than 31, 32, 33, 34, 35, 40, 45, 50, 66, 60, 65, 70, 75, 80, 85, 90, 95, 100 or more. In some embodiments, CNVs/CNV-subregions can be of interest if the CNVs/CNV-subregions impact occur within intergenic regions and are associated with an OR from about 30-100, 30-90, 30-80, 30-70, 30-60, 30-50, 30-40, 40-100, 40-90, 40-80, 40-70, 40-60, 40-50, 50-100, 50 90, 50-80, 50-70, 50-60, 60-100, 60-90, 60-80, 60-70, 70-100, 70-90, 70-80, 80-100, 80-90, or 90-100.

In some embodiments, a CNV/CNV-subregion can be of interest if the CNV/CNV-subregion overlaps a known gene, and is associated with an OR of at least 10. In some embodiments, a CNV/CNV-subregion can be of interest if the CNV/CNV-subregion overlaps a known gene, is associated with an OR of at least 6, and if the OR associated with the sum of LOAD cases and the sum of NVE subjects affecting the same gene (including distinct CNV-subregions) is at least 6.

### Methods of Treatment

Several drugs have been reported in the context of UCD or HA and may be used for treatment of AD or at least for ameliorating one or more symptoms of AD. Several drugs have been reported in the context of UCD or HA and may be used for treatment of LOAD or at least for ameliorating one or more symptoms of LOAD. Medications can include, but are not limited to, carglumic acid, glycerol phenylbutyrate, sodium phenylacetate, sodium benzoate and sodium phenylbutyrate. In some embodiments, an immunosuppressive therapy is classified as an experimental therapy, such as a small molecule therapy (for example, ACER-001 (taste masked sodium phenlbutyrate)), an enzyme replacement therapy (AEB 1102 (for example, pegzilarginase), BB-OTC, PRX-OTC,), a mRNA therapy (for example, ARCT-810), a gene therapy (for instance, DTX301 (AAV8), P-OTC-101, SEL-313), a microbiome metabolic therapy (for instance, KB-195), a fusogen therapy (for instance, SG328). In some cases, drugs developed for use in treatment of classical (AR) urea cycle may be used for treatment of AD. Examples of this include but are not limited to glycerol phenylbutyrate (RAVICTI 0) or taste-masked sodium phenylbutyrate (OLPRUVA™), both of which are small molecule therapies.

In some cases, earlier treatment of AD patients may include treatment of symptoms such as hyperammonemia. In some cases, an earlier treatment of AD or LOAD may include dietary plans (e.g., the Mediterranean diet, see PMID 29734664, Jin et al. Nutrients. 2018 May 4; 10(5):564) and/or supplements (e.g., medical food brands Milupa UCD 2 and UCD Anamix Junior by Nutricia North America). These may be typically available as over-the-counter (OTC) treatments and include, but are not limited to: low protein diet, low carbohydrate and high protein and fat diet, medium-chain triglyceride (MCT), sodium pyruvate, essential amino acids, L-arginine, L-citrulline (e.g., Cytolline by Solace Nutrition), D-ribose, uridine, and S-adenosyl-1-methionine. Critical care treatments may sometimes be included in the treatment regimen and include, but are not limited to, hemodialysis and liver transplants (e.g., orthotic or cells). Other therapeutic approaches under development include, but are not limited to, nitric oxide (NO) supplementation, mesenchymal stem cells, codon-optimized human OTC mRNA complexed with lipid-based nanoparticles, ammonia consuming bioengineered bacteria (e.g., SYNB 1020 by Synlogic), lactulose, autophagy enhancers, and famesoid X receptor (FXR) agonists. Another experimental therapy is taurine supplementation, which has support from rodent models in the context of taurine deficiency (mouse knockout of SLC6A6, gene alias TAUT) as a cause of HA (e.g., see PMID 30862735, Qvartskhava N. et al. Proc Natl Acad Sci USA. 2019 Mar. 26; 116(13):6313-6318) and HA due to chronic liver injury (i.e., a non-genetic cause of HA) in rats (see PMID 28959615, Heidari R et al. Toxicol Rep. 2016 Apr. 13; 3:870 879). In humans, taurine supplementation (e.g., in the form of homotaurine, which is also known as ALZ-801, Alzhemed, tramiprosate, and Vivimind) is being investigated for treatment of MCI and AD (e.g., see PMID 32733362, Manzano S et al. Front Neurol. 2020 Jul. 7; 11:614 for a review on reported findings) and clinical trials suggest specific subsets of patients may see a greater benefit, such as AD patients homozygous for the APOE4/4 variant (e.g., see PMID 29199323, Abushakra S et al. J Prev Alzheimers Dis. 2016; 3(4):219-228). In MCI patients, homotaurine was found to favorably impact cytokine profiles, which was correlated with an improvement of episodic memory (see PMID 35515001, Toppi E et al. Front Immunol. 2022 Apr. 19; 13:813951). Evidence suggests that AD and/or MCI patients testing positive for one or more deleterious variants in a UCD/HA gene may see a greater benefit from homotaurine supplementation than AD and/or MCI patients without a deleterious variant in a UCD/HA gene. Since lipopolysaccharides (LPSs) from bacteria (e.g., that reside in the gastrointestinal tract of humans) are neurotoxic and have been linked to AD (e.g., see PMID 36293528, Zhao Y et al. Int J Mol Sci. 2022 Oct. 21; 23(20):12671), another potential treatment for AD or to prevent AD in those at risk for developing AD is to administer antibiotics (e.g., see PMID 34946536, Hurkacz M et al. Molecules. 2021 Dec. 9; 26(24):7456). Examples of antibiotics (reviewed by Hurkacz M et al. 2021) that may be useful for treating or preventing AD include, but are not limited to, tetracyclines (e.g., doxycycline and minocycline), cephalosporins (e.g., ceftriaxone), and antibiotics in the ansamycin family (e.g., rifampicin, a rifamycin derivative that is a subclass of the ansamycin family). A hypothesis for the mechanism of action of antibiotic therapy in treatment of AD or preventing AD is reduction of blood levels of ammonia due to secretion by the gut bacteria. Since HA can result from genetic (e.g., presence of one or more deleterious variants in one or more UCD or HA genes) and/or non-genetic factors (e.g., acute/chronic liver injury or gut bacteria), treatment modality for AD patients or subjects at risk of developing AD could include determining if the patient has a UCD/HA genetic subtype. If an AD patient (or MCI patient who is at risk for developing AD) is found to have HA due to genetic and non-genetic factors, it may be be beneficial to treat both sources of HA (e.g., with a urea cycle agent and an antibiotic).

The present disclosure also includes kits that can be used to treat a condition in animal subjects. These kits comprise one or more immunosuppressive medications and in some embodiments instructions teaching the use of the kit according to the various methods and approaches described herein. Such kits can also include information, such as scientific literature references, package insert materials, clinical trial results, and/or summaries of these and the like, which indicate or establish the activities and/or advantages (or risks and/or disadvantages) of the agent. Such information can be based on the results of various studies, for example, studies using experimental animals involving in vivo models and studies based on human clinical trials. Kits described herein can be provided, marketed and/or promoted to health providers, including physicians, nurses, pharmacists, formulary officials, and the like.

In some aspects a host cell can be used for testing or administering therapeutics. In some embodiments, a host cell can comprise a nucleic acid comprising expression control sequences operably-linked to a coding region. The host cell can be natural or non-natural. The non-natural host used in aspects of the method can be any cell capable of expressing a nucleic acid of the disclosure including, bacterial cells, fungal cells, insect cells, mammalian cells and plant cells. In some aspects the natural host is a mammalian tissue cell and the non-natural host is a different mammalian tissue cell. Other aspects of the method include a natural host that is a first cell normally residing in a first mammalian species and the non-natural host is a second cell normally residing in a second mammalian species. In another alternative aspect, the method uses a first cell and the second cell that are from the same tissue type. In those aspects of the method where the coding region encodes a mammalian polypeptide, the mammalian polypeptide may be a hormone. In other aspects the coding region may encode a neuropeptide, an antibody, an antimetabolite, or a polypeptide or nucleotide therapeutic.

Expression control sequences can be those nucleotide sequences, both 5′ and 3′ to a coding region, that are used for the transcription and translation of the coding region in a host organism. Regulatory sequences include a promoter, ribosome binding site, optional inducible elements and sequence elements used for efficient 3′ processing, including polyadenylation. When the structural gene has been isolated from genomic DNA, the regulatory sequences also include those intronic sequences used for splicing of the introns as part of mRNA formation in the target host.

### Formulations, Routes of Administration, and Effective Doses

Yet another aspect of the present disclosure relates to formulations, routes of administration and effective doses for pharmaceutical compositions comprising an agent or combination of agents of the instant disclosure. Such pharmaceutical compositions can be used to treat a condition (e.g., multiple sclerosis) as described above.

Compounds of the disclosure can be administered as pharmaceutical formulations including those suitable for oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999).

In various embodiments, the pharmaceutical composition includes carriers and excipients (including but not limited to buffers, carbohydrates, mannitol, polypeptides, amino acids, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In some embodiments, the pharmaceutical preparation is substantially free of preservatives. In other embodiments, the pharmaceutical preparation can contain at least one preservative. General methodology on pharmaceutical dosage forms is found in Ansel et al., Pharmaceutical Dosage Forms and Drug Delivery Systems (Lippencott, Williams, & Wilkins, Baltimore Md. (1999)). It can be recognized that, while any suitable carrier known to those of ordinary skill in the art can be employed to administer the compositions of this disclosure, the type of carrier can vary depending on the mode of administration.

Compounds can also be encapsulated within liposomes using well-known technology. Biodegradable microspheres can also be employed as carriers for the pharmaceutical compositions of this disclosure. Suitable biodegradable microspheres are disclosed, for example, in U.S. Pat. Nos. 4,897,268, 5,075,109, 5,928,647, 5,811,128, 5,820,883, 5,853,763, 5,814,344 and 5,942,252.

The compound can be administered in liposomes or microspheres (or microparticles). Methods for preparing liposomes and microspheres for administration to a subject are well known to those of skill in the art. U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, and along with surfactants if required, and the material dialyzed or sonicated, as necessary. A review of known methods is provided by G. Gregoriadis, Chapter 14, “Liposomes,” Drug Carriers in Biology and Medicine, pp. 2.sup.87-341 (Academic Press, 1979).

Microspheres formed of polymers or polypeptides are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

The concentration of drug can be adjusted, the pH of the solution buffered and the isotonicity adjusted to be compatible with intravenous injection, as is well known in the art.

The compounds of the disclosure can be formulated as a sterile solution or suspension, in suitable vehicles, well known in the art. The pharmaceutical compositions can be sterilized by conventional, well-known sterilization techniques, or can be sterile filtered. The resulting aqueous solutions can be packaged for use as is, or lyophilized, the lyophilized preparation being combined with a sterile solution prior to administration. Suitable formulations and additional carriers are described in Remington “The Science and Practice of Pharmacy” (20th Ed., Lippincott Williams & Wilkins, Baltimore MD), the teachings of which are incorporated by reference in their entirety herein.

The agents or their pharmaceutically acceptable salts can be provided alone or in combination with one or more other agents or with one or more other forms. For example, a formulation can comprise one or more agents in particular proportions, depending on the relative potencies of each agent and the intended indication. For example, in compositions for targeting two different host targets, and where potencies are similar, about a 1:1 ratio of agents can be used. The two forms can be formulated together, in the same dosage unit e.g., in one cream, suppository, tablet, capsule, aerosol spray, or packet of powder to be dissolved in a beverage; or each form can be formulated in a separate unit, e.g., two creams, two suppositories, two tablets, two capsules, a tablet and a liquid for dissolving the tablet, two aerosol sprays, or a packet of powder and a liquid for dissolving the powder, etc.

The teen “pharmaceutically acceptable salt” means those salts which retain the biological effectiveness and properties of the agents used in the present disclosure, and which are not biologically or otherwise undesirable.

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p toluenesulfonic acid, acetic acid, fumaric acid, succinic acid, lactic acid, mandelic acid, malic acid, citric acid, tartaric acid or maleic acid. In addition, if the agent(s) contain a carboxyl group or other acidic group, it can be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

A pharmaceutically acceptable ester or amide refers to those which retain biological effectiveness and properties of the agents used in the present disclosure, and which are not biologically or otherwise undesirable. Typical esters include ethyl, methyl, isobutyl, ethylene glycol, and the like. Typical amides include unsubstituted amides, alkyl amides, dialkyl amides, and the like.

In some embodiments, an agent can be administered in combination with one or more other compounds, forms, and/or agents, e.g., as described above. Pharmaceutical compositions with one or more other active agents can be formulated to comprise certain molar ratios. For example, molar ratios of about 99:1 to about 1:99 of a first active agent to the other active agent can be used. In some subset of the embodiments, the range of molar ratios of a first active agent: other active agents are selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio of a first active:other active agents can be about 1:9, and in some embodiments can be about 1:1. The two agents, forms and/or compounds can be formulated together, in the same dosage unit e.g., in one cream, suppository, tablet, capsule, or packet of powder to be dissolved in a beverage; or each agent, form, and/or compound can be formulated in separate units, e.g., two creams, suppositories, tablets, two capsules, a tablet and a liquid for dissolving the tablet, an aerosol spray a packet of powder and a liquid for dissolving the powder, etc.

If necessary or desirable, the agents and/or combinations of agents can be administered with still other agents. The choice of agents that can be co-administered with the agents and/or combinations of agents of the instant disclosure can depend, at least in part, on the condition being treated. Agents of particular use in the formulations of the present disclosure include, for example, any agent having a therapeutic effect for hyperammonemia, including, e.g., drugs used to treat hyperammonemia.

The agent(s) (or pharmaceutically acceptable salts, esters or amides thereof) can be administered per se or in the form of a pharmaceutical composition wherein the active agent(s) is in an admixture or mixture with one or more pharmaceutically acceptable carriers. A pharmaceutical composition, as used herein, can be any composition prepared for administration to a subject. Pharmaceutical compositions for use in accordance with the present disclosure can be formulated in conventional manner using one or more physiologically acceptable carriers, comprising excipients, diluents, and/or auxiliaries, e.g., which facilitate processing of the active agents into preparations that can be administered. Proper formulation can depend at least in part upon the route of administration chosen. The agent(s) useful in the present disclosure, or pharmaceutically acceptable salts, esters, or amides thereof, can be delivered to a subject using a number of routes or modes of administration, including oral, buccal, topical, rectal, transdermal, transmucosal, subcutaneous, intravenous, and intramuscular applications, as well as by inhalation.

For oral administration, the agents can be formulated readily by combining the active agent(s) with pharmaceutically acceptable carriers well known in the art. Such carriers enable the agents of the disclosure to be formulated as tablets, including chewable tablets, pills, dragees, capsules, lozenges, hard candy, liquids, gels, syrups, slurries, powders, suspensions, elixirs, wafers, and the like, for oral ingestion by a subject to be treated. Such formulations can comprise pharmaceutically acceptable carriers including solid diluents or fillers, sterile aqueous media and various non-toxic organic solvents. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Generally, the agents of the disclosure can be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use can contain agent(s) of this disclosure with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents can be used to bring the agents into solution, due to, for example, the presence of large lipophilic moieties. Alternatively, emulsions, suspensions, or other preparations, for example, liposomal preparations, can be used. With respect to liposomal preparations, any known methods for preparing liposomes for treatment of a condition can be used. See, for example, Bangham et al., J. Mol. Biol. 23: 238-252 (1965) and Szoka et al., Proc. Natl Acad. Sci. USA 75: 4194-4198 (1978), incorporated herein by reference. Ligands can also be attached to the liposomes to direct these compositions to particular sites of action. Agents of this disclosure can also be integrated into foodstuffs, e.g., cream cheese, butter, salad dressing, or ice cream to facilitate solubilization, administration, and/or compliance in certain subject populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinyl pyrrolidone (PVP). If desired, disintegrating agents can be added, such as the cross linked polyvinyl pyrrolidone, agar, or alginic acid or a salt thereof such as sodium alginate. The agents can also be formulated as a sustained release preparation.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active agents.

Pharmaceutical preparations that can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active agents can be dissolved or suspended in suitable liquids, such as fatty oils, liquid paraffin, or liquid polyethylene glycols. In addition, stabilizers can be added. All formulations for oral administration should be in dosages suitable for administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or can contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well-known suspending agents. Suitable fillers or carriers with which the compositions can be administered include agar, alcohol, fats, lactose, starch, cellulose derivatives, polysaccharides, polyvinylpyrrolidone, silica, sterile saline and the like, or mixtures thereof used in suitable amounts. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

A syrup or suspension can be made by adding the active compound to a concentrated, aqueous solution of a sugar, e.g., sucrose, to which can also be added any accessory ingredients. Such accessory ingredients can include flavoring, an agent to retard crystallization of the sugar or an agent to increase the solubility of any other ingredient, e.g., as a polyhydric alcohol, for example, glycerol or sorbitol.

When formulating compounds of the disclosure for oral administration, it can be desirable to utilize gastroretentive formulations to enhance absorption from the gastrointestinal (GI) tract. A formulation which is retained in the stomach for several hours can release compounds of the disclosure slowly and provide a sustained release that can be preferred in some embodiments of the disclosure. Disclosure of such gastro-retentive formulations are found in Klausner E. A., et al., Pharm. Res. 20, 1466-73 (2003); Hoffman, A. et al., Int. J. Pharm. 11, 141-53 (2004), Streubel, A., et al. Expert Opin. Drug Deliver. 3, 217-3, and Chavanpatil, M. D. et al., Int. J. Pharm. (2006). Expandable, floating and bioadhesive techniques can be utilized to maximize absorption of the compounds of the disclosure.

The compounds of the disclosure can be formulated for parenteral administration (e.g., by injection, for example, bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example, solutions in aqueous polyethylene glycol.

For injectable formulations, the vehicle can be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation can also comprise polymer compositions which are biocompatible, biodegradable, such as poly(lactic-co-glycolic) acid. These materials can be made into micro or nanospheres, loaded with drug and further coated or derivatized to provide superior sustained release performance. Vehicles suitable for periocular or intraocular injection include, for example, suspensions of therapeutic agent in injection grade water, liposomes and vehicles suitable for lipophilic substances. Other vehicles for periocular or intraocular injection are well known in the art.

In some embodiments, the composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

When administration is by injection, the active compound can be formulated in aqueous solutions, specifically in physiologically compatible buffers such as Hanks solution, Ringer's solution, or physiological saline buffer. The solution can contain formulatory agents such as suspending, stabilizing and/or dispersing agents. Alternatively, the active compound can be in powder form for constitution with a suitable vehicle, e.g., sterile pyrogen-free water, before use. In some embodiments, the pharmaceutical composition does not comprise an adjuvant or any other substance added to enhance the immune response stimulated by the peptide. In some embodiments, the pharmaceutical composition comprises a substance that inhibits an immune response to the peptide. Methods of formulation are known in the art, for example, as disclosed in Remington's Pharmaceutical Sciences, latest edition, Mack Publishing Co., Easton P.

In addition to the formulations described previously, the agents can also be formulated as a depot preparation. Such long acting formulations can be administered by implantation or transcutaneous delivery (for example, subcutaneously or intramuscularly), intramuscular injection or use of a transdermal patch. Thus, for example, the agents can be formulated with suitable polymeric or hydrophobic materials (for example, as an emulsion in an acceptable oil) or ion exchange resins, or as sparingly soluble derivatives, for example, as a sparingly soluble salt.

In some embodiments, pharmaceutical compositions comprising one or more agents of the present disclosure exert local and regional effects when administered topically or injected at or near particular sites of infection. Direct topical application, e.g., of a viscous liquid, solution, suspension, dimethylsulfoxide (DMSO)-based solutions, liposomal formulations, gel, jelly, cream, lotion, ointment, suppository, foam, or aerosol spray, can be used for local administration, to produce for example, local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations can also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983.

Pharmaceutical compositions of the present disclosure can contain a cosmetically or dermatologically acceptable carrier. Such carriers are compatible with skin, nails, mucous membranes, tissues and/or hair, and can include any conventionally used cosmetic or dermatological carrier meeting these requirements. Such carriers can be readily selected by one of ordinary skill in the art. In formulating skin ointments, an agent or combination of agents of the instant disclosure can be formulated in an oleaginous hydrocarbon base, an anhydrous absorption base, a water-in-oil absorption base, an oil-in-water water-removable base and/or a water-soluble base. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Ointments and creams can, for example, be formulated with an aqueous or oily base with the addition of suitable thickening and/or gelling agents. Lotions can be formulated with an aqueous or oily base and can in general also containing one or more emulsifying agents, stabilizing agents, dispersing agents, suspending agents, thickening agents, or coloring agents. The construction and use of transdermal patches for the delivery of pharmaceutical agents is well known in the art. See, e.g., U.S. Pat. Nos. 5,023,252, 4,992,445 and 5,001,139. Such patches can be constructed for continuous, pulsatile, or on demand delivery of pharmaceutical agents.

Lubricants which can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

The compositions according to the present disclosure can be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely, (W/O or water in oil), microemulsions or alternatively microcapsules, microparticles or lipid vesicle dispersions of ionic and/or nonionic type. These compositions can be prepared according to conventional methods. Other than the agents of the disclosure, the amounts of the various constituents of the compositions according to the disclosure are those conventionally used in the art. These compositions in particular constitute protection, treatment or care creams, milks, lotions, gels or foams for the face, for the hands, for the body and/or for the mucous membranes, or for cleansing the skin. The compositions can also consist of solid preparations constituting soaps or cleansing bars.

Compositions of the present disclosure can also contain adjuvants common to the cosmetic and dermatological fields, such as hydrophilic or lipophilic gelling agents, hydrophilic or lipophilic active agents, preserving agents, antioxidants, solvents, fragrances, fillers, sunscreens, odor-absorbers and dyestuffs. The amounts of these various adjuvants are those conventionally used in the fields considered and, for example, are from about 0.01% to about 20% of the total weight of the composition. Depending on their nature, these adjuvants can be introduced into the fatty phase, into the aqueous phase and/or into the lipid vesicles.

In some embodiments, ocular viral infections can be effectively treated with ophthalmic solutions, suspensions, ointments or inserts comprising an agent or combination of agents of the present disclosure. Eye drops can be prepared by dissolving the active ingredient in a sterile aqueous solution such as physiological saline, buffering solution, etc., or by combining powder compositions to be dissolved before use. Other vehicles can be chosen, as is known in the art, including but not limited to: balance salt solution, saline solution, water soluble polyethers such as polyethyene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

The solubility of the components of the present compositions can be enhanced by a surfactant or other appropriate co-solvent in the composition. Such cosolvents include polysorbate 20, 60, and 80, Pluronic F68, F-84 and P-103, cyclodextrin, or other agents known to those skilled in the art. Such co-solvents can be employed at a level of from about 0.0 1% to 2% by weight.

The compositions of the disclosure can be packaged in multidose form. Preservatives can be preferred to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In the prior art ophthalmic products, such preservatives can be employed at a level of from 0.004% to 0.02%. In the compositions of the present application the preservative, preferably benzalkonium chloride, can be employed at a level of from 0.001% to less than 0.01%, e.g., from 0.001% to 0.008%, preferably about 0.005% by weight. It has been found that a concentration of benzalkonium chloride of 0.005% can be sufficient to preserve the compositions of the present disclosure from microbial attack.

In some embodiments, the agents of the present disclosure are delivered in soluble rather than suspension form, which allows for more rapid and quantitative absorption to the sites of action. In general, formulations such as jellies, creams, lotions, suppositories and ointments can provide an area with more extended exposure to the agents of the present disclosure, while formulations in solution, e.g., sprays, provide more immediate, short-term exposure.

In some embodiments relating to topical/local application, the pharmaceutical compositions can include one or more penetration enhancers. For example, the formulations can comprise suitable solid or gel phase carriers or excipients that increase penetration or help delivery of agents or combinations of agents of the disclosure across a permeability barrier, e.g., the skin. Many of these penetration-enhancing compounds are known in the art of topical formulation, and include, e.g., water, alcohols (e.g., terpenes like methanol, ethanol, 2-propanol), sulfoxides (e.g., dimethyl sulfoxide, decylmethyl sulfoxide, tetradecylmethyl sulfoxide), pyrrolidones (e.g., 2-pyrrolidone, N-methyl-2-pyrrolidone, N-(2-hydroxyethyl)pyrrolidone), laurocapram, acetone, dimethylacetamide, dimethylformamide, tetrahydrofurfuryl alcohol, L-a-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamethylene lauramide, proteolytic enzymes, a-bisabolol, d-limonene, urea and N,N-diethyl-m-toluamide, and the like. Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. In some embodiments, the pharmaceutical compositions can include one or more such penetration enhancers.

In some embodiments, the pharmaceutical compositions for local/topical application can include one or more antimicrobial preservatives such as quaternary ammonium compounds, organic mercurials, p-hydroxy benzoates, aromatic alcohols, chlorobutanol, and the like.

In some embodiments, the pharmaceutical compositions can be orally- or rectally delivered solutions, suspensions, ointments, enemas and/or suppositories comprising an agent or combination of agents of the present disclosure.

In some embodiments, the pharmaceutical compositions can be aerosol solutions, suspensions or dry powders comprising an agent or combination of agents of the present disclosure. The aerosol can be administered through the respiratory system or nasal passages. For example, one skilled in the art can recognize that a composition of the present disclosure can be suspended or dissolved in an appropriate carrier, e.g., a pharmaceutically acceptable propellant, and administered directly into the lungs using a nasal spray or inhalant. For example, an aerosol formulation comprising an agent can be dissolved, suspended or emulsified in a propellant or a mixture of solvent and propellant, e.g., for administration as a nasal spray or inhalant. Aerosol formulations can contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that the agent or combination of agents of the present disclosure is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants useful in the present disclosure include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359; Byron et al., U.S. Pat. No. 5,190,029; and Purewal et al., U.S. Pat. No. 5,776,434. Hydrocarbon propellants useful in the disclosure include, for example, propane, isobutane, n-butane, pentane, isopentane and neopentane. A blend of hydrocarbons can also be used as a propellant. Ether propellants include, for example, dimethyl ether as well as the ethers. An aerosol formulation of the disclosure can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present disclosure can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of an agent of the disclosure in (substantially) pure propellant or as a mixture of propellant and solvent. The solvent can be used to dissolve the agent and/or retard the evaporation of the propellant. Solvents useful in the disclosure include, for example, water, ethanol and glycols. Any combination of suitable solvents can be use, optionally combined with preservatives, antioxidants, and/or other aerosol components.

An aerosol formulation can also be a dispersion or suspension. A suspension aerosol formulation can comprise a suspension of an agent or combination of agents of the instant disclosure. Dispersing agents useful in the disclosure include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can similarly be formulated as an emulsion. An emulsion aerosol formulation can include, for example, an alcohol such as ethanol, a surfactant, water and a propellant, as well as an agent or combination of agents of the disclosure. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

The compounds of the disclosure can be formulated for administration as suppositories. A low melting wax, such as a mixture of triglycerides, fatty acid glycerides, Witepsol S55 (trademark of Dynamite Nobel Chemical, Germany), or cocoa butter is first melted and the active component is dispersed homogeneously, for example, by stirring. The molten homogeneous mixture is then poured into convenient sized molds, allowed to cool, and to solidify.

The compounds of the disclosure can be formulated for vaginal administration. Pessaries, tampons, creams, gels, pastes, foams or sprays containing in addition to the active ingredient such carriers as are known in the art to be appropriate.

It is envisioned additionally, that the compounds of the disclosure can be attached releasably to biocompatible polymers for use in sustained release formulations on, in or attached to inserts for topical, intraocular, periocular, or systemic administration. The controlled release from a biocompatible polymer can be utilized with a water soluble polymer to form an instillable formulation, as well. The controlled release from a biocompatible polymer, such as for example, PLGA microspheres or nanospheres, can be utilized in a formulation suitable for intra ocular implantation or injection for sustained release administration, as well any suitable biodegradable and biocompatible polymer can be used.

In one aspect of the disclosure, the subject's carrier status of any of the genetic variation risk variants described herein, or genetic variants identified via other analysis methods within the genes or regulatory loci that are identified by the CNVs or SNVs described herein, can be used to help determine whether a particular treatment modality, such as any one of the above, or a combination thereof, should be administered. Whether a treatment option such as any of the above mentioned treatment options is administered can be determined based on the presence or absence of a particular genetic variation risk variant in the individual, or by monitoring expression of genes that are associated with the variants of the present disclosure. Expression levels and/or mRNA levels can thus be determined before and during treatment to monitor its effectiveness. Alternatively, or concomitantly, the status with respect to a genetic variation, and or genotype and/or haplotype status of at least one risk variant for LOAD presented herein can be determined before and during treatment to monitor its effectiveness. It can also be appreciated by those skilled in the art that aberrant expression levels of a gene impacted by a CNV or other mutations found as a consequence of targeted sequencing of the CNV-identified gene can be assayed or diagnostically tested for by measuring the polypeptide expression level of said aberrantly expressed gene. In another embodiment, aberrant expression levels of a gene may result from a CNV impacting a DNA sequence (e.g., transcription factor binding site) that regulates a gene whose aberrant expression level is involved in or causes LOAD, or other mutations found as a consequence of targeted sequencing of the CNV-identified gene regulatory sequence, can be assayed or diagnostically tested for by measuring the polypeptide expression level of the gene involved in or causative of LOAD. In some embodiments, a specific CNV mutation within a gene, or other specific mutations found upon targeted sequencing of a CNV-identified gene found to be involved in or causative of LOAD, may cause an aberrant structural change in the expressed polypeptide that results from said gene mutations and the altered polypeptide structure(s) can be assayed via various methods know to those skilled in the art.

Alternatively, biological networks or metabolic pathways related to the genes within, or associated with, the genetic variations described herein can be monitored by determining mRNA and/or polypeptide levels. This can be done for example, by monitoring expression levels of polypeptides for several genes belonging to the network and/or pathway in nucleic acid samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In some embodiments, the genetic variations described herein and/or those subsequently found (e.g., via other genetic analysis methods such as sequencing) via targeted analysis of those genes initially identified by the genetic variations described herein, can be used to prevent adverse effects associated with a therapeutic agent, such as during clinical trials. For example, individuals who are carriers of at least one at-risk genetic variation can be more likely to respond negatively to a therapeutic agent, such as an immunosuppressive agent. For example, carriers of certain genetic variants may be more likely to show an adverse response to the therapeutic agent. In some embodiments, one or more of the genetic variations employed during clinical trials for a given therapeutic agent can be used in a companion diagnostic test that is administered to the patient prior to administration of the therapeutic agent to determine if the patient is likely to have a favorable or an adverse response to the therapeutic agent.

The genetic variations described herein can be used for determining whether a subject is administered a pharmaceutical agent, such as a urea cycle agent. Certain combinations of variants, including those described herein, but also combinations with other risk variants for LOAD, can be suitable for one selection of treatment options, while other variant combinations can be suitable for selection of other treatment options. Such combinations of variants can include one variant, two variants, three variants, or four or more variants, as needed to determine with clinically reliable accuracy the selection of treatment module. In another embodiment, information from testing for the genetic variations described herein, or other rare genetic variations in or near the genes described herein, may be combined with information from other types of testing for selection of treatment options.

### Kits

Kits useful in the methods of the disclosure comprise components useful in any of the methods described herein, including for example, primers for nucleic acid amplification, hybridization probes for detecting genetic variation, or other marker detection, restriction enzymes, nucleic acid probes, optionally labeled with suitable labels, allele-specific oligonucleotides, antibodies that bind to an altered polypeptide encoded by a nucleic acid of the disclosure as described herein or to a wild type polypeptide encoded by a nucleic acid of the disclosure as described herein, means for amplification of genetic variations or fragments thereof, means for analyzing the nucleic acid sequence of nucleic acids comprising genetic variations as described herein, means for analyzing the amino acid sequence of a polypeptide encoded by a genetic variation, or a nucleic acid associated with a genetic variation, etc. The kits can for example, include necessary buffers, nucleic acid primers for amplifying nucleic acids, and reagents for allele-specific detection of the fragments amplified using such primers and necessary enzymes (e.g., DNA polymerase). Additionally, kits can provide reagents for assays to be used in combination with the methods of the present disclosure, for example, reagents for use with other screening assays for LOAD.

In some embodiments, the disclosure pertains to a kit for assaying a nucleic acid sample from a subject to detect the presence of a genetic variation, wherein the kit comprises reagents necessary for selectively detecting at least one particular genetic variation in the genome of the individual. In some embodiments, the disclosure pertains to a kit for assaying a nucleic acid sample from a subject to detect the presence of at least one particular allele of at least one polymorphism associated with a genetic variation in the genome of the subject. In some embodiments, the reagents comprise at least one contiguous oligonucleotide that hybridizes to a fragment of the genome of the individual comprising at least genetic variation. In some embodiments, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one genetic variation, or a fragment of a genetic variation. Such oligonucleotides or nucleic acids can be designed using the methods described herein. In some embodiments, the kit comprises one or more labeled nucleic acids capable of allele-specific detection of one or more specific polymorphic markers or haplotypes with a genetic variation, and reagents for detection of the label. In some embodiments, a kit for detecting SNP markers can comprise a detection oligonucleotide probe, that hybridizes to a segment of template DNA containing a SNP polymorphism to be detected, an enhancer oligonucleotide probe, detection probe, primer and/or an endonuclease, for example, as described by Kutyavin et al., (Nucleic Acid Res. 34:e128 (2006)). In other embodiments, the kit can contain reagents for detecting SNVs and/or CNVs.

In some embodiments, the DNA template is amplified by any means of the present disclosure, prior to assessment for the presence of specific genetic variations as described herein. Standard methods well known to the skilled person for performing these methods can be utilized, and are within scope of the disclosure. In one such embodiment, reagents for performing these methods can be included in the reagent kit.

In a further aspect of the present disclosure, a pharmaceutical pack (kit) is provided, the pack comprising a therapeutic agent and a set of instructions for administration of the therapeutic agent to humans screened for one or more variants of the present disclosure, as disclosed herein. The therapeutic agent can be a small molecule drug, an antibody, a peptide, an antisense or RNAi molecule, or other therapeutic molecules as described herein. In some embodiments, an individual identified as a non-carrier of at least one variant of the present disclosure is instructed to take the therapeutic agent. In one such embodiment, an individual identified as a non-carrier of at least one variant of the present disclosure is instructed to take a prescribed dose of the therapeutic agent. In some embodiments, an individual identified as a carrier of at least one variant of the present disclosure is instructed not to take the therapeutic agent. In some embodiments, an individual identified as a carrier of at least one variant of the present disclosure is instructed not to take a prescribed dose of the therapeutic agent. In some embodiments, an individual identified as a carrier of at least one variant of the present disclosure is instructed to take an agent that ameliorates hyperammonemia.

Also provided herein are articles of manufacture, comprising a probe that hybridizes with a region of human chromosome as described herein and can be used to detect a polymorphism described herein. For example, any of the probes for detecting polymorphisms or genetic variations described herein can be combined with packaging material to generate articles of manufacture or kits. The kit can include one or more other elements including: instructions for use; and other reagents such as a label or an agent useful for attaching a label to the probe. Instructions for use can include instructions for screening applications of the probe for making a diagnosis, prognosis, or theranosis to LOAD in a method described herein. Other instructions can include instructions for attaching a label to the probe, instructions for performing in situ analysis with the probe, and/or instructions for obtaining a nucleic acid sample to be analyzed from a subject. In some cases, the kit can include a labeled probe that hybridizes to a region of human chromosome as described herein.

The kit can also include one or more additional reference or control probes that hybridize to the same chromosome or another chromosome or portion thereof that can have an abnormality associated with a particular endophenotype. A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes. Kits for use in self-testing can also be provided. Such test kits can include devices and instructions that a subject can use to obtain a nucleic acid sample (e.g., buccal cells, blood) without the aid of a health care provider. For example, buccal cells can be obtained using a buccal swab or brush, or using mouthwash.

Kits as provided herein can also include a mailer (e.g., a postage paid envelope or mailing pack) that can be used to return the nucleic acid sample for analysis, e.g., to a laboratory. The kit can include one or more containers for the nucleic acid sample, or the nucleic acid sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the nucleic acid sample can be coded, for example, with a bar code for identifying the subject who provided the nucleic acid sample.

In some embodiments, an in vitro screening test can comprise one or more devices, tools, and equipment configured to collect a nucleic acid sample from an individual. In some embodiments of an in vitro screening test, tools to collect a nucleic acid sample can include one or more of a swab, a scalpel, a syringe, a scraper, a container, and other devices and reagents designed to facilitate the collection, storage, and transport of a nucleic acid sample. In some embodiments, an in vitro screening test can include reagents or solutions for collecting, stabilizing, storing, and processing a nucleic acid sample.

Such reagents and solutions for nucleotide collecting, stabilizing, storing, and processing are well known by those of skill in the art and can be indicated by specific methods used by an in vitro screening test as described herein. In some embodiments, an in vitro screening test as disclosed herein, can comprise a microarray apparatus and reagents, a flow cell apparatus and reagents, a multiplex nucleotide sequencer and reagents, and additional hardware and software necessary to assay a nucleic acid sample for certain genetic markers and to detect and visualize certain genetic markers.

The present disclosure further relates to kits for using antibodies in the methods described herein. This includes, but is not limited to, kits for detecting the presence of a variant polypeptide in a test nucleic acid sample. One preferred embodiment comprises antibodies such as a labeled or labelable antibody and a compound or agent for detecting variant polypeptides in a nucleic acid sample, means for determining the amount or the presence and/or absence of variant polypeptide in the nucleic acid sample, and means for comparing the amount of variant polypeptide in the nucleic acid sample with a standard, as well as instructions for use of the kit. In certain embodiments, the kit further comprises a set of instructions for using the reagents comprising the kit.

### Computer-Implemented Aspects

As understood by those of ordinary skill in the art, the methods and information described herein (genetic variation association with LOAD) can be implemented, in all or in part, as computer executable instructions on known computer readable media. For example, the methods described herein can be implemented in hardware. Alternatively, the method can be implemented in software stored in, for example, one or more memories or other computer readable medium and implemented on one or more processors. As is known, the processors can be associated with one or more controllers, calculation units and/or other units of a computer system, or implanted in firmware as desired. If implemented in software, the routines can be stored in any computer readable memory such as in RAM, ROM, flash memory, a magnetic disk, a laser disk, or other storage medium, as is also known. Likewise, this software can be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above can be implemented as various blocks, operations, tools, modules and techniques which, in turn, can be implemented in hardware, firmware, software, or any combination of hardware, firmware, and/or software. When implemented in hardware, some or all of the blocks, operations, techniques, etc. can be implemented in, for example, a custom integrated circuit (IC), an application specific integrated circuit (ASIC), a field programmable logic array (FPGA), a programmable logic array (PLA), etc.

Results from such genotyping can be stored in a data storage unit, such as a data carrier, including computer databases, data storage disks, or by other convenient data storage means. In certain embodiments, the computer database is an object database, a relational database or a post-relational database. Data can be retrieved from the data storage unit using any convenient data query method.

When implemented in software, the software can be stored in any known computer readable medium such as on a magnetic disk, an optical disk, or other storage medium, in a RAM or ROM or flash memory of a computer, processor, hard disk drive, optical disk drive, tape drive, etc. Likewise, the software can be delivered to a user or a computing system via any known delivery method including, for example, on a computer readable disk or other transportable computer storage mechanism.

The steps of the claimed methods can be operational with numerous other general purpose or special purpose computing system environments or configurations. Examples of well-known computing systems, environments, and/or configurations that can be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system can be described in the general context of computer-executable instructions, such as program modules, being executed by a computer. Generally, program modules include routines, programs, objects, components, and/or data structures that perform particular tasks or implement particular abstract data types. The methods and apparatus can also be practiced in distributed computing environments where tasks are performed by remote processing devices that are linked through a communications network. In both integrated and distributed computing environments, program modules can be located in both local and remote computer storage media including memory storage devices. Numerous alternative embodiments could be implemented, using either current technology or technology developed after the filing date of this application, which would still fall within the scope of the claims defining the disclosure.

While the risk evaluation system and method, and other elements, have been described as preferably being implemented in software, they can be implemented in hardware, firmware, etc., and can be implemented by any other processor. Thus, the elements described herein can be implemented in a standard multi-purpose CPU or on specifically designed hardware or firmware such as an application-specific integrated circuit (ASIC) or other hard-wired device as desired. When implemented in software, the software routine can be stored in any computer readable memory such as on a magnetic disk, a laser disk, or other storage medium, in a RAM or ROM of a computer or processor, in any database, etc. Likewise, this software can be delivered to a user or a screening system via any known or desired delivery method including, for example, on a computer readable disk or other transportable computer storage mechanism or over a communication channel, for example, a telephone line, the internet, or wireless communication. Modifications and variations can be made in the techniques and structures described and illustrated herein without departing from the spirit and scope of the present disclosure.

Unless otherwise explained, all technical and scientific terms used herein have the same meaning as commonly understood by one of ordinary skill in the art to which this disclosure belongs. The following references contain embodiments of the methods and compositions that can be used herein: The Merck Manual of Diagnosis and Therapy, 18th Edition, published by Merck Research Laboratories, 2006 (ISBN 0-911910-18-2); Benjamin Lewin, Genes IX, published by Jones & Bartlett Publishing, 2007 (ISBN-13: 9780763740634); Kendrew et al., (eds.), The Encyclopedia of Molecular Biology, published by Blackwell Science Ltd., 1994 (ISBN 0-632-02182-9); and Robert A. Meyers (ed.), Molecular Biology and Biotechnol ogy: a Comprehensive Desk Reference, published by VCH Publishers, Inc., 1995 (ISBN 1-56081-569-8).

Standard procedures of the present disclosure are described, e.g., in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl (eds.), Academic Press Inc., San Diego, USA (1987)). Current Protocols in Molecular Biology (CPMB) (Fred M. Ausubel, et al., ed., John Wiley and Sons, Inc.), Current Protocols in Protein Science (CPPS) (John E. Coligan, et al., ed., John Wiley and Sons, Inc.), Current Protocols in Immunology (CPI) (John E. Coligan, et al., ed. John Wiley and Sons, Inc.), Current Protocols in Cell Biology (CPCB) (Juan S. Bonifacino et al., ed., John Wiley and Sons, Inc.), Culture of Animal Cells: A Manual of Basic Technique by R. Ian Freshney, Publisher: Wiley-Liss; 5th edition (2005), and Animal Cell Culture Methods (Methods in Cell Biology, Vol. 57, Jennie P. Mather and David Barnes editors, Academic Press, 1st edition, 1998), which are all incorporated by reference herein in their entireties.

It should be understood that the following examples should not be construed as being limiting to the particular methodology, protocols, and compositions, etc., described herein and, as such, can vary. The following terms used herein is for the purpose of describing particular embodiments only, and is not intended to limit the scope of the embodiments disclosed herein.

Disclosed herein are molecules, materials, compositions, and components that can be used for, can be used in conjunction with, can be used in preparation for, or are products of methods and compositions disclosed herein. It is understood that when combinations, subsets, interactions, groups, etc. of these materials are disclosed and while specific reference of each various individual and collective combinations and permutation of these molecules and compounds cannot be explicitly disclosed, each is specifically contemplated and described herein. For example, if a nucleotide or nucleic acid is disclosed and discussed and a number of modifications that can be made to a number of molecules including the nucleotide or nucleic acid are discussed, each and every combination and permutation of nucleotide or nucleic acid and the modifications that are possible are specifically contemplated unless specifically indicated to the contrary. This concept applies to all aspects of this application including, but not limited to, steps in methods of making and using the disclosed molecules and compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art can recognize, or be able to ascertain using no more than routine experimentation, many equivalents to the specific embodiments of the method and compositions described herein. Such equivalents are intended to be encompassed by the following claims.

It is understood that the disclosed methods and compositions are not limited to the particular methodology, protocols, and reagents described as these can vary. It is also to be understood that the terminology used herein is for the purpose of describing particular embodiments only, and is not intended to limit the scope of the present disclosure which can be limited only by the appended claims.

Unless defined otherwise, all technical and scientific terms used herein have the meanings that would be commonly understood by one of skill in the art in the context of the present specification.

It should be noted that as used herein and in the appended claims, the singular forms “a,” “an,” and “the” include plural reference unless the context clearly dictates otherwise. Thus, for example, reference to “a nucleotide” includes a plurality of such nucleotides; reference to “the nucleotide” is a reference to one or more nucleotides and equivalents thereof known to those skilled in the art, and so forth.

The term “and/or” shall in the present context be understood to indicate that either or both of the items connected by it are involved. While preferred embodiments of the present disclosure have been shown and described herein, it can be obvious to those skilled in the art that such embodiments are provided by way of example only. Numerous variations, changes, and substitutions can now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

### EXAMPLES

### Example 1—Standard CNV Analysis

The data presented in herein was generated on the basis of a comparison of copy number variants (CNVs) identified in 2 cohorts: 1,000 Normal individuals (Normal Variation Engine—NVE); 100 Late-Onset Alzheimer's Disease (LOAD) cases, which are samples obtained from the Oxford Project to Investigate Memory and Ageing (OPTIMA) cohort (University of Oxford, UK). All OPTIMA LOAD cases were diagnosed with AD on the basis of autopsy findings, as opposed to clinical suspicion in living individuals.

Genomic DNA sample hybridization—NVE and LOAD cohorts

Genomic DNA samples from individuals within the Normal cohort (NVE ‘test’ subjects) and from the LOAD cohort (LOAD ‘test’ subjects) were hybridized against a single, sex-matched reference individual as follows. Reference DNA samples were labeled with Cy5 and test subject DNA samples were labeled with Cy3. After labeling, samples were combined and co-hybridized to Agilent 1M feature oligonucleotide microarrays, design ID 021529 (Agilent Product Number G4447A) using standard conditions (array Comparative Genomic Hybridization—aCGH). Post-hybridization, arrays were scanned at 2 μm resolution, using Agilent's DNA microarray scanner, generating tiff images for later analysis.

All tiff images were analyzed using Agilent Feature Extraction (FE) software, with the following settings:
Human Genome Freeze: hg18:NCB136:March2006
FE version: 10.7.3.1
Grid/design file: 021529DF20091001
Protocol: CGH_107_Sep09

This procedure generates a variety of output files, one of which is a text-tab delimited file, containing ˜1,000,000 rows of data, each corresponding to a specific feature on the array. This *.txt file was used to perform CNV calling using DNAcopy, an open-source software package implemented in R via BioConductor (bioconductor.org/packages/release/bioc/html/DNAcopy.html). Losses or gains were determined according to a threshold log 2ratio, which was set at −/+0.35. In other words, all losses with a log 2ratio value <=−0.35 were counted, as were all gains with a log 2ratio >_+0.35. All log 2ratio values were determined according to Cy3/Cy5 (Test/Reference). A minimum probe threshold for CNV-calling was set at 2 (2 consecutive probes were sufficient to call a CNV). A CNV list was thus generated for each individual in the 2 cohorts.

There was a total of 161,508 CNVs in the NVE cohort of 1,000 individuals (an average of 162 CNVs per individual). Using custom scripts, these CNVs (many of which appeared in multiple individuals) were ‘merged’ into a master list (NVE-master) of non-redundant CNV-subregions, according to the presence or absence of the CNV-subregion in individuals within the cohort. Using this approach, the NVE-master list has 13,918 distinct CNV-subregions, some of which are uniquely present in a single individual and some of which are present in multiple individuals. For example, consider 3 individuals within the NVE cohort with the following hypothetical CNVs:
Chr1:1-100,000;
Chr1:10, 001-100,000;
Chr1:1-89,999;

In the master list, these would be merged into 3 distinct CNV subregions, as follows:

| | | |
|---|---|---|
| CNV-subregion 1 | Chr1: 1-10,000 | Patients A, C |
| CNV-subregion 2 | Chr1: 10,001-89,999 | Patients A, B, C |
| CNV-subregion 3 | Chr90,000: 1-100,000 | Patients A, B |

There was a total of 16,051 CNVs in the LOAD cohort of 100 individuals (an average of 161 CNVs per individual). Using custom scripts, these CNVs (many of which appeared in multiple individuals) were ‘merged’ into a master list (LOAD-master) of non-redundant CNV-subregions, according to the presence or absence of the CNV-subregion in individuals within the cohort. Using this approach, the LOAD-master list has 3,388 distinct CNV-subregions, some of which are uniquely present in a single individual and some of which are present in multiple individuals.

CNV-subregions of interest were obtained after: Annotation using custom designed scripts in order to attach to each CNV region relevant information regarding overlap with known genes and exons; a calculation of the odds ratio (OR) for each CNV-subregion, according to the following formula:

$$OR = ( LOAD / ( 100 - LOAD ) ) / ( NVE / ( 1000 - NVE ) )$$

where: LOAD=number of LOAD individuals with CNV-subregion of interest and NVE=number of NVE individuals with CNV-subregion of interest

As an illustrative example, consider the CNV subregion that is the subject of this application, namely chr2:211070190-211076620 (NCBI36/hg18) in Table 1, which is found in 1 individual in the NVE cohort and 3 individuals in the LOAD cohort. The OR is:

(3/(100−3))/(1/(1000−1))=30.90 (to 2 decimal places)

Note that, by convention, if NVE=0, it is set to 1, in order to avoid dealing with infinities. This has the effect of artificially lowering OR values in cases where none are seen in the NVE.

Note that in instances wherein the same CNV is detected in multiple individuals, the CNV subregion will be equivalent to the originally detected CNV. Similarly, if a CNV is detected in only one affected individual (e.g., 1 LOAD case) and there are no overlapping CNVs found in the NVE cohort, then the CNV subregion will also be equivalent to the originally detected CNV. This was the situation for all the CNVs reported in Table 1. Therefore, p-values and ORs were simply reported in Table 1, rather than in a second table in which CNV subregions would have been reported.

### Example 2—Single Probe CNV Analysis

In addition to automated CNV calling using the DNAcopy algorithm, CNVs were ascertained from a Single Probe database consisting of CGH microarray experiments on 1,000 Normal subjects (controls) and 100 LOAD cases. The Single Probe database consists of 1 million data points for each CGH experiment, in an indexed MySQL database (db). Thus, for the 1,100 experiments, there are a total of 1.1 billion data points. Furthermore, for each probe, the standard deviation (stdev) of the ratio reported by the probe was calculated. The stdev for each probe were analyzed according to gender because some probes may have subtle homology to ChrX (less often ChrY), such that ratios between male and female experiments are subtly different. The stdev analysis facilitated the removal of ‘noisy’ probes from further analysis (see further below for a discussion of what may cause ‘noise’).

Single Probe analysis allows for the identification of CNVs that affect only one probe on the array (e.g., see Table 1). Note that the distance between probes on the array averages to 3Kb (3Gb genome size/1 million probes), such that, even a CNV of 6Kb may affect only one probe.

Conversely, a CNV of 100 bp could also be detected in this manner. However, such an analysis also allows for the detection of CNVs affecting 2 or more probes, some of which are missed by automated calling via DNAcopy (e.g., see Table 1, chr13:99,956,726-99,957,786, which is a 2-probe het loss reported by Agilent probes A_16_P40071581 and A_16_P02848281).

One issue with calling CNVs on the basis of the behavior of a single probe is that some probes are ‘noisy’. There are two main reasons why a probe may exhibit ‘noise’:

The probe itself may not yield high signals of Cy3, Cy5 or both. The weaker the Cy dye signals, the greater the noise inherent in a probe. In our database, there is a good correlation between high stdev and low Cy dye signal intensity;

The probe (average length 60 bp) may overlie a common population variant (e.g., single nucleotide variant), such that those DNA from individuals harboring such a variant will hybridize less well to the probe than DNA that is wild-type in sequence.

These factors were taken into account when considering whether a single-probe CNV was worthy of consideration. Only probes with low stdev and which did not overlie a variant that was common in the population were taken forward for further analysis. This analysis was based on manual assessment as well as using in house developed scripts to search for variants in the region of the probe.

### Example 3—Targeted Analysis of CNVs in UCD/HA Genes

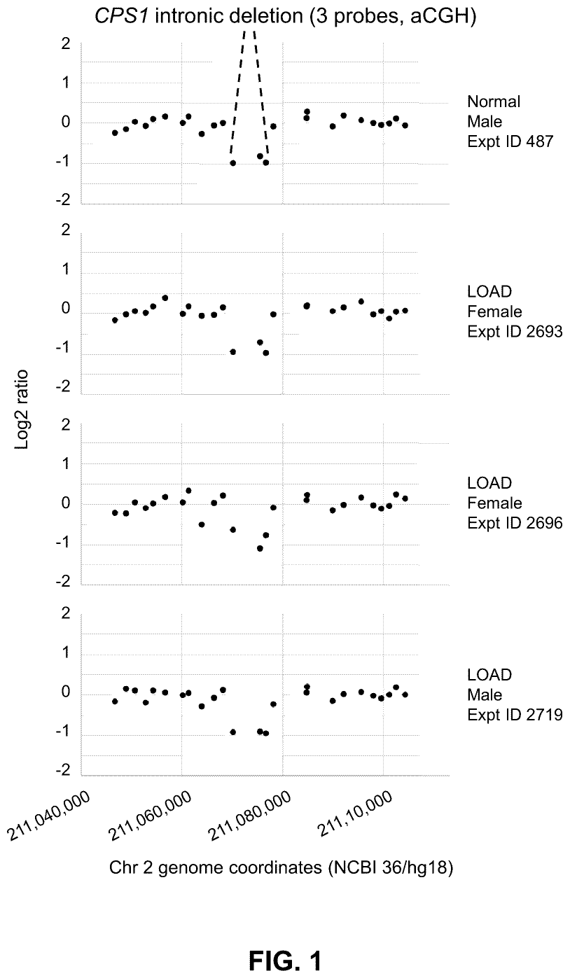

Based on the statistically significant finding of 3 LOAD cases with a CPS1 deletion (Table 1, SEQ ID 3; FIG. 1) and the hypothesis that hyperammonemia may be a contributing factor in causing Alzheimer's, we focused our CNV analyses (single-probe or otherwise) on genes that are relevant to ammonia metabolism (urea cycle or other genes). For example, a frequently used resource for rare genetic disorders is the Online Mendelian Inheritance in Man (OMIM): omim.org. To this end, the following algorithm was implemented:

A list of urea cycle disorder (UCD) genes was generated (e.g., see PubMed PMIDs 20301396 and 25735860), see Table 8. A list of hyperammonemia (HA) genes was generated (e.g., by searching OMIM and UCSC transcript annotation for “ammonia” or “hyperammonemia”), see Table 9 (other genes linked to “ammonia” or “hyperammonemia” that are not reported in Table 8). A non-redundant (nr) list of genes comprising both lists (UCD+HA) was generated (i.e., genes in Table 8 plus Table 9). The nr list of UCD+HA genes was used to generate a file of CNVs (standard+single-probe), from the total list of all CNVs found in this study (100 LOAD+1,000 NVE), which were located within a region 250kb upstream and 250kb downstream of a UCD/HA gene. Note that Tables 1-3 may also include genes whose location is within 250kb of a UCD/HA gene but are not relevant to either UCD or HA (e.g., see SEQ ID 23 in Table 1, where the TSPAN7 gene was directly impacted by a CNV gain but the potentially relevant UCD/HA gene OTC is adjacent to this CNV. Hence, some genes cited in Tables 1-3 do not appear in Tables 8 or 9.

Only CNVs found in at least one LOAD case were retained. For each CNV (standard+single-probe), Fisher's Exact Test (FET) was used to calculate p-values by comparing numbers of the relevant CNV in cases (LOAD) and control subjects (NVE). Only CNVs with a p-value <0.1 were retained.

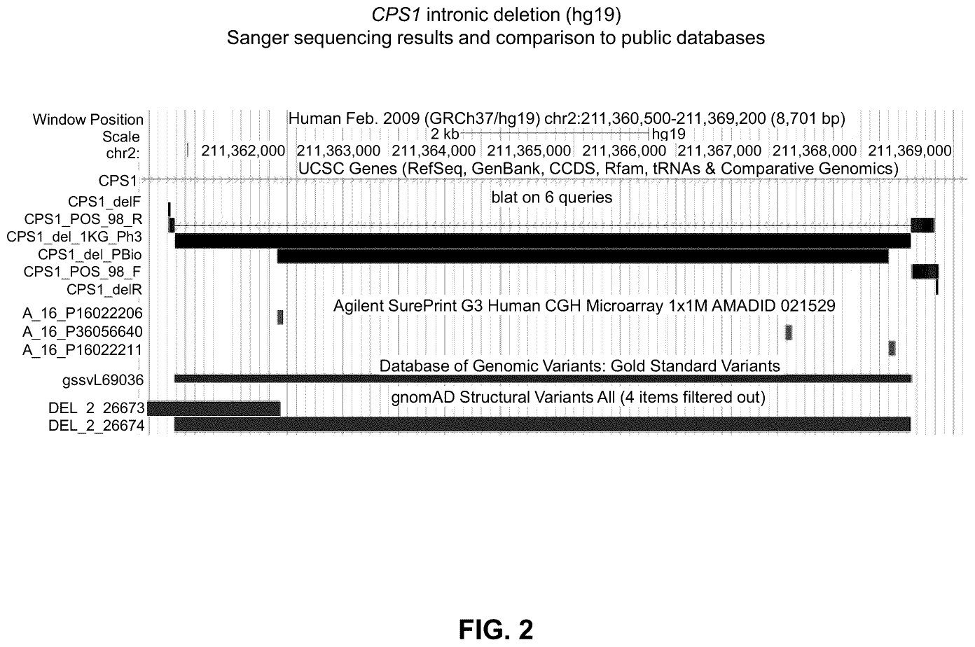

It is well known to those skilled in the art that genetic variants (SNVs, CNVs, etc.) do not need to impact an exon in order to impact the expression of a given gene. Several genome-wide experiments have been performed and reported in the public databases that assist one in determining the potential relevance of a genetic variant. These databases include, but are not limited to, the ENCODE data sets [PMID 22955616, ENCODE Project Consortium Nature 2012 Sep. 6; 489(7414):57-74; PMID 29126249, Davis C et al. Nucleic Acids Res. 2018 Jan. 4; 46(D1):D794-D801] and the GeneCards GeneHancer data sets [PMID 27322403, Stelzer G et al. Curr Protoc Bioinformatics. 2016 Jun. 20; 54:1.30.1-1.30.33; PMID 28605766, Fishilevich S et al. Database (Oxford). 2017 Jan. 1; 2017:bax028]. Examples of annotation from these public resources are shown in FIGS. 2-4, 8, 12, and 13.

### Example 4—Analysis of Frequencies of a CPS1 Deletion Using Publicly Available Data

While Table 1 presents the full list of CNVs (standard or based on single-probe analysis) relevant to UCD/HA genes, particular focus has been placed on the 3-probe intronic deletion affecting CPS1, which was found in 3/100 LOAD cases versus 1/1,000 NVE samples.

Further support for disease-associated variants, such as the CPS1 deletion, can be obtained using publicly available data. These include, but are not limited to: 1,000 Genomes Project Phase 3 (1KG Ph3), see PubMed PMID 26432245; Genome Aggregation Database (gnomAD) for structural variants (SV), see PubMed PMID 32461654; Autism database called MSSNG (research.mss.ng) that includes genetic data on unaffected family members of autism patients, see PubMed PMID 32317787); and Database of Genomic Variants (DGV), see PubMed PMID 24174537.

Table 7 contains a statistical analysis of the frequencies of the 3-probe CPS1 deletion seen in Population Bio's proprietary database (PBio Data), as well as other population databases. Genome coordinates of the aCGH detected deletion in different genome freezes is as follows:
hg18: chr2:211,070,190-211,076,620
hg19: chr2:211,361,945-211,368,375
hg38: chr2:210,497,221-210,503,651

Below is a list of the data sources:
gnomAD: gnomad.broadinstitute.org/variant/DEL_2_26674?dataset=gnomadsv_r2_1

1,000 genomes, phase 3 data: genome-euro.ucsc.edu/cgi-bin/hgc?hgsid=257297933 ht6Q6jUmfsyJJcriAkYpbgIZcueG&c=chr2&1=2 1136 1944&r=2 11368375 &o=211360862&t=211368609&g=tgpPhase3&i=%2D %2F %3CCNO %3E Database of genomic variants: genome-euro.ucsc.edu/cgi-bin/hgc?hgsid=257303105 tddEbMm4LnGWfbbECciYjos6OAmb&c=chr2&1=211360856&r=21136861 0&o=211360856&t=211368610&g=dgvGold&i=gssvL69036 MSSNG autism database: research.mss.ng

The results in Table 7 show corroborating statistical analyses for the CPS1 deletion (PBio data); for comparison, the p-value (0.00273) and OR (30.90) originally reported in Table 1 (SEQ ID 3) are also reported in Table 7. The various association tests with publicly available data had p-values of 0.00049-0.03485 and ORs of 5.93-39.59, thereby providing further support for the CPS1 deletion first identified in our cohorts of 100 LOAD cases vs. 1,000 NVE controls.

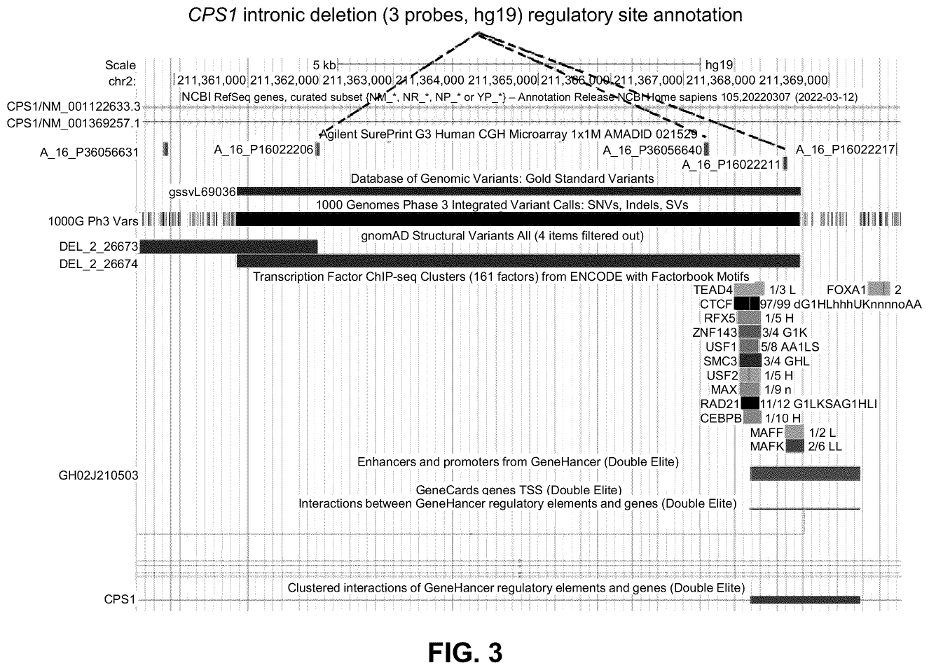

Those skilled in the art will note that the precise size of the deletion differs between the one described by Population Bio and that described by the other data sources. This is because the one identified by Population Bio is defined on the basis of Agilent probes involved in the deletion, while the deletion in the other data sources is based on whole genome sequencing analysis which may provide greater accuracy for deletion endpoints. This is illustrated in FIG. 2, which demonstrates the extent of the deletion reported by all the different data sources (data not shown for the MSSNG deletion, but it is essentially equivalent to the other public data, see genome coordinates in Table 7). As would be expected if the aCGH detected CPS1 deletion is the same as reported by WGS methods, the genome coordinates of the deletion from the public data do not extend to the ‘next’ Agilent probe (see FIG. 3 where 5 Agilent probes are displayed but only the middle 3 Agilent probes lie within the WGS detected deletion).

In summary, FIG. 1 illustrates the observed CNV (aCGH data) in 3/100 LOAD cases and FIGS. 2-4 illustrate the position of the CNV in the context of the gene CPS1, and, in particular, a transcription factor binding site, as well as providing evidence for the functional significance of the deletion, which overlaps a regulatory element that interacts with a promoter element (exon 1) of the CPS1 gene.

### Example 5—Creation of a CPS1 Deletion Assay Using Standard Polymerase Chain Reaction (PCR)

In order to test whether the CPS1 deletion seen in the LOAD cohort is recurrent (i.e., the deletions are identical in the 3 individuals) and, if so, to facilitate future analysis of larger cohorts for the presence of the CPS1 deletion, we developed a simple PCR-based assay that determines the presence or absence of the deletion, without recourse to a method that uses ratiometric analysis (such as array-based CGH, which is expensive, or other methods such as multiplex ligation dependent amplification—MLPA, which is not as simple).

Primers were designed to amplify a product, using standard PCR conditions, if and only if the precise CPS1 deletion observed in the aCGH experiments were present in the individual sample being tested. Those skilled in the art will note that this approach converts the detection of a deletion from a ratiometric method (wherein it is necessary to distinguish a signal that is 50% of normal from one that is 100% of normal—namely detection of a 2:1 fold change) to a binary method (wherein the presence of the deletion results in an amplifiable PCR product, while the absence does not) that allows for an unequivocal result.

The primers (F=forward, R=reverse for detecting the CPS1 deletion, designated as CPS1_del) are as follows (see also Table 6, SEQ IDs 112 and 113):

CPS1_delF
CAGATACTATTTTTGCCAACATGC
CPS1_delR
AGGCAGTGACCCATCAGTATATGT

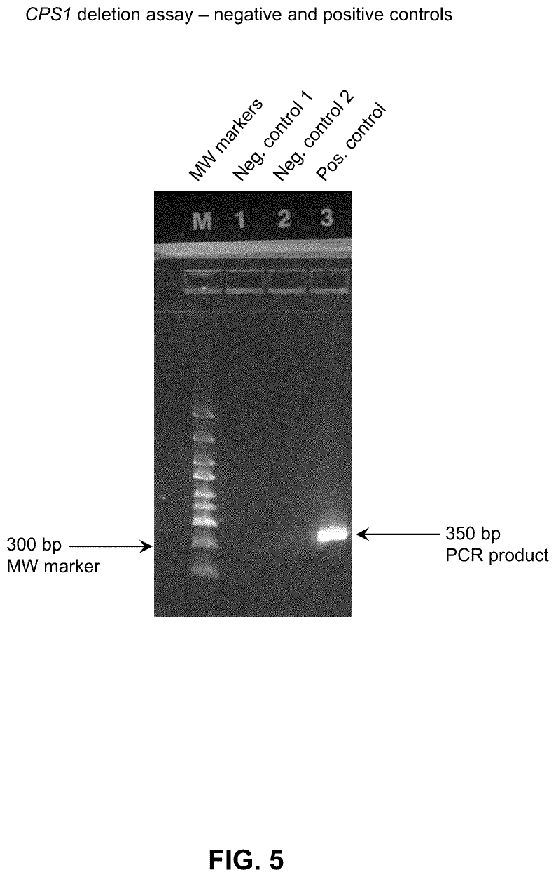

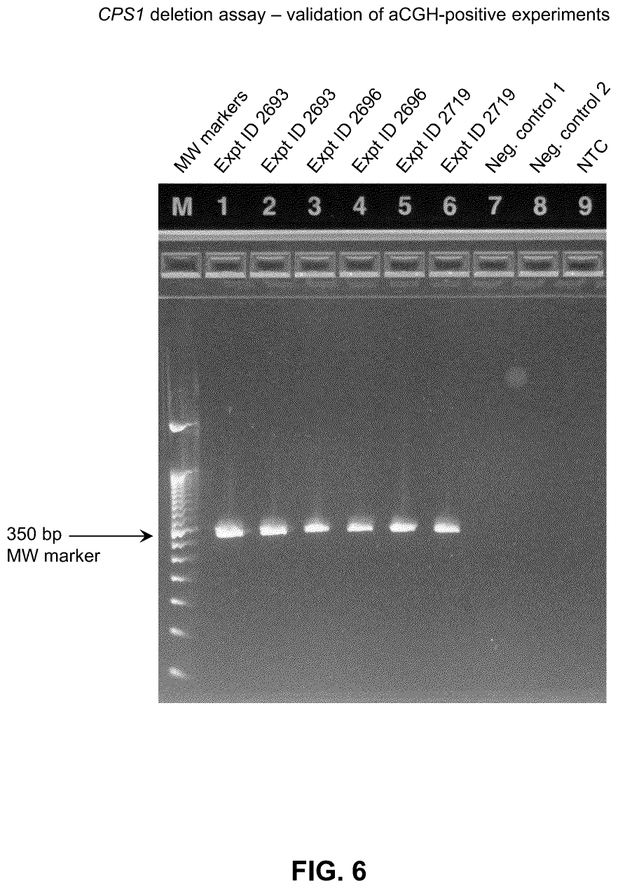

The expected PCR product size when deletion is present is 350 bp. When the deletion is absent, these primers will not generate any product, under standard PCR conditions, since they are ˜8 Kb apart in the genome. FIG. 5 demonstrates the presence of the PCR product in a LOAD case with the deletion (Pos. control based on aCGH data), along with an absence of the band in 2 samples without the deletion (Neg. controls 1 and 2 based on aCGH data). FIG. 6 demonstrates that the 3 LOAD cases with the CPS1 deletion (Expt IDs 2693, 2696, and 2719; each run in duplicate) all amplify the same size PCR product, confirming that the deletions are identical in the 3 cases.

### Example 6—Intronic and Intergenic Variants Near UCD/HA Genes

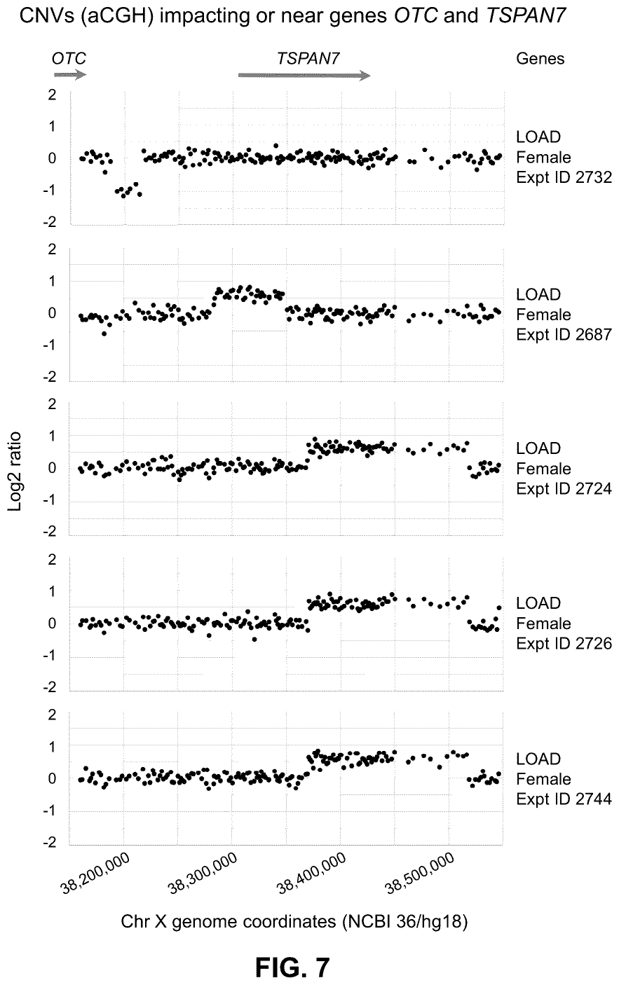

It is increasingly recognized by those skilled in the art that disease-associated genetic variants (causal or protective) do not need to reside in exons in order to impact the expression of given gene. In fact, regulatory sites (e.g., enhancers and promoters) are frequently located in intronic and intergenic regions and can occur a considerable genomic distance from the gene whose expression they regulate. Large-scale projects such as the Encyclopedia of DNA Elements (ENCODE; e.g., Davis C et al., 2018) and GeneHancer (Fishilevich S et al., 2017) have mapped these regulatory regions on a genome-wide scale, which are available in public databases. Examples of LOAD-associated variants in intronic or intergenic regions of UCD/HA genes are shown in FIGS. 7-13 and are reported in Tables 1 and 10-12.

### Example 7—Targeted Analysis of Other Variants in UCD/HA Genes and CPS1-Relevant Regions

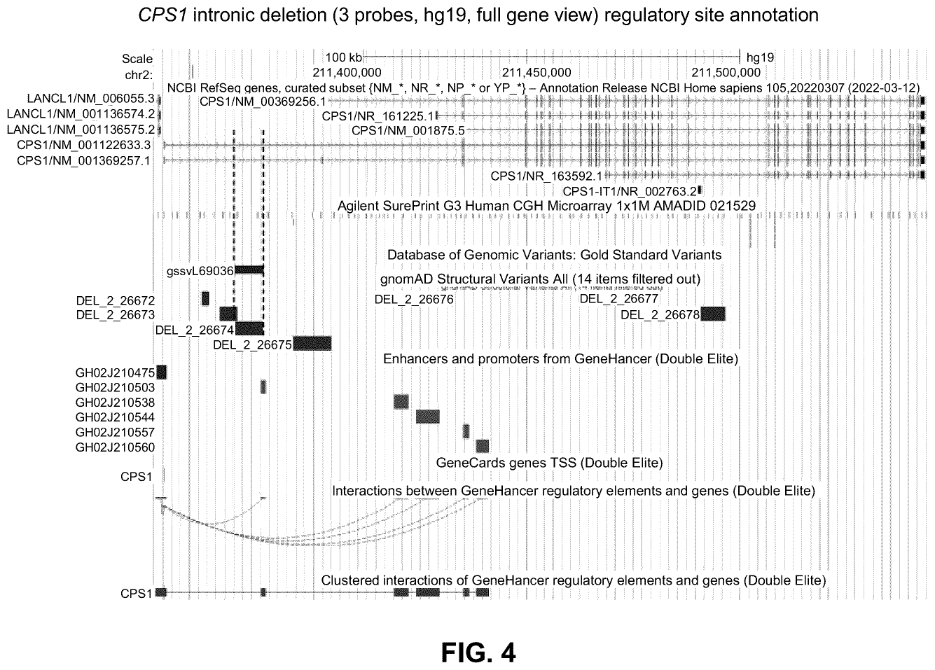

Based on a methodology (e.g., see U.S. Pat. Nos. 8,862,410 and 10,059,997) for using CNVs first to identify disease-relevant genes and then search for further disease-relevant variants, we modified the approach described in Example 3 to identify associated sequence variants (e.g., SNVs and indels) in HA/UCD genes (see Tables 8 and 9). For this analysis, we used publicly available WGS data from the Alzheimer's Disease Neuroimaging Initiative (ADNI) project. Given the strength of the association (see Tables 1 and 7) for the CPS1 intronic deletion (SEQ IDs 3 and 116 in Table 6, depending on whether the deletion was detected by aCGH or WGS), which overlaps with an enhancer regulatory site (GeneHancer GH02J210503, see FIGS. 3 and 4), we also assessed these regions for variants that may be modulating the expression level of the CPS1 gene.

ADNI GATK SNV/Indel whole chromosome VCF files were downloaded for all chromosomes, as was a file listing all samples sequenced. Analysis was confined to cases of European (EUR) origin. Comparison data from gnomAD was also restricted to EUR origin, specifically the Non-Finnish European (NFE) subset. A bed file was generated that included the chr, start and stop positions for:
Table 8 UCD genes
Table 9 HA genes
CPS1 intronic deletion (using 1KG Ph3 coordinates)
GeneHancer Regulatory Element GH02J210503

In house scripts (including the use of bcftools) were used to extract all variants that lie within the coordinates delineated in the bed file. Multi-allelic variants were split into individual rows. This normalized VCF file was converted into a format suitable as input for the Variant Effect Prediction (VEP) pipeline. After VEP was run, gnomAD 2.1 exome data was queried using ‘bcftools query’ to generate data used for direct comparisons. Final analysis files were created and run through in-house stats scripts to generate final values for Fishers Exact test (FET) p-values and odds ratios (ORs).

Analysis was restricted to deleterious variants in individuals of EUR origin. All analyses were based on comparisons between numbers of ADNI cases that were heterozygous or homozygous for the variant of interest and gnomAD cases that were either heterozygous or homozygous (using het+hom totals). The ADNI cases were assessed for two subsets: Alzheimer's disease (AD) and mild cognitive impairment (MCI), which is often a precursor condition for AD patients (although not all patients with MCI will develop AD). Variants satisfying the following two criteria were considered of interest: 1) potentially causal variants for which p-values in ADNI AD and/or ADNI MCI cases were <0.05 but >0.05 in ADNI normal (controls), and for which the OR was >1; and 2) potentially protective variants for which p-values in ADNI AD and/or ADNI MCI cases were <0.05 but >0.05 in ADNI normal (controls), and for which the OR was <1.

Tables 10-12 list the associated sequence variants for the ADNI data sets (AD and MCI subsets). Potentially causal variants (Tables 10 and 11) represent the vast majority of associated variants. Whereas potentially protective variants (2 total) were only found in the MCI subset (Table 12). Only 1 variant (GRCh37/hg19 gnomAD nomenclature: 3-49455323-C-T) is significant in both subsets (AD and MCI) and hence is reported in both Tables 10 and 11.

### Example 8—Treatments for Subjects With UCD and/or HA

There are multiple treatment options for patients with a UCD or HA. For example, see Ah Mew et al. 2003/2017, Haberle et al. 2019, Soria et al. 2019, and Haberle 2020. Since HA has been linked to AD (Branconnier et al., 1986; Seiler, 2002; Adlimoghaddam et al., 2016; Jin et al., 2018) and we report the presence of UCD and HA variants in LOAD cases (see Tables 1 and 10), it is likely that one or more of these UCD/HA therapies will also benefit AD patients. Table 13 lists FDA-approved therapies that could prove useful in AD patients and Table 14 lists other candidate therapies that are in various stages of drug development (pre-clinical through phase 3). Since mild cognitive impairment (MCI) is often a precursor symptom for those that develop AD, we also report UCD/HA gene variants in ADNI MCI cases that were associated as causal (see Table 11, ORs >1) or protective (see Table 12, ORs <1). While causal variants are typically the focus of association studies, the role of protective variants in modifying disease (e.g., later onset and/or less severe disease course) is increasingly appreciated by those skilled in the art. For example, in AD, protective variants have been reported for the genes APOE (Le Guen Y et al., 2022) and PLCG2 (Solomon S et al., 2022).

Those skilled in the art think that earlier treatment of AD patients (e.g., in MCI patients who go on to develop AD) may have a greater benefit from therapies for their symptoms (e.g., if hyperammonemia was found as a symptom for AD and/or MCI and/or the patient was found to harbor a deleterious variant in a UCD/HA gene). In addition to a variety of drug treatment approaches (e.g., see Tables 13 and 14), dietary plans (e.g., the Mediterranean diet, see PMID 29734664, Jin et al. Nutrients. 2018 May 4; 10(5):564) and supplements (e.g., medical food brands Milupa UCD 2 and UCD Anamix Junior by Nutricia North America) are additional options for treating hyperammonemia, depending on which UCD/HA gene is impacted. These are typically available as over-the-counter (OTC) treatments and include, but are not limited to: low protein diet, low carbohydrate and high protein and fat diet, medium-chain triglyceride (MCT), sodium pyruvate, essential amino acids, L-arginine, L-citrulline (e.g., Cytolline by Solace Nutrition), D-ribose, uridine, and S-adenosyl-l-methionine. Critical care treatments are sometimes warranted and include, but are not limited to, hemodialysis and liver transplants (e.g., orthotic or cells). Other therapeutic approaches under development include, but are not limited to, nitric oxide (NO) supplementation, mesenchymal stem cells, codon-optimized human OTC mRNA complexed with lipid-based nanoparticles, ammonia consuming bioengineered bacteria (e.g., SYNB 1020 by Synlogic), lactulose, autophagy enhancers, and famesoid X receptor (FXR) agonists. Taurine supplementation (e.g., in the form of homotaurine, which is also known as ALZ-801, Alzhemed, tramiprosate, and Vivimind) is another therapeutic approach under investigation for treatment of AD and/or MCI. Since taurine supplementation has been shown to be beneficial in rodent models for treatment of HA due to acute/chronic liver injury (i.e., a non-genetic cause of HA; e.g., see PMID 28959615, Heidari R et al. Toxicol Rep. 2016 Apr. 13; 3:870 879) as well as taurine deficiency (i.e., a genetic cause of HA; e.g., see see PMID 30862735, Qvartskhava N. et al. Proc Natl Acad Sci USA. 2019 Mar. 26; 116(13):6313-6318), those skilled in the art would infer that AD and/or MCI patients with HA would particularly benefit from taurine supplementation, with or without one or more deleterious UCD/HA variant if they had acute/chronic liver disease. For some patients with AD or at risk of developing AD who are found to have HA, both genetic (presence of one or more deleterious variants in one or more UCD/HA gene) and non-genetic factors (e.g., due to a chronic bacterial infection) may be the reason and the patient would benefit from treatment with a urea cycle agent and an antibiotic. Furthermore, transplant recipients of donor organs (e.g., liver) from individuals with UCD/HA due to a genetic cause may increase the risk of AD and/or MCI in the future. Therefore, it may be useful to screen donor organs for UCD/HA deleterious variants in order to prevent development of AD/MCI in the transplant recipient.

### Example 9—Tables Referenced in This Study (and see Example 12 for Tables 15-18)

TABLE 1
CNVs of interest in this study

| CNV | | | | | | | | | | | | | UCD/HA Gene | |
|---|---|---|---|---|---|---|---|---|---|---|---|---|---|---|
| Analysis | | CNV Start | CNV Stop | CNV | CNV | Normals | LOAD | | | LOAD | RefSeq | Gene | Impacted or | |
| Method | Chr | (hg18) | (hg18) | Size | Type | (n = 1,000) | (n = 100) | p-value | OR | Expt ID | Gene Symbol | Region | Adjacent | SEQ ID |
| single | 1 | 180387776 | 180387835 | 59 | het | 1 | 2 | 0.02311 | 20.39 | 2708 | | Inter. | GLUL | 1 |
| probe | | | | | loss | | | | | | | | | |
| single | 1 | 180387776 | 180387835 | 59 | het | 1 | 2 | 0.02311 | 20.39 | 2770 | | Inter. | GLUL | 1 |
| probe | | | | | loss | | | | | | | | | |
| single | 1 | 180402819 | 180402878 | 59 | het | 0 | 1 | 0.09091 | 30.17 | 2738 | | Inter. | GLUL | 2 |
| probe | | | | | loss | | | | | | | | | |
| std | 2 | 211070190 | 211076620 | 6430 | het | 1 | 3 | 0.00273 | 30.90 | 2693 | CPS1 | Int. | CPS1 | 3 |
| | | | | | loss | | | | | | | | | |
| std | 2 | 211070190 | 211076620 | 6430 | het | 1 | 3 | 0.00273 | 30.90 | 2696 | CPS1 | Int. | CPS1 | 3 |
| | | | | | loss | | | | | | | | | |
| std | 2 | 211070190 | 211076620 | 6430 | het | 1 | 3 | 0.00273 | 30.90 | 2719 | CPS1 | Int. | CPS1 | 3 |
| | | | | | loss | | | | | | | | | |
| single | 2 | 211157863 | 211157922 | 59 | het | 0 | 1 | 0.09091 | 30.17 | 2675 | CPS1 | Int. | CPS1 | 4 |
| probe | | | | | loss | | | | | | | | | |
| single | 2 | 211188302 | 211188361 | 59 | het | 0 | 1 | 0.09091 | 30.17 | 2742 | CPS1 | Int. | CPS1 | 5 |
| probe | | | | | loss | | | | | | | | | |
| std | 6 | 70494075 | 70500457 | 6382 | het | 0 | 1 | 0.09091 | 30.17 | 2713 | LMBRD1 | Int. | LMBRD1 | 6 |
| | | | | | loss | | | | | | | | | |
| single | 6 | 70508442 | 70508501 | 59 | het | 0 | 1 | 0.09091 | 30.17 | 2748 | LMBRD1 | Ex. | LMBRD1 | 7 |
| probe | | | | | loss | | | | | | | | | |
| single | 7 | 65189910 | 65189957 | 47 | hom | 0 | 1 | 0.09091 | 30.17 | 2742 | ASL | Int. | ASL | 8 |
| probe | | | | | loss | | | | | | | | | |
| single | 7 | 95586936 | 95586995 | 59 | het | 0 | 1 | 0.09091 | 30.17 | 2760 | | Inter. | SLC25A13 | 9 |
| probe | | | | | loss | | | | | | | | | |
| std | 8 | 74863205 | 74865354 | 2149 | hom | 0 | 1 | 0.09091 | 30.17 | 2691 | UBE2W | Ex. | TMEM70 | 10 |
| | | | | | loss | | | | | | | | | |
| single | 9 | 132329289 | 132329333 | 44 | gain | 0 | 1 | 0.09091 | 30.17 | 2676 | ASS1 | Ex. | ASS1 | 11 |
| probe | | | | | | | | | | | | | | |
| single | 12 | 108493317 | 108493376 | 59 | het | 0 | 1 | 0.09091 | 30.17 | 2710 | MMAB | Int. | MMAB | 12 |
| probe | | | | | loss | | | | | | | | | |
| single | 13 | 99956726 | 99957786 | 1060 | het | 0 | 1 | 0.09091 | 30.17 | 2736 | PCCA | Int. | PCCA | 13 |
| probe | | | | | loss | | | | | | | | | |
| std | 14 | 22287112 | 22288620 | 1508 | het | 0 | 1 | 0.09091 | 30.17 | 2672 | | Inter. | SLC7A7 | 14 |
| | | | | | loss | | | | | | | | | |
| std | 17 | 6949119 | 6950605 | 1486 | gain | 0 | 1 | 0.09091 | 30.17 | 2758 | ASGR2 | Int. | ACADVL | 15 |
| std | 17 | 17636988 | 17661982 | 24994 | het | 0 | 1 | 0.09091 | 30.17 | 2674 | MIR33B, | Ex. | ATPAF2 | 16 |
| | | | | | loss | | | | | | RAI1, | | | |
| | | | | | | | | | | | SREBF1 | | | |
| std | 19 | 978045 | 996312 | 18267 | het | 0 | 1 | 0.09091 | 30.17 | 2702 | ABCA7, | Ex. | ATP5F1D | 17 |
| | | | | | loss | | | | | | CNN2 | | | |
| single | 23 | 38095453 | 38095512 | 59 | het | 0 | 1 | 0.09091 | 30.17 | 2700 | | Inter. | OTC | 18 |
| probe | | | | | loss | | | | | | | | | |
| single | 23 | 38109666 | 38109725 | 59 | hom | 0 | 1 | 0.09091 | 30.17 | 2764 | OTC | Int. | OTC | 19 |
| probe | | | | | loss | | | | | | | | | |
| std | 23 | 38193273 | 38213781 | 20508 | het | 0 | 1 | 0.09091 | 30.17 | 2732 | | Inter. | OTC | 20 |
| | | | | | loss | | | | | | | | | |
| std | 23 | 38283722 | 38346557 | 62835 | gain | 0 | 1 | 0.09091 | 30.17 | 2687 | TSPAN7 | Ex. | OTC | 21 |
| single | 23 | 38293309 | 38293368 | 59 | gain | 0 | 1 | 0.09091 | 30.17 | 2766 | | Inter. | OTC | 22 |
| probe | | | | | | | | | | | | | | |
| std | 23 | 38370935 | 38516645 | 145710 | gain | 2 | 3 | 0.00637 | 15.43 | 2724 | TSPAN7 | Ex. | OTC | 23 |
| std | 23 | 38370935 | 38516645 | 145710 | gain | 2 | 3 | 0.00637 | 15.43 | 2726 | TSPAN7 | Ex. | OTC | 23 |
| std | 23 | 38370935 | 38516645 | 145710 | gain | 2 | 3 | 0.00637 | 15.43 | 2744 | TSPAN7 | Ex. | OTC | 23 |
| single | 23 | 38396272 | 38396331 | 59 | gain | 0 | 1 | 0.09091 | 30.17 | 2766 | TSPAN7 | Int. | OTC | 24 |
| probe | | | | | | | | | | | | | | |
| std | 23 | 102665128 | 102695017 | 29889 | gain | 0 | 1 | 0.09091 | 30.17 | 2706 | | Inter. | TAFAZZIN | 25 |

Table 1 lists all CNVs of interest, both standard and those derived on the basis of single probe analysis. The CNVs listed are the original CNVs identified in this study. The column headers in Table 1 are as follows:

CNV Analysis Method—standard (std) analysis uses the DNAcopy algorithm and single probe analysis uses the custom single probe database; Chr—Chromosome (Chr) harboring the CNV; CNV Start (hg18)—start location of the CNV (NCBI36/hg18 genome coordinates); CNV Stop (hg18)—stop location of the CNV (NCBI36/hg18 genome coordinates); CNV Size—size of the CNV (in the case of CNVs inferred on the basis of a single probe, the size of the relevant Agilent probe is displayed); CNV Type—heterozygous deletion (het loss), homozygous deletion (hom loss), or gain; Nonnals (n=1,000)−in the NVE cohort of 1,000 subjects, the number of normal subjects found with the CNV; LOAD (n=100)−in the LOAD cohort of 100 cases, the number of LOAD cases found with the CNV; p-value—statistical significance using FET (a cutoff of <0.1 was used); OR—odds ratio (OR) value; LOAD Expt ID—Experimental identifier (Expt ID) of the LOAD case whose genome harbors the CNV; RefSeq Gene Symbol—gene symbol used by NCBI's Reference Sequence (RefSeq) database (if blank, the CNV is located in an intergenic region of the genome); Gene Region—lists if the CNV is located in an exonic (Ex.), intronic (Int.), or intergenic (Inter.) region of a gene; UCD/HA Gene Impacted or Adjacent—lists which UCD/HA gene (see Tables 8 and 9) is impacted by the CNV (i.e., is located in an exonic or intronic region) or is adjacent to the CNV (i.e., the CNV is intergenic but a UCD/HA gene is located within 250Kb, see Example 6); SEQ ID—Sequence identification number in the Sequence Listing. All coordinates are based on NCBI36/hg18.

TABLE 2
Genes impacted by exonic or intronic CNVs

| RefSeq | | | | |
|---|---|---|---|---|
| Gene | Gene | NCBI | | |
| Symbol | Alias | Gene ID | Gene Full Name | RefSeq Summary |
| ABCA7 | AD9; | 10347 | ATP-binding cassette | The protein encoded by this gene is a member of the superfamily of ATP- |
| | ABCX; | | sub-family A member | binding cassette (ABC) transporters. ABC proteins transport various |
| | ABCA- | | 7 | molecules across extra- and intra-cellular membranes. ABC genes are |
| | SSN | | | divided into seven distinct subfamilies (ABC1, MDR/TAP, MRP, ALD, |
| | | | | OABP, GCN20, White). This protein is a member of the ABC1 subfamily. |
| | | | | Members of the ABC1 subfamily comprise the only major ABC subfamily |
| | | | | found exclusively in multicellular eukaryotes. This full transporter has |
| | | | | been detected predominantly in myelo-lymphatic tissues with the highest |
| | | | | expression in peripheral leukocytes, thymus, spleen, and bone marrow. |
| | | | | The function of this protein is not yet known; however, the expression |
| | | | | pattern suggests a role in lipid homeostasis in cells of the immune system. |
| | | | | [provided by RefSeq, Jul 2008] |
| ASGR2 | HL-2; | 433 | asialoglycoprotein | This gene encodes a subunit of the asialoglycoprotein receptor. This |
| | HBXBP; | | receptor 2 | receptor is a transmembrane protein that plays a critical role in serum |
| | ASGPR2; | | | glycoprotein homeostasis by mediating the endocytosis and lysosomal |
| | ASGP-R2; | | | degradation of glycoproteins with exposed terminal galactose or N- |
| | CLEC4H2 | | | acetylgalactosamine residues. The asialoglycoprotein receptor may |
| | | | | facilitate hepatic infection by multiple viruses including hepatitis B, and is |
| | | | | also a target for liver-specific drug delivery. The asialoglycoprotein |
| | | | | receptor is a hetero-oligomeric protein composed of major and minor |
| | | | | subunits, which are encoded by different genes. The protein encoded by |
| | | | | this gene is the less abundant minor subunit. Alternatively spliced |
| | | | | transcript variants encoding multiple isoforms have been observed for this |
| | | | | gene. [provided by RefSeq, Jan 2011] |
| ASL | ASAL | 435 | argininosuccinate | This gene encodes a member of the lyase 1 family. The encoded protein |
| | | | lyase | forms a cytosolic homotetramer and primarily catalyzes the reversible |
| | | | | hydrolytic cleavage of argininosuccinate into arginine and fumarate, an |
| | | | | essential step in the liver in detoxifying ammonia via the urea cycle. |
| | | | | Mutations in this gene result in the autosomal recessive disorder |
| | | | | argininosuccinic aciduria, or argininosuccinic acid lyase deficiency. A |
| | | | | nontranscribed pseudogene is also located on the long arm of chromosome |
| | | | | 22. Alternatively spliced transcript variants encoding different isoforms |
| | | | | have been described. [provided by RefSeq, Jul 2008] |
| ASS1 | ASS; | 445 | argininosuccinate | The protein encoded by this gene catalyzes the penultimate step of the |
| | CTLN1 | | synthase 1 | arginine biosynthetic pathway. There are approximately 10 to 14 copies of |
| | | | | this gene including the pseudogenes scattered across the human genome, |
| | | | | among which the one located on chromosome 9 appears to be the only |
| | | | | functional gene for argininosuccinate synthetase. Mutations in the |
| | | | | chromosome 9 copy of this gene cause citrullinemia. Two transcript |
| | | | | variants encoding the same protein have been found for this gene. |
| | | | | [provided by RefSeq, Aug 2012] |
| CNN2 | none | 1265 | calponin 2 | The protein encoded by this gene, which can bind actin, calmodulin, |
| | | | | troponin C, and tropomyosin, may function in the structural organization |
| | | | | of actin filaments. The encoded protein could play a role in smooth muscle |
| | | | | contraction and cell adhesion. Several pseudogenes of this gene have been |
| | | | | identified, and are present on chromosomes 1, 2, 3, 6, 9, 11, 13, 15, 16, 21 |
| | | | | and 22. Alternative splicing results in multiple transcript variants encoding |
| | | | | different isoforms. [provided by RefSeq, Jan 2015] |
| CPS1 | PHN; | 1373 | carbamoyl-phosphate | The mitochondrial enzyme encoded by this gene catalyzes synthesis of |
| | GATD6; | | synthase 1 | carbamoyl phosphate from ammonia and bicarbonate. This reaction is the |
| | CPSASE1 | | | first committed step of the urea cycle, which is important in the removal of |
| | | | | excess urea from cells. The encoded protein may also represent a core |
| | | | | mitochondrial nucleoid protein. Three transcript variants encoding |
| | | | | different isoforms have been found for this gene. The shortest isoform may |
| | | | | not be localized to the mitochondrion. Mutations in this gene have been |
| | | | | associated with carbamoyl phosphate synthetase deficiency, susceptibility |
| | | | | to persistent pulmonary hypertension, and susceptibility to venoocclusive |
| | | | | disease after bone marrow transplantation. [provided by RefSeq, May |
| | | | | 2010] |
| LMBRD1 | NESI; | 55788 | LMBR1 domain | This gene encodes a lysosomal membrane protein that may be involved in |
| | LMBD1; | | containing 1 | the transport and metabolism of cobalamin. This protein also interacts with |
| | MAHCF; | | | the large form of the hepatitis delta antigen and may be required for the |
| | C6orf209 | | | nucleocytoplasmic shuttling of the hepatitis delta virus. Mutations in this |
| | | | | gene are associated with the vitamin B12 metabolism disorder termed, |
| | | | | homocystinuria-megaloblastic anemia complementation type F. [provided |
| | | | | by RefSeq, Oct 2009] |
| MIR33B | MIRN33B; | 693120 | microRNA 33b | microRNAs (miRNAs) are short (20-24 nt) non-coding RNAs that are |
| | mir-33b; | | | involved in post-transcriptional regulation of gene expression in |
| | hsa-mir- | | | multicellular organisms by affecting both the stability and translation of |
| | 33b | | | mRNAs. miRNAs are transcribed by RNA polymerase II as part of capped |
| | | | | and polyadenylated primary transcripts (pri-miRNAs) that can be either |
| | | | | protein-coding or non-coding. The primary transcript is cleaved by the |
| | | | | Drosha ribonuclease III enzyme to produce an approximately 70-nt stem- |
| | | | | loop precursor miRNA (pre-miRNA), which is further cleaved by the |
| | | | | cytoplasmic Dicer ribonuclease to generate the mature miRNA and |
| | | | | antisense miRNA star (miRNA*) products. The mature miRNA is |
| | | | | incorporated into a RNA-induced silencing complex (RISC), which |
| | | | | recognizes target mRNAs through imperfect base pairing with the miRNA |
| | | | | and most commonly results in translational inhibition or destabilization of |
| | | | | the target mRNA. The RefSeq represents the predicted microRNA stem- |
| | | | | loop. [provided by RefSeq, Sep 2009]. |
| MMAB | ATR; cob; | 326625 | metabolism of | This gene encodes a protein that catalyzes the final step in the conversion |
| | cblB; | | cobalamin associated | of vitamin B(12) into adenosylcobalamin (AdoCbl), a vitamin B12- |
| | CFAP23 | | B | containing coenzyme for methylmalonyl-CoA mutase. Mutations in the |
| | | | | gene are the cause of vitamin B12-dependent methylmalonic aciduria |
| | | | | linked to the cblB complementation group. Alternatively spliced transcript |
| | | | | variants have been found. [provided by RefSeq, Apr 2011] |
| OTC | OCTD; | 5009 | ornithine | This nuclear gene encodes a mitochondrial matrix enzyme. The encoded |
| | OTC1; | | transcarbamylase | protein is involved in the urea cycle which functions to detoxify ammonia |
| | OTCD; | | | into urea for excretion. Mutations in this enzyme lead to ornithine |
| | OTCase | | | transcarbamylase deficiency, which causes hyperammonemia. [provided |
| | | | | by RefSeq, May 2022] |
| PCCA | none | 5095 | propionyl-CoA | The protein encoded by this gene is the alpha subunit of the heterodimeric |
| | | | carboxylase subunit | mitochondrial enzyme Propionyl-CoA carboxylase. PCCA encodes the |
| | | | alpha | biotin-binding region of this enzyme. Mutations in either PCCA or PCCB |
| | | | | (encoding the beta subunit) lead to an enzyme deficiency resulting in |
| | | | | propionic acidemia. Multiple transcript variants encoding different |
| | | | | isoforms have been found for this gene. [provided by RefSeq, May 2010] |
| RAI1 | SMS; | 10743 | retinoic acid induced | This gene is located within the Smith-Magenis syndrome region on |
| | SMCR | | 1 | chromosome 17. It is highly similar to its mouse counterpart and is |
| | | | | expressed at high levels mainly in neuronal tissues. The protein encoded |
| | | | | by this gene includes a polymorphic polyglutamine tract in the N-terminal |
| | | | | domain. Expression of the mouse counterpart in neurons is induced by |
| | | | | retinoic acid. This gene is associated with both the severity of the |
| | | | | phenotype and the response to medication in schizophrenic patients. |
| | | | | [provided by RefSeq, Jul 2008] |
| SREBF1 | HMD; | 6720 | sterol regulatory | This gene encodes a basic helix-loop-helix-leucine zipper (bHLH-Zip) |
| | IFAP2; | | element binding | transcription factor that binds to the sterol regulatory element-1 (SRE1), |
| | SREBP1; | | transcription factor 1 | which is a motif that is found in the promoter of the low density |
| | bHLHd1 | | | lipoprotein receptor gene and other genes involved in sterol biosynthesis. |
| | | | | The encoded protein is synthesized as a precursor that is initially attached |
| | | | | to the nuclear membrane and endoplasmic reticulum. Following cleavage, |
| | | | | the mature protein translocates to the nucleus and activates transcription. |
| | | | | This cleaveage is inhibited by sterols. This gene is located within the |
| | | | | Smith-Magenis syndrome region on chromosome 17. Alternative promoter |
| | | | | usage and splicing result in multiple transcript variants, including SREBP- |
| | | | | 1a and SREBP-1c, which correspond to RefSeq transcript variants 2 and 3, |
| | | | | respectively. [provided by RefSeq, Nov 2017] |
| TSPAN7 | A15; | 7102 | tetraspanin 7 | The protein encoded by this gene is a member of the transmembrane 4 |
| | MXS1; | | | superfamily, also known as the tetraspanin family. Most of these members |
| | CD231; | | | are cell-surface proteins that are characterized by the presence of four |
| | MRX58; | | | hydrophobic domains. The proteins mediate signal transduction events that |
| | CCG-B7; | | | play a role in the regulation of cell development, activation, growth and |
| | TM4SF2; | | | motility. This encoded protein is a cell surface glycoprotein and may have |
| | XLID58; | | | a role in the control of neurite outgrowth. It is known to complex with |
| | TALLA-1; | | | integrins. This gene is associated with X-linked cognitive disability and |
| | TM4SF2b; | | | neuropsychiatric diseases such as Huntington's chorea, fragile X syndrome |
| | DXS1692E | | | and myotonic dystrophy. [provided by RefSeq, Jul 2008] |
| UBE2W | UBC16; | 55284 | ubiquitin conjugating | This gene encodes a nuclear-localized ubiquitin-conjugating enzyme (E2) |
| | UBC-16 | | enzyme E2 W | that, along with ubiquitin-activating (E1) and ligating (E3) enzymes, |
| | | | | coordinates the addition of a ubiquitin moiety to existing proteins. The |
| | | | | encoded protein promotes the ubiquitination of Fanconi anemia |
| | | | | complementation group proteins and may be important in the repair of |
| | | | | DNA damage. There is a pseudogene for this gene on chromosome 1. |
| | | | | Alternative splicing results in multiple transcript variants. [provided by |
| | | | | RefSeq, Aug 2012] |

For all genes listed in Table 1 (namely, those relevant to CNVs of interest), Table 2 represents a non-redundant list of the genes impacted by exonic or intronic CNVs. The column headers in Table 2 are as follows: RefSeq Gene Symbol—official HGNC-provided gene symbol used by NCBI's Reference Sequence (RefSeq) database, which is based on the HUGO Gene Nomenclature Committee (HGNC) wherein HUGO refers to the Human Genome Organization; Gene Alias—list of previously used gene symbols (entries with none have no gene aliases reported in the HGNC database); NCBI Gene ID—NCBI's unique gene identifier number; Gene Full Name—official HGNC-provided full name for the gene; RefSeq Summary—RefSeq-provided summary of the gene's function and/or biology.

TABLE 3
Non-redundant list of transcript variants for
each gene impacted by an exonic or intronic CNV

| RefSeq | | | |
|---|---|---|---|
| Gene | RefSeq Accession | | |
| Symbol | Number | Transcript Definition | SEQ ID |
| ABCA7 | NM_019112 | Homo sapiens ATP binding cassette subfamily A | 26 |
| | | member 7 (ABCA7), mRNA. | |
| ASGR2 | NM_001181 | Homo sapiens asialoglycoprotein receptor 2 | 27 |
| | | (ASGR2), transcript variant 1, mRNA. | |
| ASGR2 | NM_001201352 | Homo sapiens asialoglycoprotein receptor 2 | 28 |
| | | (ASGR2), transcript variant 4, mRNA. | |
| ASGR2 | NM_080912 | Homo sapiens asialoglycoprotein receptor 2 | 29 |
| | | (ASGR2), transcript variant H2′, mRNA. | |
| ASGR2 | NM_080913 | Homo sapiens asialoglycoprotein receptor 2 | 30 |
| | | (ASGR2), transcript variant 2, mRNA. | |
| ASGR2 | NM_080914 | Homo sapiens asialoglycoprotein receptor 2 | 31 |
| | | (ASGR2), transcript variant 3, mRNA. | |
| ASL | NM_000048 | Homo sapiens argininosuccinate lyase (ASL), | 32 |
| | | transcript variant 2, mRNA. | |
| ASL | NM_001024943 | Homo sapiens argininosuccinate lyase (ASL), | 33 |
| | | transcript variant 1, mRNA. | |
| ASL | NM_001024944 | Homo sapiens argininosuccinate lyase (ASL), | 34 |
| | | transcript variant 3, mRNA. | |
| ASL | NM_001024946 | Homo sapiens argininosuccinate lyase (ASL), | 35 |
| | | transcript variant 4, mRNA. | |
| ASS1 | NM_000050 | Homo sapiens argininosuccinate synthase 1 (ASS1), | 36 |
| | | transcript variant 1, mRNA. | |
| ASS1 | NM_054012 | Homo sapiens argininosuccinate synthase 1 (ASS1), | 37 |
| | | transcript variant 2, mRNA. | |
| CNN2 | NM_001303499 | Homo sapiens calponin 2 (CNN2), transcript variant | 38 |
| | | 3, mRNA. | |
| CNN2 | NM_001303501 | Homo sapiens calponin 2 (CNN2), transcript variant | 39 |
| | | 4, mRNA. | |
| CNN2 | NM_004368 | Homo sapiens calponin 2 (CNN2), transcript variant | 40 |
| | | 1, mRNA. | |
| CNN2 | NM_201277 | Homo sapiens calponin 2 (CNN2), transcript variant | 41 |
| | | 2, mRNA. | |
| CPS1 | NM_001122633 | Homo sapiens carbamoyl-phosphate synthase 1, | 42 |
| | | mitochondrial (CPS1), transcript variant 1, mRNA; | |
| | | nuclear gene for mitochondrial product. | |
| CPS1 | NM_001369256 | Homo sapiens carbamoy1-phosphate synthase 1 | 43 |
| | | (CPS1), transcript variant 4, mRNA; nuclear gene for | |
| | | mitochondrial product. | |
| CPS1 | NM_001369257 | Homo sapiens carbamoyl-phosphate synthase 1 | 44 |
| | | (CPS1), transcript variant 5, mRNA; nuclear gene for | |
| | | mitochondrial product. | |
| CPS1 | NM_001875 | Homo sapiens carbamoyl-phosphate synthase 1, | 45 |
| | | mitochondrial (CPS1), transcript variant 2, mRNA; | |
| | | nuclear gene for mitochondrial product. | |
| CPS1 | NR_161225 | Homo sapiens carbamoyl-phosphate synthase 1 | 46 |
| | | (CPS1), transcript variant 6, non-coding RNA. | |
| CPS1 | NR_163592 | Homo sapiens carbamoyl-phosphate synthase 1 | 47 |
| | | (CPS1), transcript variant 3, non-coding RNA. | |
| LMBRD1 | NM_001363722 | Homo sapiens LMBR1 domain containing 1 | 48 |
| | | (LMBRD1), transcript variant 2, mRNA. | |
| LMBRD1 | NM_001367271 | Homo sapiens LMBR1 domain containing 1 | 49 |
| | | (LMBRD1), transcript variant 3, mRNA. | |
| LMBRD1 | NM_001367272 | Homo sapiens LMBR1 domain containing 1 | 50 |
| | | (LMBRD1), transcript variant 4, mRNA | |
| LMBRD1 | NM_018368 | Homo sapiens LMBR1 domain containing 1 | 51 |
| | | (LMBRD1), mRNA. | |
| MIR33B | NR_030361 | Homo sapiens microRNA 33b (MIR33B), | 52 |
| | | microRNA. | |
| MMAB | NM_052845 | Homo sapiens metabolism of cobalamin associated B | 53 |
| | | (MMAB), transcript variant 1, mRNA. | |
| MMAB | NR_038118 | Homo sapiens metabolism of cobalamin associated B | 54 |
| | | (MMAB), transcript variant 2, non-coding RNA. | |
| OTC | NM_000531 | Homo sapiens ornithine transcarbamylase (OTC), | 55 |
| | | transcript variant 1, mRNA; nuclear gene for | |
| | | mitochondrial product. | |
| PCCA | NM_000282 | Homo sapiens propionyl-CoA carboxylase subunit | 56 |
| | | alpha (PCCA), transcript variant 1, mRNA; nuclear | |
| | | gene for mitochondrial product. | |
| PCCA | NM_001127692 | Homo sapiens propionyl-CoA carboxylase subunit | 57 |
| | | alpha (PCCA), transcript variant 2, mRNA; nuclear | |
| | | gene for mitochondrial product. | |
| PCCA | NM_001178004 | Homo sapiens propionyl-CoA carboxylase subunit | 58 |
| | | alpha (PCCA), transcript variant 3, mRNA; nuclear | |
| | | gene for mitochondrial product. | |
| PCCA | NM_001352605 | Homo sapiens propionyl-CoA carboxylase subunit | 59 |
| | | alpha (PCCA), transcript variant 4, mRNA; nuclear | |
| | | gene for mitochondrial product. | |
| PCCA | NM_001352606 | Homo sapiens propionyl-CoA carboxylase subunit | 60 |
| | | alpha (PCCA), transcript variant 5, mRNA; nuclear | |
| | | gene for mitochondrial product. | |
| PCCA | NM_001352607 | Homo sapiens propionyl-CoA carboxylase subunit | 61 |
| | | alpha (PCCA), transcript variant 6, mRNA; nuclear | |
| | | gene for mitochondrial product. | |
| PCCA | NM_001352608 | Homo sapiens propionyl-CoA carboxylase subunit | 62 |
| | | alpha (PCCA), transcript variant 7, mRNA; nuclear | |
| | | gene for mitochondrial product. | |
| PCCA | NM_001352609 | Homo sapiens propionyl-CoA carboxylase subunit | 63 |
| | | alpha (PCCA), transcript variant 8, mRNA; nuclear | |
| | | gene for mitochondrial product. | |
| PCCA | NM_001352610 | Homo sapiens propionyl-CoA carboxylase subunit | 64 |
| | | alpha (PCCA), transcript variant 9, mRNA; nuclear | |
| | | gene for mitochondrial product. | |
| PCCA | NM_001352611 | Homo sapiens propionyl-CoA carboxylase subunit | 65 |
| | | alpha (PCCA), transcript variant 10, mRNA; nuclear | |
| | | gene for mitochondrial product | |
| PCCA | NM_001352612 | Homo sapiens propionyl-CoA carboxylase subunit | 66 |
| | | alpha (PCCA), transcript variant 11, mRNA; nuclear | |
| | | gene for mitochondrial product. | |
| PCCA | NR_148027 | Homo sapiens propionyl-CoA carboxylase subunit | 67 |
| | | alpha (PCCA), transcript variant 12, non-coding | |
| | | RNA. | |
| PCCA | NR_148028 | Homo sapiens propionyl-CoA carboxylase subunit | 68 |
| | | alpha (PCCA), transcript variant 13, non-coding | |
| | | RNA. | |
| PCCA | NR_148029 | Homo sapiens propionyl-CoA carboxylase subunit | 69 |
| | | alpha (PCCA), transcript variant 14, non-coding | |
| | | RNA. | |
| PCCA | NR_148030 | Homo sapiens propionyl-CoA carboxylase subunit | 70 |
| | | alpha (PCCA), transcript variant 15, non-coding | |
| | | RNA. | |
| PCCA | NR_148031 | Homo sapiens propionyl-CoA carboxylase subunit | 71 |
| | | alpha (PCCA), transcript variant 16, non-coding | |
| | | RNA | |
| RAI1 | NM_030665 | Homo sapiens retinoic acid induced 1 (RAI1), | 72 |
| | | mRNA. | |
| SREBF1 | NM_001005291 | Homo sapiens sterol regulatory element binding | 73 |
| | | transcription factor 1 (SREBF1), transcript variant 1, | |
| | | mRNA. | |
| SREBF1 | NM_001321096 | Homo sapiens sterol regulatory element binding | 74 |
| | | transcription factor 1 (SREBF1), transcript variant 3, | |
| | | mRNA. | |
| SREBF1 | NM_001388385 | Homo sapiens sterol regulatory element binding | 75 |
| | | transcription factor 1 (SREBF1), transcript variant 4, | |
| | | mRNA. | |
| SREBF1 | NM_001388386 | Homo sapiens sterol regulatory element binding | 76 |
| | | transcription factor 1 (SREBF1), transcript variant 5, | |
| | | mRNA. | |
| SREBF1 | NM_001388387 | Homo sapiens sterol regulatory element binding | 77 |
| | | transcription factor 1 (SREBF1), transcript variant 6, | |
| | | mRNA. | |
| SREBF1 | NM_001388388 | Homo sapiens sterol regulatory element binding | 78 |
| | | transcription factor 1 (SREBF1), transcript variant 7, | |
| | | mRNA. | |
| SREBF1 | NM_001388389 | Homo sapiens sterol regulatory element binding | 79 |
| | | transcription factor 1 (SREBF1), transcript variant 8, | |
| | | mRNA. | |
| SREBF1 | NM_001388390 | Homo sapiens sterol regulatory element binding | 80 |
| | | transcription factor 1 (SREBF1), transcript variant 9, | |
| | | mRNA. | |
| SREBF1 | NM_001388391 | Homo sapiens sterol regulatory element binding | 81 |
| | | transcription factor 1 (SREBF1), transcript variant | |
| | | 10, mRNA. | |
| SREBF1 | NM_001388392 | Homo sapiens sterol regulatory element binding | 82 |
| | | transcription factor 1 (SREBF1), transcript variant | |
| | | 11, mRNA. | |
| SREBF1 | NM_001388393 | Homo sapiens sterol regulatory element binding | 83 |
| | | transcription factor 1 (SREBF1), transcript variant | |
| | | 12, mRNA. | |
| SREBF1 | NM_001388394 | Homo sapiens sterol regulatory element binding | 84 |
| | | transcription factor 1 (SREBF1), transcript variant | |
| | | 13, mRNA. | |
| SREBF1 | NM_004176 | Homo sapiens sterol regulatory element binding | 85 |
| | | transcription factor 1 (SREBF1), transcript variant 2, | |
| | | mRNA. | |
| SREBF1 | NR_170943 | Homo sapiens sterol regulatory element binding | 86 |
| | | transcription factor 1 (SREBF1), transcript variant | |
| | | 14, non-coding RNA. | |
| SREBF1 | NR_170944 | Homo sapiens sterol regulatory element binding | 87 |
| | | transcription factor 1 (SREBF1), transcript variant | |
| | | 15, non-coding RNA. | |
| SREBF1 | NR_170945 | Homo sapiens sterol regulatory element binding | 88 |
| | | transcription factor 1 (SREBF1), transcript variant | |
| | | 16, non-coding RNA. | |
| SREBF1 | NR_170990 | Homo sapiens sterol regulatory element binding | 89 |
| | | transcription factor 1 (SREBF1), transcript variant | |
| | | 17, non-coding RNA. | |
| TSPAN7 | NM_004615 | Homo sapiens tetraspanin 7 (TSPAN7), mRNA. | 90 |
| UBE2W | NM_001001481 | Homo sapiens ubiquitin-conjugating enzyme E2W | 91 |
| | | (putative) (UBE2W), transcript variant 1, mRNA. | |
| UBE2W | NM_{——}001271015 | Homo sapiens ubiquitin-conjugating enzyme E2W | 92 |
| | | (putative) (UBE2W), transcript variant 3, mRNA. | |
| UBE2W | NM_018299 | Homo sapiens ubiquitin-conjugating enzyme E2W | 93 |
| | | (putative) (UBE2W), transcript variant 2, mRNA. | |
| UBE2W | NR_073119 | Homo sapiens ubiquitin-conjugating enzyme E2W | 94 |
| | | (putative) (UBE2W), transcript variant 4, non-coding | |
| | | RNA. | |
| UBE2W | NR_073120 | HHomo sapiens ubiquitin-conjugating enzyme E2W | 95 |
| | | (putative) (UBE2W), transcript variant 5, non-coding | |
| | | RNA. | |
| UBE2W | NR_073121 | Homo sapiens ubiquitin-conjugating enzyme E2W | 96 |
| | | (putative) (UBE2W), transcript variant 6, non-coding | |
| | | RNA. | |

For all genes listed in Table 1, Table 3 represents a non-redundant list of the transcript variants for each gene impacted by an exonic or intronic CNV. The column headers in Table 3 are as follows: RefSeq Gene Symbol—official HGNC-provided gene symbol used by NCBI's Reference Sequence (RefSeq) database; RefSeq Accession Number—all presently known transcript variants for each gene in Table 2 are listed (hence Table 3 has more entries than Table 2); Transcript Definition—brief description of the type (e.g., mRNA or non-coding RNA) and number of the transcript variant; SEQ ID—Sequence identification number in the Sequence Listing.

TABLE 4
Non-redundant list of UCD/HA genes that are adjacent (within 250 Kb) to an intergenic CNV

| RefSeq | | NCBI | | |
|---|---|---|---|---|
| Gene | Gene | Gene | | |
| Symbol | Alias | ID | Gene Full Name | Gene Summary |
| GLUL | GS; | 2752 | glutamate- | The protein encoded by this gene belongs to the |
| | GLNS; | | ammonia ligase | glutamine synthetase family. It catalyzes the synthesis |
| | PIG43; | | | of glutamine from glutamate and ammonia in an ATP- |
| | PIG59 | | | dependent reaction. This protein plays a role in |
| | | | | ammonia and glutamate detoxification, acid-base |
| | | | | homeostasis, cell signaling, and cell proliferation. |
| | | | | Glutamine is an abundant amino acid, and is important |
| | | | | to the biosynthesis of several amino acids, pyrimidines, |
| | | | | and purines. Mutations in this gene are associated with |
| | | | | congenital glutamine deficiency, and overexpression of |
| | | | | this gene was observed in some primary liver cancer |
| | | | | samples. There are six pseudogenes of this gene found |
| | | | | on chromosomes 2, 5, 9, 11, and 12. Alternative |
| | | | | splicing results in multiple transcript variants. |
| | | | | [provided by RefSeq, Dec 2014] |
| OTC | OCTD; | 5009 | ornithine | This nuclear gene encodes a mitochondrial matrix |
| | OTC1; | | transcarbamylase | enzyme. The encoded protein is involved in the urea |
| | OTCD; | | | cycle which functions to detoxify ammonia into urea |
| | OTCase | | | for excretion. Mutations in this enzyme lead to |
| | | | | ornithine transcarbamylase deficiency, which causes |
| | | | | hyperammonemia. [provided by RefSeq, May 2022] |
| SLC25A13 | CTLN2; | 10165 | solute carrier | This gene is a member of the mitochondrial carrier |
| | NICCD; | | family 25 | family. The encoded protein contains four EF-hand |
| | CITRIN; | | member 13 | Ca(2 +) binding motifs in the N-terminal domain, and |
| | ARALAR2 | | | localizes to mitochondria. The protein catalyzes the |
| | | | | exchange of aspartate for glutamate and a proton across |
| | | | | the inner mitochondrial membrane, and is stimulated |
| | | | | by calcium on the external side of the inner |
| | | | | mitochondrial membrane. Mutations in this gene result |
| | | | | in citrullinemia, type II. Multiple transcript variants |
| | | | | encoding different isoforms have been found for this |
| | | | | gene. [provided by RefSeq, May 2009] |
| SLC7A7 | LPI; | 9056 | solute carrier | The protein encoded by this gene is the light subunit of |
| | LAT3; | | family 7 member | a cationic amino acid transporter. This sodium- |
| | MOP-2; | | 7 | independent transporter is formed when the light |
| | Y + LAT1; | | | subunit encoded by this gene dimerizes with the heavy |
| | y + LAT-1 | | | subunit transporter protein SLC3A2. This transporter is |
| | | | | found in epithelial cell membranes where it transfers |
| | | | | cationic and large neutral amino acids from the cell to |
| | | | | the extracellular space. Defects in this gene are a cause |
| | | | | of lysinuric protein intolerance (LPI). Alternative |
| | | | | splicing results in multiple transcript variants. |
| | | | | [provided by RefSeq, Jul 2011] |
| TAFAZZIN | EFE; TAZ; | 6901 | tafazzin, | This gene encodes a protein that is expressed at high |
| | BTHS; | | phospholipid- | levels in cardiac and skeletal muscle. Mutations in this |
| | EFE2; | | lysophospholipid | gene have been associated with a number of clinical |
| | G4.5; | | transacylase | disorders including Barth syndrome, dilated |
| | Taz1; | | | cardiomyopathy (DCM), hypertrophic DCM, |
| | CMD3A; | | | endocardial fibroelastosis, and left ventricular |
| | LVNCX | | | noncompaction (LVNC). Multiple transcript variants |
| | | | | encoding different isoforms have been described. A |
| | | | | long form and a short form of each of these isoforms is |
| | | | | produced; the short form lacks a hydrophobic leader |
| | | | | sequence and may exist as a cytoplasmic protein rather |
| | | | | than being membrane-bound. Other alternatively |
| | | | | spliced transcripts have been described but the full- |
| | | | | length nature of all these transcripts is not known. |
| | | | | [provided by RefSeq, Jul 2008] |

For all intergenic CNVs listed in Table 1 (i.e., those with no gene listed in the RefSeq Gene Symbol column), Table 4 represents a non-redundant list of the UCD/HA genes that are adjacent (within 250 Kb) to an intergenic CNV. The column headers in Table 4 are as follows: RefSeq Gene Symbol-official HGNC-provided gene symbol used by NCBI's Reference Sequence (RefSeq) database, which is based on the HUGO Gene Nomenclature Committee (HGNC) wherein HUGO refers to the Human Genome Organization; Gene Alias—list of previously used gene symbols (entries with none have no gene aliases reported in the HGNC database); NCBI Gene ID—NCBI's unique gene identifier number; Gene Full Name—official HGNC-provided full name for the gene; RefSeq Summary—RefSeq-provided summary of the gene's function and/or biology.

TABLE 5
Non-redundant list of the transcript variants for each UCD/HA gene
adjacent to an intergenic CNV for all genes listed in Table 4.

| RefSeq | | | |
|---|---|---|---|
| Gene | RefSeq Accession | | |
| Symbol | Number | Transcript Definition | SEQ ID |
| GLUL | NM_001033044 | Homo sapiens glutamate-ammonia ligase (GLUL), | 97 |
| | | transcript variant 2, mRNA. | |
| GLUL | NM_001033056 | Homo sapiens glutamate-ammonia ligase (GLUL), | 98 |
| | | transcript variant 3, mRNA. | |
| GLUL | NM_002065 | Homo sapiens glutamate-ammonia ligase (GLUL), | 99 |
| | | transcript variant 1, mRNA. | |
| OTC | NM_000531 | Homo sapiens ornithine transcarbamylase (OTC), | 55 |
| | | transcript variant 1, mRNA; nuclear gene for | |
| | | mitochondrial product. | |
| SLC25A13 | NM_001160210 | Homo sapiens solute carrier family 25 member 13 | 100 |
| | | (SLC25A13), transcript variant 1, mRNA; nuclear | |
| | | gene for mitochondrial product. | |
| SLC25A13 | NM_014251 | Homo sapiens solute carrier family 25 member 13 | 101 |
| | | (SLC25A13), transcript variant 2, mRNA; nuclear | |
| | | gene for mitochondrial product. | |
| SLC25A13 | NR_027662 | Homo sapiens solute carrier family 25 member 13 | 102 |
| | | (SLC25A13), transcript variant 3, non-coding RNA. | |
| SLC7A7 | NM_001126105 | Homo sapiens solute carrier family 7 member 7 | 103 |
| | | (SLC7A7), transcript variant 2, mRNA. | |
| SLC7A7 | NM_001126106 | Homo sapiens solute carrier family 7 member 7 | 104 |
| | | (SLC7A7), transcript variant 3, mRNA. | |
| SLC7A7 | NM_003982 | Homo sapiens solute carrier family 7 member 7 | 105 |
| | | (SLC7A7), transcript variant 1, mRNA. | |
| TAFAZZIN | NM_000116 | Homo sapiens tafazzin, phospholipid- | 106 |
| | | lysophospholipid transacylase (TAFAZZIN), | |
| | | transcript variant 1, mRNA. | |
| TAFAZZIN | NM_001303465 | Homo sapiens tafazzin, phospholipid- | 107 |
| | | lysophospholipid transacylase (TAFAZZIN), | |
| | | transcript variant 5, mRNA. | |
| TAFAZZIN | NM_181311 | Homo sapiens tafazzin, phospholipid- | 108 |
| | | lysophospholipid transacylase (TAFAZZIN), | |
| | | transcript variant 2, mRNA. | |
| TAFAZZIN | NM_181312 | Homo sapiens tafazzin, phospholipid- | 109 |
| | | lysophospholipid transacylase (TAFAZZIN), | |
| | | transcript variant 3, mRNA. | |
| TAFAZZIN | NM_181313 | Homo sapiens tafazzin, phospholipid- | 110 |
| | | lysophospholipid transacylase (TAFAZZIN), | |
| | | transcript variant 4, mRNA. | |
| TAFAZZIN | NR_024048 | Homo sapiens tafazzin, phospholipid- | 111 |
| | | lysophospholipid transacylase (TAFAZZIN), | |
| | | transcript variant 6, non-coding RNA. | |

For all genes listed in Table 4, Table 5 represents a non-redundant list of the transcript variants for each UCD/HA gene adjacent to an intergenic CNV. The column headers in Table 5 are as follows: RefSeq Gene Symbol—official HGNC-provided gene symbol used by NCBI's Reference Sequence (RefSeq) database; RefSeq Accession Number—all presently known transcript variants for each gene in Table 4 are listed; Transcript Definition—brief description of the type (e.g., mRNA or non-coding RNA) and number of the transcript variant; SEQ ID—Sequence identification number in the Sequence Listing.

TABLE 6
Relevant sequence information for the
CPS1 deletion reported in Table 1

| | | PCR Primer | |
|---|---|---|---|
| | Sequence | Sequence | SEQ |
| Sequence Name | Item | (5′ to 3′) | ID |
| CPS1_delF | PCR | CAGATACTATTT | 112 |
| | primer | TTGCCAACATGC | |
| CPS1_delR | PCR | AGGCAGTGACCC | 113 |
| | primer | ATCAGTATATGT | |
| CPS1_POS_98_F* | Sanger | n/a | 114 |
| | sequence | | |
| CPS1_POS_98_R* | Sanger | n/a | 115 |
| | sequence | | |
| CPS1_del_PBio | aCGH data | n/a | 3 |
| CPS1_del_1KG_Ph3 | WGS data | n/a | 116 |

*POS_98 is deletion-positive LOAD case Expt ID 2693 (see FIG. 1 and Table 1)

Table 6 lists the relevant sequence information for the CPS1 deletion reported in Table 1 (SEQ ID 3, also listed in Table 6). This includes the PCR primer sequences used to develop a CPS1 deletion assay, Sanger sequence data (experiments were performed to verify the deletion breakpoints), and the CPS1 deletion reported in a public database (IKG Ph3 refers to the 1000 genomes project Phase 3 data, the deletion ID is esv3594156, see Table 7 for further details). The column headers in Table 6 are as follows: Sequence Name—name of sequence; Sequence Item—type and/or source of the sequence information; PCR Primer Sequence (5′ to 3′)—sequences for the PCR primers; SEQ ID—Sequence identification number in the Sequence Listing.

TABLE 7
Statistical analysis for the prevalence of the deletion affecting an intron of CPS1

| | | Genome | | Normals | | LOAD | | | | | |
|---|---|---|---|---|---|---|---|---|---|---|---|
| Statistical | Deletion | Coordinates | Size | Cohort | Normals | Cohort | LOAD | | | OR | OR |
| Comparison | ID | (GRCh37/hg19) | (bp) | Size | Positive | Size | Positive | p-value | OR | LCL | UCL |
| LOAD (EUR) | PBio_Data | chr2: 211361945- | 6,430 | 1,000 | 1 | 100 | 3 | 0.00273 | 30.90 | 3.18 | 299.90 |
| vs. NVE | | 211368375 | | | | | | | | | |
| (EUR) | | | | | | | | | | | |
| LOAD (EUR) | esv3594156 | chr2: 211360864- | 7,745 | 2,504 | 2 | 100 | 3 | 0.00052 | 38.69 | 6.39 | 234.23 |
| vs. 1 KG Ph 3 | | 211368609 | | | | | | | | | |
| (all) | | | | | | | | | | | |
| LOAD (EUR) | esv3594156 | chr2: 211360864- | 7,745 | 500 | 2 | 100 | 3 | 0.03485 | 7.70 | 1.27 | 46.70 |
| vs. 1 KG Ph 3 | | 211368609 | | | | | | | | | |
| (EUR) | | | | | | | | | | | |
| LOAD (EUR) | DEL_2_26674 | chr2: 211360860- | 7,750 | 3,812 | 19 | 100 | 3 | 0.01754 | 6.17 | 1.80 | 21.21 |
| vs. gnomAD | | 211368610 | | | | | | | | | |
| (EUR, all) | | | | | | | | | | | |
| LOAD (EUR) | DEL_2_26674 | chr2: 211360860- | 7,750 | 3,663 | 19 | 100 | 3 | 0.01943 | 5.93 | 1.73 | 20.38 |
| vs. gnomAD | | 211368610 | | | | | | | | | |
| (EUR, non- | | | | | | | | | | | |
| neuro) | | | | | | | | | | | |
| LOAD (EUR) | DEL_2_26674 | chr2: 211360860- | 7,750 | 1,660 | 6 | 100 | 3 | 0.01164 | 8.53 | 2.10 | 34.61 |
| vs. gnomAD | | 211368610 | | | | | | | | | |
| (EUR, | | | | | | | | | | | |
| controls) | | | | | | | | | | | |
| LOAD (EUR) | NA | chr2: 211360861- | 7,749 | 6,079 | 17 | 100 | 3 | 0.00384 | 11.03 | 3.18 | 38.25 |
| vs. MSSNG | | 211368610 | | | | | | | | | |
| (unaffected) | | | | | | | | | | | |
| LOAD (EUR) | gssvL69036 | chr2: 211360857- | 7,753 | 2,562 | 2 | 100 | 3 | 0.00049 | 39.59 | 6.54 | 239.65 |
| vs. DGV Gold | | 211368610 | | | | | | | | | |
| Standard* | | | | | | | | | | | |

*Overlaps with 1 KG Ph 3 (all)

Table 7 demonstrates statistical analysis for the prevalence of the deletion affecting an intron of CPS1 in different databases (including Population Bio's proprietary database). This deletion is observed in other population databases but at lower frequency than observed in our LOAD cohort. Using Fisher's Exact Test (FET, two-tailed), Table 7 shows a p-value of <0.05 for all comparison categories, suggesting that the observed increase in frequency of the CPS1 deletion (SEQ ID 3) in LOAD samples is significant (see also Example 4).

Abbreviations/definitions for Table 7: NVE—normal variation engine, which is a Population Bio (PBio) proprietary database of copy number variation in normal individuals; LOAD—Late-Onset Alzheimer's Disease, which are samples from the OPTIMA cohort; IKG—1,000 Genomes Project (see PubMed PMID 26432245); gnomAD—Genome Aggregation Database (see PubMed PMID 32461654); MSSNG—Autism database (research.mss.ng and see PubMed PMID 32317787); DGV—Database of Genomic Variants (see PubMed PMID 24174537). The gnomAD structural variant (SV) calls are generated from a set of whole genome sequenced (WGS) samples that largely overlaps those used in gnomAD v2.1 variant (e.g., SNVs, indels) database. The current SV release includes 10,847 (allele number=21,694) unrelated subjects, of which 3,812 have European (EUR) ancestry. gnomAD SVs v2.1 (non-neuro): Only samples from individuals who were not ascertained for having a neurological condition in a neurological case/control study; 8,342 unrelated subjects, of which 3,663 have EUR ancestry. gnomAD SVs v2.1 (controls): Only samples from individuals who were not designated as a case in a case/control study of common disease; 5,192 unrelated subjects, of which 1,660 have EUR ancestry.

The column headers in Table 7 are as follows: Statistical Comparison—which groups of cases (with LOAD) and controls (aka normal subjects) are being compared; Deletion ID—name attributed to the deletion in the relevant database; Genomic Coordinates (GRCh3 7/hg 19)—deletion extent (start and stop positions) in hg19 coordinates; Size—deletion size in base pairs (bp); Nonnals Cohort Size—Total number of normal (unaffected) subjects in the cohort that were evaluated for the deletion; Nonnals Positive-Number of unaffected individuals with the deletion; LOAD Cohort Size—Total number of LOAD (affected) cases in the cohort that were evaluated for the deletion; LOAD Positive—Number of affected individuals with the deletion; p-value—measure of statistical significance using FET; OR—odds ratio, a measure of effect size; OR LCL—odds ratio, lower confidence limit (95%); OR UCL—odds ratio, upper confidence limit (95%).

TABLE 8
Non-redundant list of UCD genes only

| RefSeq | | | | | | | | Gene Start − | Gene Stop + |
|---|---|---|---|---|---|---|---|---|---|
| Gene | | | Disease | Disease Name | | Gene Start | Gene Stop | 250 Kb | 250 Kb |
| Symbol | Gene Full Name | OMIM | Model | (OMIM) | Chr | (hg18) | (hg18) | (hg18) | (hg18) |
| ARG1 | arginase 1 | 207800, | AR | Argininemia | 6 | 131833665 | 131947165 | 131583665 | 132197165 |
| | | 608313 | | | | | | | |
| ASL | argininosuccinate | 207900, | AR | Argininosuccinic | 7 | 65178222 | 65197119 | 64928222 | 65447119 |
| | lyase | 608310 | | aciduria | | | | | |
| ASS1 | argininosuccinate | 215700, | AR | Citrullinemia | 9 | 132310169 | 132366482 | 132060169 | 132616482 |
| | synthase 1 | 603470 | | | | | | | |
| CPS1 | aarbamoyl- | 237300, | AR | Carbamoylphosphate | 2 | 211050651 | 211252076 | 210800651 | 211502076 |
| | phosphate | 608307 | | synthetase I deficiency | | | | | |
| | synthase 1 | | | | | | | | |
| NAGS | N- | 237310, | AR | N-acetylglutamate | 17 | 39437516 | 39441962 | 39187516 | 39691962 |
| | acetylglutamate | 608300 | | synthase deficiency | | | | | |
| | synthase | | | | | | | | |
| OTC | ornithine | 300461, | XLR | Ornithine | X | 38096783 | 38165643 | 37846783 | 38415643 |
| | transcarbamylase | 311250 | | transcarbamylase | | | | | |
| | | | | deficiency | | | | | |
| SLC25A13 | solute carrier | 603859, | AR | Citrullinemia, type II, | 7 | 95587468 | 95789395 | 95337468 | 96039395 |
| | family 25 | 605814 | | neonatal-onset; | | | | | |
| | member 13 | | | Citrullinemia, adult- | | | | | |
| | | | | onset type II | | | | | |
| SLC25A15 | solute carrier | 238970, | AR | Hyperornithinemia- | 13 | 40261548 | 40284596 | 40011548 | 40534596 |
| | family 25 | 603861 | | hyperammonemia- | | | | | |
| | member 15 | | | homocitrullinemia | | | | | |
| | | | | syndrome | | | | | |

Table 8 presents the non-redundant list of UCD genes only. The column headers in Table 8 are as follows: RefSeq Gene Symbol—gene symbol used by NCBI's Reference Sequence (RefSeq) database; Gene Full Name—official HGNC-provided full name for the gene; OMIM—OMIM (Online Mendelian Inheritance in Man) entry numbers for the gene and phenotype; Disease Model—indicates if inheritance is autosomal dominant (AD), autosomal recessive (AR), or X-linked recessive (XLR); Disease Name (OMIM)—name of the type of UCD as listed in OMIM; Chr—chromosome that harbors the UCD gene; Gene Start (hg18)—genome position corresponding to the start of the gene (based on human assembly NCBI 36/hg18); Gene Stop (hg18)—genome position corresponding to the end (stop) of the gene (based on human assembly NCBI 36/hg18); Gene Start—250 Kb (hg18)—genome position corresponding to the start of the gene (based on human assembly NCBI 36/hg18) minus 250 Kb; Gene Stop+250 Kb (hg18)-genome position corresponding to the end (stop) of the gene (based on human assembly NCBI 36/hg18) plus 250 Kb.

TABLE 9
Non-redundant list of other HA genes not listed in Table 8

| RefSeq | | | | | | | | Gene Start − | Gene Stop + |
|---|---|---|---|---|---|---|---|---|---|
| Gene | | | Disease | Disease Name | | Gene Start | Gene Stop | 250 Kb | 250 Kb |
| Symbol | Gene Full Name | OMIM | Model | (OMIM) | Chr | (hg18) | (hg18) | (hg18) | (hg18) |
| ACADM | acyl-CoA | 607008, | AR | Acyl-CoA | 1 | 75962704 | 76025848 | 75712704 | 76275848 |
| | dehydrogenase | 201450 | | dehydrogenase, | | | | | |
| | medium chain | | | medium chain, | | | | | |
| | | | | deficiency of | | | | | |
| ACADVL | acyl-CoA | 609575, | AR | VLCAD | 17 | 7061168 | 7069309 | 6811168 | 7319309 |
| | dehydrogenase very | 201475 | | deficiency | | | | | |
| | long chain | | | | | | | | |
| ALDH18A1 | aldehyde | 138250, | AR | Cutis laxa, | 10 | 97355688 | 97406458 | 97105688 | 97656458 |
| | dehydrogenase 18 | 219150 | | autosomal | | | | | |
| | family member A1 | | | recessive, type | | | | | |
| | | | | IIIA | | | | | |
| AMT | aminomethyltransferase | 238310, | AR | Glycine | 3 | 49429215 | 49435122 | 49179215 | 49685122 |
| | | 605899 | | encephalopathy | | | | | |
| ATP5F1D | ATP synthase F1 | 603150, | AR | Mitochondrial | 19 | 1192745 | 1195824 | 942745 | 1445824 |
| | subunit delta | 618120 | | complex V | | | | | |
| | | | | (ATP synthase) | | | | | |
| | | | | deficiency | | | | | |
| ATPAF2 | ATP synthase | 608918, | AR | Mitochondrial | 17 | 17821448 | 17883248 | 17571448 | 18133248 |
| | mitochondrial F1 | 604273 | | complex V | | | | | |
| | complex assembly | | | (ATP synthase) | | | | | |
| | factor 2 | | | deficiency, | | | | | |
| | | | | nuclear type 1 | | | | | |
| CA5A | carbonic anhydrase | 114761, | AR | Carbonic | 16 | 86472653 | 86527687 | 86222653 | 86777687 |
| | 5A | 615751 | | anydrase VA | | | | | |
| | | | | deficiency | | | | | |
| CPT1A | carnitine | 255120, | AR | CPT deficiency, | 11 | 68278664 | 68368454 | 68028664 | 68618454 |
| | palmitoyltransferase | 600528 | | hepatic, type IA | | | | | |
| | 1A | | | | | | | | |
| CPT2 | carnitine | 600650, | AR | CPT II | 1 | 53435052 | 53452457 | 53185052 | 53702457 |
| | palmitoyltransferase | 608836 | | deficiency, | | | | | |
| | 2 | | | infantile | | | | | |
| CYC1 | cytochrome c1 | 123980, | AR | Mitochondrial | 8 | 145221930 | 145224416 | 144971930 | 145474416 |
| | | 615453 | | complex III | | | | | |
| | | | | deficiency, | | | | | |
| | | | | nuclear type 6 | | | | | |
| DLAT | dihydrolipoamide S- | 608770, | AR | Pyruvate | 11 | 111401342 | 111440338 | 111151342 | 111690338 |
| | acetyltransferase | 245348 | | dehydrogenase | | | | | |
| | | | | E2 deficiency | | | | | |
| ETFA | electron transfer | 608053, | AR | Glutaric | 15 | 74267951 | 74391126 | 74017951 | 74641126 |
| | flavoprotein subunit | 231680 | | acidemia IIA | | | | | |
| | alpha | | | | | | | | |
| ETFB | electron transfer | 130410, | AR | Glutaric | 19 | 56540235 | 56561454 | 56290235 | 56811454 |
| | flavoprotein subunit | 231680 | | acidemia IIB | | | | | |
| | beta | | | | | | | | |
| ETFDH | electron transfer | 231675, | AR | Glutaric | 4 | 159812570 | 159851344 | 159562570 | 160101344 |
| | flavoprotein | 231680 | | acidemia IIC | | | | | |
| | dehydrogenase | | | | | | | | |
| FBXL4 | F-box and leucine | 605654, | AR | Mitochondrial | 6 | 99423132 | 99502603 | 99173132 | 99752603 |
| | rich repeat protein 4 | 615471 | | DNA depletion | | | | | |
| | | | | syndrome 13 | | | | | |
| | | | | (encephalomyopathic | | | | | |
| | | | | type) | | | | | |
| FH | fumarate hydratase | 136850, | AR | Fumarase | 1 | 239727434 | 239749722 | 239477434 | 239999722 |
| | | 606812 | | deficiency | | | | | |
| GLUD1 | glutamate | 138130, | AD | Hyperinsulinism- | 10 | 88799939 | 88844580 | 88549939 | 89094580 |
| | dehydrogenase 1 | 606762 | | hyperammonemia | | | | | |
| | | | | syndrome | | | | | |
| GLUL | glutamate-ammonia | 138290, | AR | Glutamine | 1 | 180613856 | 180627964 | 180363856 | 180877964 |
| | ligase | 610015 | | deficiency, | | | | | |
| | | | | congenital | | | | | |
| HADHA | hydroxyacyl-CoA | 600890, | AR | Mitochondrial | 2 | 26267008 | 26321044 | 26017008 | 26571044 |
| | dehydrogenase | 609015 | | trifunctional | | | | | |
| | trifunctional | | | protein | | | | | |
| | multienzyme | | | deficiency | | | | | |
| | complex subunit | | | | | | | | |
| | alpha | | | | | | | | |
| HADHB | hydroxyacyl-CoA | 143450, | AR | Trifunctional | 2 | 26319542 | 26366837 | 26069542 | 26616837 |
| | dehydrogenase | 609015 | | protein | | | | | |
| | trifunctional | | | deficiency | | | | | |
| | multienzyme | | | | | | | | |
| | complex subunit beta | | | | | | | | |
| HLCS | holocarboxylase | 253270, | AR | Holocarboxylase | 21 | 37042797 | 37284406 | 36792797 | 37534406 |
| | synthetase | 609018 | | synthetase | | | | | |
| | | | | deficiency | | | | | |
| HMGCL | 3-hydroxy-3- | 246450, | AR | HMG-CoA lyase | 1 | 24000962 | 24037697 | 23750962 | 24287697 |
| | methylglutaryl-CoA | 613898 | | deficiency | | | | | |
| | lyase | | | | | | | | |
| IVD | isovalery1-CoA | 243500, | AR | Isovaleric | 15 | 38484978 | 38515438 | 38234978 | 38765438 |
| | dehydrogenase | 607036 | | acidemia | | | | | |
| LMBRD1 | LMBR1 domain | 612625, | AR | Methylmalonic | 6 | 70439370 | 70633849 | 70189370 | 70883849 |
| | containing 1 | 277380 | | aciduria and | | | | | |
| | | | | homocystinuria, | | | | | |
| | | | | cblF type | | | | | |
| MCCC1 | methylcrotonoyl- | 210200, | AR | 3- | 3 | 184215700 | 184316557 | 183965700 | 184566557 |
| | CoA carboxylase 1 | 609010 | | Methylcrotonyl- | | | | | |
| | | | | CoA | | | | | |
| | | | | carboxylase 1 | | | | | |
| | | | | deficiency | | | | | |
| MCCC2 | methylcrotonoyl- | 210210, | AR | 3- | 5 | 70911114 | 70990289 | 70661114 | 71240289 |
| | CoA carboxylase 2 | 609014 | | Methylcrotonyl- | | | | | |
| | | | | CoA | | | | | |
| | | | | carboxylase 2 | | | | | |
| | | | | deficiency | | | | | |
| MCEE | methylmalonyl-CoA | 608419, | AR | Methylmalonyl- | 2 | 71190322 | 71210877 | 70940322 | 71460877 |
| | epimerase | 251120 | | CoA epimerase | | | | | |
| | | | | deficiency | | | | | |
| MLYCD | malonyl-CoA | 606761, | AR | Malonyl-CoA | 16 | 82490221 | 82542551 | 82240221 | 82792551 |
| | decarboxylase | 248360 | | decarboxylase | | | | | |
| | | | | deficiency | | | | | |
| MMAA | metabolism of | 251100, | AR | Methylmalonic | 4 | 146739644 | 146800635 | 146489644 | 147050635 |
| | cobalamin associated | 607481 | | aciduria, vitamin | | | | | |
| | A | | | B12-responsive | | | | | |
| MMAB | metabolism of | 607568, | AR | Methylmalonic | 12 | 108475903 | 108495768 | 108225903 | 108745768 |
| | cobalamin associated | 251110 | | aciduria, vitamin | | | | | |
| | B | | | B12-responsive, | | | | | |
| | | | | cblB type | | | | | |
| MMACHC | metabolism of | 609831, | AR | Methylmalonic | 1 | 45738559 | 45751641 | 45488559 | 46001641 |
| | cobalamin associated | 277400 | | aciduria and | | | | | |
| | C | | | homocystinuria, | | | | | |
| | | | | cblC type | | | | | |
| MMADHC | metabolism of | 611935, | AR | Homocystinuria, | 2 | 150134397 | 150152538 | 149884397 | 150402538 |
| | cobalamin associated | 277410 | | cblD type, | | | | | |
| | D | | | variant 1; | | | | | |
| | | | | Methylmalonic | | | | | |
| | | | | aciduria and | | | | | |
| | | | | homocystinuria, | | | | | |
| | | | | cblD type; | | | | | |
| | | | | Methylmalonic | | | | | |
| | | | | aciduria, cblD | | | | | |
| | | | | type, variant 2 | | | | | |
| MMUT | methylmalonyl-CoA | 251000, | AR | Methylmalonic | 6 | 49506032 | 49538925 | 49256032 | 49788925 |
| | mutase | 609058 | | aciduria, mut(0) | | | | | |
| | | | | type | | | | | |
| OAT | ornithine | 258870, | AR | Gyrate atrophy | 10 | 126075862 | 126097535 | 125825862 | 126347535 |
| | aminotransferase | 613349 | | of choroid and | | | | | |
| | | | | retina with or | | | | | |
| | | | | without | | | | | |
| | | | | ornithinemia | | | | | |
| PC | pyruvate carboxylase | 266150, | AR | Pyruvate | 11 | 66372464 | 66482433 | 66122464 | 66732433 |
| | | 608786 | | carboxylase | | | | | |
| | | | | deficiency | | | | | |
| PCCA | propionyl-CoA | 232050, | AR | Propionicacidemia | 13 | 99539270 | 99980692 | 99289270 | 100230692 |
| | carboxylase subunit | 606054 | | | | | | | |
| | alpha | | | | | | | | |
| PCCB | propionyl-CoA | 232000, | AR | Propionicacidemia | 3 | 137451872 | 137539428 | 137201872 | 137789428 |
| | carboxylase subunit | 606054 | | | | | | | |
| | beta | | | | | | | | |
| PDHA1 | pyruvate | 300502, | XLD | Pyruvate | X | 19271932 | 19289757 | 19021932 | 19539757 |
| | dehydrogenase E1 | 312170 | | dehydrogenase | | | | | |
| | subunit alpha 1 | | | E1-alpha | | | | | |
| | | | | deficiency | | | | | |
| SLC22A5 | solute carrier family | 212140, | AR | Carnitine | 5 | 131733301 | 131759204 | 131483301 | 132009204 |
| | 22 member 5 | 603377 | | deficiency, | | | | | |
| | | | | systemic | | | | | |
| | | | | primary | | | | | |
| SLC25A20 | solute carrier family | 613698, | AR | Carnitine- | 3 | 48869363 | 48911341 | 48619363 | 49161341 |
| | 25 member 20 | 212138 | | acylcarnitine | | | | | |
| | | | | translocase | | | | | |
| | | | | deficiency | | | | | |
| SLC25A42 | solute carrier family | 610823, | AR | Metabolic crises, | 19 | 19035803 | 19084839 | 18785803 | 19334839 |
| | 25 member 42 | 618416 | | recurrent, with | | | | | |
| | | | | variable | | | | | |
| | | | | encephalomyopathic | | | | | |
| | | | | features and | | | | | |
| | | | | neurologic | | | | | |
| | | | | regression | | | | | |
| SLC7A7 | solute carrier family | 222700, | AR | Lysinuric | 14 | 22312271 | 22368869 | 22062271 | 22618869 |
| | 7 member 7 | 603593 | | protein | | | | | |
| | | | | intolerance | | | | | |
| TAFAZZIN | tafazzin, | 300394, | XLR | Barth syndrome | X | 153293054 | 153303259 | 153043054 | 153553259 |
| | phospholipid- | 302060 | | | | | | | |
| | lysophospholipid | | | | | | | | |
| | transacylase | | | | | | | | |
| TANGO2 | transport and golgi | 616830, | AR | Metabolic | 22 | 18384537 | 18434687 | 18134537 | 18684687 |
| | organization 2 | 616878 | | encephalomyopathic | | | | | |
| | homolog | | | crises, | | | | | |
| | | | | recurrent, with | | | | | |
| | | | | rhabdomyolysis, | | | | | |
| | | | | cardiac | | | | | |
| | | | | arrhythmias, and | | | | | |
| | | | | neurodegeneration | | | | | |
| TMEM70 | transmembrane | 612418, | AR | Mitochondrial | 8 | 75047226 | 75057572 | 74797226 | 75307572 |
| | protein 70 | 614052 | | complex V | | | | | |
| | | | | (ATP synthase) | | | | | |
| | | | | deficiency, | | | | | |
| | | | | nuclear type 2 | | | | | |
| TUFM | Tu translation | 602389, | AR | Combined | 16 | 28761233 | 28765170 | 28511233 | 29015170 |
| | elongation factor, | 610678 | | oxidative | | | | | |
| | mitochondrial | | | phosphorylation | | | | | |
| | | | | deficiency 4 | | | | | |
| UQCRC2 | ubiquinol- | 191329, | AR | Mitochondrial | 16 | 21872110 | 21902482 | 21622110 | 22152482 |
| | cytochrome c | 615160 | | complex III | | | | | |
| | reductase core | | | deficiency, | | | | | |
| | protein 2 | | | nuclear type 5 | | | | | |
| YARS2 | tyrosyl-tRNA | 610957, | AR | Myopathy, lactic | 12 | 32771691 | 32800098 | 32521691 | 33050098 |
| | synthetase 2 | 613561 | | acidosis, and | | | | | |
| | | | | sideroblastic | | | | | |
| | | | | anemia 2 | | | | | |

Table 9 presents the non-redundant list of other HA genes (i.e., those not listed in Table 8). The column headers in Table 9 are as follows: RefSeq Gene Symbol—gene symbol used by NCBI's Reference Sequence (RefSeq) database; Gene Full Name—official HGNC-provided full name for the gene; OMIM-OMIM entry numbers for the gene and phenotype; Disease Model—indicates if inheritance is autosomal dominant (AD), autosomal recessive (AR), X-linked dominant (XLD), or X-linked recessive (XLR); Disease Name (OMIM)—name of the type of UCD as listed in OMIM; Chr—chromosome that harbors the UCD gene; Gene Start (hg18)—genome position corresponding to the start of the gene (based on human assembly NCBI 36/hg 18); Gene Stop (hg18)—genome position corresponding to the end (stop) of the gene (based on human assembly NCBI 36/hg18); Gene Start—250 Kb (hg18)—genome position corresponding to the start of the gene (based on human assembly NCBI 36/hg18) minus 250 Kb; Gene Stop+250 Kb (hg18)—genome position corresponding to the end (stop) of the gene (based on human assembly NCBI 36/hg18) plus 250 Kb.

TABLE 10
Variants identified in ADNI's WGS data assessed in the subset of Alzheimer's disease (AD) cases (causal variants (OR > 1))

| | | | | | gnomAD | gnomAD | AD | AD | | | | | | | |
|---|---|---|---|---|---|---|---|---|---|---|---|---|---|---|---|
| Analysis | | Position | | | Total | Positive | Total | Positive | | | OR | OR | RefSeq | Gene | SEQ |
| Category | Chr | (hg19) | REF | ALT | [symbol] | [symbol] | [symbol] | [symbol] | p-value | OR | LCL | UCL | Gene Symbol | Region | ID |
| HA | 1 | 45974696 | A | G | 56,596 | 3 | 177 | 1 | 0.01241 | 107.18 | 11.10 | 1035.45 | MMACHC | Ex. | 117 |
| HA | 2 | 26437421 | G | A | 56,801 | 3 | 177 | 1 | 0.01237 | 107.57 | 11.14 | 1039.20 | HADHA | Ex. | 118 |
| HA | 2 | 150438722 | C | A | 56,827 | 13 | 177 | 1 | 0.04261 | 24.83 | 3.23 | 190.85 | MMADHC | Ex. | 119 |
| CPS1 del | 2 | 211361375 | CTACA | C | 7,707 | 0 | 177 | 1 | 0.02245 | 131.01 | 5.32 | 3227.24 | CPS1 | Int. | 120 |
| CPS1 del | 2 | 211362642 | A | ATC | 6,746 | 1 | 174 | 2 | 0.00185 | 78.43 | 7.08 | 869.11 | CPS1 | Int. | 121 |
| CPS1 del | 2 | 211363902 | G | T | 7,710 | 12 | 177 | 2 | 0.03818 | 7.33 | 1.63 | 33.00 | CPS1 | Int. | 122 |
| CPS1 del | 2 | 211367103 | AT | A | 7,083 | 22 | 80 | 2 | 0.02900 | 8.23 | 1.90 | 35.60 | CPS1 | Int. | 123 |
| CPS1 del | 2 | 211367235 | T | TTA | 7,219 | 1 | 177 | 1 | 0.04729 | 41.01 | 2.55 | 658.35 | CPS1 | Int. | 124 |
| GH02J210503 | 2 | 211369053 | C | A | 7,715 | 1 | 177 | 1 | 0.04436 | 43.83 | 2.73 | 703.59 | CPS1 | Int. | 125 |
| HA | 3 | 48929487 | G | A | 56,878 | 1 | 177 | 1 | 0.00619 | 323.16 | 20.13 | 5187.29 | SLC25A20 | Ex. | 126 |
| HA | 3 | 49455323 | C | T | 56,774 | 2 | 177 | 1 | 0.00930 | 161.28 | 14.56 | 178687 | AMT | Ex. | 127 |
| HA | 3 | 136016902 | G | A | 56,873 | 184 | 177 | 3 | 0.02078 | 5.31 | 1.68 | 16.78 | PCCB | Ex. | 128 |
| HA | 3 | 136046050 | G | A | 56,720 | 2 | 177 | 1 | 0.00930 | 161.13 | 14.54 | 1785.18 | PCCB | Ex. | 129 |
| HA | 3 | 136048854 | A | G | 56,810 | 5 | 177 | 1 | 0.01849 | 64.55 | 7.50 | 555.36 | PCCB | Ex. | 130 |
| HA | 3 | 182740282 | G | T | 56,824 | 10 | 177 | 1 | 0.03363 | 32.28 | 4.11 | 253.52 | MCCC1 | Ex. | 131 |
| HA | 4 | 146576485 | C | T | 56,869 | 4 | 177 | 1 | 0.01542 | 80.77 | 8.98 | 726.32 | MMAA | Ex. | 132 |
| HA | 6 | 49403267 | C | T | 56,816 | 12 | 177 | 1 | 0.03963 | 26.90 | 3.48 | 207.96 | MMUT | Ex. | 133 |
| HA | 6 | 99374800 | C | G | 56,769 | 10 | 177 | 1 | 0.03367 | 32.25 | 4.11 | 253.28 | FBXL4 | Ex. | 134 |
| UCD | 7 | 65554101 | A | G | 56,656 | 16 | 177 | 2 | 0.00143 | 40.46 | 9.23 | 177.28 | ASL | Ex. | 135 |
| UCD | 7 | 95813678 | C | A | 56,846 | 12 | 177 | 1 | 0.03961 | 26.91 | 3.48 | 208.07 | SLC25A13 | Ex. | 136 |
| HA | 11 | 66618208 | T | C | 56,321 | 1 | 175 | 1 | 0.00619 | 323.68 | 20.16 | 5195.77 | PC | Ex. | 137 |
| HA | 11 | 111896242 | G | A | 56,265 | 9 | 177 | 1 | 0.03092 | 35.52 | 4.48 | 281.82 | DLAT | Ex. | 138 |
| HA | 11 | 111916647 | G | A | 56,728 | 33,119 | 177 | 118 | 0.02668 | 1.43 | 1.04 | 1.95 | DLAT | Ex. | 139 |
| HA | 11 | 111921965 | A | G | 56,687 | 10 | 177 | 1 | 0.03371 | 32.20 | 4.10 | 252.91 | DLAT | Ex. | 140 |
| HA | 14 | 23243579 | C | T | 56,804 | 2 | 177 | 1 | 0.00929 | 161.37 | 14.57 | 1787.82 | SLC7A7 | Ex. | 141 |
| HA | 16 | 28855653 | G | A | 56,851 | 1 | 177 | 1 | 0.00620 | 323.01 | 20.12 | 5184.83 | TUFM | Ex. | 142 |
| HA | 16 | 83940677 | C | T | 56,645 | 3 | 177 | 1 | 0.01240 | 107.28 | 11.10 | 1036.34 | MLYCD | Ex. | 143 |
| HA | 16 | 83948709 | T | A | 56,648 | 7 | 177 | 1 | 0.02465 | 45.97 | 5.63 | 375.64 | MLYCD | Ex. | 144 |
| HA | 16 | 83948889 | A | G | 56,461 | 1 | 177 | 1 | 0.00624 | 320.80 | 19.99 | 5149.26 | MLYCD | Ex. | 145 |
| HA | 17 | 7125598 | G | C | 56,850 | 3 | 177 | 1 | 0.01236 | 107.66 | 11.14 | 1040.10 | ACADVL | Ex. | 146 |
| UCD | 17 | 42084765 | C | G | 56,479 | 7 | 177 | 1 | 0.02472 | 45.84 | 5.61 | 374.52 | NAGS | Ex. | 147 |
| UCD | 17 | 42084825 | C | A | 56,384 | 3 | 177 | 1 | 0.01246 | 106.78 | 11.05 | 1031.57 | NAGS | Ex. | 148 |
| HA | 21 | 38132112 | C | T | 56,883 | 9 | 177 | 1 | 0.03059 | 35.91 | 4.52 | 284.92 | HLCS | Ex. | 149 |

[symbol]

indicates data missing or illegible when filed

Table 10 lists variants identified in publicly available WGS data reported by ADNI. Variants were assessed in the subset of Alzheimer's disease (AD) cases and were compared to publicly available gnomAD population data for the statistical analyses. Only causal variants (OR>1) are reported. The column headers in Table 10 are as follows: Analysis Category—four categories were assessed: CPS1 del are variants that overlap with SEQ IDs 3 (see Table 1) or 116 (see Table 6); GH02J210503 are variants that overlap with CPS1 GeneHancer enhancer GH02J210503 (e.g., see FIGS. 3 and 4); HA are variants that overlap with HA genes (see Table 9); UCD are variants that overlap with UCD genes (see Table 8); Chr—Chromosome (Chr) harboring the variant; Position (hg19)—genome location of the variant (NCBI37/hg19 genome coordinates); REF—allele found in the Reference (REF) human genome assembly; ALT—alternate (ALT) allele found in the gnomAD subjects or AD cases; gnomAD Total Subjects—total number of gnomAD (v2.1) WES subjects assessed for the variant; gnomAD Positive Subjects—number of gnomAD (v2.1) WES subjects with the variant; AD Total Cases—total number of AD cases in the ADNI WGS data assessed for the variant; AD Positive Cases—number of AD cases in the ADNI WGS data with the variant; p-value-statistical significance using FET (a cutoff of <0.05 was used); OR—odds ratio (OR) value; OR LCL-odds ratio, lower confidence limit (95%); OR UCL—odds ratio, upper confidence limit (95%); RefSeq Gene Symbol—gene symbol used by NCBI's Reference Sequence (RefSeq) database; Gene Region—lists if the variant is located in an exonic (Ex.) or intronic (Int.) region of a gene; SEQ ID—Sequence identification number in the Sequence Listing.

TABLE 11
Variants identified in ADNI's WGS data assessed in the subset of mild cognitively impaired (MCI) cases (causal variants (OR > 1))

| | | | | | gnomAD | gnomAD | MCI | MCI | | | | | | | |
|---|---|---|---|---|---|---|---|---|---|---|---|---|---|---|---|
| Analysis | | Position | | | Total | Positive | Total | Positive | | | OR | OR | RefSeq | Gene | |
| Category | Chr | (hg19) | REF | ALT | Subjects | [symbol] | Cases | [symbol] | p-value | OR | LCL | UCL | Gene Symbol | Region | SEQ ID |
| HA | 1 | 53675699 | A | G | 56,787 | 1 | 342 | 1 | 0.01194 | 166.53 | 10.39 | 2667.95 | CPT2 | Ex. | 150 |
| HA | 1 | 53676305 | C | T | 56,799 | 2 | 342 | 1 | 0.01785 | 83.28 | 7.53 | 920.64 | CPT2 | Ex. | 151 |
| HA | 1 | 53676401 | T | G | 56,777 | 100 | 342 | 3 | 0.02424 | 5.02 | 1.58 | 15.89 | CPT2 | Ex. | 152 |
| HA | 1 | 53676688 | T | C | 56,836 | 6 | 342 | 1 | 0.04113 | 27.78 | 3.33 | 231.34 | CPT2 | Ex. | 153 |
| HA | 2 | 26455041 | C | G | 56,777 | 1 | 342 | 1 | 0.01194 | 166.50 | 10.39 | 2667.48 | HADHA | Ex. | 154 |
| CPS1 del | 2 | 211365470 | C | T | 7,706 | 0 | 342 | 1 | 0.04250 | 67.70 | 2.75 | 1665.01 | CPS1 | Int. | 155 |
| CPS1 del | 2 | 211365748 | A | G | 7,707 | 5 | 342 | 2 | 0.03281 | 9.06 | 1.75 | 46.87 | CPS1 | Int. | 156 |
| CPS1 del | 2 | 211367441 | C | A | 7,691 | 4 | 342 | 2 | 0.02419 | 11.30 | 2.06 | 61.93 | CPS1 | Int. | 157 |
| UCD | 2 | 211454831 | G | A | 56,638 | 77 | 342 | 3 | 0.01253 | 6.50 | 2.04 | 20.70 | CPS1 | Ex. | 158 |
| UCD | 2 | 211507223 | T | A | 56,730 | 0 | 342 | 1 | 0.00599 | 498.36 | 20.27 | 12255.94 | CPS1 | Ex. | 159 |
| UCD | 2 | 211527868 | C | T | 56,845 | 0 | 342 | 1 | 0.00598 | 499.37 | 20.31 | 12280.78 | CPS1 | Ex. | 160 |
| HA | 3 | 49455323 | C | T | 56,774 | 2 | 342 | 1 | 0.01786 | 83.24 | 7.53 | 920.23 | AMT | Ex. | 127 |
| HA | 3 | 182788881 | C | T | 56,873 | 1 | 342 | 1 | 0.01192 | 166.78 | 10.41 | 2671.99 | MCCC1 | Ex. | 161 |
| HA | 4 | 159611545 | G | A | 56,848 | 0 | 342 | 1 | 0.00598 | 499.40 | 20.31 | 12281.43 | ETFDH | Ex. | 162 |
| HA | 4 | 159616692 | T | C | 56,792 | 1 | 342 | 1 | 0.01194 | 166.54 | 10.40 | 2668.18 | ETFDH | Ex. | 163 |
| UCD | 7 | 95761141 | G | A | 56,660 | 7 | 342 | 1 | 0.04701 | 23.73 | 2.91 | 193.43 | SLC25A13 | Ex. | 164 |
| UCD | 7 | 95818665 | G | A | 56,860 | 1 | 342 | 1 | 0.01192 | 166.74 | 10.41 | 2671.38 | SLC25A13 | Ex. | 165 |
| HA | 10 | 97376235 | A | T | 35,695 | 1 | 342 | 1 | 0.01889 | 104.67 | 6.53 | 1677.01 | ALDH18A1 | Ex. | 166 |
| HA | 10 | 126091623 | A | T | 56,351 | 3 | 342 | 1 | 0.02391 | 55.08 | 5.71 | 530.87 | OAT | Ex. | 167 |
| HA | 11 | 66617482 | G | A | 56,704 | 0 | 342 | 1 | 0.00600 | 498.14 | 20.26 | 12250.32 | PC | Ex. | 168 |
| HA | 11 | 111899575 | C | A | 56,881 | 1 | 342 | 1 | 0.01192 | 166.80 | 10.41 | 2672.36 | DLAT | Ex. | 169 |
| HA | 11 | 111930627 | A | C | 52,799 | 4 | 342 | 1 | 0.03177 | 38.71 | 4.31 | 347.21 | DLAT | Ex. | 170 |
| HA | 12 | 109998857 | C | T | 56,461 | 2 | 340 | 1 | 0.01785 | 83.27 | 7.53 | 920.57 | MMAB | Ex. | 171 |
| HA | 12 | 110011282 | C | G | 50,227 | 2 | 342 | 1 | 0.02015 | 73.64 | 6.66 | 814.11 | MMAB | Ex. | 172 |
| HA | 13 | 100861707 | G | C | 56,851 | 6 | 342 | 1 | 0.04112 | 27.78 | 3.34 | 231.40 | PCCA | Ex. | 173 |
| HA | 15 | 76578804 | A | C | 56,771 | 3 | 342 | 1 | 0.02374 | 55.49 | 5.76 | 534.83 | ETFA | Ex. | 174 |
| HA | 15 | 76603710 | G | A | 30,861 | 26 | 341 | 2 | 0.03734 | 7.00 | 1.65 | 29.60 | ETFA | Ex. | 175 |
| HA | 17 | 7126099 | A | C | 56,816 | 1 | 342 | 1 | 0.01193 | 166.61 | 10.40 | 2669.31 | ACADVL | Ex. | 176 |
| HA | 17 | 7127359 | C | T | 56,856 | 5 | 342 | 1 | 0.03534 | 33.34 | 3.89 | 286.17 | ACADVL | Ex. | 177 |
| HA | 17 | 17924457 | G | A | 56,505 | 4 | 342 | 1 | 0.02972 | 41.42 | 4.62 | 371.58 | ATPAF2 | Ex. | 178 |
| UCD | 17 | 42084786 | A | T | 56,510 | 1 | 342 | 1 | 0.01200 | 165.72 | 10.34 | 2654.93 | NAGS | Ex. | 179 |
| UCD | 17 | 42085014 | C | G | 50,285 | 2 | 342 | 1 | 0.02013 | 73.73 | 6.67 | 815.05 | NAGS | Ex. | 180 |
| UCD | 17 | 42085026 | G | A | 50,261 | 1 | 342 | 1 | 0.01347 | 147.39 | 9.20 | 2361.34 | NAGS | Ex. | 181 |
| HA | 19 | 19218779 | C | T | 56,600 | 59 | 342 | 3 | 0.00625 | 8.48 | 2.65 | 27.19 | SLC25A42 | Ex. | 182 |
| HA | 19 | 51857610 | A | G | 56,344 | 1 | 341 | 1 | 0.01200 | 165.71 | 10.34 | 2654.94 | ETFB | Ex. | 183 |

[symbol]

indicates data missing or illegible when filed

Table 11 lists variants identified in publicly available WGS data reported by ADNI. Variants were assessed in the subset of mild cognitively impaired (MCI) cases and were compared to publicly available gnomAD population data for the statistical analyses. Only causal variants (OR>1) are reported. The column headers in Table 11 are as follows: Analysis Category—four categories were assessed (but only three were found with relevant variants, none were found for the GH02J210503 category): CPS1 del are variants that overlap with SEQ IDs 3 (see Table 1) or 116 (see Table 6); GH02J210503 are variants that overlap with CPS1 GeneHancer enhancer GH02J210503 (e.g., see FIGS. 3 and 4); HA are variants that overlap with HA genes (see Table 9); UCD are variants that overlap with UCD genes (see Table 8); Chr-Chromosome (Chr) harboring the variant; Position (hg 19)—genome location of the variant (NCBI37/hg 19 genome coordinates); REF—allele found in the Reference (REF) human genome assembly; ALT—alternate (ALT) allele found in the gnomAD subjects or MCI cases; gnomAD Total Subjects—total number of gnomAD (v2.1) WES subjects assessed for the variant; gnomAD Positive Subjects—number of gnomAD (v2.1) WES subjects with the variant; MCI Total Cases—total number of MCI cases in the ADNI WGS data assessed for the variant; MCI Positive Cases—number of MCI cases in the ADNI WGS data with the variant; p-value—statistical significance using FET (a cutoff of <0.05 was used); OR—odds ratio (OR) value; OR LCL—odds ratio, lower confidence limit (95%); OR UCL—odds ratio, upper confidence limit (95%); RefSeq Gene Symbol—gene symbol used by NCBI's Reference Sequence (RefSeq) database; Gene Region—lists if the variant is located in an exonic (Ex.) or intronic (Int.) region of a gene; SEQ ID-Sequence identification number in the Sequence Listing.

TABLE 12
Variants identified in ADNI's WGS data assessed in the subset of mild cognitively impaired (MCI) cases (protective variants (OR < 1))

| | | | | | | gnomAD | MCI | MCI | | | | | RefSeq | | |
|---|---|---|---|---|---|---|---|---|---|---|---|---|---|---|---|
| Analysis | | Position | | | gnomAD | Positive | Total | Positive | | | OR | OR | Gene | Gene | SEQ |
| Category | Chr | (hg19) | REF | ALT | Total | Subjects | Cases | Cases | p-value | OR | LCL | UCL | Symbol | Region | ID |
| CPS1 del | 2 | 211364167 | A | T | 7696 | 103 | 342 | 0 | 0.02302 | 0.11 | 0.01 | 1.73 | CPS1 | Int. | 184 |
| CPS1 del | 2 | 211367151 | AAGAG | A | 7008 | 202 | 342 | 3 | 0.02666 | 0.30 | 0.09 | 0.94 | CPS1 | Int. | 185 |

Table 12 lists variants identified in publicly available WGS data reported by ADNI. Variants were assessed in the subset of mild cognitively impaired (MCI) cases and were compared to publicly available gnomAD population data for the statistical analyses. Only protective variants (OR<1) are reported. The column headers in Table 12 are as follows: Analysis Category—four categories were assessed (but only one, CPS1 del, was found with relevant variants): CPS1 del are variants that overlap with SEQ IDs 3 (see Table 1) or 116 (see Table 6); GH02J210503 are variants that overlap with CPS1 GeneHancer enhancer GH02J210503 (e.g., see FIGS. 3 and 4); HA are variants that overlap with HA genes (see Table 9); UCD are variants that overlap with UCD genes (see Table 8); Chr—Chromosome (Chr) harboring the variant; Position (hg19)—genome location of the variant (NCBI37/hg19 genome coordinates); REF—allele found in the Reference (REF) human genome assembly; ALT—alternate (ALT) allele found in the gnomAD subjects or MCI cases; gnomAD Total Subjects—total number of gnomAD (v2.1) WES subjects assessed for the variant; gnomAD Positive Subjects—number of gnomAD (v2.1) WES subjects with the variant; MCI Total Cases—total number of MCI cases in the ADNI WGS data assessed for the variant; MCI Positive Cases—number of MCI cases in the ADNI WGS data with the variant; p-value—statistical significance using FET (a cutoff of <0.05 was used); OR—odds ratio (OR) value; OR LCL—odds ratio, lower confidence limit (95%); OR UCL—odds ratio, upper confidence limit (95%); RefSeq Gene Symbol—gene symbol used by NCBI's Reference Sequence (RefSeq) database; Gene Region—lists if the variant is located in an exonic (Ex.) or intronic (Int.) region of a gene; SEQ ID—Sequence identification number in the Sequence Listing.

TABLE 13
FDA-approved compounds available for treatment of UCDs

| | Brand | | |
|---|---|---|---|
| Drug Name | Name | Manufacturer/Sponser | UCD Gene Target(s) |
| carglumic acid | Carbaglu | Recordati Rare Diseases | NAGS |
| glycerol phenylbutyrate | Ravicti | Horizon Therapeutics | ARG1, ASS1, CPS1, OTC, |
| | | (Hyperion Therapeutics), | SLC25A13, SLC25A15 |
| | | Immedica Pharma | |
| sodium phenylacetate and sodium | Ammonul | Bausch Health, Ucyclyd | ARG1, ASS1, CPS1, NAGS, OTC |
| benzoate | | Pharma | |
| | Ucephan | Immunex (Braun Medical) | ARG1, ASS1, CPS1, NAGS, OTC |
| sodium phenylbutyrate | Buphenyl | Horizon Therapeutics | ASS1, CPS1, OTC, SLC25A15 |
| | | (Hyperion Therapeutics) | |
| | Ammonaps | Immedica Pharma | ASS1, CPS1, OTC |
| | Pheburane | Eurocept, Lucane Pharma, | ASS1, CPS1, OTC |
| | | Orpharma, Proveca | |
| | Olpruva | Acer Therapeutics/Relief | ASS1, CPS1, OTC |
| | | Therapeutics | |
| sodium benzoate | generic | various | multiple |

Table 13 lists FDA-approved compounds available for treatment of UCDs. The column headers in Table 13 are as follows: Drug Name—the generic name of the drug; Brand Name—brand name(s) of the drugs; Manufacturer/Sponsor—pharmaceutical companies that market the drug (including former developers or manufacturers); UCD Gene Target(s)—gene symbols for the UCD(s) that are treatable with a given drug. Multiple formulations can be found for a given compound, such as taste-masked sodium phenylbutyrate (Olpruva/OLPRUVA™).

TABLE 14
Therapeutic approaches in development for treatment of UCDs

| | | | | Sponsor/ | | |
|---|---|---|---|---|---|---|
| Experimental | | Clinical | Other | Responsible | | UCD Gene |
| Therapies | Approach | Trial ID | Study ID | Party | Phase* | Target(s) |
| AEB1102 | enzyme | NCT03378531 | CAEB1102-102A | Aglea | Phase 2 | ARG1 |
| (pegzilarginase) | replacement | | | Biotherapeutics | | |
| | | NCT03921541 | CAEB1102-300A | | Phase 3 | |
| ARCT-810 | mRNA | NCT04416126 | ARCT- 810-01 | Arcturus | Phase 1b | OTC |
| | | | | Therapeutics | | |
| | | NCT04442347 | ARCT- 810-02 | | Phase 1b | |
| | | NCT05526066 | ARCT- 810-03 | | Phase 2 | |
| BB-OTC | enzyme | | | Enlivex | Pre- | OTC |
| | replacement | | | Therapeutics | clinical | |
| DTX301 | gene therapy | NCT05345171 | DTX301-CL301, | Ultragenyx | Phase 3 | OTC |
| | (AAV8) | | 2020-003384-25 | Pharmaceutical | | |
| KB-195 | microbiome | NCT03933410 | K020-218 | Kaleido | Phase 2 | all UCD |
| | metabolic | | | Biosciences | | |
| P-OTC-101 | gene therapy | | | Poseida | Pre- | OTC |
| | | | | Therapeutics | clinical | |
| PRX-OTC | enzyme | | | Roivant | Pre- | OTC |
| | replacement | | | Sciences | clinical | |
| | | | | (PhaseRx | | |
| | | | | asset) | | |
| SEL-313 | gene therapy | | | Selecta Bio | Pre- | OTC |
| | | | | | clinical | |
| SG328 | fusogen | | | Sana | Pre- | OTC |
| | | | | Biotechnology | clinical | |
| P-OTC-101 | gene therapy | | | Poseida | Pre- | OTC |
| | | | | Therapeutics | clinical | |

Table 14 lists therapeutic approaches in development for treatment of UCDs. The column headers in Table 14 are as follows: Experimental Therapies—name of therapy in development; Approach—type of therapeutic approach; Clinical Trial ID—ClinicalTrials.gov identifier; Other Study ID—Other study identifier; Sponsor/Responsible Party—pharmaceutical company that is the sponsor or responsible party; Phase—clinical trial phase (development stage) for the study; UCD Gene Target(s)—gene symbols for the UCD(s) that are treatable with a given drug.

### Example 10—Figures Referenced in this Study

FIG. 1 represents an example of an aCGH-detected 3-probe intronic deletion that impacts the UCD gene CPS1. The deletion was observed in 3/100 LOAD cases and 1/1,000 Normal (NVE) subjects. All 4 deletions appear to be identical, with a genomic span of 6,430 bp. Genome coordinates of the deletion are: hg18, chr2:211070190-211076620; hg19, chr2:211361945-211368375. Log 2 ratios for the y-axes (4 data tracks) report deletions as negatively shifted probes. Genome coordinates for the x-axis are based on the NCBI36/hg18 freeze.

FIG. 2 represents an example of the extent of the CPS1 intronic deletion compared to different public data sources for CNVs/SVs in the UCSC genome browser. The deletion reported by Population Bio (CPS1_del_PBio) is defined by the location of 3 aCGH probes (see FIG. 1). The deletion reported in other data sources (1KG Ph3 labeled as CPS1 del_1KG_Ph3, DGV_Gold labeled as gssvL69036, and gnomAD labeled as DEL_2_26674) is based on analysis of WGS data, which allows (in this instance) for a more accurate delineation of the endpoints. Five aCGH probes are shown (central 3 mapping to the deletion plus 1 flanking on each side). The endpoints of the WGS mapped deletion do not extend to the flanking probes, which confirms the aCGH data. Genome coordinates are based on the NCBI36/hg18 freeze.

FIG. 3 represents an example of UCSC genome browser regulatory site annotations that are encompassed by, or overlap with, the CPS1 deletion detected in 3 LOAD cases. Displayed are the 3 Agilent probes (identified by dashed lines and are the same 3 aCGH probes shown in FIG. 1), CNV/SV public data (DGV GS, 1KG Ph3, and gnomAD) that is also shown in FIG. 2, and regulatory site annotation from two public data sources: ENCODE transcription factor binding sites; GeneHancer mapping of enhancer and promoter sites. This demonstrates that the deletion overlaps an enhancer regulatory element (GH02J210503) with a functional role for expression of the CPS1 gene. Genome coordinates are based on the GRCh37/hg 19 freeze.

FIG. 4 represents an example of UCSC genome browser regulatory site annotations for the full extent of the CPS1 gene (zoomed out view of FIG. 3, the intronic deletion is demarcated by the pair of vertical dashed lines). Besides GeneHancer enhancer GH02J210503, the location of 5 other promoter/enhancer elements are shown. Also displayed are the interactions between GeneHancer regulatory elements and genes (Double Elite), which shows that enhancer GH02J210503 (located in an intron of CPS1) interacts with CPS1 promoter GH02J210475.

FIG. 5 represents an example of a PCR assay designed to detect the CPS1 intronic deletion (see FIG. 1 and FIG. 2). Shown is a 1% agarose gel with a PCR product band (350 bp in size) obtained for a positive (Pos.) control genomic DNA sample (i.e., from a subject known to harbor the CPS1 deletion) using PCR primers CPS1_delF and CPS1delR (see Table 6). No PCR product is observed for two different negative (Neg.) control genomic DNA samples, thereby demonstrating the specificity of the DNA assay to detect the CPS1 deletion.

FIG. 6 represents an example of a PCR assay used to validate the aCGH-detected CPS1 deletion found in 3 LOAD cases (see FIG. 1). Each of the 3 positive LOAD samples (Expt IDs 2693, 2696, and 2719) was run in duplicate (lanes 1-6 after the DNA ladder) on a 2% agarose gel. Two negative controls (samples known not to harbor the deletion on the basis of aCGH, lanes 7 and 8) are also shown, plus a non-template control (NTC) wherein the assay was run with no input DNA. This demonstrates that all 3 LOAD samples yield a positive product at the expected size of 350 bp (see FIG. 5).

FIG. 7 shows examples of aCGH-detected CNVs nearby UCD gene OTC (gene locations are shown at the top) in 5 LOAD cases (all female). The CNVs (a deletion in track 1, a duplication in track 2, and an identical duplication in tracks 3-5) are also nearby or directly impact the TSPAN7 gene. Log 2 ratios for the y-axes (5 tracks) report deletions as negatively shifted probes and duplications as positively shifted probes. Genome coordinates for the x-axis are based on the NCBI36/hg18 freeze. See Table 1 for the NCBI36/hg18 genome coordinates of the CNVs.

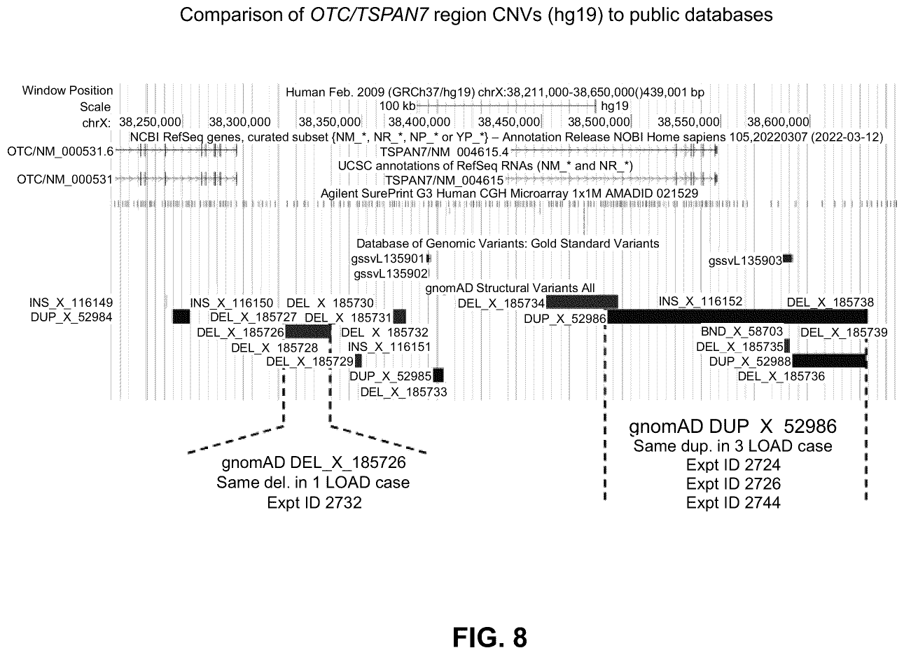

FIG. 8 represents examples of aCGH-detected CNVs (deletion and duplication) compared to public data sources for CNVs/SVs (DGV GS and gnomAD SV) in the UCSC genome browser. Two aCGH-detected CNVs (see FIG. 7) were confirmed in the gnomAD SV database: DEL_X_185726 corresponds to the deletion found in 1 LOAD case (Expt ID 2732); DUP_X_52986 corresponds to the duplication found in 3 LOAD cases (Expt IDs 2724, 2726, and 2744). The duplication found in LOAD case Expt ID 2687 is not found in public CNV/SV data sources. Genome coordinates are based on the GRCh37/hg 19 freeze. See Table 1 for the NCBI36/hg18 genome coordinates of the CNVs.

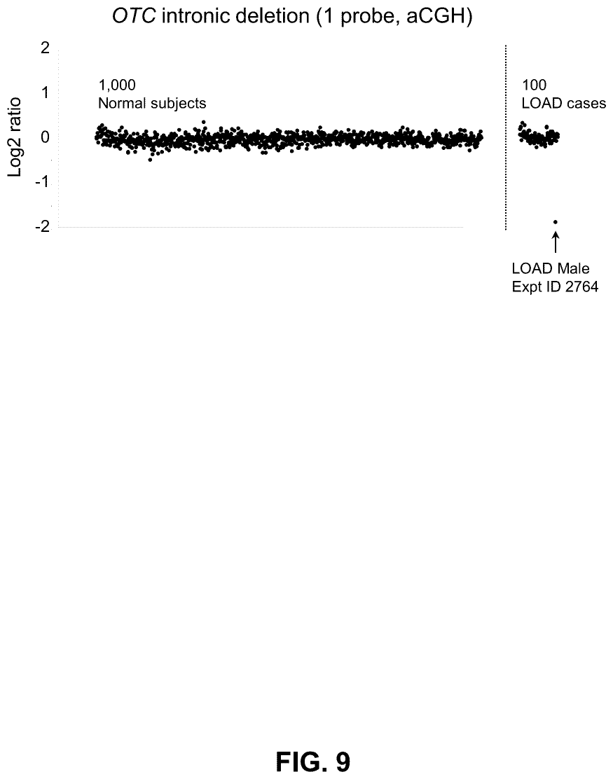

FIG. 9 represents an example of an aCGH-detected 1-probe (Agilent probe A_16 P21444361) intronic deletion that impacts the UCD gene OTC. The deletion was observed in 1/100 LOAD cases and 0/1,000 Normal (NVE) subjects (the dotted vertical line demarcates the 1,000 Normal subjects from the 100 LOAD cases). Genome coordinates for the aCGH probe: hg18, chrX:38109666-38109725; hg19, chrX:38224722-38224781. The y-axis Log 2 ratio reports deletions as negatively shifted probes. The plot demonstrates that (i) the probe is well-behaved (very little noise across 1,100 total aCGH experiments) and (ii) that there exists an individual with a very low log 2 ratio (consistent with a homozygous deletion). This individual is male and thus this result demonstrates that this individual is deleted at this locus (the log 2 ratio is lower than expected for a deletion affecting only one allele, because males only have one Chr X).

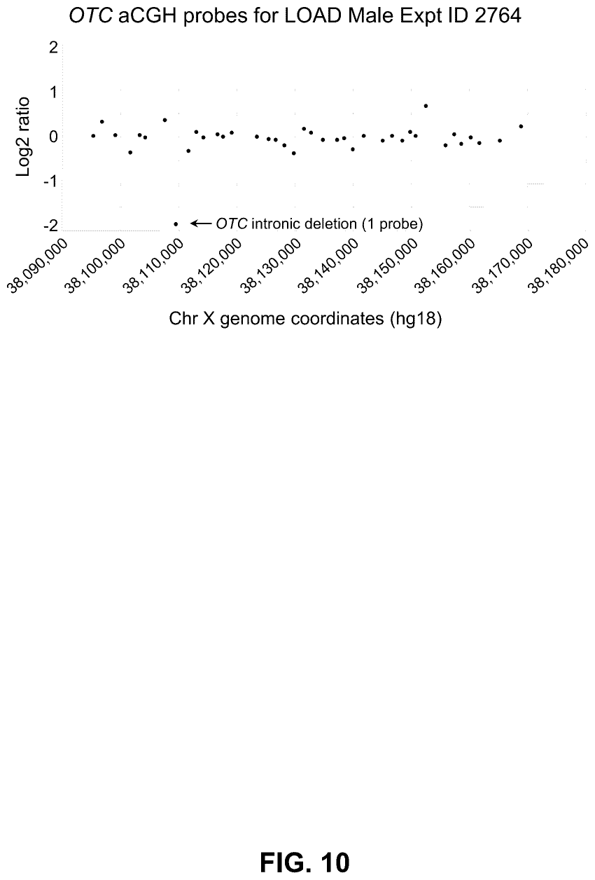

FIG. 10 represents an example of a plot of aCGH data for an individual with the deletion shown in FIG. 9. The probes (dots) in this plot are all from the same LOAD case (Expt ID 2764) and span the OTC gene (located at chrX:38,096,680-38,165,647), demonstrating the deleted probe in the context of the full extent of the gene. Genome coordinates for the x-axis are based on the NCBI36/hg 18 freeze.

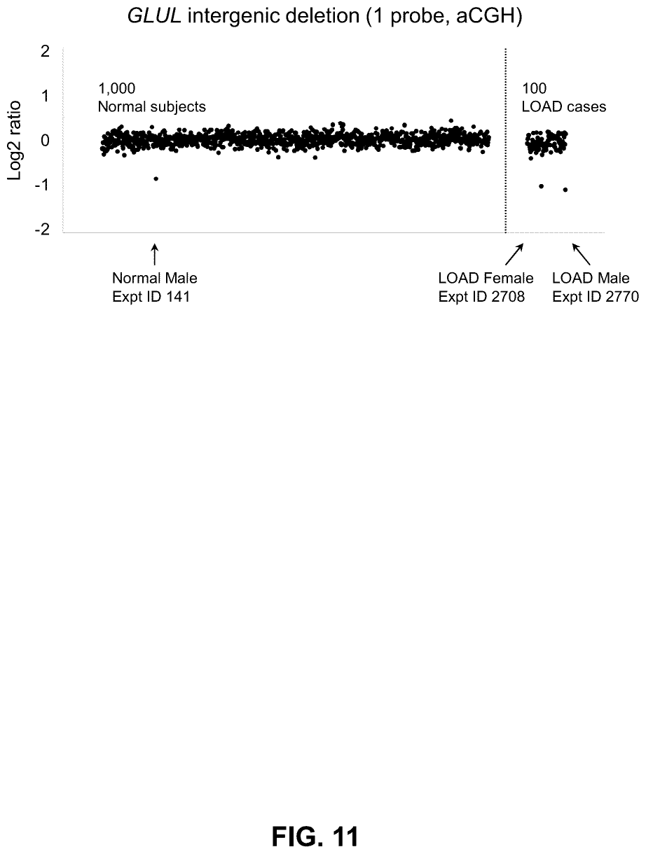

FIG. 11 represents an example of an aCGH-detected 1-probe (Agilent probe A_16 P15364898) intergenic deletion ˜226Kb downstream of HA gene GLUL. The deletion was observed in 2/100 LOAD cases and 1/1,000 Normal (NVE) subjects (the dotted vertical line demarcates the 1,000 Normal subjects from the 100 LOAD cases). Genome coordinates for the aCGH probe: hg18, chr1:180387776-180387835; hg19, chr1:182121153-182121212. The y-axis Log 2 ratio reports deletions as negatively shifted probes.

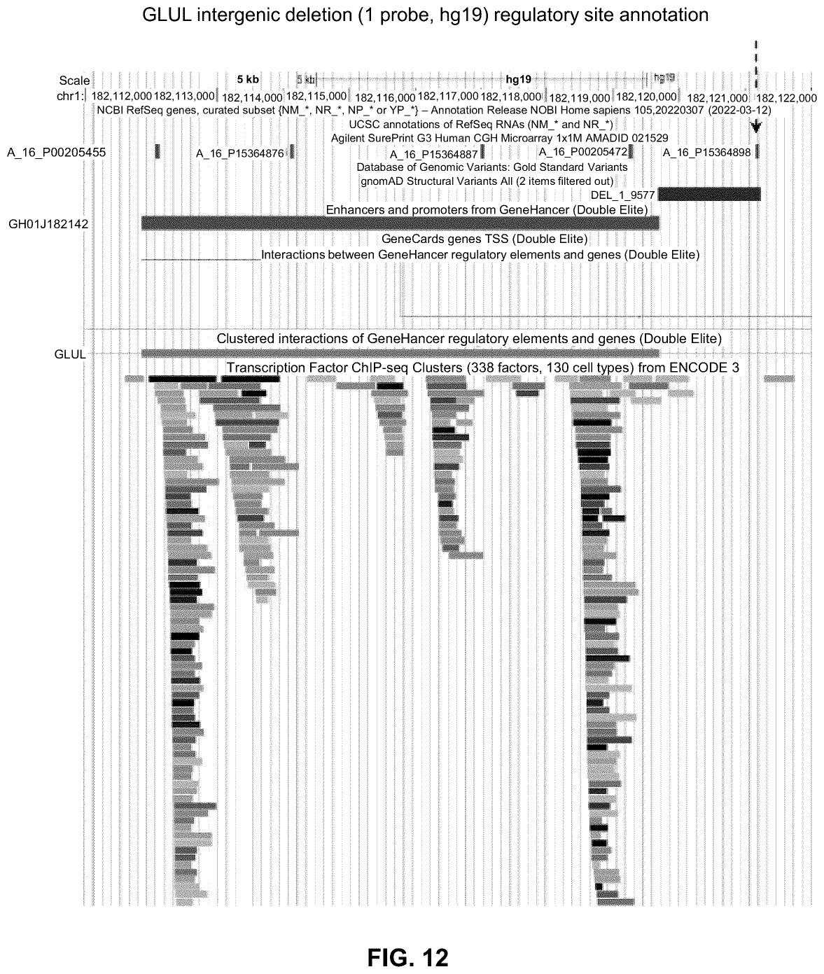

FIG. 12 represents an example of UCSC genome browser regulatory site annotations that are immediately adjacent to the intergenic deletion (˜226Kb downstream of the GLUL gene) detected in 2 LOAD cases (see FIG. 11). Displayed are the deletion-reporting aCGH Agilent probe (A_16 P15364898, see FIG. 11), CNV/SV public data (DGV GS and gnomAD), and regulatory site annotation from two public data sources: GeneHancer mapping of enhancer and promoter sites; ENCODE transcription factor binding sites. The 1-probe deletion maps to gnomAD DEL_1_9677, which is immediately adjacent to an enhancer regulatory element (GH01J182142) with a functional role for expression of the GLUL gene. There are numerous ENCODE transcription factor binding sites (bottom track) that map to this enhancer element. Genome coordinates are based on the GRCh37/hg19 freeze.

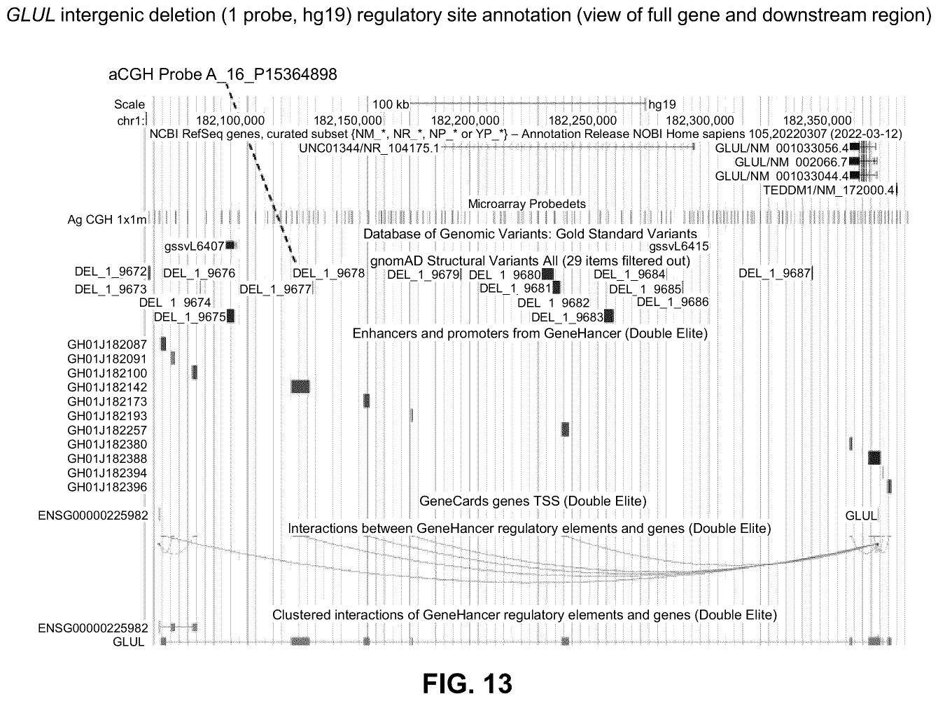

FIG. 13 represents an example of UCSC genome browser regulatory site annotations for the full extent of the GLUL gene and downstream region encompassing the intergenic deletion (zoomed out view of FIG. 12, the deletion detected by Agilent probe A16P15364898 and mapping to gnomAD DEL_1_9677). Besides GeneHancer enhancer GH01J182142, the location of 10 other promoter/enhancer elements downstream of the GLUL gene are shown. This demonstrates that enhancer and promoter elements do not need to be contained within the gene whose expression they are regulating and that they can be located far away from the gene.

### Example 11—Exemplary Gene Panels for AD/MCI Risk Prediction Tests

Typical of complex disorders, multiple deleterious variants distributed across several genes can contribute to AD and/or MCI. Therefore, to efficiently screen for multiple genes/variants, a risk prediction test can be constructed using a gene panel approach. Gene panel tests can be performed using sequencing reagents that only assay the genes in the panel or targeted interpretation of the genes in the panel can be performed using WES or WGS on a subject's genome. In some embodiments, the subject's whole exome or whole genome was previously generated (e.g., as part of the subject's electronic health record) and the gene panel can be assessed using in silico analysis.

Table 15 contains the subset of UCD/HA genes in which one or more subjects with AD (OPTIMA or ADNI cohorts) and/or MCI (ADNI cohort) had a variant with an OR>5 when compared to normal subjects in private (e.g., NVE) and/or public databases (e.g., DGV and gnomAD). Variants in these genes are reported in Tables 1, 10, or 11. Furthermore, Table 15 contains only the subset of variants (with OR>5) that impact an exon and/or regulatory element region (GeneHancer). For Table 1 variants, regions between the next probe to the left and right of the reporting probe(s) were also assessed for GeneHancer promoters and/or enhancers since the breakpoints of the CNV usually map into these regions. For example, in FIG. 3, the left breakpoint of the CPS1 deletion maps between Agilent probes A_16_P36056631 and A16 P16022206 based on public database CNVs in DGV (gssvL69036) and gnomAD SV (DEL_2_26674), which was also validated by Sanger sequencing experiments used to develop the CPS1 deletion assay (see Example 5 and FIGS. 5 and 6).

TABLE 15
UCD/HA gene variants with OR > 5

| | | | | | UCD/HA | | |
|---|---|---|---|---|---|---|---|
| | Variant | | Variant | | Gene | UCD/HA | GeneHancer |
| SEQ | Source | | Allele | Supporting | Impacted or | Gene | Regulatory |
| ID | Table | Variant Position (hg19) | Type | Public Variants | Adjacent | Region | Element |
| 117 | 10 | chr1: 45974696 | A > G | rs141429393 | MMACHC | Ex. | none |
| 150 | 11 | chr1: 53675699 | A > G | rs148035648 | CPT2 | Ex. | none |
| 151 | 11 | chr1: 53676305 | C > T | rs763703786 | CPT2 | Ex. | none |
| 152 | 11 | chr1: 53676401 | T > G | rs2229291 | CPT2 | Ex. | none |
| 153 | 11 | chr1: 53676688 | T > C | rs74315297 | CPT2 | Ex. | none |
| 1 | 1 | chr1: 182121153-182121212 | het loss | DEL_1_9677 | GLUL | Inter. | GH01J182142 (enh), |
| | | | | | | | GH01J182152 (enh) |
| 119 | 10 | chr2: 150438722 | C > A | rs549522925 | MMADHC | Ex. | none |
| 120 | 10 | chr2: 211361375 | CTACA > C | gnomAD | CPS1 | Int. | none |
| | | | | (rs1275499747) | | | |
| 3 | 1 | chr2: 211361945-211368375 | het loss | DEL_2_26674, | CPS1 | Int. | GH02J210503 (enh) |
| | | | | esv3594156, | | | |
| | | | | gssvL69036 | | | |
| 121 | 10 | chr2: 211362642 | A > ATC | gnomAD | CPS1 | Int. | none |
| | | | | (rs113465610) | | | |
| 122 | 10 | chr2: 211363902 | G > T | rs551601160 | CPS1 | Int. | none |
| 155 | 11 | chr2: 211365470 | C > T | rs557875053 | CPS1 | Int. | none |
| 156 | 11 | chr2: 211365748 | A > G | rs542708544 | CPS1 | Int. | none |
| 123 | 10 | chr2: 211367103 | AT > A | gnomAD | CPS1 | Int. | none |
| | | | | (rs1253865550) | | | |
| 124 | 10 | chr2: 211367235 | T > TTA | gnomAD | CPS1 | Int. | none |
| | | | | (rs539832276) | | | |
| 157 | 11 | chr2: 211367441 | C > A | rs776789323 | CPS1 | Int. | none |
| 125 | 10 | chr2: 211369053 | C > A | rs77459694 | CPS1 | Int. | GH02J210503 (enh) |
| 158 | 11 | chr2: 211454831 | G > A | rs147294932 | CPS1 | Ex. | none |
| 159 | 11 | chr2: 211507223 | T > A | rs990390709 | CPS1 | Ex. | none |
| 160 | 11 | chr2: 211527868 | C > T | rs1458915316 | CPS1 | Ex. | none |
| 118 | 10 | chr2: 26437421 | G > A | rs141429393 | HADHA | Ex. | none |
| 154 | 11 | chr2: 26455041 | C > G | rs146667859 | HADHA | Ex. | none |
| 128 | 10 | chr3: 136016902 | G > A | rs77820367 | PCCB | Ex. | none |
| 129 | 10 | chr3: 136046050 | G > A | rs748422725 | PCCB | Ex. | none |
| 130 | 10 | chr3: 136048854 | A > G | rs202247823 | PCCB | Ex. | none |
| 131 | 10 | chr3: 182740282 | G > T | rs138480247 | MCCC1 | Ex. | none |
| 161 | 11 | chr3: 182788881 | C > T | rs1244625468 | MCCC1 | Ex. | none |
| 126 | 10 | chr3: 48929487 | G > A | rs367835261 | SLC25A20 | Ex. | none |
| 127 | 10, 11 | chr3: 49455323 | C > T | rs149457059 | AMT | Ex. | GH03J049409 |
| | | | | | | | (pro/enh) |
| 132 | 10 | chr4: 146576485 | C > T | rs374622922 | MMAA | Ex. | none |
| 162 | 11 | chr4: 159611545 | G > A | rs748289922 | ETFDH | Ex. | none |
| 163 | 11 | chr4: 159616692 | T > C | rs1397900640 | ETFDH | Ex. | none |
| 133 | 10 | chr6: 49403267 | C > T | rs147715336 | MMUT | Ex. | none |
| 7 | 1 | chr6: 70451721-70451780 | het loss | none | LMBRD1 | Ex. | none |
| 134 | 10 | chr6: 99374800 | C > G | rs147696366 | FBXL4 | Ex. | none |
| 135 | 10 | chr7: 65554101 | A > G | rs28941472 | ASL | Ex. | none |
| 164 | 11 | chr7: 95761141 | G > A | rs139149160 | SLC25A13 | Ex. | none |
| 136 | 10 | chr7: 95813678 | C > A | rs35996658 | SLC25A13 | Ex. | none |
| 165 | 11 | chr7: 95818665 | G > A | rs142308242 | SLC25A13 | Ex. | none |
| 11 | 1 | chr9: 133339468-133339512 | gain | none | ASS1 | Ex. | none |
| 166 | 11 | chr10: 97376235 | A > T | rs200452017 | ALDH18A1 | Ex. | GH10J095612 (enh) |
| 167 | 11 | chr10: 126091623 | A > T | rs746547714 | OAT | Ex. | none |
| 168 | 11 | chr11: 66617482 | G > A | rs538706456 | PC | Ex. | none |
| 137 | 10 | chr11: 66618208 | T > C | rs1357633652 | PC | Ex. | none |
| 138 | 10 | chr11: 111896242 | G > A | rs150145390 | DLAT | Ex. | GH11J112023 |
| | | | | | | | (pro/enh) |
| 169 | 11 | chr11: 111899575 | C > A | rs145146632 | DLAT | Ex. | none |
| 140 | 10 | chr11: 111921965 | A > G | rs376141049 | DLAT | Ex. | GH11J112050 (enh) |
| 170 | 11 | chr11: 111930627 | A > C | rs782803398 | DLAT | Ex. | none |
| 171 | 11 | chr12: 109998857 | C > T | rs746219370 | MMAB | Ex. | GH12J109559 (enh) |
| 12 | 1 | chr12: 110008934-110008993 | het loss | none | MMAB | Int. | GH12J109571 |
| | | | | | | | (pro/enh) |
| 172 | 11 | chr12: 110011282 | C > G | rs1157166932 | MMAB | Ex. | GH12J109571 |
| | | | | | | | (pro/enh) |
| 173 | 11 | chr13: 100861707 | G > C | rs766245108 | PCCA | Ex. | none |
| 13 | 1 | chr13: 101158725-101159785 | het loss | DEL_13_139253 | PCCA | Int. | GH13J100506 (enh), |
| | | | | | | | GH13J100508 (enh) |
| 141 | 10 | chr14: 23243579 | C > T | rs368317701 | SLC7A7 | Ex. | GH14J022772 (enh) |
| 174 | 11 | chr15: 76578804 | A > C | rs119458969 | ETFA | Ex. | GH15J076285 (enh) |
| 175 | 11 | chr15: 76603710 | G > A | rs557160401 | ETFA | Ex. | GH15J076309 |
| | | | | | | | (pro/enh) |
| 142 | 10 | chr16: 28855653 | G > A | rs751872107 | TUFM | Ex. | none |
| 143 | 10 | chr16: 83940677 | C > T | rs762343124 | MLYCD | Ex. | none |
| 144 | 10 | chr16: 83948709 | T > A | rs376504760 | MLYCD | Ex. | none |
| 145 | 10 | chr16: 83948889 | A > G | rs777032506 | MLYCD | Ex. | none |
| 178 | 11 | chr17: 17924457 | G > A | rs761788938 | ATPAF2 | Ex. | none |
| 147 | 10 | chr17: 42084765 | C > G | rs537949245 | NAGS | Ex. | GH17J044003 |
| | | | | | | | (pro/enh) |
| 179 | 11 | chr17: 42084786 | A > T | rs1312599995 | NAGS | Ex. | GH17J044003 |
| | | | | | | | (pro/enh) |
| 148 | 10 | chr17: 42084825 | C > A | rs199923863 | NAGS | Ex. | GH17J044003 |
| | | | | | | | (pro/enh) |
| 180 | 11 | chr17: 42085014 | C > G | rs998174756 | NAGS | Ex. | GH17J044003 |
| | | | | | | | (pro/enh) |
| 181 | 11 | chr17: 42085026 | G > A | rs773335810 | NAGS | Ex. | GH17J044003 |
| | | | | | | | (pro/enh) |
| 146 | 10 | chr17: 7125598 | G > C | rs201509063 | ACADVL | Ex. | GH17J007213 |
| | | | | | | | (pro/enh) |
| 176 | 11 | chr17: 7126099 | A > C | rs727503792 | ACADVL | Ex. | GH17J007213 |
| | | | | | | | (pro/enh) |
| 177 | 11 | chr17: 7127359 | C > T | rs113994170 | ACADVL | Ex. | none |
| 182 | 11 | chr19: 19218779 | C > T | rs144256360 | SLC25A42 | Ex. | none |
| 183 | 11 | chr19: 51857610 | A > G | rs993404483 | ETFB | Ex. | GH19J051353 |
| | | | | | | | (pro/enh) |
| 149 | 10 | chr21: 38132112 | C > T | rs119103228 | HLCS | Ex. | none |
| 18 | 1 | chrX: 38210509-38210568 | het loss | none | OTC | Inter. | GH0XJ038350 |
| | | | | | | | (pro/enh) |
| 19 | 1 | chrX: 38224722-38224781 | hom loss | none | OTC | Int. | GH0XJ038362 (enh) |

The column headers in Table 15 are as follows: SEQ ID—Sequence identification number in the Sequence Listing; Variant Source Table—indicates in which table (1, 10, and/or 11) the variant was originally reported; Variant Position (hg19)—genome location of the variant (NCBI37/hg19 genome coordinates; Table 1 hg18 coordinates were mapped to hg19); Variant Allele Type—lists the REF to ALT alleles for SNVs and indels or whether the variant is a deletion (het loss or hom loss) or gain for CNVs; Supporting Public Variants—lists the rs number for SNVs (dbSNP) and indels (rs numbers reported by gnomAD correspond to the first altered position of the variant) or SV numbers from one or more databases (1KG SVs, DGV SVs, and/or gnomAD SVs); UCD/HA Gene Impacted or Adjacent—lists which UCD/HA gene (see Tables 8 and 9) is impacted by the variant (i.e., is located in an exonic or intronic region) or is adjacent to a CNV (i.e., the CNV is intergenic but a UCD/HA gene is located within 250Kb, see Example 6); UCD/HA Gene Region—lists if the variant is located in an exonic (Ex.), intronic (Int.), or intergenic (Inter.) region of a UCD/HA gene (see Tables 8 and 9); GeneHancer Regulatory Element—lists the regulatory element identifier (reported under the GRCh37/hg19 UCSC genome browser's data track titled Enhancers and promoters from GeneHancer) and in parentheses whether the element is a promoter (pro) and/or enhancer (enh).

Exemplary UCD/HA gene panels are reported in Tables 16-18 for the subset of genes with one or more AD/MCI cases with a variant (Table 16, 35 genes), two or more (Table 17, 19 genes), or three or more (Table 18, 10 genes).

TABLE 16
UCD/HA genes with ≥1 AD/MCI case per
gene for variants with OR >5

| UCD/HA Gene | Variants per gene | AD/MCI cases per gene |
|---|---|---|
| ACADVL | 3 | 3 |
| ALDH18A1 | 1 | 1 |
| AMT | 1 | 2 |
| ASL | 1 | 2 |
| ASS1 | 1 | 1 |
| ATPAF2 | 1 | 1 |
| CPS1 | 13 | 22 |
| CPT2 | 4 | 6 |
| DLAT | 3 | 4 |
| ETFA | 2 | 3 |
| ETFB | 1 | 1 |
| ETFDH | 2 | 2 |
| FBXL4 | 1 | 1 |
| GLUL | 1 | 2 |
| HADHA | 2 | 2 |
| HLCS | 1 | 1 |
| LMBRD1 | 1 | 1 |
| MCCC1 | 2 | 2 |
| MLYCD | 3 | 3 |
| MMAA | 1 | 1 |
| MMAB | 3 | 3 |
| MMACHC | 1 | 1 |
| MMADHC | 1 | 1 |
| MMUT | 1 | 1 |
| NAGS | 5 | 5 |
| OAT | 1 | 1 |
| OTC | 2 | 2 |
| PC | 2 | 2 |
| PCCA | 2 | 2 |
| PCCB | 3 | 5 |
| SLC25A13 | 3 | 3 |
| SLC25A20 | 1 | 1 |
| SLC25A42 | 1 | 1 |
| SLC7A7 | 1 | 1 |
| TUFM | 1 | 1 |

TABLE 17
UCD/HAgenes with ≥2 AD/MCI cases per
gene for variants with OR >5

| UCD/HA Gene | Variants per gene | AD/MCI cases per gene |
|---|---|---|
| ACADVL | 3 | 3 |
| AMT | 1 | 2 |
| ASL | 1 | 2 |
| CPS1 | 13 | 22 |
| CPT2 | 4 | 6 |
| DLAT | 3 | 4 |
| ETFA | 2 | 3 |
| ETFDH | 2 | 2 |
| GLUL | 1 | 2 |
| HADHA | 2 | 2 |
| MCCC1 | 2 | 2 |
| MLYCD | 3 | 3 |
| MMAB | 3 | 3 |
| NAGS | 5 | 5 |
| OTC | 2 | 2 |
| PC | 2 | 2 |
| PCCA | 2 | 2 |
| PCCB | 3 | 5 |
| SLC25A13 | 3 | 3 |

TABLE 18
UCD/HAgenes with ≥3 AD/MCI cases per
gene for variants with OR >5

| UCD/HA Gene | Variants per gene | AD/MCI cases per gene |
|---|---|---|
| ACADVL | 3 | 3 |
| CPS1 | 13 | 22 |
| CPT2 | 4 | 6 |
| DLAT | 3 | 4 |
| ETFA | 2 | 3 |
| MLYCD | 3 | 3 |
| MMAB | 3 | 3 |
| NAGS | 5 | 5 |
| PCCB | 3 | 5 |
| SLC25A13 | 3 | 3 |

### Example 12:—Identify Additional Genetic Variations

The methods and protocols described in the previous examples can be used to identify any possible genetic variations. Data can be generated by comparing genetic variations identified in 2 cohorts, such as: 1) non-diseased cohort including 1000 or more non-diseased individuals (e.g., individuals without LOAD); and 2) diseased cohort including 100 or more diseased individuals (e.g., individuals with LOAD). The individuals in the cohorts can be gender and/or ethnically matched. The genetic variations present in the non-diseased cohort and diseased cohort can be identified using CNV analysis (e.g., described in Examples 1 and 2) or sequence analysis using whole exome sequencing (e.g., described in Example 3). In other embodiments, both CNVs and SNVs (and other variants such as indels, etc.) can be obtained using whole genome sequencing.

Two new genetic variations, CNV_1 (located on gene #1) and CNV_2 (located on gene #2), are identified, for example, by comparing the sequence data with a reference sequence (e.g., UCSC NCBI36/hg18 or GRCh37/hg19). Similarly, two other new genetic variations, SNV_1 (located on gene #1) and SNV_2 (located on gene #2), are identified, for example, by comparing the sequence data with a reference sequence (e.g., UCSC GRCh37/hg19 or GRCh38/hg38). Data from a CNV database created using genome-wide CNV data on healthy subjects (or individuals without LOAD) such as the Normal Variation Engine (NVE) described herein can be used to determine if a CNV found in a LOAD cohort occurs at higher frequency or not compared to the NVE. Similarly, SNVs identified in a LOAD cohort can be interpreted for the potential relevance to LOAD using the Exome Aggregation Consortium (ExAC) [PMID 27535533, Lek M et al. Nature. 2016 Aug. 18; 536(7616):285-91] or Genome Aggregation Database (gnomAD) [PMID 32461654, Karczewski K J et al. Nature. 2020 May; 581(7809):434-443] publicly available resources; that is, to obtain frequency data (e.g., ethnic-specific frequency) for variants under consideration. In other embodiments, NVE databases for CNV assessment can be created for a variety of ethnicities (e.g., African and Latino ancestry subjects) to determine relevance of a CNV in a LOAD cohort compared to an ethnically matched CNV database on individuals without LOAD and/or using ethnic-specific data from publicly available CNV databases such as the Database of Genomic Variants (DGV) [PMID 24174537, MacDonald J et al. Nucleic Acids Res. 2014 January; 42(Database issue):D986-92] or gnomAD structural variant (gnomAD SV) data [PMID 32461652, Collins R L et al. Nature. 2020 May; 581(7809):444-451].

In one example, 5 out of the 100 diseased individuals have CNV_1, and only 2 out of the 1,000 non-diseased individuals have CNV_1. In another example, 30 out of the 1,000 diseased individuals have CNV_2, and only 6 out of the 3,000 non-diseased individuals have CNV_2. In another example, 20 out of the 500 diseased individuals have SNV_1, and only 20 out of the 5,000 non-diseased individuals have SNV_1. In another example, 60 out of the 2,000 diseased individuals have SNV_2, and only 50 out of the 50,000 non-diseased individuals have SNV_2. The p-value can be calculated using standard tests, such as the Fisher's Exact Test (FET) and data can be selected using certain significance values. For example, genetic variations with a p-value of less than 0.05 are included. In another embodiment, a p-value of less than 0.1 are included (e.g., when assessing rare variants found at less than 1% frequency in the non-diseased cohort and use of a hypothesis-driven study disease like only examining UCD/HA genes). Further, the frequency and odds ratio of the two genetic variations can be calculated and summarized in an exemplary Table 19:

TABLE 19
Exemplary frequency and odds ratio calculations
of genetic variations associated with a disease

| Genetic | | Frequency in | Frequency in non- | |
|---|---|---|---|---|
| variation | Genes | diseased cohort | diseased cohort | OR |
| CNV_1 | Gene #1 | 5/100 = 5% | 2/1,000 = 0.2% | (5/95)/(2/998) = 26.26 |
| CNV_2 | Gene #2 | 30/1,000 = 3% | 6/3,000 = 0.2% | (30/970)/(6/2,994) = 15.43 |
| SNV_1 | Gene #1 | 20/500 = 4% | 20/5,000 = 0.4% | (20/480)/(20/4,980) = 10.38 |
| SNV_2 | Gene #2 | 60/2,000 = 3% | 50/50,000 = 0.1% | (60/1,940)/(50/49,950) = 30.90 |

A subject with CNV_1, CNV_2, or both, may have an increased risk of LOAD, and thus may be administered a medication. In another embodiment, a subject with SNV_1, SNV_2, or both, may have an increased risk of LOAD, and thus may be administered a medication. Other embodiments include, but are not limited to, subjects with an increased risk of LOAD due to the presence of CNV_1 and SNV_1, CNV_2 and SNV_2, CNV_1 and SNV_2, or CNV_2 and SNV_1. Increased risk of a disease (e.g., LOAD or MCI) may result from the presence of two or more deleterious genetic variants in a subject wherein the variants occur in the same gene or two or more genes. For example, the two or more genes may belong to the same biochemical pathway, such as genes that cause UCD or HA. Medications used to treat UCD often target more than one gene (e.g., see Table 13) and thus may be administered to subjects with an increased risk of LOAD due to the presence of one or more genetic variations in a UCD gene (see Table 8) or another HA gene (see Table 9).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1.-96. (canceled)

97. A method of treating or preventing Alzheimer's disease (AD) in a subject in need thereof, comprising:
administering a therapeutically effective amount of a urea cycle agent to the subject,
wherein the subject has been identified as having one or more genetic variations that disrupt or modulate:
(i) a CPS1 gene or an ASL gene;
(ii) a CPT2 gene, a GLUL gene, a PCCB gene, an AMT gene, an ETFA gene, or a SLC25A42 gene;
(iii) a SLC25A13 gene, an ASS1 gene, a NAGS gene, or an OTC gene; or
(iv) a MMACHC gene, a MMADHC gene, a HADHA gene, a MCCC1 gene, a SLC25A20 gene, a MMAA gene, an ETFDH gene, a MMUT gene, a LMBRD1 gene, a FBXL4 gene, an ALDH18A1 gene, an OAT gene, a PC gene, a DLAT gene, a MMAB gene, a PCCA gene, a SLC7A7 gene, a TUFM gene, a MLYCD gene, an ATPAF2 gene, an ACADVL gene, an ETFB gene, or a HLCS gene.

98. The method of 97, wherein the subject has been tested for the presence of the one or more genetic variations with a genetic assay, wherein the genetic assay comprises microarray analysis, PCR, whole exome sequencing, whole genome sequencing, nucleic acid hybridization, an in silico analysis, or any combination thereof.

99. The method of 97, wherein the one or more genetic variations is within an exonic region of the gene, is within an intronic region of the gene, or is within an intergenic region that overlaps with a regulatory element of the gene.

100. The method of 97, wherein the administering is based on the identification of the subject as having the one or more genetic variations.

101. The method of claim 97, wherein the one or more genetic variations comprise a single nucleotide polymorphism (SNP), an insertion, a deletion, a single nucleotide variation (SNV), a copy number variation (CNV), or any combination thereof.

102. The method of claim 97, wherein the one or more genetic variations comprise:
(i) a CNV that is a loss of the sequence from position 211361945 to 211368375 in chromosome 2 or a complement thereof, or a CNV that is a loss of the sequence from position 182121153 to 182121212 in chromosome I or a complement thereof;
(ii) a SNV comprising chr2:211454831 G>A, chr7:65554101 A>G, chr1:53676401 T>G, chr3:136016902 G>A, chr3:49455323 C>T, chr15:76603710 G>A, or chr19:19218779 C>T; or
(iii) any combination thereof,
wherein chromosome positions of the one or more genetic variations are defined with respect to UCSC hg19.

103. The method of claim 97, wherein the one or more genetic variations comprise:
(i) a CNV that is a loss of the sequence from position 211361945 to 211368375 in chromosome 2 or a complement thereof,
(ii) a SNV comprising chr2:211454831 G>A or chr7:65554101 A>G, or
(iii) any combination thereof,
wherein chromosome positions of the one or more genetic variations are defined with respect to UCSC hg19.

104. The method of claim 97, wherein the one or more genetic variations comprise:
(i) a SNV comprising chr2:211361375 CTACA>C, chr2:211362642 A>ATC, chr2:211363902 G>T, chr2:211365470 C>T, chr2:211365748 A>G, chr2:211367103 AT>A, chr2:211367235 T>TTA, chr2:211367441 C>A, ch2:211369053 C>A, chr2:2 11507223 T>A, chr2:2 11527868 C>T, chr7:95761141 G>A, chr7:95813678 C>A, chr7:95818665 G>A, chr17:42084765 C>G, chr17:42084786 A>T, chr17:42084825 C>A, chr17:42085014 C>G, or chr17:42085026 G>A;
(ii) a CNV that is a gain of the sequence from position 133339468 to 133339512 in chromosome 9 or a complement thereof, a CNV that is a loss of the sequence from position 38210509 to 38210568 in chromosome X or a complement thereof, or that is a loss of the sequence from position 38224722 to 38224781in chromosome X or a complement thereof; or
(iii) any combination thereof,
wherein chromosome positions of the one or more genetic variations are defined with respect to UCSC hg19.

105. The method of claim 97, wherein the one or more genetic variations comprise:
(i) a SNV comprising chr1:45974696 A>G, chr1:53675699 A>G, chr1:53676305 C>T, chr1:53676688 T>C, chr2:150438722 C>A, chr226437421 G>A, chr2:26455041 C>G, chr3:136046050 G>A, chr3:136048854 A>G, chr3:182740282 G>T, chr3:182788881 C>T, chr3:48929487 G>A, chr4:146576485 C>T, chr4:159611545 G>A, chr4:159616692 T>C, chr6:49403267 C>T, chr6:99374800 C>G, chr10:97376235 A>T, chr10:126091623 A>T, chr1 1:66617482 G>A, chr11:66618208 T>C, chr11:1 1 1 896242 G>A, chr1 1:1 1 1 899575 C>A, chr1 1:111921965 A>G, chr11:111930627 A>C, chr12:109998857 C>T, chr12:110011282 C>G, chr13:100861707 G>C, chr14:23243579 C>T, chr15:76578804 A>C, chr16:28855653 G>A, chr16:83940677 C>T, chr16:83948709 T>A, chr16:83948889 A>G, chr17:17924457 G>A, chr17:7125598 G>C, chr17:7126099 A>C, chr17:7127359 C>T, chr19:51857610 A>G, or chr21:38132112 C>T;
(ii) a CNV that is a loss of the sequence from position 70451721 to 70451780 in chromosome 6 or a complement thereof, a CNV that is a loss of the sequence from position 110008934 to 110008993 in chromosome 12 or a complement thereof, or a CNV that is a loss of the sequence from position 101158725 to 101159785 in chromosome 13 or a complement thereof, or
(iii) any combination thereof,
wherein chromosome positions of the one or more genetic variations are defined with respect to UCSC hg19.

106. The method of claim 97, wherein the urea cycle agent is selected from the group consisting of carglumic acid, glycerol phenylbutyrate, sodium phenylacetate and sodium benzoate, sodium phenylbutyrate, taste-masked sodium phenylbutyrate, sodium benzoate, ACER-001, AEB1102 (pegzilarginase), ARCT-810, BB-OTC, DTX301, KB-195, P-OTC-101, PRX-OTC, SEL-313, SG328 and P-OTC-101.

107. The method of claim 97, wherein the AD is Late-Onset Alzheimer's Disease (LOAD).

108. The method of claim 97, wherein the subject has mild cognitive impairment (MCI).

109. The method of claim 97, wherein the one or more genetic variations comprise two or more genetic variations.

110. The method of claim 109, wherein a second genetic variation of the one or more genetic variations disrupts or modulates a corresponding gene according to Tables 1, 10, or 11.

111. The method of claim 97, wherein a subject has been identified as not having a genetic variation comprising a sequence as set forth in SEQ ID NOs: 184 or 185.

112. A method of identifying a subject as a subject in need of a therapy comprising one or more urea cycle agents comprising:
(a) performing an assay on a polynucleic acid sample from the subject;
(b) determining that the subject has one or more genetic variations based on the assay of (a), wherein the one or more genetic variations disrupt or modulate:
(i) a CPS1 gene or an ASL gene;
(ii) a CPT2 gene, a GLUL gene, a PCCB gene, an AMT gene, an ETFA gene, or a SLC25A42 gene;
(iii) a SLC25A13 gene, an ASS gene, a NAGS gene, or an OTC gene; or
(iv) a MMACHC gene, a MMADHC gene, a HADHA gene, a MCCC1 gene, a SLC25A20 gene, a MMAA gene, an ETFDH gene, a MMUT gene, a LMBRD1 gene, a FBXL4 gene, an ALDH18A1 gene, an OAT gene, a PC gene, a DLAT gene, a MMAB gene, a PCCA gene, a SLC7A7 gene, a TUFM gene, a MLYCD gene, an ATPAF2 gene, an ACADVL gene, an ETFB gene, or a HLCS gene; and
(c) identifying the subject as a subject in need of the therapy comprising one or more urea cycle agents based on the determination of (b).

113. The method of claim 112, further comprising identifying the subject as having Alzheimer's disease (AD) based on the determination of (b), or identifying the subject as having an increased risk of developing AD compared to a subject without the one or more genetic variations based on the determination of (b).

114. A method of predicting a subject with Alzheimer's disease (AD) or suspected of having AD as being a subject likely to have a beneficial response of to a therapy comprising a urea cycle agent, the method comprising:
(a) performing an assay on a polynucleic acid sample from the subject and identifying one or more genetic variations as being present in the polynucleic acid sample from the subject based on the assay, wherein the one or more genetic variations disrupt or modulate a gene according to Tables 1, 8, 9, 10, 11 and 15; and
(b) identifying the subject as a subject that is likely to have a beneficial response to the therapy comprising the urea cycle agent based on the identification of the one or more genetic variations as being present in the polynucleic acid sample from the subject.

115. The method of claim 114, wherein the one or more genetic variations comprise one or more genetic variations selected from the group consisting of the genetic variations listed in Tables 1, 10, 11 and 15.

116. The method of claim 114, wherein the urea cycle agent is selected from the group consisting of carglumic acid, glycerol phenylbutyrate, sodium phenylacetate and sodium benzoate, sodium phenylbutyrate, taste-masked sodium phenylbutyrate, sodium benzoate, ACER-001, AEB1102 (pegzilarginase), ARCT-810, BB-OTC, DTX301, KB-195, P-OTC-101, PRX-OTC, SEL-313, SG328 and P-OTC-101.
